SEQUENCE LISTING (7 sequences)

SEQ ID NO: 1 - DNA, 168753 bp, Artificial Sequence
  Description of Artificial Sequence/note = Synthetic Construct
gaattcattcattcatttgacaaatattcactaagcacctaccacttgtgagatactctgttaggtggggatacagtatgataactgtatggaactaacactccaagtcaaacattgaagtcaaaattctgggctggcgtacattagttaggagcccccattttagaaaatattatttcaagcaggccttgtttttatccagagagtgtatacagtataataaaactcagtacaccaagatttatatttcatggcgtgtttttctccctaagtagagaaaaagctccttcagtaaagtgattgtgttgtcaaccactgcatattcttaagtgtaccaagatgcttgactccaggcaggtgctaagagagactgttgggtgagtgaatgtatatcaatgaaaatcaaacagaggaaattttctttatatgttttagtgttcctgtggtttttttttttttttttttttttttttttttttttttttttgtgatggttactggctcttctctaagaataatggagaaaaggtgttagtttgtactgtcgcacagggcagtcgtaggagactctgaaaaagcaaataaatcaatgttaaatcagaaatgtgaatgtagtaaggggctgaagagacaggggaagagaatacatgggaaaatattgaaaaggacagagtgatcaaaaagagcagggacatgggagcattgggcagcacactgggagccatttcactttatgctcttattgtatgattgagaaaaaaatgtccttagtggttaagtggcttttcaatgccacatcagacttgttccatagcagttgaattaggggaaggtgaataagttggaggttggtgacaaggagagaagctggaacagagaggagagtcagaaccagagggaaatgagagactgagtaggcatctcagggtttttgaaggagtggattttctttgttgcagtcaggggaggtttgtctgttggctgcagaaagaagtcagaatagagatatcgtggggtaggtttgtttggaacagaaatcaaagaccaatttttctgagagaaggaaataacatctgcaaatgatatgctgtttttgctacttccattgttagctgttctcccaggtgatggcaatgcagacggtaagaactctgacaactgcccagttgggtggtgggtgaaggtctctctctctctctctgtgtgtgtgtgtgtgtgtgtgtgtgtgtgtgtgtgtgtgtgtgtgtggtttccctagcatatacctggaaagtgagagtgccaggctggttccattctgactatccatgactccaggtccctgagctcttatgaaggtggttgataaggcatgggactgtgtggcaagtgaatgtctgctaagatcatgatggatccccttttctccagattctgagttcccgcactctggcacctttctctctccatcctctttctcaatgtctttctccccattctatctgttcttttgtcgcagggctcaaggagcctctctccttccatgtcacctggatcgcatccttttacaaccattcctggaaacaaaatctggtctcaggttggctgagtgatttgcagactcatacctgggacagcaattccagcaccatcgttttcctgtgcccctggtccaggggaaacttcagcaatgaggagtggaaggaactggaaacattattccgtatacgcaccattcggtcatttgagggaattcgtagatacgcccatgaattgcagtttgaatgtgagttcagttttctccatggagagtggtgaggtgggagagaggggttgtctcaatgtttcttattcctttaggaaacagcctgactctcttctgtatcttattctattctttccaccataaaactgcaattgcatacttttgggcaagggctatcatccagaccttaattctccaaacacttctgctacttatgaactcctctctcattgattgcagggttgtgcattattatcttttcttacaggtatcccaggaccacccttctgcctgccctacaaccagataccttcagctttctcactttctcattcattctcttcagcagtctctctatatatactcaattgtaacccattgcatttaattcaacctaagttgttataatcccagttctcttacaccaggtccttgctcacccacagcattcacatcaccttctttgaccagtttgccctcttcctcatgttattaatcttttcctccacctacagctcccaccaattcctctttctctgaatccctgaccaaaccaaattttattccattatgtcaaaccctgcacacttcccccttctgcttctataatctttgcttctactcatttcattttatatttttttctccctctttcctctatttcataaccccagatccttttgagatacaggtgacaggaggctgtgagctgcactctggaaaggtctcaggaagcttcttgcagttagcttatcaaggatcagactttgtgagcttccagaacaattcatggttgccatatccagtggctgggaatatggccaagcatttctgcaaagtgctcaatcagaatcagcatgaaaatgacataacacacaatcttctcagtgacacctgcccacgtttcatcttgggtcttcttgatgcaggaaaggcacatctccagcggcaaggtcagtcctgcactctccctccaagaagttttgatttgaaaatcatacccttccaccacctccttccaatttagaataaggaagagccacccagaagtgggaaaggctgggtgcagtgcctcacatctgtaatcccagtactttgggaagctgaggctggaggacttcttaagggcaggagtcccagaccagcctgagcaacagagtgagaccagtgtctctaaaaaataaaaataagttagccagtcatggtggtacacacctgtagtcccagttacttaagaggctgaggcaggaggatcgcttgagcctaacagtttgaggttacagtgagctataatttataattatgccactgcattgcagcatagtgacagagtgagaccctgtctcaaaaaaaaaaaaaagagaaatgggaacaatgtgtctatctaaacttctaaactgttgtggagatatgaaactccaagtctgtatttgaatatcttttctgtcctttgcagtgaagcccgaggcctggctgtcccatggccccagtcctggccctggccatctgcagcttgtgtgccatgtctcaggattctacccaaagcccgtgtgggtgatgtggatgcggggtgagcaggagcagcagggcactcagcgaggggacatcttgcccagtgctgatgggacatggtatctccgcgcaaccctggaggtggccgctggggaggcagctgacctgtcctgtcgggtgaagcacagcagtctagagggccaggacatcgtcctctactggggtgagaaaaagctaaggcccaggctggaaatgccaggaagtggacctcaggcatagagggagggcaagctgaaaattttaggattttagggattatagcccaaacatgaataaaaatagataatctaagaaatggaaatgttgaaaatgagggccctgtagatgcaggaggatgctgagggttaactgaagaatccatctctgagcagggatgagagaaaaacatagaaaaaaaggagattggtaagttggatactcaaatgaaaaaaagaagcccagggaatgcagtgaaatagggagtggatgaggtaaaatgggatggattataacatccttggtgtctccccaaattcacagagcatcacagttccgtgggcttcatcatcttggcggtgatagtgcctttacttcttctgataggtcttgcgctttggttcaggaaacgctggtgagttctttgcatgtgtctttcctactcttagcctctaccccacttttcttcccatgttttttgtttttctctcctcagttcttctctccccagtccccttccctcttgctcctcaattctcagctccaccttattcagagtgacttctatctctgttctcatccagtttctgttaagacacaccatgagcctcctcgtcacccttctccttttggggtgagagaccagcagcccaagggctccagacacacctgaacacatcgtgatgatgacgtcctctcaactctctttgtaaaaattttgttatttttgcttgtttctgattaatgattgtttgtcaatataagctcaatttaattttgcaggatttgttgttctgacctgggttctgggacttttaaattcaaattttatctccagatggaatggggtcctagcaacctccacatgttcaactattaatggatcatcaggcctgttttagatatcccttactccagagggccttccctgacttacaagtgggaagcagtctcttcctggtctgaactcccgccacattttagccgtactttgctaactgtgctcctcacttcctcttcttcattgcagttatttagatcccccctttccttctaatttttcagctccttcaatgcaaagtacatgtatttttaatatatgcatccctggtgaaggatcttgcctgcatgaaacatgttctcaataaaactctgtgttgaatttatgccaaatttgtagtgttttcttattcttttccttcctggtgcttttgaagtagttgagacatttgatactcttctgcttaaaagtcttctcacttggaggaaattttacacattcctagttctatagtttgttctctgagctccagaggagaaacacaattttgcctcttactactcctatcattcaactatagagagctcttcgagttaaattattgttccaggtatgcaaggcaggatgtatatctttactattcagtggttcagggaaaagcagcagagttctcacattggagcttgttagaaaggtagaatgtgggcctcaccttaggtttactgaattataatttgtgttttggaaaatcagcaggatactcacatgtccattaaaatttgtgaggcactgatgctggtgatgcctgagatgtagtacaagtgtgtcactgaattgaaaatgatcaaaaaagggaacaagagtgttactcctgaagattgaagtgagatcaaggcaagtaatgtacctactgcaccttctcattattcatggcagttatgttgtgtgcagttgctgcaaatactgaatttgtgagcaatgagccattgttcccaggggaaatacaggtgttaggctcttgtgagatttttcacaacatttttgtcaactggtgaatacataagctggttttatgtgtgtttctgtttaaaaacatcttattttatatatgtatatataattaattcattatatgcagtatttctttataataaaacactatctacgaagtgtttaatatattcctgaccttggttaacatgatcacaaatttctttggttttcagcttcacaacattgctgtccactacacttaaaaaaaatggttgtcatttcatgaatccacaaatgtagctattttcccatagcttcagcatagcattatagatgttactttcccaagcagcctcacctacagatgggtgaatttcctcccccttttcctgcatgtactgcactgttgatttatttttatttatttatttattttatttatttatttatttattatttttatttatttatttatttatctctgtaaggcatgtcacagctttcttatgcttaggaaaatgcgacagcacttaagcactacacttgggaccatttaagattgtgaaatcaccaaccaaaaacccaaaaatgtgcagaagttggcactatatagactacaaaaaggacacttgtttactgtgtgagagctggaacatgaagacaaagcaccacctttttcgaccttagttaggaacatgttctttgggggactcaaattttgtacctcactgcatatattcatgaatgattgtgaatatgctatgaatatatatctaggggttacaaataaaatttggcaagtagacaaattagcaaatataacatttttgaatgatgaggattgactattgaccccagatggtagatggtagtatttgtcaagatatccatagctagacttcaattactttaggattcatgaattcaacaaatatttcttatgattctgcaggttgccactttcatttcttgttgtttatttctcagttttctagtatagtaacttattagagttgaacttggggaaaaggacttagctctaatttgtttaggatactgagaagaggggcccaaggcaagggaaacatccaggcagaccaggtgcagttggcagtggctgcgtatatggaaatttttggaaactgaacttgtaatgggtgaaggggaaaagaaaaatatgggatagaggagagcaaagaatgggacagctgcagtctttttacctgtgtttgacatgattggttccaacccatttgggtttgaatccaccttgccacatttgcatagtagtcatctggcttttggttggttgataccaccttcagctctagtggtcagcctaaggcaatcatgataactcattctttacaatgtgataggctaaaggatggaaggtaacctgagatttaagccaatcatcatatagatcccacttacccttgctcagagtggtggttcagggctatgtacttgttctaaaatgacactgtttacatgacgcctgattcttgtgtttgacaatagggaaaagagaggctctctcttgtcctggatggtatggtgtgaggtatagagcttgaaacagctcaagtcatttttttttcagcaccagagaaatcacacaggcaagaaagtagaggcaacggactctcagaagagtccagattaaactgtatccaaagcctgaatgaaccctggacttctcagtgatgttagatgatacatttcctttactgtttcagcctatcaagttgggttttctcctgtttgtgcttgaaagcatcctatctgatgtacatggaccctaggggacacttagctgattttactcttggttgaagtattcctctttatttccatcttcctcctcctcccttcttgggattggctatactttaaaattgatttcttttctttcgcaggttgagtattctttatctgaaatgcttggaatcacaggtgtttcagatttcagatttttgggggggctttagaacatttccatacacccaccagccgagcatccctaatctggaaatctgaaacttgaaatgctccaacgagcattttctttcagcatcgtgtcagtgctcaaaatgttttaaattctggagcattttgcatttcagattttcacattaggaatacctgacctgtagtttttttcttatatgtgtgcttatctttttaaactcttacactgtcctgtttttaactatgctgcctgaattaattactctcttgaacttttcatatatttctgaccaggaataaaagtgctgcatctgctgctaagtgagtgactttggaaaggttccctgatcttccttcttttctttggttaaatggaggcaaaaatatatactcttaggattttcataggaatgcaaaggcaacctagtgttattaaaatgccaggagtttggtctacggcctgctgcttgccacataaaaaccagttaccgaggcaatgagtattgcctttaattgactgctgcagccgaggagataggaaatcaatctcaaatccatttccttgattgactaaaattagggatttatagggcagggaagaaacagaaccctgtgtggcaaaataggaattagggagaggtaaggaagaagaattggtcaacagcaagcaggtggttgcttaagcaatcatgatgggtgaggagtctggcatctcgttgtccagatgcggtagattggtaggttttagttgtttttgacaaggtttagttccctggctgggaatgtattgctagtaacttggctgctgtgggcagggagagtctcaaaagtcctcctaagcagctgcccaacacttttttggctacaggtgggtgttggtttttctttctctggccctacctctgccagccccagctgtgtttctgattgcctaggaagagcagcctttgaaatgtgtcatctgagtctggatgggtgaatgtcttttgtgggtaccagacagtgggatcttctcctctcaatttggggaattctggaggaatttccatttgcaggttgaacaagcccaactgattgatattggaactcgttggttgctgcagcaattggacagtgttttagtgattgcttgtttatttgtgtgtgttaatatagtcacaggaaatcagaatttgataaactgataatattttgtagcactgtttggcttcagtgttctttggaatctggagaagtttggcttctccatgggtcatgctgttgagtgagacagtggaacggtgttatggtgttacatgtatctaagcttttaggctgctgttctaagcagggttggacctggttagtacatgatgttcttctatagtgctgtttggccctagtggtctttggagcctggggaaatttagcctttaataatcaaacggccatggaaactgctttacccaaattttggttcaaagctttcattggattacatattggggcaaacaaacttgagccatgtgaacatgttcctaaaccagtggtttggtattgctatctcatggctagagttccaaggtaaaagctattggatctttgtgtgtgtatgtgtatacatgtttatatgtgtttatgtgtatgtacatttattatgttgtatgttgtgtctaccaaattggcttataaataaaagagcatcttatacactaaataaataaatctaaggaattttcaattttatgtgacttaagtaaatgtttatttaaaaagctggctttaaagtcattagtaataaaaaaataagaatgtcttaacagtgttagaatacattttcgtctgggttttataattgtctttgctagatactttgaggtgtcagtgtttggcacagagggttataaaagtataaacccagccaaaacaaaatgatctttgtttgtgtgccttctttggcaaataagactaagtaaatgttgttagtttaataaaaacagttgaatcttctaagttattggcaaaaatacctatgtatttaagttgcttacttaggtgagcacctaatattcataggctacaaataatggtcaacagagcaataacctgaaatgacgactacctttgtctaatatctcagttttcagaacccacttatataaatttttttttttctttttttgagacggagtctcgctctgtcgcccaggttggagtccagtggcgcgatctcggctcactgcaagctctgcctcccgggttcgcgccattctcctgcctcagcctcccgagtagctgggactacaggcgcccgccaccaggcccggctaattttttgtatttttagtagggacggggtttcaccgtgttagccaggatggtcttgatctcctgacctcgtgatccgcctgcctcggtctcccaaagtgctgggattacaggcgtgagccactgcgcctgacctataaattgtttaaaaagatgaaagaattgaatacatgtaaatgggatacatgcttgtaggtgagctttttgtgtaatttaaaatcttaaaattatttttgatgcttcttggatgtctggctcatttccagttaagaaagggttatgatatggagaaacatgtttttataaattgtggaatgttttcatctataaaatgcaaatatttgatagacagttaaagatttcttgcttcctagttttcactaaaatttaaggttactaagaataagaattgtagttaatatataatttagtatataaaacctgccaaagaaggtgttttcttattgagaaatggaataattttgtctaattcaaatgttttctaaagtttgatcccaattatggactcgaaaaacttatttatgaaataaggtagaaaggaaccagcaagtagggaagagagatgtgaagaaagttaagaacataaagatgtatttttggcaagaaaggttaaaaagaaaagaggataattttatacaagtatcttttcttttagacttttattttaggtttggggtacgtgtgcaggttttttatatagtaaactcatgttgtggaagtttgttgtacagattatgtcatcacccaggtactaagcttagtacccaatagttatttttttctgctcctctccctcctctcatcctccatcctcaaggaggccccagtgcgtgttgcgtgttgttttcttcttggtgctcatgaactctaatcatttagttcccacttaaaagtaagaatatgtggtattttgttttctgttcttgcatcagtttgctaaagataacggcctccagctccttccatgttcccacaaaagatatgatctcattcttttttatggctgcatagtattacatggtgcataggtactacatttcctttatctaatctgtcgttgatgggcatttaggttgaatccatgtttgctattgtgaatagtgcttcagtgaacatttgtgtgcatgtgtctttacggtagaatgatttatatttctctgggtatatacctagtaatggattgctgggttgaatggatatttctgttttcagctgttggaggaatcaccatacttctttccacaatggttgaactaatttacactcccaccaacagtgtatgtgtgtcccctgagaaaacaatctgacagcattttttttttcctaaaaataaatgaattttttaaaaaaaatttaggacaaaacagaaagttcaagcatgacacagatgccttgttgaagtcatatgttgttttctttctgtttctctgtgtgtttatcttcatgtacataaacagaaaataaaaagttgaaaagtttatatagtaaatatcctttaaaaactgatagaaaattggagacatttgactaattaacattgttcaccgttaaagctcttagtcttgatgaaggtacaataagaattattataaataaatacattggcagtttgataattctttttaaatatagttaagcctgaagccagatttaacatggagtcaaattttacataaatgcttgcattgctttgtttcacactgtatttgttattctacatagaaagtactagcactaaggtacttattggttacatgcctaaagtgaatttcttaattgcacaaaatgtagagtggtactggtggacttaaacacattaattagtatatcagaaacagaatatcatatttttttaggccctggataacactgtagactccaaggtaaactaagtaagagaaaatttgggagttattttcctgttggttttgcttttaattttcatttatttgctgtttgttctcctttgggttttacttatatttacatacatataaaacaattggtgttttttagtttctactgtaaggcttgtatttggttctatgaatagtcattttgtttcctatgcatttccaaaggcttatcatttgctctatttatctaaaattcctaagctgtctttgtcaagcttccaaaaattggcagaggacagcagccatttaaaatttggactggggctgggtgcagtggctcatgcctgtaatcccagcactttgggaggccaaggtggctggatcacctgaggtcaggagttccagtccagactggacaacatggcaaaaccctgtctcttctaaaaatacaaaaattagctgggggtgggattacagtaatcccagctactcaggaggctgaggcaggagaatagcttgaacccaggagatggaggttgtggtgagccaagattgcaccactgcactccagcctgggtgacagaagtgagactctgtcccaaaaaaaaatagttgaattttactttgtaaaattaagaatttctcttcaacaaactacacactgagaaaaattaatgcgtagacaattaagaaaactatttggaattctaacaataaaatgggtatatataacatatatgagatatacacccatatatgaatttggagatgtttgttgattaagtatacatgtgtagaaacaacagaccagactgttgcgaaggtcttcaagaaagtgctgaatgcaatgcatcaatacacgaagcatacagtacaatacctgtattgcaattagagttgtacacatcaaatagtaccactttttttcttttataattgagattttattggttgtgttgagggtcagtacacagatatttcaattcttaatatatgtactgaaaatctaaaaagccatgtattgtaattattttttaaagttgttccagtaactttcctgcttaaaatttggaggcaaattttccttaagaggctaccaagtaccagtatcttcacatgttgataagctgttaacagatgtcccaccaactcacaattgaatagcatatacactacacactcaaatttttagtatttcacagcatggtaacaaagttattagaaaaacaggactaccaccaccaaagatgttacagagtgcacacaattctgacagggagagccattatgaaggagtggtttcctttaggaaacaattctactgacaaaaaacatgggactagaagtaatttaaaatgttcaagacattaaaggcaggactgtgactccacattgccatttagtacgctttgtattataggatataaaagctaaccccccatccatggaatgttaagctgacacccaagacaaagcctcccataattcgatattccatactattttctggtaccaaaaataaacaaccagagaatgatttcacctttaaaaaaaaagcacttgcacttaaaaaatgaggtgagatgggattcactctttcttaaaaatgtttctagagctactgaaaaacttttgtttacaaaatagttcataaaaatattcccccaggttgtacaagaagggagacagggaagaccgataagacatggaatgtaatactaatcagacttggcttctttctctgcagcttcagaggctggactctcctcgattttagtttttccgttttctgaagtaaatctttagtttcttgattacccacgtcggcctgttttccctttgctcctctttccccttttgtttgcaattttttgtctgaagatttgtccttttctttttcttttttttgacgaagtcttgcgtctcgctgtcacccaggctggagtgcagtagtgcgatctcgcctcactgcaacctccgcctcccgggttcaagcgattcttctgccttcgcctctggagtagctgggattacaggcgcccaccaccacacctggctaatttttgtatttttagtaggcacggggtttcaccatgttggccagtttggtctcaaactcctgacctcaggtgatttgcccacctcggcctcccaaagtgctgggattataggcctgaaccaccacgcctgacctcctgctgccttttccggcttcatttccacttttgcaggagaagctttagctgccaacggcactgatctcctcttgggctcttccttcatggctccttcatcccggtggtgcagaacgtgtgtgccaggtgcctgcggccgctgcacgtaagagccctggtgaagctaggctgcctggccactgccgctcctcccgcctgagctgctgagaccccgacaccatgtgttaaaagaacacataggcacagaaggatgacacattaatacattaaaatgtggggaagggaaggaagtggggataataagtaaataaaataatatacagaggaagcccgctcaggccaaatgatatccacgccatgaatagaagagaatgaactcaacaatctatacttaagattccagagaagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatttcatagtggttttgatttgcatttctctgatggccagtgatgatgagcatttcttcatgtgttttttggctgcatagatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgacggtagtttcttttgctgtgcagaagctctttagtttaattagatcccatttgtcaattttgtcttttgttgccattgcttttggtgttttggacatgaagtccttgcccatgcctatgtcctgaatggtaatgcctaggttttcttctaaggtttttatggttttaggtttaacgtttaaatctttaatccatcttgaattgatttttgtataaggtgtaaggaagggatccagtttcagctttctacatgtggctagccagttttcccagcaccatttagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaaaaagaaaaaaaaaataaaaataaaaataatttcttactactgttaaaaaaaaaaaaaaaaaaaagattccagagaagacagaacacaatacaacaaatcaaaaattggaaaattatgatgcatagaatgaagaaactgaattgggataaaagggtcgcaccatgggtaacttcccagggaaaaataataaaaatgcatgatattgtgtaaaatacttctgtgtctaagtacagaatcaaaatatctgcaacattaaaactcgggttgaaaatgtcagggtttttcaccctgataccaaaaccaggaaaagacaataataataatgatttaaaaaaaactgcagaccaatatccctaatgaacgtaggtgcagaaattctcaacaaaatactagctaaccgaatccaacagcatatcaaaaagttaatatatcatgatcaactgggttttgtatcagggatgcagggatggtttaacatatgcaagtcaataaatatgatacatcacgtaaacagaattaaaaacaaacatatgatcatctcaatagatgcagaaaaagcatttgataaaatctagcattcctttatgataaaaccctccaaaaaataggcatagaaaggacttacctcaaagtaataagagccatatatgacaaacccaccgccaacatcatactgaatggggaaaatttgaaagcattccccttgataagtggaacaagacaaggatgcccactttcaccacttctattcaatatagtactgaaagtcctagccagagcaatcagacaagagaacaaataaaggacatccaaattggaaaagagatcaaactgtcactgttcgcccacgagttgatcacatacttagaaaaacctaaatacgcatccaaaaagctcctagatctgacaaacaaattcagtaaagtctcaggatacaaaaccaatgtacacaaatcagtagcactgctattcaccaacagtgaccaagccgagaatcaaataaaaaaactcaatccctttcacaacagctgcaaaaaataaaataaaatacttaggaacattcttaataaaggatcttgaaagatctctacaagaaaaactacaaaacactgctgaaagaaatcatagatgacacaaacaaatggtaacacatcccatgcttggggatgggtagaattaatattgtgaaaatgaccttgctgcccaaagcaatctacagattcaatgcaattcccatcaaaatatctttataatttttcacagaactagaaaaaagaatcctaaaatccatatggatccaaaaaagagcccacatagccaaaacaatactatgcaaaagaacaaatctggaggcatcacttcattcaactttaaagtacactacaaggctatatttaccaaaacagcatggtactggcataaaaatagacacgcagaccaatggagagcatagaaaacccagaaataaagccatatatttaacaaccaactgatctttgacaaagcatacagaaacataaagtaggagaaatgacaccctattgaagaaaaggtgcttggataactgacaagccacatgtagaagaatggaactggattcccatctctcagcttatgcaaaaatgaactgaagatggatcaaggacttaaatataatatcccaaaccatgaaaactctgtaagataacatcagaaaaactcttctgcacattggctcaggaaaataatttatgactaaggccccaaaagcaaatgcaacaataacaaaaataaatatatggaacctaattaaactaaaaagcttctgcacagaaaaagaaataatcagtggagtacacagacaacccactgagtgggagaaaatactcatagactatgcatctgacacaggactagtatccagaatctacatgggactcaaataaatgagcaagaaaatgacaaaaattccaccaaaaaatgggcaaagaatgtgaatagacaattctcaaaagaagatatacaaacagctaacaaacattaaaaaaatgctgaacatcactaatcatcagggaaatgaaaattaaaaccacaaagagataccaccttactcctacaagtatggccataattaaaaagtaaaaaaacaaaagatgttggcctggatgtggtaagaagggaacacttatacactgtggttggggatgtaaattagtacaaccactatggaaaacagtatggagattccttaaagaactcaaagtagaactaccattcgatccagcaatcccactactgggtatctacccaaaggaaatgagaccattatatgaaaaagacacatgcacatgcatgtttatagcagcgcaattcacaattgcaaagatatggaaccaacctaagtgcccattgaccaatgagtggataaagaaaatgtggtagatatttaccatggaatactactaagccataaaacagaacaaaataatgtcttttgcagcaacttggatggagctggaggccatttttctaaatgaagtgattcaggaatgaaaaatcaaatattgtatgtctcacttataagtgggagctaaactatgaggacacaaaggcattaagagtgatataatggactttggatacttagcaggggggagattgggagggggtgagggataaaagactacatattggataccgtgtacactgctgtgtgacgggtgcactaaaatctcagaaatcaccactaaataacttatccatgtaacaaaaaaccacctgcatcccaagaactaatggaaaacaaacaaaaaacaaatttttgtattctaccagtagatgacattagagattaaaatgccatttctgcatatgcttgttattaaaaattttgtcttatggcttcccccaaatcacgcagcagctcagaaacctatccttaaattcacaataactttttggctctaaaatattaaaaatatattgataacaatccaggaatagaatccagggtaatatatggccaataaaataagatgaagccaagaacagtgtaaatttttaaagaagtgccacgataccctatactctggttagatgtgggtcacacattgttggaggtccatgataatcaagacaggatcagttacatacttgcagtgtctagaagatagacaatgggctgtttaggagaattatagctattcctggtcctgaggccacaaggacatttcttccatcagaggagaaactgcttaacgcagtgaacaatgttctcaggaaagtcctttcagtaaattttatgaggtccataggtctttttgtgattaagtctcttaaagtaagatcattatgtcagcctatgacaaagactgggaaaagtaattctacacagcataacaggatctagtccagacatgcagctttctgatgttctagcaaacccaagggtagattttgttatctgtagaaatgaatagattccacatctttcaaagatactgtttatattgaactgatttaaattatttttgaagcagcagcaacatttagattatgtattctaacatataccggatgtaaaactattttctatcatctgttaaatacagaacagtaactttgacaataggttgacttacaagtggaattggaattggaaatttcttgtggcttctgcaaatatctactcaaggacttctgcttgaatctgaggcttgctcccggaaggctgatagcacccatagagagcaccaagattcttcttgtaatataattttgttttttttttccaaatacaaaggcactagttgacataactacaacccccctcatcccaatgcacaaccaactgtacttcatagagaatgccagatatggctttagacaggaaactatttgaaacagatatgaatcttaatatgagtttaaagtgagaaaaaagaatactatgagtatgaagtaagaaaagcagccatgggtccttgagctatttccagattgcccattcaagtaaaactggttattaactatacacctgcatagctaaataaataaatgacttgccaaatgggctatatggcagagaacactggttttctcccagtatatgtttgtcacttcctctagaataactgaaacctgctgattattagctgggcagaataaaagctatgtttcttggcaccctttgagctgattgtggtcatgtgattacaacctgccaaatgtgatgtataacttctgcagaaagcagctctgaaatcatgggaagtcgatgtgctcacttcttcccttcctcttattggctggatgaaaggtggatttgatggaaagaactctggcagtcacctcagggtatgggaagaccactggaatgaggactaactgtggtaaagaccaaaacaaaacaaaaaaataaaaactctcccccgcaaaagaaggaaattggaaactgagttcctgacaccatggaatgccacatcaaccctgcattgcctactcctgtctttttataagagagagaaatgtggttgcatttttatttatttatttaattattttgagatggagtcttgctctgtagcccaggctggagtgcaatagcgtgatctcggctcactgcaacctccacctcccgggttcaagcaagtctcctgcctcagcctcctgagtagctgggactacaggcacatgccaccatgcccagctaatttttgtatttttagtggagatggggtttcctcatattggccaggctggtctccaactcgtgagctcaagtaatccacccacttcggcctcccaaagtgctgagattacaggcatgagctactgtgcctggctccatcttttaaaagatactgtcagtttgaattttctgtcactcacaaccaaatttaactaattcaggatgtcagctgagtatcaattaatcatcaatagcattggtctatgaaatgcttatttgaggctctttatttttctcattatgatgacctgtgtgggttacttaatttttgctaagacaaaaccaaattttgataatagcacccaagttatttgtatttaagttcactttcacttttgtctttatattttctaaagcaagggagaaaatgcaactagccgttccaaaatttatatgaaaacaaacttgttagaaatcagagggaacttctgggaaaatgatagagtgaaatatttcataacccatttcacttaggtaaatcccaagtagagaaagaaacttcatctatactgaatctagtaaagagctataacttacaacaaaagccaaggagtttctattttttatgttttcaccctttctactttgccagaaacactgaatagagttttgtctttctgatttaaagagaggaagagagaaaaaaatgccaactctgtagacaccttcagagttcagttttatcataaaaaactgtttgaataattagacctttacattcctgaagataaaataaacatgtaatcttttatcttattttgttcaacaaaattgttcagaaaatcaaagtggtaaaaacaatgtaaaatttaacattttaataccgatgttgtacactgttttacttaacattttggagagtaattgccttgctcttcaactcaggaaaacactttttttgtggctaatgtaattggtttctgtaacaacatcagcaaataaaaggatgctgattattgaaaagaaagttataataaaagttggtataaatttttagcacacatttttctgattttgagtatgatgaaaggttgatatctacagttttaatagaaaaagttcaattcaggagagcgtaaacaaagctactaggacctttgttagaagacgactatagggaataaagtattatggaaatggagaataaaataatttgggaggaatcttttggtggagccaacctttagaaaacaggctgtggccatgagtctttgaccttgttggtgtgcagtggcctatgtcgttgaatacgactggcttttatatgctgtgcgactgcctccttaccactgtggtctgaacaggcagctgtaggactttgtttctagtggccatttgaggtttttattgtggtaagttgtggctgtttaagatgggtttgcttaagatgagtgttaagtggctctatgtagatgagtgtctcaggtcagttgccgaggaaacactcagatgagcctcggcctgcagaatgcttactggggagtgttctatgaaacagcactaccaaaagaaagagggaagcaggactgagcagtgagacaaggaactgtgatgcagttgtaacagaggatctaacaacctcatagagagttctggagctaagatggcccttcagaggttttctgaaatgaagtaatgggctcatacctttgtacttctatattcgatgtgggctttccctagggaaagaatgtccccttaggcaaagtggctcccttctacataaggcagtttttagaaggacacatctgtgagtagtcagcagccaacactccctgaagctgaggaataagcatcttcttcctgaagaggctgtattgctggctgaccacagcacccactagagcaagggtatgtccttatggaagtatggatttacttgctggaccttaaataaatgcctcataaacggtgtcaatattgtagacacaggccaaggccagagcaaatcttaggaaatatgcatgggattctacaagcagtaaccttaactaggctgcctcagcatgatggctaaagcaaaaattctattaatggaaccagagacctcttagatatcagttcttcttcttcttctccttctccttctccttcttctttcttttttttttttgatggagtctctctctgtcgcccaggctggaatgcagtggtgcaacctcggctcactgcaatctccgcctcccgggttcaagcgattcccctgcctcagcatctcgagtagctgggactacaggcaagcaccatcatgcctggctaattttttgtattttgtagagacggagtttcaccacgttgatcacgatggtcttgatatcctgaccttgtgatctgccctcctcaccctcccaaagtgctgggattacaggtgtgagccaccgtgccgagctgacatcggatcttctaaaacaccaggcagcaaaataattattgccagtttttgccttgattttcagtccagactgaaaggtctgcagtctagatgagatacctcaaatttggtttgtctcctacattattttaaactggtttcagggtcaagaaggagtcccaggaatgaactcaccttgactttatgtggcttgtacggaggcccaggaatcatctcccactacataggttcaatttgcctacacctgttattgcttagatctttgagacacaagttgtggcttcacacatataaggatggtaaattctattaataaataactaaaaaggaaatagaaagacttcttttttttaaattatactttaagttctagggtacatgtgcacaacatgcaggtttgttatgtatgtatacacgtgccatgttggtttgctgcacccattaactcatcatttacattaggtatttctgttaatgctatccttcccccatccacccaccccacaacaggccccagtgtgtgatgttccccgccccatgtccaagtgttctcattgttcaattcccacctatgagtgagaacatgcggtgtttggttttctgtccttgcgatagtttgctcagaatgatgggaaacagaagtacttctgtttctagaaatgttatactacataatttggaccaactctcttatcaaggatgtctagaaaaaacaataaaaatataaaataaaacctgcctcaaggcatcaaagaacccacaaaagggagatggattattgggttaatatctgggagaagagagaaactcatagtgatgaaccctgtatttggggctgcttttctccccagaggcatctactgattccatgagaggcactaatgagactgaaaagctaagcttttaacagacttgtaaatcttcaaagatataattgaagcccacattttaaattgggaacttaaggggttaattccagcagtaagtataaaacagaaggagaataacctgtgcagggattacaacacagttccaaattatcttaatctctgaaattaataaagcaattttgatccaatgcccttaaataaatatagagcaagataacagcatcctgggtcccaaattatttctacattttttaaattttaattttaatttatttatttttttattattatactttaagttctagggtacatgtgcacagcatgcaggtttgttatatatgtatacatgtgccatgttggtgtgctgcacccattaactcgtcatttacattaggtatatctcctaatgctatccctcccccctccccctaccccccaacaggccccggtgtgtgatgtccctcttcctgtgtccatgtgtcctcattgttcaattcccacctgtgagaacatgcagtgtttggttttttgtccttgcgatagtttgctgagaatgatggtttccagcttcatccatgtcgctacaaaggacatgacctcatcattttttatggctgcatagtattccatgttgtatatgtgccacattttcttaatccagtctatcattgttggacatttgggttggttccaggtctttgctattgtgaatagtgccacaataaacatacgtgtgcatgtgtctttatagcagcatgatttataatcctctgggtatatacccagtaatgggatggctgggtcaaatggtatttctagttctagatccgtgaggaatcgccacactgtcttccacaatggttgaaccagtttacagtcccaccagcagtgtaaaagtgttccttgttaaattgatccctttaccattatgtaatggccttctttttcttttgatcttgttggtttaaagtctgttttatcagagactaggattgcaacccctgccttgttttcttttccatttgtgtggtagatcttcctccatccctttattttgagcctatgtgtgtctctgcatgtgagatgggtttcctgaatacagcacactaatgggtcttgacactttatccaatttgtcagtctgtgtcttttaattggagcatttagcccatttacatttaagattaatattgttatgtgtgaatttgatcctgtcattttgatgttagctggttattttgctcattagttgatgcagtttcttcctagcattgatggtctttacaatttggcatgtttttgcagtggctggtaccagttgttcctttccatgtttagtgcttccttcaggagctcttgtagggcaggcctggtgctgacaaaatctctcagcatttgcttgtctgtaaaggattttatttctccttcacttatgaagcttattttggctggatatgaaattctgggttgaaaattcttttctttaagaatgttgaatattggtccccactctcttttggcttgtagaatttctgccgagagatcagctgttagtctgatgggcttccctttgagggtaacccgacctttctctctggctgcccttaacattttttccttcatttcaactttggtgaatctgacaattatgtgtcttggagttgctcttctcgaggagtatctttgtggtgttctctgtatttcctgaatttgaatgttggcctgcctttctaggttggggaagttctcctggataatatcctgcagagtgttttgcaacttagttgcattctccctgtcactttcaggtacaccaatcagatgtagatttggtcttttcacatagtcccatatttcttggaggctttgtttgtttctttttattcttttttctctaaacttcccttctcacttcatttcattcatttgatcttccatcactgataccctttcttccagttgatcaaattggctactgaagcttgtgcattcatcacatagttctcgtgccatggttttcagctccatcaggtcctttaaggacttctctgcattggttattctagttagccattgtctaatttcttttcaagattttcaacttctttgccatgggttcgaacttccctctttagctccgagaaatttgatcatctgtagccttcttctctcaattcatcaaagtcattctccatccatctttgctccattgctggtgaggtgctgcattcctttggaggaggagaggtgctctgatttttagaattttcagtttttctgctctgttttttccccatctttgtggtttatctacctttggtctttgatgatggtgacgtacagatggggttttggtgaggatgtcctttctatttgttagttttccttctaacagtcaggaccctcagcagcaggtctgttgtagtttgctggaggtccactccagaccctgtttgcccgtgtcagcagtggaggctgtagaacggcgaatattggtgaacagcaaatgttactgcctgatcattcctctggaagttttgtctcagaggggtacccggctgtctgaggtgtcagtctgcccctactgggaagcctcccagttaggctacttgggggtcagggacccacttgaggaggcagtctttcccttctcagatctgaagctgcatgctgggagaaccactactctcttccaagctgtcagacagggacatttaagtctgcagaggattctgctgccttttgttcagctatgccctgccccaagaggtggagtctacagaggcaggcaggcctccttgagctatggtgggctccacccagtttgagcttcctggccactttgtttacctactcaagcctcagcaatggggggtgcctctcccccagcctcactgctgcattgcggttagatctcagactgctgtgctagcaatgagtgaggctccgtgggcttaggaccctccgagccaggtgcgggatataatctccttgtgtgccatttgctaagaccattggaaaagtgcagtattagggtgggagtgacccgattttccaggtgctgcttgtccccccttcccttggctaggaaagggaattccctgaccccttgcacttcccaggtgaggcaatgcctcgctctgcttcagctcacactcggtgcactgcacccagtgtcctgcacccactgtccgacaatctccggtgagatgaacccagtatctcagttggaaatgcagaaatcacccattttctgcgtcactcatgctgggagctatagactggagctgttcctatttggccatcttggaaccacccctatttctacatttttaaagtaaatgaactatatgaaattattgacatatgtgaatgacatgtgatatgagtggttcagtttttggcaggacaaagtcaggaatccagagcatcatccaagaaaatgtattactattgtcttcacttgcaaatatcacagcaaattatggtggaacatttgtatagatgactaatattaccatcaaataatatagcatattacttagggtataaaatacaaaactaaacaattaggattatggtagatatgatgaatcaagtaaatttaattaaagtaagaaattctatatattgtccactaaggtttgacgacatatcctcaaatataagctaggtatagcagattattattattaatttatgtatttccaagtgcatataattccatttaaaacactttttgtgtgttgaataaatttcccagtgtatggaattactagaaaaattatatacaacattaatagacaagttacaaaagttatatacaacattaatagacaagttacaaaagttaaatataaacaaaataatttgaagattcaggatatgacaatgccaccatgcatggtaaatataattggcaacatctaaaagtgtgtgaaaaaattcaatgtcattctatattctttgatccattaacagtcttttttttctctcctaaacaaatgacatgtttgcagtatgttaaaaggcaggaaaaattaagcaaaaagccccaagaccttcatatggtctttattcgcctgctgtaaacattaagtactttcttttttctttttttaattatattttaagctctcgaatacttgtgcagaatgtgcaggtttgttaaatagatatacacgtgccatggtggtttgctgcacccactaacccatcatccagattaggtatttctcctaatgctatccctgccttaacccctcatcctctgacaggccccagtgtgtgatgttccctgccctgtgtcaatgtgttctcattgttcaactcccacttatgagtgagaacatgtatgtttggttttctgttcctgtgttagtttgctgagaatgatggtttccagcttcatccatgtccctgcaaaggacatgaaatcatccttttttatggctgcataatattccatggtgtatatgtgccacattttcttgatccagtctatcattgatgggcatttgggttggttccaagtctttgttattgtgattagtgccacaataaacatacatgtgcatgtgtctttacagtagaatgatttataatcctttgggtatatatccaacaatgagattgcaggatcaaatggtatttctagttctagatccttgaggaatcaccacactgtcttccacaatggttgaactaatttacactcccaccaacagtgtaaaagcattcttatttctccacatcctctccggcatctgttgtttcctgactttttaatgatcatcatctttgtggtgttctctgtatttcccgaatttgaatgttggccagtcttgctaggctggggaagttctcctggataatatactgaagagtgttttcaaacttggtttcattttccccatcactttcaaatacaccaatcaaatgtaggtttggtcttttcacatagaccaacatttcttgtaggctttgttagttccttttcattcttttttctcaatcttttcttaacgatttatttcattaagttgatcttcaatctctgatatcctttctttcacttgatcgattcagctattgatacttgtgtatgcttcacaaggttctcttgcagtgtttttcagctccatcatgccatttatgttcttctctaaactggttattttagttagcaattcctgtaaccttttttcaaggctcttagcttccttgctttgggttagaatatgcttttttagcttggaggagtttgctattacccactttctgaagcctacttttgtcaaattgttaaactcattctccatccagttttgtttccttgctggcaaggagttgtgttccttcagaggagaagaggcatactcttttttagaattttcagcctttttgttcttgtttttccttatcttcgtagatttatctacctttggtctttcgttttggtgaccttcggatggggtttttgtgtggacatactttttgttgatgttgatgctttttcctttctgtttgttagttttccttctaacagttaggcccctctgctgcaagtctgttggagtttgctggaggtccactccagaccctgtttgcctgggtgtcaccagcggaggctgcagaacagcaaagattgctgcctgttacttcctctggaagctttgtcacagaggggcacctgccagatgccagcaggagctctgctgtatgaagtgtctgtcgacccctgctgggaggtgtctcctaggcaggaggcatggtggtcagggacccacttgagaaggcagcctgtcccttagcagagcttgagcactgtgctgggagatctggtgctctcttcagagctggcaggcaggaacatttaagtctgctgaagttgtgcccacagctgccatttccccaggtactctgtcccagggagatgggagttttacttataagcccctgactggggctgctgcctttctttcagagatgcccttcccagagaggaggaatctagataggctcattcacagtctttttaaaacttttgtagtaaaagatatagtaaaatttatctctaaatagtttaaaaatatcatacaagagtagtattatctttcagtcttgacaaaacattggattgcattgctaatagaaattaatataaaattataaccatggttgattatatgatggggaagttgagctgacaccattgtattcctagggattcaattagtgatggtctgcaatacttctttagaaattttgaagatattaataaaatcctggattctagaaaacaagccaaaagtataagtacttccaaaaatactttgaaattgatgtgtttaatactggtttggcatgatatgtaagtaaaattaataccataaacagaatgatattaaaatcccaataattccttgcaaacttcaaaggattatgacatttttaaatgaaaaatgaggtgataagattcataagaagatatcactgatacttaacaacctgcagtttttaaaaattttgactccaaatcctgtaatataaagtcagtctttgctttgcatgttaatgcaggactgtaaaaattgatcatgcaagctaaaatcatgtgaaaggatctcaataatcaagagagagaattatgatcaccctgtgacttgaaacatctttgtcaaaatattcaaaacccacttactattggttataaatatatagggaagtgaaaaacagtacaatataatttacttagcacactatactttaaaacattaaaacatttggaattaaagtctttttcccttgtattaacttaccaagaatgttttgagcaatgtttgccttcatcttctgttacttaaagatacagagtatctttgtattccttggtgaattttcatactccttttaatgtttggatcagcttctgatattttatactttgtgctttccctcttggggaatatctctgagagttcctttaatgggaagttttctgccagtgtcacttcatttaagacaccttcattcttttcacaacaatcacttccctcctccaggttcctctagcttcacctctaaagtctctcaaacagtagcattctcagcattcccacggtcagctatttcctctatagcttcttttatatttaattcaaatctcacttctagcattgtcccttttcttctttgcacattcatctttgttgcccaatttcctctttctattattcactcatgtcagatgggtatgggtttattactgagacacaatgagaccacaggactacacacctacatgctttgctgttttgcacagcacagaataatagtcaaactgacaaactttgacatatgtaaagcaattggtcactgattatgatgtggatttgttacttctatggtgactcatggactgaagacctagtggtagtttatacttcgtgcagttactaagagttactatactgtggcaactgaaatttgtggcacacatttcaaacttgttgttggggaatttgtgttatttaactaaaactttgctgactgaaatttgtgcattttgaaactgggcaaaacaaagacagcctatatcagaaaaatgaagaagactgtgttgattttgttgaatatttttgtgtttgctttgaaattatatactactagaaataagttttgtgtttgcttctgagtgatatagtacttctacaagcttggaactattttgtattgttgatttcttggatcagacattttaagacaaatagaatacatttaatctctaaactcctgtgagagcaaacctgtgattaaaactttttgtaagaaaaaaaaaaaaaggaaatctagccagagcttaaggcgaccacaagattccttcttgtcttttggttcagttggatttttttttttcctgaccaattttattgaggatacagtagaagatttcttgcacttttatttggtggtctcaacatgatcttatgacctatgtggatactgtggtcaaagccctcaggaacccactcttgtcaatgtcttgggaagctcaagtgttagcttctatttccctatctagttcttaatttcttatttgttttgttttttgcagactttcttatcttgtgagttcaatgcatacatgtataaataatttttaaaattatagcttacccagtatatctatgtgttttgtagtttgtttgtttcttctctttttttgagatggagtctcactctgtaacccaggctggagtgcagtggtgcaatctcggctcactgcaacccccacctcttgggttcaagcgattctcctgcctcagtctcctgagtagctgggattacagtcacacaccatcacacctggctaatttttgtatttttagtagagatggggtttcaccatgttggtaaggctagtctcgaactcctgacctcatgatccacctgcctcagcctcccaaagtgctgggattacaggcatgagcctccataaacacagaaatctaaggactaagtgaaaagcaatattttaggcttcaaaatttagccagggtggtgcttagaatgaatgggtagggagtttaggtagaattggtgataagatagagaggagacccaagggattcaaacaaaggataagtagaaagcaatatgagatcatagaaaagttaaacattaagacaaagaagcccagaagagacagaaaatactgtataaacagaaatgaacagaagcacaggacaagtcaaataatattaaagttgaaatggaactagaaaatatcaagttcctccaattttgtcattttacagtggggaattagtgcttctagtaagtggtaaatctaggagttttcatagctctcagactgggacctttttggctgttgcagagggcagagggtggaggatatacgctagccacaggaactatgcttggaggccagaaagaaggagtgaatcacttcaataagagttgaggaggaaatgaaaggccatgtaagagtcacgtggctgtgggaggcttgtggggtttccgtaatggggaagtaggcagagggcaaggtttctgctgggaggaagggatgacagagctgtgtggggtgggaacatgacactggagttctgaggaaatctctctctctctcggggtgcaaaagtcacccactagagtggtcttagggaagtgacaggcaagaagagcccaggcgaaggcaaacacaacaacgtctgtggtctgagcaaaaggacaagaaaagggaagttggggaaggacaggaagagcactgggctccctcgggacaacccagcccctctgttcaggctgggttctaatcatagggtgtcctacccacttcagattattctcttaggattttgtgtgttcagagagcttaataaccacttactcctctccccattgcattttatatcaatttcagattcttgcatctatatttataagtattcaccagtgtaaagagtctgccagtttcccttcttacactacctccacccttcctttttagggatagatgtacataaactcttgtgtataaaattgtcatacttaggagtctgggttatttttcatgaaagcaatttgaatattttaggctgatagacattctaaggatcacgttttccatggcagctctggggcagagcactgcatggacatggagggatatggactgtgacacaccggcagggactgtggctgtgatctctgtcccacaggatgaaagcaggactacatatcagtcagatcagagaggaaaaaccaggtggagggagagaaatgacatggtgactgtgtggagcaaaggagagggaagttttcggagaacacacatttctaggtatctatttgaatgcgtattgccatgatgggggtgagggtgttacttactcttctaggcaccctgagggggaatttcccataaatactttttgtttttttttcctcaccccttaatctttcctctgaaaacagactgatttatctcactgataaattcaggagggaaactgggcaagtatatatcagcacctcctactgccctttacctctctgggcaagttgctccccaaaggctcagtcttgctctcctcctccaggccctggctctcacaggaacagctgctcaggtggggaacttagggttttatcagcattgcctgaaataagtgagtgtattagtcttcttgggctgtgctaacacaaggcaatcagctgggtggcttaaacaacaggaagttattctctcacagctctagaggctagaagttcaaagtctccatctggaagactcagtttctagtgaaggttctcttcctggtttatacagacaactgccttctcactatgagctcatatggcctttccatggtgggtgcacacacacacacacacacagagacagagagcgacagggagagacagagagacagagaaggaaagtggaagggcactccagtgtctcttcttataaggacactaagcctattggatcagggtcttaccgtttagacatgagattgacattacttcttagaggccccatttccaaatataatcacactgagagttagggattcaacatgtgattttttttttctttgggagggtacacaaactttcagtccataacaatgagaaggccactccatttagggtccagtatgagcaagatcacttggattacacacttaaagcaaattatctagtggcactatcagaagtcaagattgaccattagatccatctatctcatgttattttcaacttgtttgcttctgaattcaagagggcaaaaaggatgatacttactgggttttataatattatccaatatagctaggttatgttgcagttataaataaactttagaacttggaggcttaacacaatgaagtttatttctcatgcagttaaactgcatgtagatcaggctgctcttctccatctcatagtcttgccatttagaaatttgactgccaagttggtaccacagggtaagatgatcagatgtgatattttcagtggtcaggactggagctggctaatgcttccaacaccacacattccattgcccagaactcagtccatgaccttaacccaactattaggaaataggacagttgcatctttcctgtaggaacatggaaaaaaatgtgaataaacacataatgtggtatctactattgttttctaaaaccacaaaccaagtatttgccattaacagcacaagatgagcaaagataacacctagtgcttatgaatgatttgggtaggatttgaactgggtattgctcttaaagatattatagttcatagaaattgccacatatctgtggccatgataggaagagctcattgagaatgacttctggcctcactattaacgttgacaaacctaagaaatagtgtatatatcaaagtatcatgttgtacacagtaaatacatagaataaatataaatttaaaacagaaacaatgttacaaatgggattttcctttttaaggcacggtcttgctctgttacccaggctggagtacagtgacatgaacatggctcactacagccctgactttcagggctcaaaagatcttcctgcctctgcctcccatttagctgggactacaggcatgtgccaccacatttggccaactttttaatttttttatcgagatgaggtctaactttgttgcccaggctggtctcaaactcctgggctcaagtgatcttcccaccttggtctcccaaagtgctgggattacaggcatgagtcattgtgcctagcacaaattcatgcccagattttaagtgactgctccaatgcattcagtgaataattaatatgatttaagaaacactataataataaatgtatttcctggatctcaaaaaaactgtgttcatgtatttacacatattaaccagccagggtcactgataagtcactgaatacttcatataaactacttaaatcaaacattgacaaatgctgccctttttatacaatgtgttttggtcagatcacaaaacaacttaaatttggatttttgtggtctccccatgtatgagtcctcttgaaaagctgcatgttctgactatcacaatcgtataccattccacactgtacagaattttcagtatttagttgagatgcatgctgttcttccaggacttcaaattatgaacttcccagggggttagtcagtcaaaatgctgctctagagaatatagttctaagtagatttgtatctgggattctatacattgaaattaaaatcacaatgcattttaattttttgtttaaacatctgcagtttaaaactctttgatagaagagattacattgttaacctctgtgtgtttatatgcctagtaagacactggtatcaaccatgtactatgtaattttttagtgaatgactatacagattaaatgcagtagaagggcttctttatatttgttatggtcctatgcattctgaatggctgcaatttaatctctaaggataatgtagagtggactgtagtttatataattgtgcagggaaatcttggaagatgtaaaaagcaacgtctccaatttataaaagaatcagtgagaatgaggaaataagaaagatgggtgaaaagaggtgaaattggaaagagaaaactttgaggacattcaaaagggggaaagaaagaatggagtgatgagaagaggggacactatggagagtggaggcaaagttaagtggagaaactggaagaactgtgatctattaaaacaagcaggaactgaaaaaatgacatggtttctcctttttatcttacaaatgacatgcaaaatccagagggacaaaatgacctttctttcaatgccatacaccactttgtctaaggcagttgaggaagggaagtagatataatggagcttagtggcagagcagctggaatcctgagagaagagaataactttagttcagagcaggtggggaaatgagagattgagtaggaggaccaaggttgagggaagcagatcttcttagttgctgtcagcggctgatggggaagattgttggtagaaggaagtcagaatataggtacagagggataagtttgctaagaacagagatcagcaaacagcttttctgagagaaagaaacatctgcaaatgacatgctgtttctgcagtttctgctgctagctcttcttctcccaggtggtgacaatgcagacggtaagaacatcgctgtcagctgcaaggttacatgtatctgggtagagtgtgctgggctttcccgagatggatcaatagagatgccagaatgcttgccatctgagcatacctgtctccaggtccagtttactcttttggaggatggtggcagagcatgggctctgtgtaaggaaaatgttatagctaaagtagtcattaatgtttctctgtggtaactggttcaatttccatttcctctccattttccgttaaaacaatcagggcccaggaaaatgatgtggtttctcctttttatgttacaaatgacacgcaaggtccagagggatgaagcaacctttcaatgccacgcaccactttgtctaaggcagttgaggaagggaagtagataagatggagcttagtgggagagcagctaaaatcctaagggaagagagtaactttagttcagagcagatggggaaatgagagattgagtaggaggcctaaggttaagggaagcagattgtcttagttgctgtcagtggctgatggggaagattgtctgttggtagaaggaagtcagaatatagatgtagagggataagtttgctaagaacagagatcagcaaatggcttttctgagagaaggaaacaactgcaaatgacatgctgtttctgtagtttccgctgctagttcttctcctccccggtggtgacaatgcagatagtatgaacatctctgtcagctgcaaggttacatgtatctgggttgagcatgatgggctttcccaagatggatcaatgaagaagccagaatggttgccatttgagcatacctctctccaggtccagtttactcttttagaggatggtggcagagcatggggctctgtgtaaggaaaatggtatagctaaagtagtcattaatgtttctctgtggtaactggttcaccttccattttctctccattttccttgcctctctttttttttttttccttacactacttgcccttcttccaccaaaagcatcccaggaacacgtctccttccatgtcatccagatcttctcatttgtcaaccaatcctgggcacgaggtcagggctcaggatggctggacgagttgcagactcatggctgggacagtgaatcaggcacaataattttcctgcataactggtccaagggcaacttcagcaatgaagagttgtcagacctagagttgttatttcgtttctacctctttggattaactcgggagattcaagaccatgcaagtcaagattactcgaaatgtaagttcaatcatctaaattatgggagttctttagaatgttctatttcagggaaatattctctactaatttttgggccatttcccattatcccatcccaccataaaattctgcatagatgaaccttttaagggatattcctgatgttgactccctcaaatttcctaggctcttgctatttttggaattttttttcttgtgctttgttatcttccacctgtggtctccctctgtatcctacaatccagagatacacagctgtcccactctgtgcttgactcccttaaaataaaatctattcccccattcaatttcttcaaagatctatgtccacagttcaatgaccattatgaccaatttcatgcgtcatggaatgctatcctcctacctccctcacaaaggctcccccttcaccctctatcctatattatgtggctcacctatcatttttccacatcttccttggccactttttcttgttaattcaaggcatttatctcttattgtccccttccctatccccagcaatttttctctcttgtttctgatcaaatatgtctttggctcactctcacatccatgtaaaactttcttgcttcacttcctgatacagccctaggtttttatactgttgttttcacttttttgtttgaactctttttctcattcctcccttcctccagatccctttgaagtacaggtgaaagcgggctgtgagctgcattctggaaagagcccagaaggcttctttcaggtagctttcaacggattagatttactgagtttccagaatacaacatgggtgccatctccaggctgtggaagtttggcccaaagtgtctgtcatctactcaatcatcagtatgaaggcgtcacagaaacagtgtataatctcataagaagcacttgcccccgatttctcttgggtctcctggatgcagggaagatgtatgtacacaggcaaggtcagtagtttcagccccttcctctaagatttttctattcaagtactgccccttctgttcccacccttcaaactcaggacaagaaagaggacaagaagcaggggggttgctgggtaatagtttcttagaggcaggaaaaagataactgtgcatattcatgtggatgtgtgtatgtggatgtaagttgtgtgtaagtttcttcatcagaacactttttctgctctctgcagtgaggccagaagcctggctgtccagtcgccccagccttgggtctggccagctgttgctggtttgtcatgcctccggcttctacccaaagcctgtttgggtgacatggatgcggaatgaacaggagcaactgggcactaaacatggtgatattcttcctaatgctgatgggacatggtatcttcaggtgatcctggaggtggcatctgaggagcctgctggcctgtcttgtcgagtgagacacagcagtctaggaggccaggacatcatcctctactggggtaagactggaggttggaagtgtaggtaggtggttcttgagcctagaggttaggggagaggaaattttgaggatagcagacctaggtgagggattgtaggaagaaatgtatagggtaatttaaagaatagtggagtaagacctaagggatacatggatactagagatttaaggggaaatgaggaatccttccttgagtaagttttggaatagagtgactgaaataggataactgatgcaactcatccaatgcatatgttaagtaaggaaatcaatagatggaatagaatcagcagtgagtttaaaatggcatccatgtatctttccaatatgtgcaggacaccacttttccatgaattggattgccttggtagtgatagtgcccttggtgattctaatagtccttgtgttatggtttaagaagcactggtgagttttttgtatttcctcctctcctcccagtttcttatttcacattccatttttcactctcttagcttttctctattgactactccaccttatcctcagttaccacctccaacttattcagggtttctcccattcctgttccttcacagctcatatcaggacatcctgtgagactcttccccctgactcccccattgtgttaagaacccagcaacccaggagcctagtacaatatagtgatgccatcccgtcgactctccatttaaattgtttctctttctgcataataaacatttgttaataaaaaccaaattctaatttggaatgtttttcttggaatctccactttttatatagcactcaaccttcaaagcccatttctgatgtcatttcctccaacgatcttccttgacccatactgaacccagagagcccctcaactgttatgtgcatgcaacacttcctaccctgtgttgcagctacttgagtctttcatttcacatttttaagcttttaggaggcaaggatcatgataaatgtgcctttcctgcatatgataggctcagtgaaacacaaaacaaaacatcatatctaagttggattaactgtcatgttgacaatttttctcattatattccttcccaatgttgttgtagcattggaaatattcatgcttctctttttgtttaaatgcttcatacctagcagataaattccactctcctttagctccctcattttctccttaaacatttaaggaaaagtgcagccttgtgccttgatacacttattttctaaacacagagaattacaaatgcaaatagacagagagtgtgcaagttacatgtgatgatcagtggtgcagatatgtcacagaatacttaagaaaaggaacaagagatttggtcctcagggttggagtgggatcaaggcaaccgttgtgctgtatatggaagacaaatttcctaaaggactgtgttatctgagatagccttggctaaactttagtccctttaggtttcattcactaattcaactaatattttttgggtactgagttgagacaggcacatacagcttctaaactggtggtggataaaacaaatgagattgtgtccactctcctgaagttgcaatgtttgcttcctcaatctgtccatgtttcttttctattgttttcatgtttttatttatttcaaaaaattttaaatagattttattgatgtataatttatagtaaaatttgaatctttaagtgtacagttctatttattttgataattatataagcccatgtaaacaaaatcacaatcaagatattaaatgtcataacccctcatacctgtttgtagtcaagcacaccaacaacctggcattcttgcaactactgatatactttatgtcacttaagcttaaacttgtattttctatgattttattaagatggaaataaacatatctacttggtattgccttttatttttcttcagtttagcatatggttgtaagtaatccatgctgctcagtgtattagtaaccattccttttcattgctgaatagaattccatgtatgaatataactttttttggaggaggaggtggcttcccagatctggcagggcctttgtctccttgtgcccactcacccagcttgggtcctgcttccattgctcatttccttggcaccaggaggccctaggtgactctcctgcacccttagtcaccctgtgacctgcagatacagagagatcaatagagaggattctaggacacccaggaaactgggaaataaagccaaaaatatataaaattaaacttatttttgggagaagagacaagatggctgactagatgcagccaggaagagcttctcccaccaagagacaagaccatcaagaagagcagtatactctgagaagatcattggaaagaaggcattgagagtggatggagggagaatgcagaccctgggctgaaggggggaagaagctgcgaaccctacacagggttgccaaacactaggacttattcttggtcctcagtggcttctagggaaggggttggttaaataggcatgaagtggcccactctatccatgaacctctggaatcctagctgcaggagacccccacgacccacacagacatttgagttggcagggaaagctacttgcagagttggcagggacaggactccagcttctatggaacccagagggtttggtgtgagaacagctacagtggagcatggttagggatgtccattacccgaggattggcatgctcctctaggtggctttgacccctttgttgactatcagacctggacagaatagggctaccatgatatttggatggggctagactgatctgagccccccaccccatgtctgctggcctctcccaggatccctgcctgtctgtacctgcttgcagcacagcctcagatgttcaactgaggcagttctcagagaccactgccataggtctttcactggctgaccccaccaaaccaccaaatagcttctgtcactggcctcacatcagcatgcacctgccttcagcacccacaactgctttgctggcatacatacacagacatcactgccttgctaccacttctgtgcacatatgcatggagacctcaccactgctgcactgtccccactagcctacatgcgtgcatgtaccctgccacactaccatccctggtgtacatgtgcatggaccttctaccctgctgccccaccaccaccagcatgtgagtgagtgtggaccccaccatgccactgccccactgctgccagtacatgcatgtgagagcagacccccaccgccactgcccttatgaaatgcttttctgggacccccccaccctgcccaccataggagtgttgttgctagtggactgggagcaccttggccccctccagtgaagcaggtgctcagtctcaaggggccaaagaacaaggttctgggcctggtcccaggccctcagtcttagagcatatagcccaggagtgctgagctgagccatggccccctgaaatcatcaagaaataaagctagctcactgacccaaacctataccatagtcaaagcctcaagggcatcaaagaatataaaagcaaaaggccccatcaaaaggacagcaactttaaacgtttaaggaacatcagcccacatagatgagaaagaacccgtggaagaactcaggcagctctaaaagtcagagtgtcttcttacttccaaatgaacacactagctccccaacaatctttcctaaccagactaaaatggctgagataatagacatagattttagaatctggatggcaatgaaaatcatgagattcaggagaaagttgaaatggaatccaaagaatttaaggaatccagtaaaataatacaataactgaaagacaaaatagccattttaagaaagaactaagctgatttgacagagctaaaaaattcactaaagaattttataatacaattggaagtattaaaggcagaatagaccacactgagggaagaatctcagagctcaaagaccagttcttctaattaactcagtcacacaaaataaagaaaaaataatacagaaggatgaacaaaatctctgagaaatatgaggttatgtaaagagaccaaacctgtactcactgatgtccctgaaagagagagagagagagtgtgcaaacaattgaaaacatatatgaggatattgtccatgacaattttctcaacttcactatagaggtcaacattcaaattcaagaaattcagagaactcctgagagatactacactagaaaaccatcctcaagatacacaataataagattatccaaggtcaatgtgaaagaaaaaaatactaaaggcaaccagagaggaggagcaggtcacctacaaagggaccccaatcaagctaacagcatacctttcagcagaaaccttagaagccagaagacattgagggcctatattcagcattgttaaagaaaagaaattctgacccaaaatttcatatccagcccaagtaagatttatatgttaaagagaaattcagcaaagcaaatgctaaggaaatttattaccaccagacctgccttataagaagtcattaagggagtgctaaacatggaaataaaagactcttaccagccaccacataaatgcacttaagtaaacagaccagtgacactataaaggaactacacaattaagtctacataacaaccagctaacaataatgacaaaatcaacttctcacttatcaatatttaccttgaaggtaaatgagctaaacaccccaagacacagagtggcaagttgaataaagaaacaagaccctggctcatgcctgtaatcctaacactttgaggctaaggcaggccgattgcttgatcctatgagtttgagaccaacctaggcaacatggaaaaaccccatttctacaaaaaaaccaagaaaattagctaagaatggtggcacatgcctgtagtcccagctactgaggaggctgaggtaggaggatcacccaagcctggggaggtggaggctgcagtaagttgtgatcatgccactgcacttcggcctaagtaacagagtgagaccctgtctcacgttaaaaaagacaaaaaaaaaaaaaataaacaagacccacctgtaagttatcttcaagagacccatctcacatgcaaggacacccataggccaaaactaaaggatggcaaaaaatctgtcaagcatacaggaaagaaaaaagagcaggggttgctatttttatttcagacaaaataaactttaaaccaacaactgtcaaaaaggacatagaaaaacattatttaatgatagagggttgaatttaataagaagacttagctatcctcaatatatatgcacccaacacaggagcacacaggttcataaaacaagtacttagagacccacaaagaagcttatgttatcatacaataatggtggtagacttcaacacccaactgacagcattagatagatcatcaagagagaacactgataaaggtatttggaacttaaactcgacacttgaccaaatggacctaataaacattgacagaacgctccacccaaaaccaaaagaatatacattcttcccatgcctccagctttgttccttttgcttaggattgtcttggcaatgtgggctctttttgatttcatatgaaagatcaggtagtttttccaattctgtgaagaaagtcattggtagcttgatagggatggcattgaatttataaattactttgggcagtatggccattttcacgatattgattcttcctatcgatgagcatggaatgttcttccatttgtttgtgtcctcttttatttcattgagcagtggtttgtagttctccttgaagaggtccttcacatcccttgtaagttggattcctaggtattttattctctttgaagcgattgtgaatgggagttcactcatgatttggctctctgtttgtctgttattggtgtgtaggaaagcttgcgatttttgcacgttgattttgtatcctgagactttgctgaagttgcttatcagtttaaagagattttgggctgaggtgatggggttttctaaatatacaatcatgtcatctgcaaacagggacaatttgacttcctctgttcctaattaaataccctttatttctttctcttgcctgattgccctggccagacttccaatactatgttgaataggagtggtgagagagggcatccttgtcttgtgccagttttcaaagggaatgcttccagttttgcccattcagtatgatattggctgtgggtttgtcataaataggtcttagtattttgagatatgttccatcaatatctagtgtattgggagttttttagcatgaacagctgttgaattttgtcaaaggccttttctgcatctattgagataatgatgtggtttttttctttagttctgtttatgtgatggattacatttattgatttgcatatgttgaaccagccttgcatcccagggatgaagctgatttgatcatggtggataagctttttgatgtgctgctggattttgtttgccagtattttactgaggattttgcatcagtgttcatcggggacattggtctaaaactctcagcatcaggattatgctggcctcaaaaaatgagtaagggaggattccctctttttctattgattggaatagtttcagaaggaatggtaccagctcctctttgtacctttagtagaatttgtctgtgaatccatctggtcctggactttttttggttggcaggctattaattattgcctcaatttcagagcctgttattggtctattcagagattcaacttcttcctggtttactcttgggagggtgtgtgtgtcgaggaatttatccatttcttctagattttctagtttatttgcacagagatgtttatattatcctctgatggtagtttgtatttttgtgggatcagtggtgatatcccctttatcattttttattgtgtctatatgattcttctctcttttcttctttattagtcttgctagtggtctatcaattttgctgatcttttcaaaaagccaggtcctggattcattgatcttttgaagattttttttgtgtctttatctcattcaggtctgctctgatcttagttatttcttgtcttcggctagcttttgaatatgtttgctcttgcttctctagttcttttaatgtgatgttaggtgtcaattttagatctttcctgctttatcttgtgggcatttagtgctataaatttccctctacacactgctttacatgtgtcccagagattctggtacattgtgtctttgttctcattggtttcctagagcatctttatttctgccttcattttgttatttaccaagtagtcattcaggactacagtttccatgtagttgtgtggttttgagtgagtttcttaatcctgagttctaatttgattgcactgtggtccgagagacagtttgttgtgatttcttttctttttacatttgctgaggagtgctttacttccaattttgtggtcaattttagaataagtatgatgtggtgctgagaagaatatatactctgttgatttggggtagagagtaccgtagatgtctattaggtctgcttggtgcagagctgagttcaagtcctggatatccttgttaaccttctgtcttgttgatctgcctaatattgacaatggaatgttaaagtctcccattattattgtgtgggagtataagtctcttcgtaggtctgtaaggactcattatatgaatctgggtgctcctgttttgggtgcatatatatttaggatagttagctcttctttttgaattgattcctttaccattatgtaatggccttctttgtctctctctctctcttttttttttttttttgagacagagtctcactctgtcacccaggctggagtacagtggtgcgatctcagctcactgcaagctcttcctcctgtgttcataccattctcctgcttgagcctcctgagtagctgggactacaggtgcccaccaccatgcccagctaattttttgtatttttaatatagatggggtttcaccatattagccaggatggtctcaattgcctgacttcatgatctgcccacctcagcctcccaaattgctggaattacaggtgtgagccaccgtgcctttgtctcttttgatctttattggttaaagtctgttttatcagagactgggattgcaacccctgctttttttttgctttccatttgcttggtagagtgtcctccatccctttattttgagcctatgtgtgtctctgcacttgagatgggtctcctgaatacagcacactgatgggtcttgactctttatccaatttgtcagtctgtgtcttttaattgtcatatttagctcatttatatttaaggttaatattaagtgtgaatttgattctgtctttatgatgttagctggttattttgctcattagtttatgcagtttcttcatagcatcaatggtctttacaatttggcatgtttttggagtgtctggtactggttgtttctttccatgtttagtgcttccttcaggagctcttgtaaggcaggtctggtggtgacaaaatctcttagcatttgcttgtctgtaaaggattttatttctccttcacttatgatgcttagtttgggtggatatgaaattctcggttgaaaattcttttctttaagaatgttgaatattggcccccactctcttctggctggtagtgtttctgcagagaaatcagctgttagtctcatgggcttccctttgtggaacccgacttttctttctggctgcccttaacgtttttccttcatttcaaccttcgtgagtctgataattatgtgtcttggggttgctcttctcatggagtatctttgtggtgttctctgtatttcctgaatttgaatgttggcctgccttgctaggttggggatgttctcctggataacatcatgagcagtgttttccagcttggttccattctccctgtcactttcaggtacacaaatcaaacatagatttttttcttttcacatagtaccatatatgcagaatacacctgtcttctgcatcaatcaagctggtagttgcagaccggagctcttcctattcagccatcttgggggaagtcgcaattttcaacacattcaaaaaaaccaaaatcataccaaccacactcttgcaccacagcacaacaaaaatagaaatcaagatcaagaagagctctgaaaaactatacaattaaatagcaattaaacaatttctggtcctgaaagacttttgggtaaacaacgaagtcagaaatcaggaaattgttctaacaaatgaaaacaaagatactacatgccagagtctctgagacacagctaaaacagagttaagaggaaagtttatagcactaaatgcccacatcaaaaagttagaaagatctcaaattagtaacataatctcacatctaaaggaactagaaaaataaggaaaaaaaacctaaagctagcagaagacaagaaataaccaaaatcagagctgaactaaataaaactgagaactgaaaactatatagaagataaacaaaaaaattggtaatttgaaggaataaataaaattggtagaccacagactagaataataaaggagaaaaagatgagaaaatccacataaacccaatcagaaatgacaaagggggcattgccaccaacctcatagaaatacaaaaaccactcagatactgttatgaacacctctatgcacgtaaactatgaaacctagaagaaatggaaaaattcttgaaaatatacattcccaaaattgaaccaggaagaaattgaaaccctgaacagatcaataatgagttccagagttgaatcagtaataaaaaggttgccaatcagaaaaagccttggaacagagagatccatggctgaattctaacaggtatataaagaaaagctggtatcaattctttgaaaactattccaaaaattgagggggagtgattattccctaactaattctatgagataagtatcattgtgataccaaaacctggtagagacacaataaaaaaagaaaacattagctaattttcctgaagaatatatatgcaaaaatcctcaacaaaatattagcaaagcaaatccaggatcatatcaaaaagctaatctgctattatcaaatgggcttcatccctgggactcaaggttggttcaacatacagaaatcagtaaatgattcatcacataagcagaactaaaaacaaaaaacacatgattatctcaacagatgcagaacagtcttttgataaaattccacatctcttcaggttaaaaacccttaagaaactaggcattaaaggaacacacctcaaaacaattagaggcatctatgacaaacccacagccaacaacgtactgaataaacaaaagcttgaagcattccctttgaaaactagaacaaggcaatgatgtccactcttaccactgctattcaacataatactggaagtcctagccagagcaatcagggaagagaaaggcattcaaataaaaagagaggaagttaaattatctctttcttcagacaatatgaatctatacctagaaaaccccatagtctctgtgcaaagggttctaaatctgataaacaattttagttaatcttcaaggtataaaatcaatgtacaagaatcagtagcttctctatatatcaataaagtccagaccgagagctaaatcaagaacacaatcacattcactataatcacaaaaagaataaaatgcttaggaatatagctaaccaagaaagtgaaagatctctacaacaagaattataaagcactgctaaaaaatcagagattatacaactgaatgtaaaaatatttcatgctcatggattgaaagaattaatgttgttaaaattatcatattgctgaaagcaattcagagactcagtgctactcctatcaaagtaccaatgacattttttcatagaatttgaaaaaactattctaaaattcatacagaatcaaaaaagagtctggggggtgcaaccaagatggccgaataggaacagctctagtctagagctcccagcatgagcgacacagaagacgggtgatttctgcatttccaactgaggtacccggctcatctcactgaggagtgtcagaaagtgggtgcaggacagtgggtgcagttcacccagcgagagccaaagcaggatgaggcatcgcctcacctgggaagcacaaggggtcagggaattccttttcctaatcaaagaaaggggtgacagatagcaccaggaaaatcgggtcattcccaccctaatactgtgcttttccaatggtcttagcaaagggcacacaaggagattatatcccatgccaggcctggagggtcctatgcccacggagcctcactcattgctagcacagcagtctgagatcaaactgcaaggcagcagagaggctgggggaggggcgcccaccattgcctaggcttgagtaggtaaacaaaggccaggaagctcgaatggggtgtagcccaccgcagctcaaggaggcctgcctgcctctgtagactccatgtctgggggcagggcacagccaaaccaaaggcagcagaatcctctgcagacttaaatgttcctgtctgacagcttggaagagagtagtggttctcccagcatgcagctggagatctgagaatggacagagtgactcctcaagtgggtcactgatgcccaagtagcctaactgggaggcacctcccagtaggggcagactgacacctcacacggcagggtactcctctgagacaaaacttccagaggaataatcggcagcaacatttgctgttcaccaatatctgctgttctgcagcctctgctgctgatacccaggaaaacagggtctggagtagacctccagcaaactccaacagacctgctgctgagggtcctgactgttagaaggaaaactaacaaacagtaaggacatccacaccaaaaccccatctgtatgtcaccatcatcaaagaccaaaggtagataaaaccacaaagatggggaaaaaacagagcagaaaaactggaaactctaaaaatcagagtgcctctcctcctccaagggaacgcagctcctcaccagcagtggaacaaagcaggatggagaatgaccttgacgagttgagagaagaaggcatcagacgatcaaactactccgagctaaaagaggaagttcgaactcatcgcaaagaagttaaaaatcttgaaaaaaaattagatgaatggctaactagaataagcaatgcagagaagtctttaaaggaccggatggagctgaaaacaaaggcaagagaactacatgatgaataacaagcctcagtagccgatgggatcaagtggaagaaagggtatcagtgatggaagatcaaatgaatgaaacgaagtgagaagtttagacgaaaaagaataaaaagaaattaacaaagcctccaagaaatatgggactatgtgaaaagaccaaatctatgtctgattggtgtacctgaaagtgacagggagaatggaaccaagttggaaaacactctgcaggatattatccaggagaacttccccaatctagcaaggcaggccaacattcaaactcaggaaatacagagaatgccacaaagatactcctcaagaagagcaactccaagacacataattgtcagattcaccaaagttgaaatgaaggaaaaaatgttaagggcagccagagagaaaggtggggttacccacaaagggaagcccatcagactaacagctgatctctcagcagaaactctacaagccagaagagagtgggggccaatattcaacattcttaaagaaaagaattttcaaaccagaatttcatatgtagccaaactaagcttcataagtgaaggagaaataaaatagtttacagacaagcaaatgctgagagattttgtcatcaccaggcctgccctacaagagctcccgaaggaagcactaaacatgaaaaggaacaagtggtaccagccactgcaaaaacatgccaaattgtaaagactgtcgaggctaggaagaaactgcatcaactaatgagcaaaataaccacctaacatcataatgacaggatcaaattcacacataacaatattaactttaaatgtaaatgggctaaatgctccaattaaaagacacatactggcaaattggatagagtcaagacccatcagtgtgctgtattcaggaaacccatagcaagtgcagagacacacataggctcaaaataaagggatggaggaagacctaataagcaaatggaaaacaaaaaaaggcagtggttgcaaacctagtctctgataaaacagactttaaaccaacaaagatcaaaagagacaaagaaagccattacataatggtaaagggatcaattcagcaagaagagctaactatcctaaatatttatccacccaatacaggagcacccagattcataaagcaagtgattagaggcctagaaagagacttagactcccacacaataataatgggagattttaacaccccactgtcaacattagacaggtcaacgagacagaaagttaacaaggatatccaggaactgaactcagctctgcaccaagtggacctaatagacatctacagaactctccaccccaaatcaacagaatagacattcttctcagcaccacaccgcacttattccaaaattgaccacatagttggaagtaaaggtctccttagcaaatgtaaaagaacagaaattataacaaactgtctctcagaccacagtgcaatgaaactaggattcagcataaagaaattcactcaaagccgctcaactacatggaaaccgagcaacctgctcctgaatgactactgggtacataatgaaatgaaggcagaaataaagatgttctttgaaaccaacgagaacaaagacacaacataccagaatctctgggacacattcaaggcagtgtgtagagggaaatttatagcactaaatgcccactagagaaagcaggaaagatctaaaattgacaccataacatcacaattaaaagaactagagaagcaagagcaaacacattcaaaagctagcagaaggcaagaaataactaagatcagagctgaactgaaggaaataagagacacaaaaacccttcaaaaaatcaatgaatccaggagctgcttttttgaaaagatcaacaaaattgacagactgctagcaagactaatacagaagaaaagagagcagaatcaaatagacgcaataaaagatgataaaggggatatcaccactgatcccacagaaatacagactaccatcagagaataatactataaatacctctacgcaaataaactagaaagtctagaagaaatggataaattcctcgacacgtacacccccccaagactaaatcaggaagaatttgaatccctgaatagaccaataacaggctctgaaattgaggcaataattaatagcttaccaatcaaaaaaagtccaggacccaatggattcactgccaaattctaccagaagtacaaggaggagctggtaccattccttctgaaattattccaatcaatagaaaaagagggaatcctacctaactcattttatgaggccagcattatcctgataccaaaacctggcagagacataacaaaaaaagacaattttagaccaatatccctgacgaacatcgatgcagaaattctcaataaaatactggcaaaccgaattcagcagcatatcaaaaaccttatccaccctgataaagtgggcttcatcttgaaaagtcctttgacaaaattcaacaacccttcatgctaaaaactctcaataaattaggtattgatgggatgtatctcaaaataataagagctatttatgacaaacccacaggcaatatcatactgaatgggcaaaacctggaagcattcccttggaaaactggcacaagacagggatgccctctctcaccactcctattcaacatagtgttggaagttctggccagggcaattaggcaggagaaggaaataaagggtattgaattaggaaaagaggaagtcaaattgtccctgtttgcagatgacatgattgtatatctagaaaaccccatcgtctcagccccaaatctccttaagctgataggcaacttcagcaaagtctcaggatacaaagtcaatgtgcaaaaatcacaagcattcttacacaccagtaacagacagagagccaaaccatgagtgaactcccattcacaattgcttcaaaaagaataaaatacctaggaatccaacttacaagggatgtgaaggacctcttcaaggagaactacaaaccactgctcagtgaaataaaagaagatacaaacaaatggaagaacattccatgctcatgggtaggaagaatcaatatcatgaaaatggccatactgcccaaggtaatttgtagattcaatgccatctccatcaagttaccaatgactttcttcacagaattggaaaaaactactttaaagttcatatggaaccaaaaagagcccgcattgccaagtcaatcctaagccaaaagaataaagctggaggcatcacactacctgacttcaaactatactacaaggctacagtaaccaaaacagcatggtactggtaccaaaacagagatatagaacaatggaacagaacagagccctcagaaataacgccacatatctataaccatctgatctttgacaaagctgacaaaaaccagcaatggggaaaggattccctatttaataaatggtgctgggaaaactggttagccatatgtagaaagctgaaactggatcccttccttacaccttatacaaaaatcaattcaagatggattaaagacttacatgttagacctgaaaccgtaaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaacttttgcaacctactcatctgacaaagggctaatatccagaatctactatgaactcaaacaaatttacaagaaaaaaacaaacaaccccatcaaaaagtgggcaaaggatatgaacagacacttctcaaaagaagacctttatgcagccaaaggacacatgaaaaaacggtcatcatcactggccatcagagaaatgcaaatcaaaaccacaatgagataccatctcacagcctttagaatggcgatcattaaaaggtcaggaaacaagaggtgtggagaggatgtggagaaataggaacacttttacactgttggtgggactgtaaactagttcagccattgtggaggtcagtgtggcgattcctcagggatctagaactagaaataccatttgacccagccatcccattactggttatatacccaaaggattataaatcatgctgctataaagacacatgcacacatatgtttattgaggcactattcacaatagcaaagacttggaaccaacccaaatgtctaacaatgatagactggattaagaaaatgtggcacatatacaccatggaatactatgcagccataaaaaatgatgagttcatgtcctttgtagggacatggatgaaactggaaaccacctttctcagcaaactattgcaaggacaaaaaacaaaacactgcatgttctcactcaccggtgggaattgaacaatgagaacacatggacacaggaaggggaatatcacataccggggcctgttgtggggtggggggaagggggagggatagcattaggagatatacctaatattaaataacgagtcaatggctgcagcacaacaacatggcacatgtatacatatgtaactaacctgcacattgtgcacatataccctaaaacttaacgtataataataaaaaaaaaagagtctgaacacccaaagcaatcttaagcaaaaagaacaaagctggaagcaccacactatctgacttcaaactatagtacaaggctacagtaaccaaaacagcatggtactagtagaaaaacaaacacatagataaatgaacaggttagagaacccagaaataaatctgcacaccttacaaccatctgatctttgacaatgtcaacagtaagaagcaatggggtgaagacttcctattcaataaatggtgttgtgctaactggctagccatatgcagaatattgaaaatttacatcttcctttcaccacatacaaaagttaattcaagatggattaaagacttaaatgtaaaacctaaaactacagaaatcttggaagaaaacctgtgaaattccattctggacataggccctggcaaggatttcatgatgaagacttcaaaagaaactgcaatgtaaacaaaaattaaatggaacttaattaaactaaagagtttctgcacagcaaacaaaattgtcaacagaataaacagacaacttacagaatggaagaaaatacttgcaaactatgtgtatgacaaagatctaatacccagcatctataaggaacttaaattaacaagcaaaaaaccacaacctccttaaaaaataggcaaagaatatcaacagacacttctcaaaagaagacatatacatagccaacaagcatatttaaaaattctcaacatcactaatcatcagagaaatgcaaatcaaaaccacaatgagataccatctcacaccagtcagaatggctattattaaaatgtcaaaaaagtaacagattctagcaaggttgcagagaaaagagaatacttacactctgctggtgggaatgtaaattagttcagccactgtgaagggcagtttggtgatttctcaaagaacttaaaacagaacttaaaaactacctatgggtactatgctcattacctgtgtgacgaaacattctgtacaccaagcccccacaacatgcaatttacccatgtaacaaaccttcaaatgtagctgccgaacctaaaataagaggtggaaaaaaataatttttaaaaatctatctggtctttaatgggtatgtagtatatttttagtttgagactttttaaataaaatctatttttgtttgttttgtttttaatgtgttttttgtttgtttgcttttgtttagttacctgcttagactaattcaaactggggtgaagtgatatctcattgtggttttgatttgcatttccctactgattagtcatgttgagcatattatatttacctattggtcattcgtatgtcttcttttcagagttgtctaatcaggtcttttgccctttaaaatttaaataacttacttttttgccattgagctctttgagttccttatatattctggatcttaatccctttggatacgtagtttgcagatattttctaccattctgtagcttatttcctttgctgtgcagagcttgttagttagataaaatttcatttgtctattttgttttgtattaccagtgcttttgaggtcttatcgaaaatcttttcccagaccaacatgaagcattcccctatattttcttcttgtagtttcacaattttgggtcttacatttaagtctttaacccatcttcatttgattttgtatatggtgagatatagggctctagttttgttcttctgcatatggatattcagttttcccagcactatttattgaagagactgttctgtccccaatgtgtgtcttggcacttttgttaaaaataggttggctgtaaaggcatggatttatttctgggttatatattctgttccgctgatctatgtatctgtttttataccagtaccatgctgttttggttactatagttttgtagtatattttgaaatcaggtagtgtgatttctacagatttgccctttttgctcaagattactttggcttttggggtctttgatggttcaatattaatttagaatcatcttttctatttctatgaagtatgtcattggtattttgatagagattacattgaatctgtagattgctttgggtagtatgggcattttaacaatgccaattatttcaatctgtgaacataaaatacgatatgatttggccctgtgtccccacccaaatctcatcttgtagccaccataattcccatatgttgtgggagggacctagtgggagataattgaatcatggggacaagtctttcctgtgctgttctcatgttagtgaatgagtctcatgagatatgatagttttaaaaacagtagtttccatgcacaaactctcttctctcttgctgctgctgtgaaagaagtgccttttaccttctgccatgactgtgaggccaccccagctacgtggaactgtgagtccaaataaacctctttcttttgtaaattatctagtctcagatatgtctttattagcaggacgaaaatgaactagtacaaaatacctttccaataatttttgtccttttcaatttctttcatcagtgttttatagatttttttgtagagtttttacctcctttgttacatttattcttaagtatcttatattttgtagctattgtacatggaattgttttcttgatttatttttcagatattttgatgttggtgaaggaaatattactgctttttgtatatgcatttaatatcctgaaattttactgaatttattagtgctcatggctttttgatgaagtaattagtattttctgtatataaagttgtgatctccacaaacagggataatttgacttccttctttttaattgtatgccccgtatttctttctcttgcctaattaccctggctaggagttccagtacaatgttgactagaagtagtgaaagtggacatctttgcttagttccagattttagagaaatttctttcatcttttccccattttgttttatattagctgtgggtttctcatatgtggcttttattgtgtttaggtacgttccttatatacctaatttattgataacttttatcatagtgggatgttgaattctatcaaatgctttctctgtttctattgaaatgattacgtgcttttgtcctttattctattaatgtgatgcagtacatttattaatctgaatactttgaatcatccttgcatccctggaatgaatgccacttgattttaaaggctggtcttttaagtattctgttaaatttagtttgctagcattttcttgaggatttttgcatgcgtttcattagggatattggcctgtagtttttcctattttattttatttttttgagaaagtgtctcactctgtcacccaagctggagtgcagtggcacaatcacatctcaccacagcctcaacctcctgggcttgggtgatcctccaacctcagcctcctgagtagctaggactacaggcgcccaccaccatttttggctaatatttgtactttttttagagacaaggtttcatcatgttgacaggctggtcttgaactcctgggctcaagtgatccaccctccttggcttcccaaagtgctaggattacaggcatgagcaaccacttctgtcctgtaattttcttttttgtttttgttgtgtcatctagtttttatatcgggctaatgaatgctagtctcaaataataagtctggacaaatttactctcattcagtttttttgggggcagtttgagaagaacaggcattagttatttttaaaatatttggtagaattcagcagtgaggtcatcagctcctgtgctttctttaatgggagactttaaactattgtttcaatcttgttactcattattggcctgtttgaattttctgtttcttcatgattcaatcttgtggttgtatgagtccaggcatttacctatttcttctaagttttacaatttgttttcatatagttgttcataatggtctcttatggtcctttgtatttatattatattatttgtaaagtctccttttatatctatgattttatttttttgactcctctttttttcttaatttgtctagttaaaagtttgtcaggttttgtttttgatttttgagacagagtctagctccattgcccaggctggagtgcagtggcacgatctctgctcactgcaacctctgcctcccaggttcaagtgattctcctgattcagcctccctagtggctggaattacaggggcctgccaccacaatcagctaatttttgtatttttagtagagatggggtttcaccatgttgctcaggctggtgttgaactcttaacaagtgatccaccctcctcggcctcccaaagtgctgggattaagtggtgagccaccgtgcctggccaagtgttttatattttgaaaaaacgaagtcttctttgttattcttttgtgttgttttttaaggctctatttaatttatttctgtttttacatttatcgttgcttctcttctactagttttggcttgatgttgttcttgtttttctagttccttgagcaacatgaggttatctgaaatctttctactttttaaatgtaagcctttattgctgtagacttccctcttagaaatgcttttgctgtattccataggttttgttttgttttttttatttttttatttttttattattatactttaagttttagggtaatgtgcacattgtgcatgttagttacgtatgtatacatgtgccatgctggtgcgcggcacccactaactcgtcatctagcattaggtatatctcccaatgctatccctaccccctccccccaccccacaacagtccccagagtgtgatgttccccttcctgtgtccatgtgatctctttgttcaattcccacctatgagtgagaatatgcggtgtttggttttttgttcttgcgatagtttactgagaatgatgatttccaatttcatccatgtccctacaaaggacatgaactcatcattttttatggctgcatagtattccatggtgtatatatgccacattttcttaatccagtctatcattgttggacatttgggttggttccaagtctttgctattgtgaataatgccgcaataaacacacgtgtgcatgtgtctttatagcagcatgatttataatcctttgggtatatactcagtaatgggatggctgggtcaaatggtatttctagttctagatccctgaggaatcgccacactgacttccacaatggctgaactagtttacagtcccatcaacagtgtaaaagtgttcctatttctccacatcctccccagcacctgttgtttcctgactttttaatgattgccattctaactggtgtgagatggtatctcattgtggttttgatttgcatttctctgatggccagtgatgatgagcattttttaatgtgttttttggctgcataaaggtcttcttttgagaagtgtctgttcatgtcctttgcccactttttgatggggttgtttgtttttttcttgtaaatttgtttgagttcattgtagattctcgatattagccctttgtcagatgagtaggttgcaaaaattttctcccattttgtaggttgcctgttcactctgatggtagtttcttttgctgtgcagaagctctttagtttaattagatcccatttgtcaattttggcttttgttgccattgcttttggtgttttagacatgaagtccttgcccatgcctatgtcctgaatggtattgcctaggttttcttgtagggtttttatggttttagctctaacgtttaagtctttaatccatcttgaattaatttttgtatgaggtgtaaggaagggatccagtttcagctttctacatatggctagccaattatcccagcaccatttattaaatggggaatcctttccccattgctggtttttctcaggtttgtcaaagatcagatagttgtagatatgtggcattaattttgagggctctgttctgttccattgatctatatctctgttttggttccagtaccatgctgttttggttactgtagccttgtagtatagtttgaagtcaggtagtgtgatgcctccagctttgttcttttggcttacgattgacttggcaatgcgggctcttttttggttccatatgaactaaaaatagttttttccaattctgtgaagaaaggcattggtagcttgatggggatggcattgaatctgtaaattaccttgggcagtatggccattttcatgatattgattcttcctacccatgagcatggaatgttcttccatttgtttttatcctcttttatttcattgagcagtggtttgtagttctccttgaagaggtccttcacatcccttgtaagttggattcctaggtattttattctctttaaagcaattgtgaatgggatttcactcatgatttggctctctgtttgtctgttgttggtgtataagaatgcttgtgatttttgtacattgattttgtatcctgagattttgctgaagttgcttatctgcttaaggagattttgggctgagacaatggggttttctagatatacaatcatgtcgtctgcaaacagggacaatttgacttcctcttttcctaattgaataccctttatttccttctcctgcctaattgccctggccagaacttccaacactatgttgaataggagtggtgagagagggcatccttgtcttgtgccagttttcaaagtgaatacttccagtttttgcccattcagtatgatattggctgtgggtttgtcatagatagctcttattattttgaaatacgtcccatcaatacctaatttattgagagttttgagcatgaagggttgttgaattttgtcaaaggtcttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggatttggtttggcagtattttattgaggatttttgcatcaatgttcatcaaggatattgattggtctaaaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactcttttttgttggtaaggtattgattattgccacaatttcatatcctgttattggtctattcagagattcaacttcttcctggtttagtcttgggagagtgtatgtgtcgaggaatttatccatttcttcttgattttctagtttatttgcgtagaggtgtttgtagtattctctgatggtagtttgtatttccgtgggatcggtggtgatatcccctttatcatttttttattgtgtctatttgattcttctctctttttttctttattagtcttgctagtggtctatctattttgttgatcctttcaaaaaaccagctcctggattcattaattttttgaagggtttttgtgtctctatttccttcagttctgctctgattttagttatctcttgccttctgctagcttttgaatgtgtttgctcttgcttttctagttcttttaactgtgatgttaggatgtcaattttggatgtttcctgctttctcttgtgggcatttagtgctataaatttccctctacacactgctttgaatgcgtcccagagattctggtatgttgtgtgtttgttcttgttcatttcaaagaacatctttatttctgccttcatttcattatgtacccagtagtcattcagaagcaggttgttcagtttccatgtagttgtgcggttttgagtgagattcttaattctgagttctaatttgattgcactgtggtctgagagatagtttgttataatttctgttcctttacatttgctgaggagagctttactgccaactatgtgatcaattttggaataggtgtggtgtggtgctgaaaaaaatgtatattctgttgatttggggtggagagttctgtagatgtctattaggtccgcttggggcagagctgagttcaattcctgggtatccttgttgactttctgtcttgttgatctgtctaatcttgacagtggggtgttaaagtctcccattattaatgtgtgggagtctaagtctctttgtaggtcactcaggacttgctttatggatctgggtgctcctgtattgggtgcatatatatttaggatagttaactcttcttgttgaattgatccctttaccattctgtaatggtcttctttgtctcttttgatctttattagtttaaagtcggttttatcagagactaggattgcaatccctgcctttttttgttttccatttgcttggtagatcttcctccatccttttatttttagcctgtgtgtgtctctgcacgtgagatgggtttcctgaatacagcacactgatgggtcttgactctttatccaatttgccagtctgtgtcttctaattggagcatttagtccatttacatttaaagttaatattgttatgtgtgaatttgatcctgtcattatgatgttaggtggttattttgttcattagttgatgcagtttcttcctagtcttgatggtctttacattttggcatgattttgcagcagcttgtaccggttgttcctttccatgtttagcgcttccttcaggagctcttttagggcaggcctgttggtgacaaaatctttcagcatttgcttgtctgtaaggtattttatttctccttcacttatgaagcttagtttggctggatatttaattctgggttgaaaattcttttctttaagaatgttgaatattggcccccactctcttctggcttgtagggtttctgccaagagatccgctgttagtctgatgggcttccctttgagggtaacccgacctttctctctggctgcccttaacattttttccttcatttcaactttggtgaatctgacaattatgtgtcttggagttgctcttctcgaggagtatctttgtggtgttctctgtatttcctgaatctgaacgttggcctgccttgctagattggggaagttctcctggataatatcctgcagagtgttttccaacttggttccattctccccgtcactttcaggtacaccaatcagacgtagatttggtcttttcacatagtcccatatttcttggaggctttgttgatttctttttattcttttttctctaaacttcccttctcacttcatttcattcatttcatcttccatcaatgataccctttcttccagttgatcgcatcagctcctgaggcttctgcattcttcacgtagttcttgagccttggttgtcagctccatcactcctttaagcacttctctgtgttggttattctagttatacattcttctaaatttttttccaagttttcaacttctttgcctttggtttgaatgtcctcctgtagctcagagtaatttgatcgtctgaagccttcttctctcagctcgtcaaagtcgttctctgtccagctttgttccattgctggtgaggaactgcgttcctttggaggaggagaggcgctctgctttttagagtttccagtttttctgttctgttttttccccatctttgtggttttatctattttggtctttgatgatggtgatgtacagatgagtttttgatgtggatgtcctttctgtttgttagttttccttctaacagacaggaccctcagctgcaggtctgttggagtaccctgccgtgtgaggtgtcagtgtgcccctgctggggggtgcctcccagttaggctgctcgggagtcaggggtcagggacccgcttgaggaggcagtctgcccgttctcagatctccagctgcgtactgggagaaccactgctctcttcaaagctgtcagacagggacatttaagtctgcagaggttacagctttttgtttgtctgtgccctgcccccagaggtggagcctacagaggcaggcaggcctccttgagctgtggtgggctccacccagttcgagcttcctggctgctttgtttacctaagcaagcctgggcaatggtgggcgcccctgcctcagcctcactgccgccttgcagtttgatctcagactgcggtgctagcaatcagctagactctgtgggcgtaggaccctccgaagcaggtgtgggatataatctcatggtgtgccgttttttaagtccgttggaaaagcgcagtattcgggtgggagtgacccgattttccaggtgccgtccgtcacccctttctttgactaggaaagggaactccctgaccccttgcacttcccgagtgaggcaatgcctcaccccgcttcagctggcacacggtgtgcgcacccactgacctgcacccactgtctggcactccctagtgagacgaacccggtacctcagatggaaatgcagaaatcacccatcttctgcgtcactcaggctgggagctgtagaccggagctcttcctatttggccatcttggctcctccccctccataggtttttgtgtgttgtgttcccatttccattttttcaagaaatattgaaattttctttttgatttttttcactgaaccactggttgttcagctgtatgttgtctaatttccatgtatttgtagtttccaaagttcctcctcttattgatttctagttctataccattattgttgaaaaaaacattttttgatttttaattttttaatatgttaagacttttattgcagcttaacataagatatatcctggagaatgttccatgtactgtggagaagaatgtatattctgcaactactggatagaatgttctttaagcatctgttaggttcatttggtcaggagttcagtttaactctcattttttgttgatattctgtctggattatctgtttatcactgaagataagatgttcaagtgttatactattgttgtattgtacgctatatctccctgtaggcctattagcatttgctttatatatttgtatgttcaattgttgggtacatgcatatttataattgttatattctctttctgtatttacccctgtatcagtttttaattagcatatttgtctttttttacagtttttttggcttaaagtctattatgtctgatataagtatagctacttctgctccattttgctctccatttgcatggaatatctttttctatcccttcactttcagtctatttgtgttcttctgcgtaaagtgaggctcttataggcagcatatagttgagtctttaaaaaaatcaatttagctactttatgttgtttaataggaaaatttaatccatttacattcaaggtaactaccaataggtaaggaggtactactgacattttgttacttctttcctagttattttgtagatccttccttcttttctaccactcttgctatatccttttgtggttaagtgatattctccaacattatgctttgattcactgctgtttatctttagtgtatctattataggtttttactttgtggttatcatgaggctttgaaatacatattttgtttacatcaagttgttttattttattttaaatttcctacttttattttaagtccaggggtacatgtgcaggatgtataggcttattacataggaaaacgtgtgccatggtggtttactgcacagatcatcccattacccaggtattaaccccagcatccattagctattcttcctgatcttctccctcctctcatccaccacactcaggccccagtgtgtgttgctccccaccatgtgtccatgtataacaagttattttaagctaataggaacttaattttaatcagaaaagaaaggaaacaaaaatattgtacactctaactcctttcttctcaagtattttgaattttcatgtcccaatttacatcttttgtatggtttatcttttaataaagtattttagttatcatttttaagagctttgtatttttgtattcatactaaagttataagtggtttacataccataattacaatgttagatatcccaaatttatctgtattcctacatttattcatttatttaaaggcagggtcttactctatcacccaggctggagtgctgtgactcaatcttggctcactgcaacctctgcctcctaggctcaaggaattatcctatttcagcctcttgaatagctgggactacagacacaagccaccatggctggctatttattttttttctgtttttttttttttgtgtgtgtgtgttttctcgtaagatgcggttttgccatgttgcccaggctgaactcaaatccttgggttttttagtcaatccacttgcctcagctttctaaagtggtggggttacagccatgagctaccatgcctggcctgtatacttacctttaccagtgagttttataccttttgatattttcttgttacacattagcattttttttctttcaagttgaagaacttcctttgttatttcttgtaagacagatctggtggtgttgcatttacttggcttttgtttgtctggaagagcctttatctctccttcatttctgaaggacatatttgccagttactttattcttggttggcattctttttctttcagcattctgaatatattgccctacactttttggcctgcaaggtttctgctgaaaagtctgctgccaggcatatcaacattcccttacatgttatttgcttcttttctattactgcttttagaatgctctgtttttatttcgagagtcaaagggtatgtcttgaggtagttccatttgtgttgaatatgattgatggcctttgaccttcctttacttgagcatttacatctttttccaggattgtaaaattttcttttatgatttctgtgaataagccgtctatttccttgtctttttctgtaccctcttgaactccaatgacccaagtatttgctctttcaatagtatcccatagactccatgaacttcacttgtttctttttttttattctttttcccactttggccatatttcaaatagtctatcttagatttcactgatttttttttcttctgcttgattaattctgctgttaatgctcgtattgcatttcttatttcattaattgtatcttttagctctaggatttctgttagatttttcattactttagtccctgttaaacttctctgataaatttctgaactgcttttctctgtgttttactgaaattcactaagcttccttaacaagctattctgaattccttgagagataatacttctgcatcactttagggtttgtcactggcatcgtgttttgtctgtttggtgaggtcaaaattccctggttgttcttgatgcttgtgtatgtacattgatgtatttgctctgaaaaaaggtatttatttcagtcttcacagtctggctttgtttgtgcctgtttttcttcactgggcctctccagaatttaagcaaacctaatgttagatcgcatgactgctgcagccatttcagcactagaggaaactctaagcctaggttcactgtgagtctcatgagggctgtcaggttggcatggagcccttatgtagaaggacgtggtgaagacccaaggagggtgcccaagctgtgtgaggaagctggccagggacctaagtccaggagcctgtctcattggctcagatgtgggaggagctggagctgagactgggtcccctcaggatctgctttgggacaaaggctggtggtcccaccaaggagtttcagaaaaatgggtgattgcctggctatgagataagggtaagtctttctctaggtatccttgtgtgagcagtaataaactcagatcacagctgagtgggtctggagccaaattacagggtaagtttcagattcactgccaacatttaacggacagaagagattttctgccaagttattagtgtgcatgattcctcctagacacattggtagatgtttttggttcaagactcaagacaaaattggagtgtagccaagccttttgttaacaaggatgtctccaagtttgaaacctgcagtatcattggtgggtctgctatccaggtgctggtctgcaatcccaaagctgcctacctagggccttgattccactgaattttcacagattactccctgattcctgaggcttctacagagagacttttgtctgtggatgagtagataattcttggagttgggggatatgagtgggttacctcctattccaccatctttctgacatagtgtttgtatttctattctgactataacatatatagtgggattagatgaatacataattacattgattcatcggaaaccaagattttccgtgtgggagaaaagagaagtagaagtgagactgatgagttaaacacatagttttgaatttgaagtgaaagtgtaaatatgagcttgtgatgatatttatcctcaaaaaaatttccacaggctaatttttttttaggttttcccccttagaaggcctagaaactaggattaacccagtaatttccgtatcacattttttccaactaacaaggaccagagatccttgctaatttctagtgtagggcaagaagtttcggcaagaaccttgagcaactttgccacatcaggaagtaatacagtcttgatgacagaggtgtattgatggagctcaggaagaattgacttgaaaagaatctcattgtcacagataggacaatttgagcttcaataaaacaactactatgatagatacaagcatttcaaataagctgaaattcatgagtatacaattatgcaaaaaaatattgatcacttgtggagaatgccatataaagcattcattctcttgagaacgggcaaataaatagaaagaataactatcttattaccttgattccatcaaggtaataagatagttactgaagataatgtctctttaggaatattctaagtaataagtgaagaatgtttaatatatttttctttcacccctaggccattcattcataagatatgacccagggcactttgaggtccctgactccttttctgagggtccatagttcccaataaatcctaacaatactaccaggttaaatttttaattctattcttctcatgattttatgttgttctccagagactacatggaatatgatattgcatcagattggatgccaaagcagatatgaaaatgtagctgttttcttattaagccagacattaaagagaattgccaaaatactaaataaataaataaatagatgcagctgtaaagccacgcttctcactacatttttgaaaaacatttaaaaataaagatgtaatttttgttgatatgtaatccatgtttaattgtagagaagtttataaatattttaattattagtttattagtttttacttctaagatatatctagctatctgtctggacatacattttatacacacacacacacacacacacacgcacacacacaatttccatcacacagagaaaagcttttctggtgatctcaatctttttttattgtggtaaaatacacatatataagatttaccattttgccatttttaagtgtacaattctgtgttattaagtacattcacactgttgtgccaccttcaatataacttgtacatatgtaaaatgtgtaagagttgagacctattaatctagtaatgaatgaagaccattaagcattatatgcctcttgtgagatatacatagtataataaaacctgaatctgagcacatcacaaatctatttatttattttttattttttcttgagacggagtcttgctctgttgcccaagctggagtgcagtggcgcgatctcggctcactgcaagctccgcctcccgggttcatgccattctcctgcctcagcctccctgagtagctgggactacaggtgcccaccatcatgcccggctaattttttttgtagttttagtagagatggggtttcaccgtgttagccaggatggtctcaatcaactgacctcgtgatccgcccgcctcggcctcccaaagtgctgggattacaggcgtgagccaccatgcccggcccacaaatctatttatatatagtaaataaagagaacaaaggggcatgttaaatgacattatagctatgtaatcagcaaaatccaaactgtggatgattttacaagaagaatgatgaaatttcttaaaaaacaaattctaggataaaaatgagagaagaatcctacagattaaaaacggtttatgaaacatcaactaattttaatgcatgaatcttatttttattccaatttaatgaataaactgtaaaaacaattcatgagacaattggaaaaaatttgaatgctgaacaactatatatttaatgatattaagaaatcttttatttggggatgtcataatgtaatttcggctatatgttaaaaaagtctatatgttacagaaagacatgttgaaatagttaaggatgaaatgatacaatatctaaaatttattttaatcatgagttgataattttttcaaaactgtctaatgagcaaaggatagtttttatactgctttatgtttgcacatataaaattattattattattattattatttttggagacagggtcttgctctgtcacccaggctggagtgcagtggcacaatcttggctcactgcaacctccacttgctgggttcaagtgattctcctgcctcagccttccaagtagctgggactacagccatgcaccaccatgcccggctaatttttgtatttttagtagagacagatttttgccatgttggtcagcctggtctcaaactcctgacctcaggtgatccacctgcctcggcctcccaaagtgctgggattacaggcgtgagccaccacgcttggcccctataaaattattaattaatggaccagcgctggctggtccattttacagagtgcagattggtctattttacagagcactgattggtgcattttacaaacctcttgctagctacagagcactgattggtgtgtttttacagagcattgattggtgcattttacaaaactcttgctagctacagaaaagttataggagtccccactcaacccagggagtgcagctggcttcacctctcactatgaccctttaaaagctagactatagctgtagtaagccaagggtggttttctccaaaggagtttggaacagaaataaagaaaacataatattttttggctaagtggaaactattttagcaagatgaggagaatctacaatatcaactaaaaagaaaaaaaaatggaagaaagtagatgactctgaaagaaaacacttaacttggcagttacagcaactcagatagtttttactgagaaaatgaatttgcagctaagagatcttgccagagatctacaaatagggaacactgtcctcaaggaacttacaaagagaaggggaggaaaaacaaaaataactccaatgctgtgagtaaagtgtaaagtggatctctgcatagttccttttattatagcactaggaaagtcttcctgaggaagaaacatctcagctgagccttcagggctagaaattcatccaggcagagacagagaagaaattacatccaaaaggctctgcatctctgcaaatacagtgattctggcataattatatttggttttctttgacaaatgatatttaaccagaagcagaaaaagaaaagttttaaattaggaagccaaagggagtcaccattaagtcatatgtattatatttcatttattttgagacacattttttctaacattttaatgtgtgtaaaatcagggtgaatcacactgttagtacagtctcataataaatttacagttttaagtacttttttgctgatgtttaaataataatttattttaaaatacatgaaaactctgatttcatcaaatttgaaaatcatttgaaatatgataagattgacttttgggctgatatcttgggcttcttggtacttattgcgaatgggagaggagacatgatgatggctcatgggatattctgatatgacctgttaaaaacagaagaacaaaagagctccacataaaagatgtttgtacacccatgctcatagtaatattattcatgatagttaaaagtgaaagcaacccaagtgtccatagatagacgaatggatgaacaaaatgtgttatacgcatatggtggaatattgttcagtttcaaaaaaggaaggaagccctttcatatgctacaatgaggatattacgctaaatgaaataagtcacaaaaagacaaatatttacaattccacttatataaggtacctagaatagtagaattcatagagacagaatgtagagctgtcctaaccagggcctggagaagaggggaaatgggaacttgcagcgtaataggaatagagtttcagtcttgcaagacagaagagttttgaagattgcttgcacaacaatgtgaacacacttaacaccactgaactgtacacttaaaaatggtaaagatggtaaaccttatgttatgtgtattttaacagaattttttttaagtccacataaaacgaatgggagtttggggaaaggatatggtgaaaggattgaggataaacaatttttaaatagataaaatggtaaaaaacagtggaagggtgaacagaaaagacatattaaaaaaaaaaacaacaccacccacaactcaccggcgcctcatataccataatgcaaggcatagcaaaaggagcaaggaaggcactattattgccaaaacaattgagccaatggaggtggggtttcctggcaattgagagaggacaaaaggatgtcacttcagatactatacacagaattccttggccttcctttgcccacctacttccaatgtatggtcatttccaacttcctctctcatattcctggccaccatatatccatcctttgatcccctctacccagtttacaggtccttaattcctacatttttgataacttggattatctatttttactcctgttgggattggggtcagggaaatcaatcaatcaatctctccctctttcatctctcaagctctatccttccccagtgcctccctctatgcctgaagatcacttcctggcatttccagcctgggtccctgctatttcttactccagtagaggatgatgtcctggccctctaaactgctgtgcttcacccgacaggacaggccagccgcctccccatctgccacatccagggttgctcggagataccatgtccagttagcattgggcaggatgtcccctagctgagtgccctgctgctcctgctcaccccgcatccacatcacccacacgggctttgggtagaatcctgagacatggcacacaagctgcagacggccaggtccaggactggggccactggacagccaggcctcaggcttcactaaggcaggaaggagaaaaaaaagtgtcatgttataactcgagttcagaggttatgaactcagaaacctacaagcttggacagcgaccccattcactcctttgggaaccaaataacttgttatttaagctcctatcctttgatatcttcctactcttgatcttagaggaggtggtgggaagtgaaataacagcaggactaaccttgtctttgcagatctgcttttcctgcattgaggacgcccaagagatatcgggggcaggtttcatagaggagaattctcacagtttccatgataccttgatattgtatgattagtgcacagaatttctgtgccctgctgccaccttctggggaaggcacacatgaagcattcttgacactcaggaaatccaatcctcctagagctcccctcaggaagcttactatggcacctccagaatgtagctcacagcctgctatgccctggatctcaaaggggtctatgtagagggaaaagagagcaagttattaaacacaaataagaaaaaaaggcatgagaaaagaacctaggattttagattttggtggggataaagttatttggtgtaaaatgatgatgagctaaaaactaatgataacatttagacattttcagaaacaagagcattgagaaaaatgtgcatgccatgaagacaaaggtatgtgataaaggagtacagtgagtatgaaaggagaggtgaggcagagcgtggtgcagtcaggagtgcagcattccagagtgagtggaggttcccagcaagaggagggtgcggggaggagatgattgaaaagatggattaggggagaaagccacacgttagatcactcaggcacagggtaggagaagtaagggcccttgaggagaagtttcacataagacaaaccagcagctggttacaaagtggaagacagtctgagagtaagagaagagtgcagaggcagcaagaatctgaaatatgaggtgtgatggtgtgaagctggagtgagagactactcaagaaaagcatgtgcgtgcatttgggtaaaatagaaaggaagggagaagcatcagagagagcaacactttgcaaaaaagagagaaaagagagtccttgagactcttcccagttcggtggactagactcacatttcatctggaaatcaccggcaaagtcttgtacttctcgagcgaatccaaagatgtagactcggaatatctcctctaactcagcaacctccttatcactaaagttacctttagaccaaggcttcaggaatatggcagtgcctgagtcgctatcccagccatgaatctgcaaatcatccaaccagcctgagccttgagtttgtgcccaggtactattggtaaaggacgaggtctggataacatgaaaggaggtcggcccctggaaggctgtgaagagtggaaaagcaagatggtgaagataaagagagaagcaggagacaattaggaagaatgggaatgaaaatgatttagaaaacaagaacctagagtctaaagaacacaggaagtctgacacatttgactgccttaaggcttcaggttctagtcccaaccaccagaatggttgaagagggcgggagacagaaagacccagacccttagtgtccaattgcacctaggaaagccctgcttgctttttaaaatccaaacccctgcaccagtgctgggagctgccagagtgactcttaccatgttcactgttaccaccaggaaagagaacagctaacagttgaaatggcagcagcagcatttcactgggagatgcaacttcttactggcagagctggtatttgatctccaatttcagcaaagcttttcctcagtaccctgtagtgacttcttctctcttccaactgccaaatctcttcctcatccaaactgcttttcagagaaaaatgtgcctcccacagccctgagcctcttacctcaccttttatttccctcaagccctgatttttagagtttctcttaccccctgacttccagccgctctccctgtcctcaccttccttcatacctccttggaaaagcccccatatgattctgcattttcatttctccttccttagcctttattcccacatctataggataagatggtgatattaagcaattgctaagcttttagtctgctttaacccagaaggatgcttttagtttatccacctcacttagtttctccccctcccagctgccttatcttctctggcactctgttactgcctcttcccaatgttttcaatgtcttctctttcttatattgccctctttgtctggtgttccttaccccacattctaaactggctgtgagtgtgtgtgtgtgtgtgtctgtgtctgtgtgtgtatgtgtatgtgtgtgtaccgatattcacttacataaaggcaaagcatttaaaacatttcctgtactatagcactacaaaccaaaatgccagaatcaaatataataaagactgaagtttataattcaaacaaaattatactatagcaatatttgagggaaaaaatgacaattgctgctagcatgtgaccttctcttcaggagctcttggcctatggagttttgagtaacaaatattcatgacataaacagaactctaggagctatgctatgtgcacctccagcctaagtcaaagataactttttgggtaagttgccataagcggcagagcttttttcttctcttgtaatatctcataactgctgtctacatttgggcattcatatcaccacatgtggtattaataatagagcaaagaggttgcttttttttatacaaaaagttcagagatacttgttttaagatgtcacctcctatcttcttgtatgcccatgaaaaattttaagaagaatttttggattttctccatttaggaaaaaaatcacatatttgcagagatctatgtatcatgcagaaatgtggcagcaatcgattccgagtaaagagggttatcactaactaattctattatgtggataagaaaatgcaaagtgagtagtgtagtgttctctattcctggggacactactggttcacaacattaataagactggttcacaacattaataagactagccttcatccaagattattactgcctaattgataaatacagagcagctctccaaatttcaatgtatgtatctcttaacaacattcccagactccaatacatgacagaattgaatctactttttggataacagccatagacaacagcagagacttaacattggctgtcacagacatgaacattactatttttgtgtttatacgttttttattttgggtagaacacccaaaacagagctcaggaaaagctggtagacaacctgggctattttccattaaaataagtgagttcttgcaagactcccaaagttgaattgctacaaactaaatctgaaatgttcctcattttaaaaaataatccaaaagagttggcagggcacaggagactatcacaacactagccccacactcatcaaacttttttttttcttgagacagagtcttgctctgttgcccaggctggagtgcagtggcatgatctcggctcactgcagcctctgcctcttgggttcaagtgattctcctgcctcagcctcctaagtagctggaactgtaggcatgtgccactatgcccagctaatgtttgtatttttagtagagactgtgtttcaccatgttggccaggatggtcttgatctcctgaccttgtgatctgcccacctcagcctcccaaagtgccaggattacaggcatgatccaccacgcccggccactcatcaaatttttaaacaagttaggcctaaaacatgagaacagtaatccagtcaaaattatgttaccagaaaggaatattagtattgccaaagactgagttagaaattgagttgtgcctgagagatggaagatagaggaagaggtcttggtttcagtttcaacagcgtgtaagtttttgccaaatatgtgcagaatgtctacctaaataacaaacagaaaaagaggctctctaaaagaaaaagatatttatttgagaatagagcattgcaatgggaatatatatgcaatagtaaacgatgtgcatactcagggaggtaaaggaaggcaaattttttaaagaaaaaaaattaagagaattacatatttgttttgaaataattatccttgcctacaaatatcaataacaacggtgacaccagtcagaggttaaacagacagttgctggcagggcagatgtctttgaggcatttttcatgtaaggttgtgatggccttttcaaggttgtggtttttgcagagtctttcgtggtagttgttcttattaggtatacaagtgtgagaaccctctctgcatagtcttccctagctctgccagttttttttttttttccttagtgactccgttttgtttctggcaacatttgcaagaagatgactgtaaatttaaatttagttaggaaaatcgccagacaaaatgaaaattttgtaattcttcaaatgtcatatttgagggaaaaatttactttatacactgtcttttccctgaaaagtggagcaattgagatagtttgaaaggtctagaaattaaccaggttttcaagttacattctttctcacatatcatatccagtatttcatatttcctttctgtttcaaaagtacagaaatggacatggcagtgtccacagtgtccacatagcaatgtcacacatttccatactacactgtgactatgcttgtccagctctatggcatggtttgtatttccactctgcattagtaccattgttgcttgacatatgaaacctgtcttctagggaaagattatccctagtcccaatatttgtttcaaattaaaaaatcaacatgtgcacacttctaaatcgaagctttaattgatgcttctaactcatactctcctatgggcctaaagaccatgtacacttctgataagctcatcaagacacaggttttctcagatattgattgcataatctcacttatatgtggaatcttaaaagagttgagtacacagaaatagagaataaaacagtgattaccaggggcaggaagcaggtggtggtgggaggaaatgaggagatgtaggtcaaagaatacaaagtagccgatgagtagtcccttctgaattgcagaggatacattccaagacttctggtggatgcccaaaaccacaaatagtgctgaacctgattgctgccaattggagcccctttctgttcatgtcctccacccacaaatttgatggctttttaagatcttaactaagcacttaccacacactctgatcataacctttgcaatttgaggtgtgatggtaaaatttgtacaattcccttttccccatcacaattttatggaaagaagattcctttttactgtagaacatagcaacatcagcatattttgtttctttccttattaagtcaaaaacttaacaccttttcatttaaaggaagcacttaatggcttctctttggcatatttgaattacaagcatcactacttctgtgctttggggctgttattaagtaaaataagggttacttgaacacaagcactggagaccaggacagctgatctgataactgagccagctactaagtgactaagggttgggtagcatatacagtggagatatgataattaaggggtgatttgcatccttgtccagatgaagtaggctggcctgaaatttcatcatactacttagaatggtgcacaattgaaaacttatagattatttatttctggaattgcccatttaatattttcagactgccattgaccatgggtaactaaaactgtggaaaacaaaaccatgaattaaggaggggtctactgtatattataaataaaaaaatattttgaccttgctaccagtatccatgttaaattaacttattacatttaatattttatctatagatccttttggattttctaatgatatctctaaatatgaaagtttcattagatttttttcaattgttataccttttattatattttttgcccattttccctggcctatgacctccagtaaaatgataaatagaattattgatagctactttttttttttaaacagagtcttgctctgtcactcaggctggagtgcaatggtgcgatgtcagctcactgcgacctccacctcctgggttcaagcaagtctcctgcctcagcctcccaagtagctgacattacaggcatctgctaccatgtgtggctattttttttgtatttttagtagagacggggtttcactatgttggataagctggtctcaaactcctgacctcaggtgatccacctgtgttggcctcccaaagtgctgggattacaggcgtgagccaccgcacctggcccagataagattttttaagaggttaggtggttccaagatggctgaagaggaacagctccagtctatagctcccagcgtgagtgacgcagaagagggtgatttctgcgtttccaactgtggtacccggttcatctcactggggcttgtcagacagtgggtgcaggacagtgggtgtagcccaccaagcatgagacgaaacagggtgaggcatcacctcaccagggaagtgcaaggggtcagggaattcccttttcaagccaagggaagctgtgacagatggcacctggaaaatcgggtcactcccgccctaatactgagcttttccaatacttagcaaacggcacaccaggagattacatcctgcgcctggctcagagggtcccatgcccatggagcctcactcattgctagcacagcagtctgagatcgaactgcaaggctgaagcaaggatgggggagaggcacctgccattgctgaggcttgggtaggtaaacaaagtggccaggaagctcgaactgggtgtggcccaccgcagctcaaggaggcctgcctgcctctagagactccacctctgggggcggggcatagctgaacaaaaggcagcagaaacctctgcagacttaaatgtccctgtctgacagctttgaagagagtagtggttctcccagcacggagtttgagatctgagaatggtcagactgcctcctcaagtgggtctctgacccccgagtagcctaactgggaggcaccccccagcaggggcagactgacacctcacacggctgggtattcctctgagatgaagcttccagaggaacgatcaggcagcaacatttgctgttcagcaatatctgctgttctccagcctctgctgctgatatccaggcaaacagggtctggagtggacctccagcaaactccaatcaacctgcagctgagggtcctgactgttagaatgaaaactaacaaaagaaaggacatccacataaaagccccatctgtacatcaccatcatcaaagatcaaaggtagataaaaccacaaagatggggaaaaaacagagcagaaaaactggaaactctaaaattcagagcacctctctccctccaaaggaacacagctcctcaacagcaatggaacaaagctggacagagaatgactttgacaagttgagagaagaaggcttcagatgatcaaacttctcagagctaaaggaggaagttggaacccatcacaaagaagctaaaatccttgaaaaaagattagatgaatggctaactagaataaccagtttagagaagcccttaaatgacctgatggagctgaaaaccatggcacgagaactacatgacgaatgcacaagcttcagtagctgatttgatcaactggaagaaaggatatcagtgatggaagatccaatgaatgaaacaaagcaagaagagaaatttagagaaaaaagaataaaaagaaatgaatgaagcctccaagaaatatgggatgatgtgaaaagaccaaatctatgtctaactggtgtacctgaaagtgatggggacagtggaaccaagttggaaaacaatctgcaagatattatccaggagaacttccccaacctagcaaggcaggccaacattcacattcaggaaatacagataatgccacaaagatactccttgagaagagcaaccccaagacacataattttcagattcaccaaagttgaaatgaaggaaaaaatgttaagggcagccagagagaaaggtcaggttacccacaaagggaagcccatcagactaacagctgatctgttggcagaaactctgcaagccagaagagagtgggggccaatattcaacattcttaaagaaaataattttcaacccagaatttcatatccagccaaactaaggttcataagtgaaggagaaataaaatcctttatagacaagcaaatgctgagagattttgtcaccaccaggcctgccctacatgagctcctgaaggaagcactaaacatggaaaggaacaactggtaccagccacgcaaaaacatgccaaattgtaaagaccatcgatgctaggaagaaactgcatcaactaacgagtaaaataaccagctaacatcataatgacaggatcaaattcacacataacaatattaaccttaaatgtaaatgggctaaatgctccaattaaaagacacagactggaaattggataaagagtcaagacccatcagtgtgctgtattcaggagacccatctcacatacagagacacacatgggctcaaaataaagggatgggggaagatctaccaagcaaatggaaaacaaaaaaaaggcaggggttgcaatcctagtctcggataaaacagactttaagccaagaaagatcaaaagagacaaagaaggccattacataatggtaaaggggtcaatttaacaagaacagctaattatcctaaatatatatgcacccaatacaggagcacccagattcataaagcaagtccttagagacctacaaagagacttagactcccacacaataataatgggagactttaacaccccactgtagacattagacagattaacaagagagaaagttaacaaagatatccaggaattgaactcagctctgcaccaagtggacctaatcggcatctacagaactatctgccccaaaccaacagaatatacattcttctcagcaccacatcacacttattccaaaattgaccacatagttggaagtaaaggtctcctcagcaaatgtaaaagaacagaaattgttacaaactgtctctcagatcacagtgcaatcaaactagaactcagtgttaagaaactcactcaaaaccactcaactacatggaaattgaacaactggctcctgagtgactactgggtacctaatgaaatgaaggcagaaataaagatgttctttgaaaccaatgagaacaaacacacaacataccagaatctctgggaaacatttaaagcagcctgtagagggaaatttatagcactaaatgcccacaagagaaagcaagaaagatctaaaattgacatcctaatttcacaattaaaagaactagagaagcaagagcaaacacattcaaaagctagcagaaggcaagaaataactaagatcggagcagaactgaaggagatggagacacaaaaaaaccttcaaaaaatcaatgaatccaggagcttgtttttggaaaagatcaacaaaattgatagaacgctagcaagactaataaagaggaaaatagagaagaatcaaatagatgcaataaaaaatgaaaaaggggatatcaccaccaatcccacagaaatataaactgccatcagagaatactataaacacctctacgcaaataaactagaaaatctagaagaaatggataaattcctggacacatacaacctcccaagactaaatcaggaagaagttgaatctctgaacctaccaataacaggctctgaaattgaggcaataattaatagcttactgaccaaagaaagtccaggacctgacggattcacagccgagttgtaccagaggtacaaagcggagctgctaccattccttctgaaactattccaatcaatagaaaaagagggaatcctctctaactcattttatgaggccagcatcatccagataccaaaacctggcagagacacaaccaaaaaagagaattttagaccaatacctttgatgaacatcgatgcaaaaatcctcaataaaatactggcaaaccaaatccagcagcacaccaaaaagtttatccaccacgatcaagtgggcttcttccccggggtgcaaggctggttcaacatatgcaaattaataattgtaatccagcatataaacagaactaaagaaaaaaaccacatgattatctcaatagatgcagaaaaggcctttgacaaaattcaacagcccttcatgctaaaaactctcaataaactaggtatttatgggatgtatctcaaaataataaaagctatttatgacaaacccacagccaacatcatattgaatgggcaaaaattggaagcattccctttgaaaactgtcacaagacagggatgccctctttcaccactcctattcaacatagtattggaagttctggccagggcaatcaggcaggagaaagaaacaaagggtattccattaggaaaagaggaagtcaaattgttcctgtttgcagatgacatgatcgtatatttagaaaaccccatcttctcagcccaaaatatccttaagctgataaggacattcagcaaagtctcaggatacaaaatcaatgtgcaaaaatcacaagcattcctatacaccaataacagacaaacagagagtcaattcatgagtgaactcccattcacaattgcttcaaagagaataaaatacctaggaattcaacttacaaaggatgtgaaggacctcttcaagaagaacttcaaaacactgctcaatgaaataaaagaggacacaaacaaatggaagaacatttcatgctcatgggtaggaagaatcaatatcatgaaaatggccatactgcccaaggtaatttatagattcaatgccatcctcatcaaactacgagtgactttcttcacagaattggaaagaactactttaaagttcatatggaactaaagaagagcccgcattgccaagacaatcctaagccaatagaaaaaagctggaggcatcatgctaccttacttcaaagtatgctacaaggctacaataaccaaaacagcatggtactggcaccaaaacaaagacatagaccaatggaacagaacagagccctcagaaataataccacacatctacaaccatctgacctttgacaaagctgacaaaaacaagaaatggggaaaggattctctatttaataaatggctctgggaaaactgtctagccatatgtagaaagctgaaactggatcccttccttacaccttatacaaaaatcaattcaagatggactaaagacttacatgttagacctaaaaccataaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaatttttgcaatctactcatctgacaaagggctaatatccagaatcgacaatgaactcaaacaaatttacaagaaaaaaacaaacaaccccatcaaaaagtgggcgaaggatatgaacagacacttctcaaaagaagatctttatgtagccaacagacacatgaaaaaatgctcatcatcattggccatcagagaaatgcaaatcaaaaccacaaagagataccatctcacaccagttagaatggcaatcatcaaaatgtcaagaaagtacaggtgctgcagaggatgtggagaaataggaatacttttacatggttggtgggaccataaactagttcaaccattgtggaagtcagtgtggcgattcctcaaggatctagaactagaaataccatttcaccaagccatcccattacttgctatatacccaaaggattataaatcgtgctgctataaagacacatgcacacatatgtttattgtggcactattcacaatagcaaagacttggaaccaacccaaatgtccatcagtgatagactggattaagaaaacgtggcacatatacaccatggaatactatgcagccataaaaaatgatgagttcatgtcctttgtagagatatggatgaaactgaaaatcatcattcccagcaaactatcggaaagacaaaaaaccaaagaccgcatgttctcactcataggtgggaattgaacaatgagaacacatggacacagtaaggggaacatcacacaccgggggcctgtcgtggggtgggggaaggggggagggatagcattaggagatatacctaatgttaaatgacgagttaatggatgcagcacactaacatggcacatgaaaacctatgtaacaagcctgcatattgtacatgtgtaccctagaacttaatgtataataataaaaaaaaagaaaatgaagatgaggaggaaaaaaattaaaaaaaaaaagattcctttggattttccaagtatacaatcaaaaaaatttaagaggttaacaagtgtcttattttctgggagtgttttgtgtaaacttttcttaccagattaagaatattctcttctaaattagtttaaagtttttttattattattatggagaactgtttatataggtgcttatattattttattttattatgtacttatttattcatttaatttttttttttttttgaaatggagtcttgctctgtcgttcaggctggagtgcagcggagcaatcgcagctcactgcaaactccgcctcccaggttcaagcgatgcttctgccccagcctctccagtagctgggactacaggtgcccaccaccacgcctggctaatttttgtgttttttttttttgtagagatagggtttcaccatgttgtccagcctggtcttgaactcctgagctcaagtgatccacccgccttggcctcccaaaatgctggggttacaggtgtcagccactgtgcctggccaggtcttacattttaattgttaatccaccgtgagttaatttttgtaaatggtgaaaggcagaaatgcagttttattcttctgcatatggttatctgttttcccagaaccatttattggatattgagttatttatcaattactcatttttgttcactttgttaaagaacagttggttgaaaatatgatgctttatttttgatttctctattaagttccattggtctatcttgttaatttttgtatcagtaccatgctgttttggttattgtagcctaatagtaaagtttgaagtcaggtagtgtgattcctctggctttgttcttttccctttggattgctttggctatctgggcttatttttggttccatatgaattttgggatagtttttttctaagtctgtgaaaaatgatgttggtgatttgatacaaataacatcaaatctgtagattgctttgggcagtatgaacattttaataatattgattcttcaaatccatgagcatgaaatgtttttccatttgtttgtgtcccgtatgatttctttcagcagtgttttgtaattctctttggagagatcttttgcccctttcgttaaaggtattcatagatattttactttttttggtggatactgcaaatggggttgcattctttatttggctctcagcttgaatgttattggtgtatagtaatgctactaggtttagtacattgactttgtatctgaagttttattgaaattgtttgtccactccaggagacttttggtggaatctttagagttttctacagataaaactatatcatcagtgaagagaggtaatttgacttcctatttagttatttggatgctttgcattttgttctcttgactaattgctttggcttggtcttccagcactatgttgaataagagtggtgagagtgggcatccttgtcttgttccagttcttaagaggaatgcttttggtcattcagtatgatgttggctatgggtttgttgtatatggcttttattattttgaagtaagttcctttgatgcctggtttgttgacggtccagggacttttttggttggtaagtttttattgctgattcagttatggaactcgttattgctctgtttagcgtttcaaattcttcctggttctatcttgagagattatgtgtttgcaggaatttacccatttcctctagattttatagtttgtgtgcatagaggtatttatagtagtttctgggggatcttttgtatttctgtgggatcagttgtaatgtcaccttttccatttctgcttatgcttatttggatcatgtctctttctttttttttaacacttttatcttaagtttaggggtacttgcacaggcttgttacatagataaacttctgtcatgaaggtttgtggtacagattatttaatcacccaggtattcagcttagtacccactagttttttttcctgatcctctccctcctcctaccctgtaacctccaataggccccagtgtgtgttgttcccctctatgtgtccatgtattctcatcattttgctcccacttatgagaacatgtggtattttgatttctgttcctgtgtagtttgctaaggataatggcctttatttagctccatccatgtcctataaagggcattatctcattcctttttgtggctgcatagtattccatggtgtatatgtaccacgttttctttatccagtctatcattaatggacatttaggttgactccatgtcttcactattgtgaataatgctgcaataaacattcacgtgcatgtgtctttataatagaacaatttatatttccctgggtatatacccagtaatgggattgttgggttgagtggtatttctgtttttagctctttgaggaattgccccactttcttccacaatggttgaactaatttatactcccaccaacagtgtataagtgttcctttttctccactacttcaccagcatctgttatttttggactttttaataatagccattctgactgatgtgagatggtatctcattgtggttttgatttgcatttctctaatgataagtgatgttgagctttctcttatatgattgttggccacatgtatgtcttctttcgagaagtgtctgttcatgtccttttcccactttttgatgggactctttttcttgtaaatttatttaagttccttataagtgctggatttaaactttattagatcttgttgagcttccttatcatccagattctgagtcctatatctgtcatttcatacatttcagtctggttaggttcattgctaaggttcattttcaaggtaacaaaacacctcggactttttgaattgctggagttattgtgttgattccttctcgtgagggagctggcacttctttttctttttgaatttgttgccatttggatgaggtttatatatacatatattatttttatatatatatatatatatatatatatatatatatatatgtacacacacacacacacacacacacacacacatatgagaatgagttctttcagagagccaaggctctgtacaggttcctaggttgtagatagtttcctgcagtgggtttcacaggcaatgagtgttggtgaaatgcacttttgtttggtgatgtacttcaggctgcagtcaagtagatggtgcttaacagcagggctgacagtctcttagtcagctgtacacttgttttgtttttcagcgtgtttgtagcaatgctctgggaagaggagatgggggtcaatatatggctccctctacaagtccattcccaggtttgggtaacatcaccttcgattactggtgctttgtccactttttctttgtcatttttctgtgtctactttaatatctttcattaatattttgtaattttcagtgcattagtctttttttttttttgagatggagtctcactctgtcaccaggctggagtgcagtggcacgatctcggcttactgcaacctctgcctcccaggttcaagcgattctcctgcctcagcctcctgagtagctgggattacaggtgcgtggcaccacacccagctaattttagtatttttagtagagacggagtttcaccatcttggccaggatggtctggatcccttgacctcgtgatccacctgcctcggccttccaaagtgctgggattacaagcatgagccactgcgcacctctttgattaagcttattcctgagtattttattctttttatgctactataaatgagattgttttcttaatttcctttcatgatagtttattgtgtatagaaacaccattgatatgtgtgtgttgatttttgtgttctacatctttactgaatttattaattctaatcatttttcttgttgagtattaagggttttctgtgtacatcatcatgtagattttataaatcatatcgattttgtagattattgctgcagacagataattttaccttttactttagattttgatgccttttatttctttttcttatctacttgctctgtctagggcttctaacactgtgttgaatagaagtagcaagagtggacattcacgccttgtcctggatcttagagaaaagattgattatgatgttagctgtgggattttcatatatgccctttattatgggatttttttttggatatgacaccaaaagcaccagcaacaaaagcaaaaatagacaaacaggattgtttcaaactaaaaagcttctgtgcagcaaaggagacaatagagtgaagaggcaacctacagaaggggagaaaatacgtgtaaactgctgggtgccgtgtctcacgcctgtaatcccagcactttgggaggccaacgtgggcagatcacctgaggtcaggagtttgagaccagcctggccaacatgatgaaaccccatttctactaaaaatacaaaaaattagttgggtgtggtggcaggcgcctgtaatcccagctactcaggaggctgggacagaagaatcgcttgaacctgggaggcagaggttgcagtgaactgagattgtgccactgcacaccaacctgggcaacaagagtgaaactccatcaaaaaaaaaaaaaagatagtatttgcaaaccatatgtctgaaaagggtttaatagtcaaaatacataaagaactcatgcaacacaatagcaaaacaaaacaaaacaaaacaaaaccctaattaaaaatgaacaaagaatctgaatagacacttttcttaaaaaaaaagcatacaaatagccaatagatatattaaaaggtgcgcaatatcactaatcatcagagacatgcaaatcaaaaccatagttagatatcccctcctacctgttaggatggctattgttaaaaatacaaaagaaaagaagtgttgatgaggatgtggagaaaaaagaacccttgtattcttttggtagccatgtaaattggtatatccattatggcaaacagtacgcaggtgcctcaaaaaattaaaaatagaattaccatatgttccagcaatcccacttttgtaaatatgtccaaaggaaatagaatcactatctcaaagagctatctgcactctcatgttcattgcagcactattcacaatagcaaaaatgtgaaaaaaaaactgagtgttcattgacatatgaatggataaataaaatttgatatatataatggatgttattcaatctttaaaaagaaaatcctgccatttgtaacaacacaggtgaaactggaggacattatgtgaagtgaaataagccagaaacagaaagaaaaatgctgcataatcttgtgtgaggattcgaaagtagtcaaactcatggaaacagaatagaatggggatttccagggattgcagtaagggagaagtgaggagatgttggtaaaaggtagatagttccagttatgcaagataagttctggaggtctaatgcacggcaatgtgactatagtgaacaatactctattgtgtagtatttgaattgctaagagggaggatcttagatgttctcactataaaaaataagaaaaaatggccaggcgcggtggctcatgcctgtaatcccagcactttgggaggctgaggcgagcggatcacgaggtcaggagatcgagaccatcctggctaacacagtgaaactccatctctactaaaaatacaaaaaataaaaataaaaaataaaaaaattagccgggcatactggctggtgcctgtactcccatctactcgggaggctgaggcaggagaatggtgtgaacctgggaggcggagcttgcagtgagccaagatcatgctgtgtccggaattggtgggttcttggtctcactgacttcaagaatgaagccgcggaccctcacggtgagtgttacagttcttaaaggcggtgtgtccggagtttgttccttctgatgttcagatgtgctgggagtttcttccttctggtgggttcgtggtctcgctggctcaggagtgaagctgcagaccttcgcaatgagtgttacagctcttaaggcagcacgtctggagttgctcgttcctcccggtgggctcgtggtctcgctggcttcaggagtgaagctgcagaccttcgaggtgagtgttacagctcataaaggcagtgtggacccaaagagtgagcagtagcaagatttattgcaaagagcgaaagaacaaagcttccacagtgtggaaggggaccccagcgggttgccactgctagctgggggcagcctgcttttattcttttatctggccccacccacatcctgctgattggtagagccaagtagtctgttttgacagggtgctgattggcgagtttacaatccctgagctagacacaaatgttctccacgtccccaccagattagctagatacagagtgtggacacaaaggttctccaaggccccaccagagtagctagatacagagtgttcattggtgcattcacaaaccctgagctagacacagggtgctgattggtgtgtttacaaaccttgagctagagacagaatgccaattggtgtatttacaatccctgagctagacataaagattctccacgttcccaccagactcaggatcccagctggcttcacctagtggatcctgcactggggctgcagatggagctgcctgccagtcccccgccgtgaacccgcactcctcagcccttgggtggttgatgggactgggcgccgtggagcagggggctgcgctcatcggggaggctccggccgcacaggagcccatggagggggtgggagtctcaggcatggcgggctgcaggtcccgagccctgccccacggggaggcagctaaggcctggtgagaaatcaagcacagcgctggtgggctggcactgctgggggatccagtacaccctccgcagccgctggcccgggtgctaagtctctcattgctcggggccggcagggccggctggctgctccgagtgcaggcccgccaagcccacgcccaccctgcactccagctggaaccgcccgcagcccaggttcccgctcgcgcctctccctccacacctccctgcaagctgagggagccggctccggccttggccagcccagaaaggggctcctacagtgcagcggcgggctgaaaggcttctcaagtgctgccaaagtgggagcccaggcagaggaggcaccgagagcaagcaaggtctgtgaggactgccagcacgctgtcacctctcaatgccactgcactccagcctgggtgacagagtgagaatccgtctcaaaaagaaaaaaaaaagaaaaaatgtaactatgtgaagtgatggatatggtaataacttgactgtggcgattacttcacagtatatacatatatcataacatcatgttgtgcaccttaagtatacccaatttaaaaaaaattaaggtagaatataaatatataatttatcatcttcaccattttaaaatgtacagctcagtggcaataaatacttttttatactcaatttttatttgttggttgtacctcactaaagctgaaaaatatatttgaataaatgttaaagaaatgtaagtatacacaaacaagaaaataagtatgtagaaattgcaatctttcctgaaaataagccaaaagaataaaactcgtatttaaattctttgaaaaaaaattagcagatggaacattttcttacatttgtacaaatgtactcaaaaattttattttaaatcaattttaatgagttatctcccataaatttcttacatagaaagattaagttgtccaaaggcacatcttataatatgttttgagagaattttctggttttggcaaaaggggatctgcgaatgtgactggggcagcctcaggaaaacataagagggggaatttctttttaaaagagacctctggccacagacactaagtatttttagtaaaaaatgtggttaggcactgatggaatattttttttttttttttttttgagacggagtctttctctgttgcccaggctggagtgcagtggcacaatcttggctcactgcaacctctgcctcccaggttcaagcgattctcttgcctcagcttctcgaatagctgggaccataggcacgtgccaccacgccctgctaatttttttgtatttttagtagagaggggtttcaccgtgttagccaggatggtctcgatctcctgacctcctgatctgcctgcctcggcctcccaaagtgctgggattacaggagtgagccactgtgcctggcccactgatggaattttgtaaagcaggaaggaaataattgtaccatataatgttacaaaaattaaaattaatatatattttttctttggaatttatcttctggataattttaatttagaaggaatttcattattttgttaatgattatggggttgctgtcagacattatttctactttgctcagtaatttgcagttatctgtgagttctgtagcctcaaaattaaaaactagggtgtggttatctttacttacataaaagtgcaattttttgtggaataacaacattgttttattctctaaatatactttgctttattttactttgcaaatgattttaaaattctccctcttttttttttttttgaaacagagtcacactctcctgcccaggatgtagtacagtggcacaatctcagttccctgcaatctccacctcctaggctcaagcaattctcctgcctcagcctcctgagtagctggaattacaggcacacatcatggcacctggctaatttttactttttttggtagagacagggtttcgccatgttggcaaggctggtctcgaattcgcctgccttggcttcccaaagtgctgggattacgggtgtgagccaccgtgcctggcctgattcaaaaattctaatggccggggcacggttgctcacgcctgtaatctcagcactttgggaggccgaggcgggtggatcacgaggtcaggagatcgagaccgtcttggtcaacatgaagaaaccctgtctctactaaaaatacaaaaaatagatggtcatggtggctcttgcctgtaatcccagctgctcaggaggctgaggcaggagagtcgtttgaaccagggattcgggggttgcaatgagccgagatcctgtggctgcactgcagcctgggcaacagagcaagacagcaagactccgtctcaggaaaaaacaaaacaaaacaaaaattctaaggaatacagttacttgtagtattgatgtttggcataggtcattccaatctttttattatgtattattgtttttgtaggttattatatattctcttgaatatttatctttgtttactgttcataattttattatgaaaccaattttggatttacatagcgatttatagatagatttacatagcaacttatagctttgaatgtatttgtagaaaagaaagagtattgaaaataaatgtagacattctactcaggaaacaaggaaactttttttaatctttctttaaccttagaatttagaaatgataataagatgaaactaggtttggacctttttcttttgtttttttgtttgcagtttttttttctttttttaattatttttatttatttatttattttttattattatactttaagttctagggtacatgtgcacaacgtgaaggtttgttacatatgtatacatgtgtcgtgttggtgtgctgcacccattaactcgtcatttacattaggtatatctcttaatgctatccctcccccctccccccaccccacaacaggccccggtgtgtgatgttcctcttcctgtgtccatgtgttctcattgttcaattcccacctatgagtgagaacatgcggtgtttggttttttgtccttgtgatagtttgctgagaatgatggtttccagcttcatccatgtccctacaaaggacatgaactcatcattttttatggctgcatagtatttcatggtgtatatgtgccacattttcttaatccagtctatcattgttggacatttgggttggttccaagtctttgttattgtgaatagtgccacaataaacatacgtgtgcatgtgtctttatagcaacatgatttataatcctttgtgtatatacccagtaatgggatggctgggtcaaatggtatttctagttctagatccctgaggaatcgccaccctgacttccacaatggttgaactagtttacagtcccaccaacagtgtaaaagtgttcctatttctccacatcctctccagcacctctccagcacagaaagagtattgaaaataaatgtagatattctactcaggaaacaaggaaactttttatctttcttttaacttagaatttagaaatgataataagatgaaactaggtttggacctttttcaataattctgaccaccgttcagtaaatttactgtataagaaaagtagaggtttggggttgaattttgttttattttttcccaaagatgtctatctctgatatcttagggcctggcaattgtttttggtcataggtgggctctgaataaatgtcagttgaacaaatatataaaaaccaaacaaaggaatgttatttcaactgccttcagagtgtcacaacaaatatcaatgaaaaagaaacaaaaataaagactcaattaattctccagtgaacacttcagtcctctaaaggtacttccctgtgtccacccaaactgctctctaaccgaacgcaagcaaggggactccaatgctctcaggccatgaactaatgaggagcttcatgcctgccctcagtctttcttcatacccagggcaaagctggcccacagacttccagacatgctttcttcgtttgggtagagatggggatccaagtcccctatgtatttcctccagagccacctgtaaaacatgggtgaaatctacctgccctctagctcaccacttccaggtcaagcccactcatggaccactttaacagtgacagttggcttgactggggaaactgaaaaaaaggagctttctttagattttctgataatctgcattgccattgctgctcctttttcagcagcacagagagttggattatggagggttgcctgcttattatgcagctacatccaggatgctgagtgtcttctgatcatgatcaagagcagagtgaagagatacaggcggaggagaggatcacagagaggtagaagtgggagactggtggataggtcactaggcaccactgagtgactaccatcacttctcacagattagattattgacaagccaagtatggatttgatttctacaaggccaaaagctactccatcttctagccagaccacagactttgttttaagccaggactcgttttccagcagttagtgcaattagggaggttgggaggatgtacacaagagcagggagaaaatctggagccagacagtgagagaggcagaggggaaatgaaaaaccccgtgggaggctgcagcaaagcgaggcagtgtggcttctctgacagggaagtcagcagagggagaggtttgtctgtctgtacagcaagggaagtcagacgagagtgcaagagggtgtggagaggggtactgatatctgaattattagggcaggtgtcctgccaaggaatccctcctttaacagagcttcaatgctgctcctgttcctcctcttcgagggtctctgctgtcctggggaaaatacagcaggtaagaagagtgcaggtggaaagatacctatggtagggcaccagagggctgagaggaagctctggggaggtcctgggggaagggagcagtactcttctaggatgcccttggaatatgcctttcaggctagttccaggcagagaattcttgctctcagtctcagtttttgtctctgattttggagaaaggaagctggccccacaggaaaagggtattggagtatgtacaagctacctaactgtctctcatctctgggttccttttttccctttggcatcacttttcccatccctttacattctctctacttgtcatttccctctctctcagctccccaggctctacaatcctatcatctagcagcagaggagcagctgtccttccgcatgctccaaacttcctcctttgccaaccacagctgggcacacagtgagggctcaggatggctgggtgacctgcagactcatggctgggacactgtcttgggcaccatccgctttctgaagccctggtcccatggaaacttcagcaagcaggagctgaaaaacttacagtcactgttccagttatacttccatagttttatccagatagtgcaagcttctgctggtcaatttcagcttgaatgtaagttcgttgctctaagctgataatttgcctgggaacaccaactattttccaaagtggaagatagtatatagactctgaccatcatttaaccttactaaccttgttccccactctctgactcccactccctcctctgcttcacccttcaccaccacccacactccaccatatacacaaaagggcctgcatgtacatatctcaacatgaatatagcttcatgtctggctctttggaatgattgtctcctctggatcttctgcccctcattcctgccctcagactcagcctttctcaaccctctttctgcccttctttatcctttgcctgagtgttgacatggactggcctgtacctaaccactttcacgtgaattatttatgaccaatctcctatcttcctgatagccttcccatctaccactttcccattagttatttcaaagtatctttattatcttcaaattttcttcccacaaattttcttcccctttgccagtaaactctagtctccatatgatttcccagcaaatttttcttcccttgaatctctttactgtctaaattgtttgtttttcttccttgtcattctttccataatgatctctcttccctgtccactctcagaccccttcgagatccagatattagctggctgtagaatgaatgccccacaaatcttcttaaatatggcatatcaagggtcagatttcctgagtttccaaggaatttcctgggagccatctccaggagcagggatccgggcccagaacatctgtaaagtgctcaatcgctacctagatattaaggaaatactgcaaagccttcttggtcacacctgccctcgatttctagcggggctcatggaagcaggggagtcagaactgaaacggaaaggtgagcccaactctctctctcccctcttgttcctagtactataactctcatatttgaatttgcctctcatcatcattttgaaagacatagtgagagactagagaatgagatgtgtgggttcaggactgtttcttagacaagagaaagaagtgattactaaatcactcttagtattattacaaaggcacctgagtctctgagctctggcctggggtgcccttcaaaattccattttttttctatcttcttcttcctagtgaagccagaggcctggctgtcctgtggccccagtcctggccctggccgtctgcagcttgtgtgccatgtctcaggattctacccaaagcccgtgtgggtgatgtggatgcggggtgagcaggagcagcggggcactcagcgaggggacgtcctgcctaatgctgacgagacatggtatctccgagcaaccctggatgtggcggctggggaggcagctggcctgtcctgtcgggtgaaacacagcagtctagggggccatgatctaatcatccattggggtgagaaacagctgaggctctgctgggaaataatgaaaatagccctggggcttttgagtgtggggctgaggaaatgggtaggaatgctaggtacaagaagggtaaaactgggacaatcaaaataaagaaggatagagtatgacagtagttaaattttaagaaaatggaagtagagaattagacatactaacagaaaaaggaggaggaactagtgatttagtgggagagggttgggaggagatcacagacaaaggatcaggaggaattgaaatgagggctttggaaaacccagatgaaaattctaggaaggtcccacccttgtgaaatgggaaatctcagcttggtggaatagagtattttagggttggtattcttattctatccccaaccaggtggatattccatctttctcatcctgatctgtttgactgtgatagttaccctggtcatattggttgtagttgactcacggttaaaaaaacagaggtgagctttttcttgttctttgtttcttcagcctgtaatcaattcattcctttttcctctatcttctttttttttttctctgcctttcccctttatttcctttctttgagattaatatcctcctcttttcccacagttcaaataagaacattctttctccccacacacccagccctgtctttctcatgggagccaacactcaggacaccaagaattcaagacatcagttctgcttggcacaagtatcgtggatcaaaaacagagtattgaagaagtggaagacacgcctaaaccaactctggtgacatttgctttaccttatacataaaatccttgtctgcatcttcttaaacaccgtccatgtcccataagggaagcatgcttttatttaaacagtttatactagcaaagatactgacccctttaggaatactttttccccatcttccagagattttttttttcctgctttggctacatatccatcattgtttatttttgaaactataatccagatacttctttttcatggattcccgagatcacccaattgatagctcttctgtactccccaaattgaactgatcttcacaagcacattcatctcttcctactctgaacagtagttatttaggtttttgctctttttttttttttaatctcagttgcttgaaagtaggatttaggtatttgtgtctgtattcatgaccaaaaatcttatctgaattcagggccagcttcataagcatgtgacctgtgcagacacatggaatcgtatgctctgcaagaacccatgctagatttaatgctctgctgttgtcatcttggaatccttagtcattttcaaacaagagatattgtattttcatttttcactgaccccacaaattatatagctgattcagtgtgaatgtaatatttctcaataaatgctgactgaaataaatttgttgttagatcaagtgatgttgttttactccattccctctctgatatctctgcgtcatttgactgtgctcttgctaaaactcttcctctctccctttgtctctcaccagaggtgaaggaggcagatgaatgcctcagactccacagtgattggaatgttaatgaatcttcagggatatttaggaaaataataacataagggctatctcctgtcaaagtgaggagaggtttgaactggcattcctacttccctgagccatgtgtatctatgtgtgtctttgtggatgtgcatattcatgggagaaattcatattttgagcaattagaaggaggacatagggtttgagccagagctaaggcaatgaagcaaaagagtgattggatgccagttaagctacatgcttgtttttatttattagtgtggcttattattgttattattatttacagtttgtttttctttagtttttatatggattcttacatttacataatttacttcatttgagatacatttggttgagcctactggagagcaggaatctggtagaagtgcagtgtttatatacatgtccagtcaacccctcccttggatggaataattgagcagattgtgcatatttcttttctttttttttgagatggagtctcgatctgttggccaggctggagtgcagtggctggtgtcagatcactgcaagctccacctccggggttcatgccgttctcctgcctcagcctccccagtagctgggactacaggcgcctgtcaccactcccggctattttttttttaatttttagtagagacggggtttcgccatgttagccaggatggtcttgatctcctgacctcgtgatccaccccccttggcctcccaaagtgctgggattacaggcgtgagccaccgcgcccagccggttgtgcagattttttagtggaagttataaacttcatgctgggcattttcaagtaatcagcaagggtcccatgatcacacatccaggctgcactcttactcatgaacaaggtatgaggccagccttttcactgttgagagtccatcaccttccatatggcacatccaatcagtctatctagagtcaggccactagaatcgggagaggaaggtagaatcttaaagggttgtttggagcaacaaatgatgaaaggggtcagggttccatcacgtggtataactctacaggggaggacctctattagaatgaagaggttcaagtaggaacctttccgtttttggcacaaggtgtgcgaaaggcacaagctatacctctggagacaatagatctgggctgcctgcacatatacatccttcttccccataaaatgacatagaaggattaaaaaaagagccagaggacaaataatcatgttataagaagtatcagctcaacaggacttgaggaagtttaaatatcagtccataatagtgtcagaaaagctctaggtcttccctgtactgtggtaaacctttgggctgggtattttcattataactcctggatgatggtgtacttaggagcaaaatatgttgctaaattgcccccaagacagagatgttacctaatactattaacaacttcattctggctgaaactctgggacatgggacttattctagaagaccagtgtagtttttttcaatagctaagtataccaatttccattcccaccaggtataaaacaaagaaggtaatctgctttactgacagtacacatctggagctttctgcattctgttgtaaatacaaatacagtaagaatgaaagtaggtagttttggaaaaagagatggagggggtattcttgatgattgctaaatttctctagaaatagcattcattgtttgagggagaatgcattgggactggtggaatcagaatatgctggggccaaagctcagaagagtgcagtagccacatgccccggataaaccatgacctaacctgtctgctatcttctatctaatgaatgagataagctgaggatatgggagacttgataagtggagcaacataaactgaaacaggggtccaggcctccaagtgtatcacctacaaacaaagaaagatgcagcacctgctctggagcttgtgttcagatgagtaggtatagttaagggcacatgactccttcttcaggttatgcatagaaggtacctatagatttttgaaacttagactttactcttaaggaattagaactagttcataatctttcctcattaaccattttctcactctcacttccttatgtaaatggttccattatcttactagtttctcaagatccaaactttcttctgtttgcttcattattcttcctgaatagcaagttaccaaagccttctttataagttttcttatgttccattccctcttccttgatcctgccccacgtgtgaacaccactgccaaaaatgtaatgcaaattactaaacccattatcaacaacgccataatgatcacttattgagcatgagatcttattacagcaaataagagaagtatttgttttatagttaaagctgattgaataataaaaatcatttgaatggggcctgctgtaggccaggcactcttctccatactttatacatattaactcatgcaatcttcttatcaatcctgagcatataatattattcccattttatatatgaggagacaaaagtcacataattagtaagtatgtaggcaattttgctccaaaattcctgctttaactactatactgcacattcactaaataactattggaagcttgttatgtgataaatgctattatggtaatgaaatgcattaaagacacaagaatgaattaccctgtcttcacagaacttacgttggcagtgggggagctggaaagacagacaataagtaaacaaagatgttgtattaaggtgatgacatattaagattgataactgaatcaggggaaaggaatcagagagaaattatttattatttcaaaatttgtggacagaatgggccactccgagaatatggcctttgagaagggaacgaaagcaagaacaaacacaagtcatgcagtatctggggtaagaacatttccggcaaaggaactacaaagaaaagcagcattgtaatggctgttttctaaaacctgtgagaattccagtttagatggattggagtaagtgatagggaacataggaaatgaggccagtgagttcaagtcagatcacaaaggggacttataggctattgtacagtgattaaaagccattgcagggttttgttgttgttgttgttcacagtaatatactttactcaattgttataagctgtaaatagcttaaaagtttccttgactctgaaaaacaaaacaaaggattagtagtgttttaagcaaagttaaaaagattgcttcagttttttttttatagaacatctttctttaaattttattattattatacttcaagttttagggtacatgtacacaatgtgcaggtttgttacgtacgtatacatgtgctatgttggtgtgctgcacccattaactcgtcatttaacattaggtatatctcctaatgctatccctcccccctccccccacctcacaacagtccccagcgtgtgatgttccccttcctgtgtccatgtgatctcattgttcaattcccacctatgagtgagaacatgcggtgtttggttttttgtccttgcgatagtttgctgagaatgatggtttccagtttcatccatgtccctacaaaggacatgaactcatcattttttatggctgcatagtattccatggtgtatatgtgccacattttcttaatccagtctaacattgttggacatttgggttggttccaagtctttgctattgtgaatagtgcctcaataaacatatgtgtgcatgtgtctttatagcagcatgatttataatcctttgggtatatacccagtaatgggatggctgggtcaaatggtatttctagttctagatccctgaggaattgccacactgacttccacaatggttgaactagtttacagtcccaccaacagtgtaaaagtgttcctgtttctccacatcctctccagcacctgttgtttcctgactttttaatgatggccattctaactgctgtgagatggtatctcattgtggttttgatttgcatttctctgatggccagtgacgatgagcattttctcatgtgttttttggctgcataaatgtcttcttttgagaagtgtctgttcatatccttagcccacttgttgatggggttgtttttttcttgtaaatttgtttgagttcattgtagattctggatattagccctttgtcagatgagtaggttgcaaaaattttctcccattttgtaggttgcctgttcactttgatggtagtttcttttgctgtgcagaagctctttagtttaattagatcccatttgtcaattttggcttttgttgccattgcttttggtgttttagacatgaagtccttgcccatgcctgtgtctgaatggtattgcctaggttttcttctagggttgttatggttttaggtctaacatgtaagtctttaatccatcttgaattaatttttgaggttttaaataaatgtataatggttgttaggtaaaaggagatgagttccaagaagcagagagtagaatagtggttgtcagggcctaaggggaggagtcaatggagagattgtggtcaatgggcacaaagtatgtaagataaataagttctggagatctactatgcagcacaatacctatggctaacaatgctgtgttgcattcttaacatttgctaagagagatcttgtgttaagtgttcttacaacatacacaaaacattactattactaataaagggggcgcgaagagacttagggctttgatagtggtgatgttattatgggtgtatacttacctttatactcattgagttatatacattaaatattaacagctttttacatgtcaattctaccttaatactgtggttaacaaaaggagatgagttcttaaacacaagtgtcacctgggtttgccatctgtgactacaaaatgggacatgatcatatctacactcaaattacagtgatcccaatctgcttatcttcacgagtacaaacttgtccccactaaaagtatgcctcacctgtgcttacaacccagtctttctgaatcaattaccaagtaacacctgctttgtttattcctctccagttcatgcttccctttgccagctgtctgctgttctcagggttgcttggaacagcttcataactgctggtgcccatggcaagaagatgatattgattccaaggtagctaccacctatttctccaaagcttctgtccagtcctcagatggctttatgtagggatgctccatacagagacacggggtaagggctatgcttcctttctttgactcctttgatacccagttctggaagttgcttggcttgtgttgtcttcttaaagcctctatgtacaataacatcagattctgactctcatccaactcttgggtttcagtgaagaatctcttagcaactttcccttcagttttggttgactggccaaatcagctagttgagtcatagcagtaattacacaggttgcaaaggattaacataaactcaatttgttaaaatacctaaaaaatattctcaatttctgtcactcttaatgtaagtgccttgcttaccactgtatatcatatgtatgcaccccagtgcccagattatacatttaaagaaaagagaaattccatcaagggaggtagtgggttccatttcttgtatcaagaaaaaatttagattacaaacattcttcaaataaagaacaatcattctaaaatacgaaataaaaatattgagtttccaagcaaatttgtttgaatgttatagtatgtttaatttttatcattttaataaatagtagtgttttttatatatagatgccattctattgattatgggttatttatatgcatatttgtttgcataagcaggcaagactaaagagaggaaataataagcacagtaatcataaaaatgtatatattgaaaccaatacaaggagattgtttaccagcaataattatctatataattatcaagtgaaaaagtggtcctacaattaatcatagatggtcattgttgaacagaacactatgcaccagttaccattgttggaggaggactccaactacatggagagtagcaaagatcatcaccatgccaaccaccaggaatagaagtccaaacacttctgctcaatgcatcttgatatcagagtttccaagaaaaacacaacacagtcaggtcctggataggacgacactttacttacatagagaagagatagagcaaaatcactttcaatagtgtgtgtcagtcccccttggcctgtgggtccctcctggcagcccacacagttggcctgcatacatctctcttgtgctgcagcagaaggaacctttccttttccccttggagcctgaaataatgaaagctgggagtgtgctacaaaattgggccatttatgcacacactgaagcaaaaggaggagcacacgttgagtctgaaatagggaaagactttcccacataggaaaataaccctggcaaaagctgtgggggctctttgtctcctggtaagaaaatgttcctggcctaaggcccattcttatgtggccaagtcgtggagttgaaagaccacatgcataagaccacctttccttacagcccatcccgactcttgtctggccactgaaacagcagataaaacatatcaaatcacccaccttgatttggaggtgtagttgggtcccatggagcccctgaacaacttgtgcagatgcagtttgctcaaacagcctcttttattttgaaagagcactttaccagagtcaataggggaactaaaagcaggagaatggttctcatcagaaatcccaagtgcttggattaaggtaaacaaagaggtaaaaaatgagtctgggtggactgtggatacatagctgacaaggacctcactgagtgtggatattcagcttggtttctaccctccctgaattaatgatatacacatagcaagtggtgttggccataacacaactaacaccctgttctgccagcagataactgagggctattcagttatccatcacccccttggctagtgaatttagtggctctttttcttaggtggctcaggcagtaatgttaactaactccactaccgtcaatggcaaattgtgtattgtttttccaggatatgtatgcctatgttggatttgaggacacagacagtagaatagagccagctgtcagtttgttcacctggcagggtgtctcaggcaaccctgtgatgtgtcagaaatgtggatacttccaatttagtgtcattttgaaaaagcatgtgagaccctcacattcccaagcagcatgcccatgtgagggtgacaggcactcggaagtctagtatgccccacagaggaacatataatccaggggagcacatgtaacgctggtcagggagcagttgtgttaagagtctgtagtccagctgtacaaagctttgtttgatttaaccatgagttaggtggcagggtaaagttgtccaatgtatggaccttcccatactatctgtgatgtttaatttgcctttgatttttttttttttttacctctgattttatgtctgtggaagaggtaagcttcactcatggagtattgggtgccctgcatatttgaactgcactgagggtgtcactctttgactggaacctctggtgaccattagaaatcctatcaggctgctgatgggccacacatggactatttgaggaaattcagaggaaaccttctgaatatgtgggtggcagatccaatagcccttcaaatctccacctgctgctactgctgggctgatcttcaccaatatgttgtcaatccaggcactccaactacattattatacccataacacacagggtattattaaggaacaaaagggagagagattagtatccctactgactacctgcttctggtacttccagtggctggaggcattcctgtctattagttggggtgctagaagtctgagtattgcaacccaggctgtctgtcccagtcctgaggctactacctctgtgggtactcattattcttgttacttgatccagatcttttggacttgaatatccagtccagaaaggaaggtcacaccctgcacaaacttgacgtgtcggttaatgtcacctatgagaatggaagaattggatttcccagtgcctctagagtgatcttagcaccctgacagtcccgggttgaatcgcccaccccagtacaatcccataaaaaggaggactccaggagtaagtattgccttcagattagagtcagagaggtggtatttagggtacaatggcaacctgaactgcaagccatctctaagacttacactctaaggggaaagaaaaaggaaaaaaaatggttgggaatactgagtttgagattttgaagtcccagcctgatacggcttggctgtgtccccacacaaatctcatcttgaattgtagctcccctaatccccatatgttgtgggagggacctggtgggaggtaactggatcacgggggtggatctttcccatgctgttctcatgatggtgaataagtctcatgagatctgatggatgtataaagaggagttcccctgcacaagtcctctcttgcctgccatgatgtaagaagtctttttgctcttccttcatcttccaccatgattatgaggcctccccagccacgtggaactgtgaatccattaaacctctttttctttacaaattacccagtctcgagtatctctttatcagcagcatgaaaatagactaatacacagtctaatcaaatggctgcctgatctaccgtgacatccaccatatcaaacaccaagctcagtgtttcccaacatgcattgcagtatcctcttccccctacaatcaagtgcagtgtttagtgtccattctgtcctagccagatgtgggtaccaccccactagcaagccttatttgttgtcctatttttaattgttacagtctatttttaattgtttcattgtttgatgaattgtcctatttttaattgtttcattgtttgatggttccattgacttgcaagtaagagtgcacataataagccctctgcattttatgagagtgcatccactgttgttttgcttgggcagtgaaagatgttccttggtcagagtaaagcctcatatattatctataaacatgacacagtttttttcaaagggggctatggtgattctagcatccacattttgggctgtaatagcaataccacatcctgaataggtgtcaatagtggcaaggcaccaccagtgcttgtggctaggggccatgggtttgatgtggtcaatctgtcaaattcaggcaggtcctagtctttggttaatttgtccttatttttctctttggatgatgtgggtccattagcaagagccaccatgtccttaatcttggcattttctaggatcatctcttggatcttatccaatttttatatgtaagatgaatgtccattttaagtccatataggtagtaggctgcctattggccacaagcttcatcagcaggagagttaatttatattttgttttaaaagatcatttgttatatgagtagcatttttgttcctcacaggcagcaagatgctgccatagttcctgtccacacacaggcaatcctttaatagttcagtcatttagctgaccttctcctggtcaggaagctaggcagttctctgcttgggattcaagagcctatggaacatccttcgaaatatagctggaggtagctaagtccctgcagctcttgaattatgttcacctgaaaagttagcaccacaggatgctgccaagatttatcacttgtgcactttggagtagtggtctgagccatacctggacccacttgagccacagctggattgccaaggaactctgctgtggagtgcagagagctttgaaatctttctgtccccagggtcctggcattctgggcatgtgatggttggagcagccctgaagatctcagaaatgcctttgggatcattcttccattgtcttaatatcgccaggcttctgcccattcatactagtcttcttattagaaggtttcttggccacatccttggtgtcctctccttaactatcttttttattttttacagcatagccaggttgagaattttcccaatctttaatgtctgcttctcttttgattatacattctatctttttttccctttcttttcacattttactgtaagcagccaagagtagcccgactacatcccaacaccttgcttagagatttattccaccaaatatcttttttcattgtttgtaagttctgccttccacagaacactagggcataaatagaatttaatgaagttctttgccattttataacagggcttttttttccttcaggttccaataatattttcctaattttggtctgagatcttatcagaacagcctttactgtaaatatttctgccaatatttttctgatcacaatcacttaggtaatctctaagaagacaaaggctttctctacatctcttctcttcttctgagccctcacgggaatcacccttaataccctgttcacagcaatataggcgtttttttgcattaacatccaaactcttccagtttctacttactactcaattccaaagctgcttccacaactttatgtatttattaaagcagcactccacttctcagtatcaatttctgtctcagtatgttcaatctactgtaacaaaatgccttcgactaggtgatttataaacaacagaaatttattgctcacagttctgaaggctgggaagtccaagatcaaggcaccagcagatttgacgagggcatgttttttccttcaaagattgtgccttttgctatgttttcacatggtagaagaaagagataagctccctcaggcctcaataagtctgcaacagggaaagataatcccacacaaagatggtaagcctggtacaggctgtgtgggttgtttgtctcttggtaaggaagtgttccaggcccagtgaccattgttttatagcctattgtggatcaaaagaccaactgaatgagactgcccttcccaaaacaagtacatgtaataaacaaacacaaagtaaagtcaatggtatgttctaatatttaaattagccaagtgtaaaaagcagaactaactttgcaacttaaatcttcatatccaaataatgctattcaaagtatggtccaaggaatagagtcagactaagaaatatttgttactcattgttgaaaagataagtatagaaattgagagtaaatgtttagaaacttttatggaaattcaacaaagtcattttacatctgttaatttaatagtaagaaagaaaggtcttgtatgtcctgtttcatttcatttttaaaataatatgtttcactgtgttttacaaagtctacagtaaatctgggagaaaacactggtctttcaccttaggtaggttgagaagcacagacatagaatgtgagaagggggacatggtcaggtgtaaagagtacatgggtagaatgtacaggcctttgtgtgtcatgctacagtatttggatgtgatctcataagaagggaaaaaaacatgtgtttataaactatttttaagaacttcttagaaaaattgatcttgagaatgtgtccaagtttattaacatgttatcaaggaaatcagcaaagtgataaaaaaatatttaagaagtgcaagtttcagagatgttataaatttggagcggtgacaaatttaaaaaataacacttaaagaataatagtgattatagttttatttgaaaggttcataaaggcataatagccaagtaatttatttacacagagcatctgtagcaaaattatctgagatgccagatgtgcagccataaagtcagaagaaaaaaataaaagaagcaaatgagagatacaaggtatctaaaattgaaatgacgtgttttcactctcaaaactagcataagatataaaaatgtatttaacagagatctgaaaaaacaatttctgatttcaaagatgccattgccaggggatttcttctactctgagctataaacaaaaatgctgatgacacagttcactcatttcttactttaggactattttacttttttttttttttgacacatggaagagtttttgagagtagctacaatgtttgcatcttagaaacataactgagtctgcaacatagaggttgatttgaagggtgtcaacaaagagacgtctgggctgggtgttgtggctcatgccagtaattccagcatttagggaggtagctgaaaagatagcttgagcccaggcattccagacctgacggggcaacatggtgagactattctccacaaaaaggaaaaaaggacaaaaaacagatttctgtagtaatcagggtgcaaattaagaaaggattgaactaaaaataacagtggtaaatgttgctatacaatttaacctaaactagaagttaaacaatacattatggaaaaaatgccaacctgtattaaaatcaagaagaattattctctatgagctctacattaattcattcttgaaacattaccaaaataatataaagagttacaaaatggtctcagcagtcactgtgatggaattagggaaaggctctgagcctgtactggggcccttagtacatcttacatcaggaccatctgtatgggaatagcactaccatggaaaaggtttgtcttatgggcccccaagataagaagtctgagagtattaataaaagaaacagaaagttaaacattattgggattttgctcttgtcaaataaagaggcaaatagaaagttggtaattattaaaagtaattttccagtttttcagtttaagtactgcctatgataaagatgatggtccctattttaaagagaggtacagtttcatcaaaaaagctgataatatttaataaataggctgtcaaaatacatacgtcatgggaaaatagagaaagaactgaagtatatttttaaattttcagatagaaattaagggaatacaagatgtcagaaaagaactggttactgtaattagcattactgtaaatttctatgtttgtcaagaatatgagaaaattttagttttttccctaagaaagaggctattgaaagattccagttcaataacttaagacatctttggaaagaagaagaaaaattataaattgataatcatacattaaagagaacatctagaggagaacactagagtccagcagagaattgatgggaaagatctgaggtacagaaggagaagaaagtgagtagccagcttcactgagttgggccaggagcccaaaggggcttggtatagcacatgcttttcagagcccgaaagctccctgtctagagctttgagaagtgatcctgaccttagcagcagcacaaactctgtccttagcattgcaagggaagaacaaaatcccctctcactggcagagtgacctctgtgcttggcttcatgaacagagaatgaagcccttctgcccctccacacatcctctctttctgttaccagagactggaacaaatgagccaggaggctacctgtctgggggaatgagtcacagctacgactccactagcagcacaggctccacagctgggattttcccctcttcatggtgctgtagtgctgctgctacagggagcaagagagattgaaagcagtgtgtctcggggtgttggggactaccccataccacagacaccaccaacactagcccacattatttgggacctaaagagtcattccactactgctattgccatggcccttaccatgctggctgcccagaaacctgagaacctgctcacctacctggctcactgctaccactatagtcatccaagcaagccacttgggggctcaagaatcagcccactggtaaccgccaacacaggtgccagggtacaccaccctggggcacaaagataggcatgcacagcccgctgctgctactactgaagcctgaagactgaccctcctggcttcctagtccccagcacaacgtcaccacaacctccactaataactaaaacctaaccctgataggaaaccacagatacccctagcattgtttagagccaaagatatgcagagactatactactgcatacacccagagtcaaatccaaactgcccagaccaatcgatagatatgtcttcaggaaaaagttgtctctacaaaagtaaattcaaaaaataaaagaaagcaactgttacatcagatgtgcagatatcaatgtaaggacacaataaacatgaaaaagcaagaaaatacgacaccttcaagggaatgcaatcattctctagcaataagtattaataaaaacatctcagaaatctcagataaataatttaaacattgattttaaagaggatcactgagataaaagaaaattctgaaaaatattactgagaagtcaaaaaaatagtttatcgatgggaaatttgccaaatacatagatatttttaaaaagaaccaaacaaaaatcttggaactaaaaaattcattgaagaaaatgcaaaatacatttgaaatcttcaggaataaatgagatgaggcagaaaaaaatatcagaactttaagaaaagtctttgaaacaactcagacaaagttaaagaaaaagagttaaaaagatgagcaaagcgtttattacatttgggagtaaataaagtgaccaaatatatggcttatcactatccccaagtatattagtcagttctcacactgttaataaagacttgcctgaaactgggtaatctataaagaaaaagaggtttaatggattcatagttccacatgtgggggaggcctcgcaaacatgacagaaggcaaaggagaaacaaaggcatgtcttacatggtgacaggcaagagagcttgtgcaggggaactgctgtttataaaaccatcagatcttgtgaggcttattcactatcatgagaccagcacaggaaaaacctgctcccatgattcaattacctcccactgggtccctccctcaacacatggggattatgggaactacaattcaagatgagatttgggtggggacactaccaaaccatatcactgagagtgaaaagaaaatgaaagaattagaaaatctatttattatttataaatagtataaattattatttatgactggtataaaattagaaaacctatttatttttgtaaaataataggtgaaaatctctgaagtatagaaagaaatttaaacattcaagtatagggttcaattatccccagccaggtacacgacaaaaaggtcttcttcatggcacattacaatcagaccatctaagggaaatgataaagagcaaatcccccaaacagcagaagtctctagtcactcataaaggaaactgtattgtgtccggaactggtaggttcttggtctcactgacttcaagaatgaagccgcggaccctcgcggtgagtgttacagttcttaaaggcggcgtgtctggagtttgttccttctgatgttcagatgtgttcggagtttcttccttctggtgggttcgtggtctcgctggctcaggagtgaagcttcagaccttcgcaatgagtgttacagctcttagggcagcgcgtctagagttgttcgttcctcccggtgggctcgtggtctcgctggcttcaggagtgaagctgcagaccttcctggtgagtgttacagctcgtaaaggcagtgtggacccaaagagtgagcagtattaagattgattgcaaagagcgaaagaacaaagcttccacaatatggaaggggacccgagtgggttgccactgctggctccaggcagcctgcttttattctcttatctggccccacccacatgctgctgatggtagagccaagtggtctcttttgacagggtgctgattggtgcgtttacaatccctgagctagacacaaaggttctccacctccccaccagattagctagatacagagtgtggacacaaaggttctccaagtccccaccagagtagctagatacagtgtgtcaattggtgcattcacaaaccctgagctagacacagggtgctaattggtgtgtttacaaaccttgagctagatacagagtgctgattggtgtatttacaatccctgagctagacataaaggttctccaaggccccaccagagtagctagatacagaatgtccattggtgcattcacaaaccctgagctagacacagggtgctgattggtgtgtttacaaaccttgagctagatacagagtaccgattggtgtatttacaatccctgagctagacataaagattctccacgtccccaccagactcaggagcccagctggcttcacccagtggataccgcacctgggctgcaggtgcagctgcctgctagcccgcaccctgtgcccacactcctcagcccttgggtggtcgatgggactgtgcgcagtggagcaggaggtggtgctcgtcagggaggctcgggccgcacaggagcccatggagggggtgggaggctcaggcatggctggctgcaggtcccgagccttgcccctcgggaaggcagctaaggtcgggtgagaaatccagcgcagcgcagtgagccggcactgctgggggacccagtacaccctccgcagccgctggcccgggtgctaagcccctcattgcaggggagggggggccaccaagtccacgcccaccccgaaccccagctggcctgcaagcgtgtgcggcgcccggttcccgctcgcgcctctccctccacacctccctgcaagctgagggagctggctctggccttggccagcccagaaaggggctcccacagtgcagtggtgggctgaagggctccacagtgcagtggtgggctgaagggctcctcaagtgctgccaaattgggagcccaggcagaggaggcgccaagagcgagcgagtgctgtaaggactgccagcacgctgtcacctctcactgtcaggttaacagtagatttatgagcagaaatctcacaggtcagaagagaatacaaagtgtgaaagaaaaaaagaaaaaccatgccagccagggaaactataccaagcaaaattattctgcataaatgaaggagaaatactctttcctagaaaagcaaatattgaggaaaatttgttaacactaggcatgtcctataagaaatgctccagggattaaagcctataacaagttaacaaaaaaaaaaaatgctcaactgagtactaaacctggaagtgaaaggagcacatttaccatcatgaaaacacacaaaagtataaaatacactggtaaagcaatcatacaaaagaggaagagaaaggactcaaacggtaccaatctagaaatccaccaaaccacaatgacaaacaaggaaaaaataaaggaatgaagaatatacaaaccaccagaaaataatttataatgtgacaggaataaagcctcacatgtcaatgataattttgaatttaaatagattaaattctacacttatgagatatagaatggctaaatggataaaattacataatccaattatatgctgcttacaaaaatctcaccttactggtaaatacacataaagatagaaactaaaagaatgtaaaaagataagccacacaaatggaaaccaaatactagtaggagaaggtattcttatatccagtaaaacaaacttttagtcaagaacactagaaaaagacaaataatataatgataatcaatccagcaagaagatataacaattttgaaaatatgtgcatgcaacattggatcacccagatccataaagccaatattactaaatcagaagggagagatttagtgcaatacaataatagtgaaaacttcaacccctgactctcagcattaaatatatcatatagcaagaaaatcaacaaagaaacaatagctttacatcagactttaggccaatgggcctaagagatgtttacagaacaggtcacctgactgctgcagaatacaggttcttttcattagcacatggaatattctccatggtagaccatatattagaccacaaaacaagtctcaacaaattaaaaataatcaaaattatatcaagcatcttctcatgccacgttggaatgaaaccagaaatcaatactaagataagctttggaaactatacaaatacacgaaaattaaagaacatgctcctgaatgaccactgggtcaatgaataaattaacatggaaataaaaaaatttcttgaaataactgaaaatggaaacagaacataccaaaacctgtggggcacagcaaaagtagtattgagggaagtttatagcaagaaatgtccaaatcaaaagagtagaaatattacaaattaatcaagcaacacaacacagagaactaaaaaagatctaattaaaccccaaattagcagaacaaaaataatagagattaaagcaaaataaaatcaaatagaggctgcccaaaatacagaagaccaatgaaatgaaaaattgcttctttgaaaagataagcaaaatgggtaaaccactagatagactaaccaggaaaagaagagagaagattcaaagaaacaaaatcaaaaatgaaaatggagacattaaaatgaatatcacaaaaatacaaaagatcatcagagattattatgaacatctatatgctgacaaacaggaaaacctagaggaaatagagaattcctggaaatacacaggcaactgagattgaatcaggaagaaatagtaaaccagagtagatcaataatgagtagtagattgaatcaggaataaaagatcttccaacaaagaaaaactgaattgcatggattcgcagctgaattgtgccaaatgtaaaaagaagaactaaaaccaatttctgccattattccaaaaaattgaagaggagagaattctccctagtcattcaatgaggccagcatctccctgatacccaaactagaaaaggatacaacaaaaaacaaaactatagaccaatatctctaatgaacatagtcataaaaattctcaagaacaaaactagcaaacaaaactcaatagcacatccaaaaggtaatcaaaagtgggtaataaattccactgctatcaagtgggatttatatcagagatgcaaagttagttcaacatacacaactgaataaacatgatacaacatatcaacaaactgaaaaccatacaatcatcccagtagatacagagaaatcatatgataaaattcaacactcttttatgatagaaactctctacaaaccaagcatagaaggaactaccccaaaataataaaagccgtatatgacaaactcacaggtaacatcatattgaatgaagaaaagttgaaaacattccaagaacttgaacaagacaatgatgcccattttcacaactcctattcaacgtagtactagaggccctagccagagaaaatcaggcaaggaaaagaagtaaaaggtattcaaaatggaaaagaggaagtcaaattgtttctctttgctcttaatatgatcttatatctggaaaaacttaaagactccaccaagaaactcattagtttggtaaatacattcagtaataacgcaggatacaaaatgaagatatgaaattagtagcatttctatgcaccaataatgatttaaccaagaaagaaatcaagaaagccaccccatttacaatagctagaaaaacaatgaaatacttaacaaataactttagccaagaaggtgaaagatctctacagtgaaaactctaaaacgctaatgaaagaaattgtaggtgacacaaacaaattgaaaaatatcctattttcacatatcagaaggattaatatccttaaaataaccatattgccccaaataatctacagattcaatgcaatctttgtgaaaatacaaatgtcattctttatagtatcagagaaaacaatcctaacattcatatggatccaaaaaagagcctaaattgctgaagcaagcctgactggaggcatcacattatgtgacttcaaaatgtatcacaaggctatcataatgaaaaacagcatgaaatttttataaatatataaacacagaccagtggagctaataaaaaaactcagaactaaaggcatatgttaatagcaaactaatcttctacaaacctgacaagaacttacattggggaaaagacaccctattcaataaatggtgctgagaaaattggatagccacatgtggaaaaataaaactggatcactgtctctcatcttattaaaacaactcaagatggggattaaagacttaaacataaaccccaaaactataaaaatactagagaaaactctcctgtacattggtttaggcaaataatttatcactaagaccttaaatgcacaggtgacaaaaataaaaatagacacatgggactatattaaactaaagagattctgcatagaaaaagaaataacaaagtgtagagacaatctgttaaatgggagaaaatatttgcaatctactcatctggcaggggactaatatccagaatatacaaggaactcaaacaactcaacaggcaaaagaaaataatctcatgaaaaagtgggcaaagaacatgaatagacatttttcaaaagaagacatacaaatgggtaacaggatttgaaaaaaaagtttaacatcaccaatcaccagaaaaatgcaaatcaaaaccgcagtggacacaatcttatcccattcagaattgctattattaagaagtaaaacaaacaaataaaaaacagatgttgacaaagatatggagaaaaaggaactctcatatactgctggtgggaatgtaaactagcactcgagctatgaaaacaatatgaagatttctcaaaaaataacaatagaattactgttcgatctagaaatcccactatgggtatctacccagaggaatagaaaccaatatatcaaagggatacatgaacttgcatgtttcttgcagcattattcacaatagctaagatatggaatcaacctaaacatccatcaatggatgaatggataaagaaaatgtgatatgtatacacaaaatggaatactgtttggccctgaaaaagaatgatattatgtcatttgcagcaagatggatggaactggagatcatcctaagtgaaataattcaggcacaaaaagacaaatatcacatgttatcacttatatcacttatatcactaaaaaatttgatcacattgagatggagaggggaaagattgatgagagactaggaagggtgagctggaggaagaagcaggataaagggaggagttctaaagggtgcaaacataacagtaagatagaagaaacaaattcaatgtttgatggcagggtagagtgcttatacttaacatttttgtaacaaaaatgctataaaaatgttttatattcaggtgatcaacaccctaaatacaaaacctgtgggggacagcaaaagtagtgttgagagaagtttatagcaagaaatgtctaaatcaaaagagtagaaatattacaaattaatcaagcaacacaacacaaagaaccaaaagagatctaattaaaccctaaattagcagaacaaaaataatagagattaaagcaaaataaaatcaaatagaggctgcccaaaatacagaagaccatacaaatggctaatatgatttgaaaaaaatgctcaatgtcactaatcaccagataaatgcaaatcaaaaccgtaatggacacaatcttatcccattcagaatggctattattaaaaagtaaaacaaacaaataaacaaaaacagatgttgacaaagatatggaaaaaaaggaactctcatatactgctggtgggaatgtaaactagcacgcacactatgaaaacaatatgaagatttctcaaaaaataaaaataaaattactgtttgacctagaaatcccactactgggtatctactcagaggaatagaaactaatatgtcaaagggatacctgaacttgcatgtttattgcagcattattcacaatagctaagatatggaatcaacctaaacatccatcaatggacttgattactatgcattctatacatgtaacaaaatttcacatgtacctcatacatatgtagaaacaaaaaatatggtggctagatttacaattctaaaataatcatcgcccaatcagtgtatccctcacccaggaaataaacttttcgtgtctggattaatctcttagctacatggaactttattctaaaataatttttatctcatagaaagttgcctatcatcttgattcggaaaacataacagctttctctcttaatttctctatctaaatttgcctgaaattagtaaatgtttgaagtgaaccgtgccagaaatgtagacttatatatgtgttcttctttgaaaacattgttgagggctgcttaattttgaattaggggtagggattctcaactttccatttaaaaggtgatcatttggaataaaagaaaactgtttatttggtcctcaccttggtcttgttttaagaaatagtataactctctgtaggtctttatttgcttattcataaaataaggccacctggtacattatctttatcttacaaacaaatacaaacaaaaaaaaaaaccctttcatttttctgagagtagaaatacaggccctatgaacatgtggtatttatgctgcggtaaaaaagccatgcaatgtttttactgtcaagatactgggttaatctctaataatccagttgttgcttctgattgggtgccaaatgttttgttagaaattgtagagggattacagaaaggagataagaaatctctgagatctagagacctgaaccacattaccaaccaacttgacctagttgacataaaccttcaatctaacaacagcagaaagcataatcttttcaatagcacaaggaacatcaccaagaaagtctatattccgagtcattaaacaaaccttaatatatttaaaaggattgaaatcatatgaagaatgttctgtcaccataatacaattaagcaagaacacaaaaataggaaatgttctggaaaactccatatatttggaaatgcaaataaccgctgagtcaaagaaaattaaaataaaaactggaaagtattttgagttagaagaaattgaaaatacaacatatacaatttgtgagatgcagttaatgcaatgcacagagcacaaattatagctttaaataattaaaattttaaaaagcaatgtgtaaaattaataagctaagcttctaccttaaataactggaaaaaagaagaacatattaaatccaaaacaagtggaaagaaaaaaataacaacaggggaaataaatgagttaaagatgtaaaaaattagaaaagaattatttaaatcaaaagataactcttttgaaaaatcattaatacagttacatctctatccaggcaaaaaagagaagacacaagttactaatatcaggaatgaaagggaggagattgtactattcatgcagatttcataaatatgataaaagtaataaaacaactttatgctggtaagttcagcaacttagattaaagaaatttattaaaagaaacaacctagagtcactcaaaaatatatagataatctacataaccttatatatctttgaaaaacgtgaatttataattgaaatacctaacacaatgaaactatatgtccagatggctttattgttgaattctactaaatgtataagaagaaataatgctaattcatcacaatgttttccaaaacgatagaagcgtaaagaaaattaagatgagtcatagacttaagggtaatagatgaaattataaactttctaaaggaaaacatagaaaaattcgagtgactgagttaagcaaagatttttttagatagtacacaaaaaaataagagccattaaaatttgatcttcagaaaacactattgagaagcagaaaatgcagagcagggatggggagaaaatatttgtaaaacatatatatctcataaaggattagtggccgaggaatattctttaaaaaactcctatatctcgataacaggaaaaacaattcaataacaaaaacagagcaaaatatttgaatacactttttatcaaagaagatatgtaagtggctcataagttcatgaaaagatgctcatcatcattagtcaaatgctaattgaaaccacaatgaggcattactacacaaccgctagaatgcttaaaataataataataaaaaaaccctgaaagtactgtcttagtgcaggctgctacaacaaataatcaaatactgggtggcttacaaaatatagaaatttatctcccagagttctagagactggaatttccagatccgagtagcagcctagtcaggttctggtgagggtcctcttctttgctgcagactgccatcttcttgtagtatcttcacatagtgtaatactacgtgagaggggctctggtggtcccctttataaggacactaatcttgttaatgagggctccactcttacgacctaattccctcccaaaggccccagctcctaacaccatcacattgaaggttaggatttcaaaatacaaactttgggaagagacaaaaattcagtctattgaagcaccaagtgttgaagaggatgtggaacaaaaggagcttttgtacatcactgataaaattgcaaaacggtacagctatttttgaaacagttttgaaaacagtttggtagtattttacaaagttaaacatacctagtacgttattgattgatttattgtaaatgtgaatacatgtaacctagtaatctcattcctagttatttacttaagagaaataaagacaattttcttcacaaagacttgtatgtgaatattctcagcagccttatttataagagccccaaactgggagcaatccaaatgtcctttgactggtgaattcagaaataaattgtgtcataatcatacaatagagtacttctcagccataaaaagatgggggaaaaaacgctattaatatactcaacaaaatggatgactaacaagagcattatgctaagtgaaataactcagacacaagactgcataatgtatttctatactgcaatgcatatttttatttatatgaaattataaaaaaagacatgttatagtgacagaagattagtgcttgcctggggtcatggatgggggaaaggtttgagtgctaagtgtgcatggaaacttttggagtgatgaaattgaaatatcttgaatgtggtgttggttacatgactgtatccacttgtgaaaatttgatctttacatttaaaatgaatgaatttgaattttatgtattattgctagcaattcaaaagagtagttggactctcaaagttagacaaagatccctttcttctgggtgaattccgcccgtctacttaataagcagatgtgattattcaatatttgggtaggtgcaaattaagattgatttacaataaaaatgtatctgaagaaaattaaaaggtattacctgtgcaatattttgaagaatttttcttctcctttgtgtttagtgagtgtatgatattctactttattgtcatataatttatttaaaatatatcctgttgttatatttagtgtagtattgtttgaaatttactttaaaatattttacgtagcacatcgtatttgtaaattaatcttgaatttacaacatagagacagctgttcaccaagtcaatcattctattaaggaatacttccagcagctgttagttagcatttttaaacaccctattgaggagctcccattcaagaggaagattaggaaatggcaagaactttaaatattgctacctaaatctatactagataatacattgttctgcatcaaagatgcttgaaataatttttaatatgtgaaatcacaggtatatcaaagtctgaagtctaaagatatctactcatacttgtgggatgctgtaggagaattagagaagcccaggaacctaacatccttcagaatactgcccaaggcttaggattattttagttccatattttactatataacaggctctgtcttctaacctgaccagagtttgactgactatcaaggatactatattgtcatatctgcctggagagaaccctttaaacagagaacataatagaaaatttacatatattgacttaaacaaaatatgtgtccatttccttccatgaaaaagatacattcagagaggggcagtttataacttgtaggacagctttatgacatttttagggctcaggtttatgatattttattttctgagagacttaacatgtgaattccttctcatggtttaaaagagaccactggtgttttggccatcacatccacattttagttagggaggtagagaagataaatattattcccccaccgccaccactttaaagacattttctggggcatatgacattttcacttacattccacacttacaagacatcttagttacattctagctgcaaaggagctggggagtatctttatttaggttaatcatttgcccattaaaaattgaggatttcctttactaagaaagataataaatgggacattttaggaccaactagaaatctaagccctgatacattataacacagagttattttaaaaaagaactttaaacaaatgcaaattagaatcgaattttcttatcattggtaaatgttataaacaaaaacttatagaaaaggccatatttattaaagattctttatatacctatcattcaatattaagaactagacaagaatggttattattttactattaactctaatataccattgaaaatcctatgaaatatttggagacaagaaaaacaagtgaaatataaagatttgaagagaaattaaaatgttcattattataggacagtatcgtcatttgaaagttgtcaaatacaagtagtggataaactattggcactaaaataattcagtaaatttgttggacactaaatgaacaaacaaaagtgaagtgcatttctttatgtaattaactattaatagaaagtaaaatccaatatggatattacaaaaagtagcaatacaaactataaattatctagagattaatttaataaatattttttaatcactattaaataattattgtcatataaaaatatcttaaatggaaaaaatggttcacatttttggatgactgaatttaatgttataaaaagactgacttccctaaatttcaggtgaccagtgtatgtgtgtgcttgtgtgtaggtgctacttgaaaatgttaagcctaatttgaaaaagaagacatagtgagcaaagttactctgtaaggtacaaaggatactgcaaagaaatggcaatataaaaaatggaacgtcaagcagtgaagagcatcaggaatctgtgctttatagtattcaatgacatcccattctggcgtggccaggggaaatatagtgctacctaaattcctaaaatttaactttattcatagaaaaatattctaaaggaaatactgaactttcagaagaaaaaacctgagataattttggagttgaatttgtgggtaaaatagcaaattttttttcttttttttttattatactttaagttttagggtacatatgcataacgtgcaggttagttacatatgtatacatatgccatgttggtgtgctgcacccattaactccattaactcgtcatttaacattaggtatatgtcctaatgctatcctccccctgccccccaccccacaacaggccccagtgtgtgatgttccccttactgtgtccatgtgttctcattgttcaattcccacctatgagtgagaacgtgcagtgtttggttttctgttcttgcaatagtttcctgagaatgatggtttccagcttcatccatgtccctacaagggacatgaactcatcattttttatggctgcatggtattccatggtgtatatgtgccacattttcttaatccagtctatcattgttggacatttggcttggttccaagtctttgctattctgaatagtgccacaataaacacacgtgtgcatgtgtctttatagcagcatgatttataatcctttggatatatacccagtaatgggatggctgggtcaaatggcatttctagttctagatccctgaggaatcgccacactgacttccacaagtttacagtcccaccaacagtgtaaaatgttcctatttctccacatcctctccagcacctgttgtttcctgactttttaatgattaccattctaactggtgtgagatggtatctcactgtggtttggattggcatttctctgatggccagtgatcatgagcattttttcatgtgtcttttggctgcataaacgtcttcttttgagaagtgtctgttcatatacttcacccacttgttgatggggttttttttgttgtaaatttgtttgagttcattgtcgattctggatattagccctttgtcagatgagtagattacaaaaattttctcccattctgtatgttgcatgttcagtctgacagtagtttcttttgctgtgcagaagctcttcagtttaattagatcccatttgtcaattttggcttttgttgccattgcttttggtgttttagacatgaagtccatccctgggatgcaaggcttgttcaacatatgcaaaccaataaacgtaatccagcatataaacagaaccaatgacaaaaaccacatgattatctcaatagatgcagaaaaggcctttgataaaattcaacaatgcttcatgctaaaaactctcaataaattaggtattgatgggagtatctcaaaataataagagctatctatgacaaacccacagccaatatcatactgaatgggaaaaactggaagcccattccctttgaaaactggcacaagacggggatgccctctctcaccactcctattcaacatagtgttggaagttctggccagggcaatcaggcaggagaaggaaataaatggtattcaattaggaaaagaggaagtcaaattgtccctgtttgcagatgacatgattgtatatctagaaaccccattgtctcagcccaaaatctccttaagctgataggcaacttcagcaaagtctcaggatacaaaatcaatgtgcaaaaatcacaagcattcttatacaccaataacagacaaacagagagccaaatcatgagtgaactcccattcacaattgcttcaaagagaataaaatacctaggaatccaacttataagggatgtgaaggacctcttcaaggagaactacaaaccactgctcaacgcaataaaagaggatacaaacaaaaggaagaacattccatgctcatgggtgggaagaacgaatatcatgaaaatggccatattgcccaaggtaatttatagattcaatgccatccccaccaaactaccaatgactttcttcacagaattggaaaaaactactttaaagttcatatggaaccaaaaaagagcccacaacaccaagtcaatcgtaagccaaaagaacaaagctggaggcatcacactacctgacttcaaactatactacaaggctacagtaaccaaaacagcatggtactggtaccaaaacagagatatagaccaatggaacagaacagagccctcagaaataatgccgcatatctacaaccatctgatctttgacaaacctgacaaaaacaagaaatggggaaacgattccctatttaataaatggtgccgggaaaatggctagccatatggccagtttcagaaagctgaaactggatcccttccttacaccttatacaaaaattaattcaagatggattaaagacttaaatgttagacctgaaaccataaaaaccctagaagaaaacctaggtaataccattcaggacacaggcatcggcaaggacttcatgtctaaaacaccaaaagcaatggcaacaaaagccaaaattgacaaatgggatctaattaaacaaaagagcttctgcacagcaaaagaaactatcatcagagtgaacagggaacctacagaatgggagaaaattttcgcaatctactcatctgacaaagggctaatatccagaatcgacaatgaactcaaacaaatttacaacaaaaaaacaaacaaccccatcaacaagtgggcaaaggatatgaacagacacttctcaaaagaagacgtttatgcagccaaaagacacatgaaaaaatgctcatggtcactggccatcagagaaatgccaatcaaaaccacaatgagataccatctcacaccagttagaatggcaatcattaaaaagtcaggaaacaacaggtgctggagaggatgtggagaaataggaacacttttacactgttggtgggactgtaaacttgtggaagtcagtgtggtgattcctcagggatctagaactagaaataccatttgacccagccatcccattactgggtatatacccaaaggattataagtcatgctgctataaagacacatgcacacgtgtgtttattgcggcactattcacaatagcaaagacttggaaccaagccaaatgtccaaaaaatgatagactggattaagaaaatgtggcatatatgcaccatggaatactatgcagccataaaaaatgatgagttcatgtcccttgtagggacatggatgaagctggaaaccatcattctcaggaaactattgcaaggacagaaaaccaaacactgcatgttctcactcataggtgggaattgaacattgagaacacatggacacaggaaggggaacatcacacactggggcctgttgtggggtagggggcagggaagggatagcattaggagatataactaatgttaaatgaggagttaatgggtgcagcacaccaacatggcacatgtatacatatgtaactaacctgcacgttgtgcacatgtaccctaaaacttaaagtgtaattttaaaaaaagtaaaaataaataagaattttcaaatgaaaaacaaagaaacaaaaaacacccaccatatcaaatattaaaactgacaagtgttctaatatttatatattggaatcattgcaaataatcaaatttcatattaaaataagtatttttattaaaaatctattttctaaaaacagaaaatattgagaagagatgcattattttacacttttttgaatctgttataatatctggctgaacagaagaaagttggattccctatttgcttctgcattcaatttttgaaatatgctgttttagttgaagtaaagaaaatccagacttccacagatgggtaatttgaaaggggaggagaattttaatagtctttttatatagttgtggatatttttctttgatactacaccatgatctgaaaaatgataaagtttttcaaaggatattttccatgagcattctgaaaacttgtcagtgaactttctgtactgctacattaaaatccatcggtctatcttgcacactgaatggatctttctcctatgtataattttgtaaacattattaattggtcatttggaaaatattggttcactgtgttatgtagatcttccaaatgttgatatatttctttatacagtataaaaatcacattagttaatatcaccaattttcccagaaaagcctttaaatattgagacgctatcaagctcacagtactagatacaggttctctaaaaatctaattttcacttgatccattgccaattttatcattggcaacgaatactattagttattagccttgaatagacaggctcactttattcatttttgagaaatatagtttgtcagtcactctttcaaataaaaatggtgatttatgaaagataaaaataaactagttcagcacaccactcaatcacccaagtgtttttccacaaccgctgtatttcagtatgtaacagaagtgtcttatgaacacttactatttgggcacaaaaatcattaaaaagaggtatacttaagaatcaagaggtaataaaattaataatttttactacaaaaaaatttgaaaaaattgcatgtgtaataaatatgtacagactttttcttgtcatcattctttaaacaattaggtataacaactattgacatagtattaaatattataagtaatctagagataatttaaagtatacaggagtatgtgcataggttttatgcaactaccatgccattttatattaggtacctgagcattcacaaatgttggtatctgtaagagttctggaaccaatccctatggatacagagggatgtctgtaacagaaaaatatgtaataaaggatgttagagtcccttagtttcaaggtacataattagctgttgggagataattgtccatgtgtctcacttttctgctcattgtgggagcactctggaacctttgtttcagaatattttttccttagtgtttctgtggtgacagagagagataattgtctttttctttgaggtgtgcttattgtccatataaaagtttcaacttccctatgtcaaagcagatttgtgctagacaatgttaaatggccaagcaaaattgtatttaagtgcatttcaatagagaagcaagaccaaaacttggtatgaactaggcaacttcatctaaacaaggggcaagatggtttttaaaggttgaggtaagctagtagagaagtatatcacagagaagtaatatcaaaagggaaaataaggctttgatatctttgtgacaggaagtagaagttttgtgactttagtgaggcatccactgaagttaggcttctactctcccatagaaagtgggagataagggtactgtcttgtttgaagattacatttcaaagagatggctctcaggtccttaagaaaaatatttctggattctaaaattaacaaaggcttttaaaaagatttatatcacgggggggcagagaaataatttacaattacaagttttctaaagtaaaccttttaagaaaaaggagggcaggtgcctggagtcaggaaaaagcctgtctaaagttcagtcaagcctaaggaaacattaaggctgttgtagtcactaagctctgggttcctgtcctgtaatatacatcactatatgtgcagatgttccagccctcttcacatttttctctgggaactgatgctcagcacaaattttgatagtctggctaccactattactgtgagtaataagctgtcgtctgtcttgaacccaggagtcttctactggcatccatgaaattgaagcagacctaactttgtaggcttgcaacaagtgcaaaatctcagactctttaaagttcttcaaatttgattgtgtaagtaaaagtgaagatttttgaaagttttgtgggcagttcatgtacatgaagaaagaactcagtagccaggccttgtgagggaaacagaacaactcaacaactccagagtatcttagctgtagcatcttgttagtcatctacttagggaattatcactggatgatttccagccctctgtgtctctactcaagattttaaatcctgggtaagagaaaaggatttgctctgctggggtcacaagtccaaacttgtggtcaaaataatgatttcctgtaatttgtttccccagttggttttcatggagtcaggaaaagaatgttccctgaaagagggatactgacataatgaaaatagtagatctccattaaattgtactgctgggctttacagcaatgtccacgtgtttgaatacacagacatatctcttcctttatatagttcccaaaatgtccattcttaatctaataaaatattattaaataaacacctactgtgtgttagccacttttctagtcaagggatacattagtgaacaaaggcacagaatcctgcctttaaggagtttatattctagtgcacagaaagaaaaaattaaacaggacaaatcagtaaattatgtcatatattagaggtaatatgtactatagaaaaaaagtggattgaggtaaaagagtagaggtgttaggagtgatgattttaaatagggtattcaggaaggcttcattaagaaggggacatttaagccaagacatgaaagaggtgagcaagttagcagttgcacacataactgggaaagaatactcagttcaaaggcttgaagcctagagcatgcctggggttttagggagtccgaggagactgtggttctaatggagagatggagggagagaggaggagcagatggattcagagagttatctgtggggataaaggtggcagcagagtgaccagctaggaggcagttgcagtaatctatgatactccaaagttaggaaaaagatgtgaatagagaatgactcttggattaagcatcataaaataaaatgaaatgtattcactgctcctaaaaggagactacctaaagtcacaaattagccataggaaggacctctggctgatgttcatgtgctttagtcctgtcatagtaccatctcagaattacacagtctttggactaggtggaaaatttaagtaccaccatcatactgttaaataacatgaatttttgaagaaatttaaagagaaaaattttaaaaactggaaataccattaggggttataatcctcagttcttccatggattatggtgatataattgctgtttacttacgcttaatctactcatttgctcttgccatttttttaacttcatttagcttagtagttgattggcatttggtgtaaatatttattccagacaactcagacttcttggagaaactgccttgatgagattgtgggtgtcttcttttatctctaggcagggtagtgcagtaaggcctacaaaaagattctcaacattctatgtcttcttctcatcttaataatataattgtaacctaatttctatctgatgctcagataagccaccctaagtgacaccatattcctttattttcttggttgttcaatagcttcggaaacctgaaatgaccagtggtctcattgatccaatgactgcttttgaggaagtgctctttctttgggctccaaattctgtaggccaataaagaattgggttagggcagagagaaatggttgagaagaaaatattttgagtaaaattagacattgatggttgagagttgaaccctctacctcactcctagggttcccagaagcccaccatatatcaattactgtttcatattaaatgtttcttccacaattgatggcatacttcggtttcgagatgttttattgaagttaacaacatactgagttctcaataggtcatttcagcaaatgtaagacagagttgcaccaaactgaagaaaatacaaaatatcagggttaataagtattgttgtcatctagcagtgagcagtagccaggtgagcttttatgaggcaaataaatcttgccgagtatatagatagctttccccactgccaacatagcctttgtccatcgtcccattaatccacactaggtagtctgcgaagaatgatgactgatgatctgaaagtaggttaccgcatctactggattattaagattgtttgtgttttggggtcttactgatgaacatttatatagatacaatcatattcaaggattatccttagaggtttttctatctatttcttcccccaaatatcctttttccattggtctgacttttgtcctaggtttgtcagaatatggcaagactaaagcaactttccaggatttgttgtaaacctcatccttggatgtctcttcacacagataaagtgagaaaacacatgttctgcctggagggctgccccctgatggttcaggtccatctctgactgaccacagagtctacttcttcttggtattgctatatcaagcagaactagctggaaaccaggcttcggagttttcgtgccgagtcatacgttcatggaaaaactcccttaaatgccatagtttcagactgaaagaagagtaggagtatgatttttaaaaaatatgcgttctgggattggaaactatatgtacatatcatatacttgtgtatttgggtatttttttttttttacctcagtcacatatatacttcttccaattcattaaaagttgctgattatacccagttttgtcgttatacatgtcaaatatctgataatgttgtctggtcacttagtttctacccaggaccagtaagtagtaagccagaaactttttaaaaacatttttaaaatttttatttcagtagtttttggggaacaggtggtgtttggttacatgattaagttcttcagtggtgatttctgagattttggtgcacccatcacaggagcagcgtacactgtacccagtgtgtagtctttcatacctcaccccctcccaaccttcccttccagtcccaaaagtccattgtatcattatgcctttgcatcctcatagcttaactcccactggtatgtgagaacatgtgatgtttggttttccattcctgagttactttacttagaataatggtctccaactccatccaggttgctgcaaataccattattttctttctttttatggctaagtagtatcagaagctttttcttaaataaaaataaatacttgctaaaagagccagaattttaatctgacatcagtattgtagtatgaaaaatatttttctttattattcagtttcttttagttctttattagttgcaaaaataattaactatataatacaaagggaataattttaaaaaaataagcaaatttagtttttaaataataaaaaactcaaaagaaccatttacattttgaactttttgttaaaacacctatatttataataaaatattaattatttaacatttatattaatgtatgattttagaaaaacaatgggcaaaaaaataaaaaatcttacaatctcttctttcaagagagcttttctttctccacgataaaaatgtgatattcacatcattaaaagtgaaagaaaactatgacttcctcttttgagacatgaaataggtactttcctaggttacttaatggagacagcaaacataaattgttttccttaaaattaacactctattgagaccatcctggctcacacagtgaaaccccgtctctactaaaaatacaaaaaattagccaggtgtggtggtgggcacctgtagtcccagctactcgggaggctgaggcaggagaatggagtgaacccgggaggcggagcttgcagtgagcggagatcgtgccactgcactccagcctgggtgacagagagagactccgtcaaaaaaaaaaaaaaaaaaatatatatatatatatatatatatacacacacacatatataaacgctctagttaaatgtaacattttatggaacacttcaagtaatcttccttttcaagcttacattttcattgtttattactgaaaagataacagctgctatggaataccctgctagaggaagtatactgtcagtcctaatttcaaattttttttctttaatttttccatttttctagtgagaaaaccaccttcaaatcaccagaagcaattttcacaggtaggtagacacacttgaaaaggtatataggggagaccctaaagttgcaagtgtatatttgccttacaagtgatttgtcccattgttttggggaagaaaaagggtaattagatctttcaacttttttcttgtacatatttagtggatacagtgtaatgttttgatacatgtacacataatgttatgatcagattgtcttctgagtgtttgaaaggagcagctcttgtgtagtagacacttatccccatggagaaaagagtgatggaaacttggggtctgaagtgcttacacaacagaacaaattctgactcagtgttcctgaggaaagctggtggttcatacagaattgtgaatgggaattaaaggggggcatagttgaaacctggagagaacaagtgtgagagagtcttaatttacctgggagaaattaatatccctcaaagaaccatgcctgttgggaagcccattgtgatgagtgagaaaaccagcactgcctgtcagccatgtaggaacaatcctactgatgatactgtggatggcaggggggacagcagcacatagtgaaccagctgtctcttgtctttggtatggaaaccactctgattccccttttaaatgtgtaaatccacagggtttccaaaaaattcaggatagaacgcctcgagaaggaaatagtgaatttcttgctaacaaggcatctggggggttgaggaataacttggacaaacaagagttgtaactgcatacacatctttcttaaattgtagaacagtttataaaatcatatggacttagttttaaatgaaaactaataaaccatacacccacaatatcagcacacttccttttgcatgttacatttctattctcatctatagaaagatacttttttacatagttaaaaataaacaatagaatcctcacatctgcccaacaggaccaggtaattggttgattaatccaaagtccgacagagttgaaaataaaaaagatgactcatatactaaaatgttttattttaactcgaaccacttaaaatgcatatatttgtgagtatattcatgtaaggataaagcttttactttgagtgttagactacttgatcagagttcttcagagtacctctagcataagcccactcataatgcattacattcattttggtgaatttaaatataagcagcaacttagagacacttgcaaatccaaatgaataagacttctagaatttttgattttattttcttcctcatatttagtctttccaaaatactcacctggctgtattaaaaacagatgatttttttgcaatcatactttattagtttacatagtacaaaattacattttccaagtagaataaatagaataataaatacaactgttagaagtggttttgagcacaacccccaacccccaccccaacttgtaggggcagaaatatgcaggcacaatggaaagtaatctgttaaacattaagtgttcattgtgccatgaaaatcagtcagggtgctttacagagggaagagagcatgctgtttagtttgttatataaaaatattttggccaggggcggtggctcactcctgtaattccagcgcttggagagggtgaggcaggcagatctcttgaggacaggagtttgaaaccagcctagccaacatggtgaaaccccgtctctacaaaaaatacaaaaattagccgggcatggtggtgggcgcctgtggtcccagctactcgggaggctgaagctcgagaatagcttgaatccgggaggtggaggctgcagtgagccaagatggagccactgcactccagcctgggtgacgggagtgaaaccctgtctcaaaagaaatttacatgtaatttttacagtcatacaaataatgatatttcagcttttcatttgtgagatgtaacataattttaacaaatcagcacattttaattctagtatggattagtctctgggcagaacaaggatctcattaacagtaattgacttcctcaactagaatccaagcactgtgagaatgagatagttcacatatttactcaaaaagaaatcttagttgtccagatgaaaaaattcaaggcttatcggttttaatattttaaataacatttcttaaactatgtagcttgaaaatgatagtttagatttaaaatcaatcctgatcagtgtttataattcaaattatattattgtaatcaataaaatgttaggttcttataaatattacactatgatcatcatagagattatggccatttttgcctcttcagagcactgtgttctatttgatcatcaagcatgaggatgtcattattgtttatttttagtctattattaaataaaattacatgatctgaaagacttaaacatatagctaagtgaaaacaaaacaagaactataaataaaaataatgtttaacttatgattaacataataaaaatctagaattatcaacaattctgataatatcgactaacatttttgtcactgtaaaacccagtttcttagtattaatatcttgttctccatttaaggcccagtttaaattttctctcccttcttttcatcccacttattctaaatctttttctctttcagatggaataaatactcattcctttgattctcataacttttatttatattcttttaactgatctgattatcttgcattatcatatttttttcagtgttcatcttttcacctaaccagtcccaaccaccataaactatacatttgttcattttaatttttcctctttgaatgggacttggtacacagatgaatgtattactagatgaatgcctgtgtcccggataaaaatgcttccagccacttcagactctgttctgtctctccaactggaaaagcaattcttgtccttatatttgttttggttgtgtctttccttgaaaaactctcatgcttttctctttttgccaagtgtgaattctttttttttaactagacatcggacaatatagagtgtagattgacttgcattttttttctttttggaataggagaaaataaaaaggcaagagtagtcttaatccttggaaacactgtctgagacatgcattaaaggtcagaaaattttatgcctttgatttcttctgtgactatcctagttaattttattattattttttgttagctcatcttggttgccacaggcattccaagaactcttttcaatgcagtttttacctctttgttcctcagactgtagacaagaggattaagtaagggagtaaccactgtgtaggtcactgccaccaactgatccttgtctgaggcagacttggacttgggccgcagatagatgatagaggcacagccatagtggacaaagaccacagtgagatgtgaggcacaggtgacaaaggccttcttgccctcagctgaggggatcttcaggatggtgttaactatgaagccataggatatgagaattaacagaaaaggcaccataattaccaggatgctgaggctaaataaagccagctctttcacatgggtgtcagtgcaggctaacttgataacaggtgccatgtcacagaaatagtggttaaccctgttggggccacaaaaacgcatgtcacaaatgaggttggtggccaccaaagcaataaagaaacctgtggctcctgagagagaaatcaactccaaccccagccttttgtttatgatgagtgtgtacctcagagggtgacaaattgctacatagcgatcatatcccatcacagcaatgaggaggcagttggtgcaagcaaagccaaggaaaaagaacagctgggtggcacaggccatgaaggagatggtcttggtgtctgagagcaggtggaccagcagctgagggatgatgacaaaagtgtagcaggactcagaaaatgaaaggatgaatagaaagccatacatgggagtgtggagagtccagctgaagcgaataacggccatgatggtcacattggccaccaggattgtcaagtataggagaagaaagatgacaaaaagcagcagctggagctcccccaggctggagaaacccaccaggatgaactgtgtaaccacagtggttttgttgaaacctcgcatctgaagaacttctttctgtgaatgtgtttgaaagagacaaaatgataactatctttgtaatacgaacattttcccaacgccttcactttaagtatattgtcagattcataatgttaacatctctttctcttagtctatctagatatagatgcctctttaatgatttttccaaaaaattattaatctgaagtaacagagaaatgattatctacagtatataaataccccaaagttaaaaggatccaactatttgacaaagtgaaaaaattctctcagtttaagaagaacattacctttaatatggaatagaaaatttaaattatttttcttgtactgtatgtatttggtccttggatgtatattctatcctagtaatttggtttcttttcctaatatcacaatccatttaattagcattattgtaaaggcattaaaacactgtgcctcagagcatgaactcaggaacaagaatgctagagtttgaaacttgcctttgccacttagagctatgaacttcagtgtgacaatttttgtcatttgtaagacataaataataatagaacatattatataggattgttgtaagaacttagatggtatcagtaaagtacttgaaacagcacttaacacatagtaactccaatctatgtgtcagctgcttatgcagttaaggagttgagaggcaaatatcctggtttgatatttgaacactagaagagcctggatccttaaatcaggacagagtttccctgcatttgactcaagtgttcttatccttgagattctttttttaaaattgagcaaaaatttaaatatgcacagttaaagaaagaactgttacttttacattaaaaattattttctttttaatttattttttattagcaactaataattatacctatttatggggtattatgtgatgtttggatacatgtatacattgtgggatgattaaatcagggttctttaataaatctgcccttttttatctaaggagtctatagcctttgaattctctttcatgattctatgacatttgttctgtatgtttctggaactgtgaagggacccaagtgtaaaacctgggtcacaaggttatgatgtatctgtctatgaaacaaaatagattgatgatcactagggataggggtggagcaggaagacttaggttaagtcttctctctattgaagaaaggagaggaaataacgtgaacacaccttgaatctcttgcaaataaaatagccagaaagtgctaaatggaaagagaagagcctatctcaggaacttcccattattttatagtctaggttattcctcctcccagagccctaatcatttttactttcagaatcagcttcagagattcaggagaaaccagatgtaatatttgcacaactcaacatctcccaacttgttggctactttaatggctataagatgttaggagttgtttgacatgacacacttgagtttgttcattgttcatcatctctgttgtggtttatttgcttaggtagttccacacacctgctacaacagctttttcctttgtttcagaccttgcggaaaataagatgctttcatgtaagctacttcattttgactctaacatcctggtagatagcaaaagtgtctgtcgctatctcacgggtaaggcaagtgtactaagtgccctctcctgggcagagtaacccatgaacatcctcatccttgcatttaacatgttgcattgaacatacctctttatatatttggcactctttatagattataagcttctcacatgcaggaataactttttttaattaaaaaatgagtccctagaatatagcatagtttgtgatttatcatgtgctcaaatattttgacttgattagaatgtcaaaaattaaataatatgctcaagtggtagatccagtatcagaattcaagtcttctaacttcaaatccactttctacttaccaaattttatggcacccattctttaccttgtgagaagatgatctgctcatatactttttccaagggatgaaagcatagttccacactgcttgttcaagtaaaacttatttgaacatttctgacttttctggaataatccaattttaattatccaagtttttgaattatcaaattttacaatatgagctttattctctttaatcttaatattagggagataagaaaaaaaggaaaaaggggtaggaaagtaggcaaatcaacgatatgattgatgaaaacactcaaaggcaaggaaggtcaagaggatgttgcttaatttcaatcacagttttatgaaccaaacaaattgcaatgtctccctaaatcattagcttctttttctgtgactttcatgagaattttgttaaataaaaaatatcctctaatcttgtcctcaacactagtactgcttctatatcttttttcaggttgcttcatatacttgttttgcccattgtttttcatattcaaatccctgtcatgtcccatgttctccaataaaccttccctgcctactagagtccatacagatgctcattctcttcatagcccttagggactttaaaatcatgcatgtatgggaacaggttaggtaatatcttgtattactttataaccaatttgcctatgtggatgcctcataaccttacttacattctttctttgaggcagggggcatagcaacgtattctaaacaatccgcagcagctgcaaacaatactgggcacacaacagacactaattaaatacttaataaattatggcatggtttatttcattcttcttttgtttatgagttttctcatctgctatttttttctggaatatattctttgttctactataatccgtattatttttaaatttttattttttttcaaaatcatcttttactttaaaaggagaaatctacactgctgagttttcctgcctttctagttacttctgttgcttgacatctcagcatttaaatttgatatattaagatggggtgggtgtatatgtgtgtgtgtgtatatgtgtctgtatctgtgtgtgtgcactctagcagagtggaagctttttcatttcaagaaaagagcaaataaagttttactttggctcagaatcctttttataacaataaaaactgctaatgaattcacacctgaatttttggtagaaaatatttctttctttagatagtaactgacaaagaaaatagacttacctacttatgttcaaaagatcaaggaagctgccatatgactttgttgacttatactctggtaggtcttttaaatattgaaagaggcagcagaaagtcagttgtaaaatcaatgacatctgtcccagaagattaaataattggaagaaaatgcactgaagcatttcataaagggcttatatggagacttagttgtccaaacctttttaagtcttaaataagagctgaaaatagtctggtttatattaatatgtcaattcagattctatataattgtatttgagagtatttatagccttctgggattctaaggagtaaggccttctgagacttagtaagagctaaaacaagagaggaaataaatgactaaccttgaacaaggacatattatttaataccagaagaaaaaaattcaaatggcaataaaacattccatctctgcaaagaactacaagtgtagataagtattctagtgattttctgacatcaagactacaaaatgttgaaatgccaggagaagccaaattctccaaccaggagatatacaataaaactgccttgataaggcctaagagatgaaggcaggggttggtgagtgctggttgcttggcacttttgggaattacctaagtagtctctgttctcctaaatcaaaacctggaaaaaattcaaataactggatttcttaattctgtcctaggaattattgtaaggaaatgactttcatcgaggaaccattcattgagtaccatgatttagagagcaggatatgagacattatagtgatacaaagataaaatgtatgaattgatctgctctagtagagaagacagatgtaagcagagctatgtgtaataagaggtaaaatgatgttcagtgttattgtgacattaacaacagggcagctacaaggatgagcacccccagcagggattgccttgaccacagccagggccgagattcagcaaatgacttcatgaataaaaacattctggaataataagccaagtagaaatataagaattaggaataaaaagtcaatactgaatgttttgtgcctaggacactcaaaggaaaaaggagtgaggaggaagactgagaactatttcttgtagtggcttggctggtcacatgagaaaactttctgaaaacaaaattgctgggaaaaattttaaaaaattttagaaacctattgacaagttgaaggtatagtaggaatttttaggccaaaatctgaagaaaataaaatccacagaggaacactggaaattgcttctattctgaggacatttatttacctgtatgaaattacaaactcaagctgttgatttgagagaatcacaggatgaggctgacagaaattaaagaccagaatctaacaaatgggggagtcaaaaattagatattaaagagattttctagttcccatatttgatgctttgacatctggaccttcctgaccaggaaaaaagcagccctcctagggttagcagattcatagagatagaaaatgaatttaatttcatgtgcagtcaaacacatccagaagtcatacccacaattgcctatttttgcatgctcttggactgagggacactacttccttttcctaatccccctggggccaggtgtcagacaactaaggatagtcctggaacccagtgaaactgtttaaactatccaattctaaacctgctcaaaaccacttaccctgctttgcctcaaaaccactctgaagactctccaaaacttccctttgctttctgcctacaactgaactttgtgctctggtgttctccccatggcacttcatgtgtggcgtgatatgccaccttcccactcaggcactcaggaactctaagtaatcaactatctttcaatggcagtcatctctctatctgctggtcttaccctatctgaataaaaataaagtcaacattttaaaacatattgaaggagaggaacaaagttgaagaactgacactatctgacctcaagacttactgcaaagctacagtaatcaagacagtgtggtattggtgaaagaacagataaatagaaaaataaatgggctatcaagccatgaaaagtcattgaggaaccttaaatgtgtattgctaagtggaaaagagctgtctgaaaagcctatatactatgtggtcccaactacatgacattctggaaaaagcaacactattgaaataataaaatgaccactggtgttttccaaggattcagagaaggaggggagggaaaaataaatggggcccaaggcatttttaggacagtgaaactattctgtataatactctaattgtgactatgacattttgcagttctgaaaatatgtagcatgaacccaaatgtaaaatatatggacttaggttaataataatgtgtcaacattggtttaataaataaaaaatgtacagcaataattcagaatgttaataataagaaaaactgtgcaggagagggaggccacaacggaatttgatattttctgtgtattttttctgtgaacctaaatatgctctaaaaataaagtctactgaagaaaaacaaaaagaaaataagattctcatagacatgatatgtagagatacaggccaagcacagtatgattcaatttatataatgatcaaggaaaggtcacaatgatggtggtggaagtcaggatagaagttatatttataaatgcaatacctgagaggcagtacaatggaaccctacagtgttctcataatatttaatttatttattagatatgttcaatttgtaacactttcatcaagctaaacccttatgatttacgtcccatttagtgtatatattatgttttaagatatttactttagttaaagtatactaattagagtttctctttggggaatagtgagctagaaatttttagtcagcccacagactagtaatcactagaaaaactacaaaaatattggaaaaaaatcagatcgaaggcatcagactttaacaaagaattagtattcaggaaaagcaactaacctgtgatgttgtcctggtatttgggagtaattagctcccaaggagaatctgacaattccggaagatattaccaaaaagtcaagaagttgagacacaccgaggtaaagagcaaatatatagatcatgaaccactgaggagtgatggggctggtgaacttcctgaactttgaattgacatttaaaagggaatataataggaacaagattaaacaagaaaaaaatcaactttcacaggagccaaattcagtttcaaacagccttaaaccctggtaaacttgagccaagactccatttaatactctagctccataagtccctactctgaaaagtagatcatagcagcattttgggtctcttgggatctgaacaggttaatagacagtatatcttatgatccctagaaggcctgggtatttttctttatccagtacacagataccttagtagatggcaaagttctctagggaaatattggaacaagattctcaggatataattgtggccatgctgttgtgtctttcctcaaaattccccatctaccctcacaatcaaggggttttgggtttgggaattggtgttgaattggcaattgggtgcaaagacatgaattaaaacactataacttacttttatctgtttgtcagacatgaaataaatgtcaaacatcactttagttgtctgctcatctgcttcattgctagaaatcccttatccattagccatttctacagatctctgcaggacaaaactcctctcattactctgaacttcaggtttatattggttattttccatatcttctccctgacagtttcatgtaacttcctgccttccatcctctggaatactattattcttattgatagcagtgagctcaatttctctgtagcatcatcaccgtcatctctagtttactaatgacaatttctttagaacacttcaaagatgtttatattctcatcaccaatgcatttattgatgcatttattaatgcattggtaggtggttattttccaggtcctctgtgtaacagtggttagggattagccaaggaggctgtccaaataattcattccaacacatctatttctctgaatctttgaattaattcctttacaatgagttaaaagatgtgacatttccaacttattaaccatagatcaatgtttagtttaggttttgtttagtctattagctagcaaaatattaatgccacttttgcatgcatgagccaatatattaaattctgaagcccaagggaatactcccatagcaatgagtttgactgaattccaatttatgtttcctctcccttggctaaagtctttacaattctttcctaaatataccctcctaactcccacagatacaaattggaaggcattttattattattattaattattattatttttgcagtttatctgttctcagagccaaccttgtacctctttatctgattatactgtgatccagttcctgttgtaggcctagaaacaaagttaagtgatggggcaattatttcaggtgcaatccctgagtcttttatgttgagtatggttgattaccttagatgtggtgtgtgcgcctgtgtgtgtgtcgggggacgggggcggtaagggtaatttaaaggaaatgggtcatcaagatctcagcctcacatggatctgctgaaaaatgtccatttcatgtctcaaagtcttacttcttattgagcagaagagcgctggtcaggctgatatttgacagctaaatgtataatcagtttgtgtgcttcatccttagccatggctgccctaaacccctggatgatccttcacatctgtcataaaaacattttgattttttcatacatcttttaaaatgcttattacttataatttccctttcgtctatgcttactctctatgatagtaaagaaaagccagaagcttaataaatctctgaaatctctgttattgccaacaaccacatgatcacccaatgtcttgccctccctctgtaattcatcctatgctattactggtgtttatttgttaattaaaatggtattaggtcaggagtttgagaccaacctggccaacatgacgaaacctcatctctactaaaaacacaaaaaaattagctgggcgtggtggtgtgtgcttgtggtccgagctactcgggaggctgaggcaggagaatcgcttgaacctgggagacggaagttgcagtgaggggagatcatgccactgcactccagcctgggtgacagagcaagactccgtctcaaaaaaaaaaaaaaaaaaaaagtattaaatgctgggatgaattcatccttttttaccccaacactgaagttatttgtgtactcattagatatacaagcaacccaatccaagaatcccattttcaggaatttttttcttaagttactcttgctcctaatcactgtttcagccaaggtccactagaaaataaatagcacatcaaaataaaggaaattcaagagataggaaaataccatagacaatggaataatctgggattagcagcatcccagtttttgtgtcttctattttataccctgttactgtgatccatatagatcagtcttctgagaaagaaagctgggtgaagaatggtagagagtagatctggagtgaatgtgtacatactgaacattagcataatattgactaaaaattctgacaaaagtagtacaacaaaagaaaatttcaagccaatctcttgaccatgggtgctgtgaaaattaagcagaagctttaagaactatggcacagctctaccatcctgttttttctcccttagtgagaacagcatgctattctcttaagggctgtttcttcagtcttaatcacagaataaaaaaaaagaagaactgtttgagggttgatacccacacacaacttgtttaagaaacagagctttgtggttctaagccatttagggtttagcattatttgctattggagaataacctaacaaaggctgactaatagagatgtaaaaattaaataactaaaggcttacaaaaccccaaggaatgtggtatagatgtattaaaatgcgatatactaggttttataataaaattaccaatgatagaggggttcggtttataatgcatgaattttaattctttaggaagccaccagtatatatatacacacacactcagaactaaagaaagaaatgcaaaaatctagaatcatttgggaggtttaacaaatcttttaaaataataccattatctatacatacataatatatttatggctgtatatttccttctaagtactattttagttaaatatcacaagttttcatgtacaatatttaaaaatattttcttgtgaaaatatgatttctttttaagctattatttacaagtgtgctttctaagttttaaacatatggggattttctttttttctttttttattatactttaagttctggggtacatgtgcagaacatgcagtttttttacatagctatacacgtgccatggtggtttgctgcacccatcaacccgtaacctacattaggtatttctcctaatgctatccctctcctagcccctcacccccaaacaggccccagtgtgtgatgttctcctccctgtgttcatgtgttctcattgttcaactcccacttatgaatgagaacatgcggtgtttggtttcctgttcttgtgttagtttgctgagaatgatggtttccagcttcatccatgtccctgcaaaggacatgaactcatcctttcttatggttgcatagtattccatggtttatacaaacatatggggattttctagtaatatattctactcattttgtatgcaataaatatttattgcctgttatgtaccaggcacccttccagctctagacatgcatcagtgagcaaaatgaataaatatccccataaatatatttatagctacatatttctaagtacttccttttcaacatagaaaggaagaaagaccaaaagctgcatgtagagccttaaagttatataggacttgaatagttaaaataggaaacaaatttaccatacagataccaacaaaaccaaaaatttgttctttttaaaaaactgataaaattgacaaatctctagtgagactgattagtgaaacaaaagcccaatatacattatcagaaatgataaaagagctataactaccgatgctgcagagaataacaaggtaataaggacatactgtaaaggttattatatatagctatgtataatttagatgaaaataaatgaaatgttaataaaataaacaatagcatgatttattctggaagaaattggggacttatacaaacatttaacaactgaattatttgtaaacttttcaataaagaaatatccagtccccaatggcttcaacaaagattataaaatttcagtgattcagaaatatttcagagaatatacaagaaaaatcccaagtcgttttatgaggccagaaaaccttgattttaaaccttaatgggaattgttaagagagataaaaatttcataccaatattcattcataagtacatacaaaatacacacacacacacatatggcatatctaaattgtctcagtctgagtaagtgtgggtgtgtgtgagtgcaccttgtgatggaatggtgttctgctcaggcttggctcctgctttgtgcttcggccttccagaaccctgaactgaaataaacgagttggaaaatgaatgaaaaatacaaattatttcaacataaaaatttgtaaatatacaataatgatacaaatgcatgacaataaatgatgtgatctgaaagtgttcagcaagcctgccatatttgtgattgttttgaactgccttgtggtaggaggtgctctttacaatgttctcttggcaagcatttattccttaatttaacctccactactatgatcaccattacttactgattcacctaaaattgggtaaataataatcatggttttattaatcttttgcaaatgtatggatagcttaaatttattacaatatttattgtaatattattattatagtaagtgaggacttactgtacaaaggtgatttgggagggttcattaatttcaccattatcctgttctgtcttttttactccctcttccactttagctatctttcttttgctgttaagtagcaagccccccatggacactctagggttggggaggcattgtgaataaccctggccatcacttgttgacctaatatgtgtaaggaaattacttaaaactttacacaaattgtcttattcaatcttcatgatcagaaaataaaagaaaaatcttatgtcaattgtgaggtaataaatggtatgtgtgaaagtcaggatagaagtcctgggttcccccttacagtgtcaatgggagcaatggaattcttgatctagacagccctcacatgaggacctacaagacttcccgctgggcagggtttgcaatacttgcccagatcatgggttgaatgatatatcagggaagtttataagttttgtctgggcatagaaccaagtggcctaagtactaatcttgcctgccaccaagcagctgtgtgggacattggacaagctatattacctctctagccttggttttcttctctgtgaaatagagattacataaagacatttgacatctttgagattctaaaacactcctggataagatatctgactagctagtctatgcttggggagaaatagaagaaaaggttgactccaaggaatggctacagggaaagaccaagtggatttacctagctagggagttgagagatgatacaaaattatcctgcctcctgcatactttgcctggatctgggcactagcatacgaccaatttccatacgacagaccctgagaccttctgggctgtaaaaagccttataaccatctgatgctacttttgctagcctttagaggtttgttcatcagaatctgccaatttctgcacaggagaacacagggaaaatagaaatagagtggctaactggttatgcggaactagtgtcctatatttgatcagcactaatattattggcttttcaatttctttattttatatgtgggcatattcttaacttttgggaatattcaagatagtgaagattgctgagaaagtaacgtttttggtttttctctttcagttccttcaagttcaatgcatcatagcaattaggaagtattagcattggtttctagtaagggttaggatatattactgcaggttagagatgttctgtaagcaaaacaccctgagccaatagggagaaagaactgactcaaggtcagcatgaaacagggacagagaaaagtttaacaatctggaccaaggtccaaacaagtaaataaaaaatgcagtaaatcctgacacaggtatcaaaacaaagaaacagccaagccagaggcaaatgcatcattttgttatgcagaatgaagtgaaatgggtaagaatataactttaacatttgtgttatacttaatataagaaccaaataaatgagacaagaaaaagtcctttgctgtaggaaaagggcattggcattcattcactgcagatttagcttcggaaactataaaatgggagacagaagatcaagcactcatgtaggaggaggagtcagtgcccagtcagtaccaagtgaagtgtgcctgggaacaacatggccataaacagcactacgcagtttaaactgtgttatcaagaaaagcatcaggtttcccagctggtgacagactccgaaatccattcactttactcttagggtgcataatgccgtccccattaatgactacaaaaataaaacatttaaaataaataaatatattaagaagacaataaacttttatctgtactcttaccacccagatatcaatcacttctattaacatgttcacatatgtttctgacttttctatgtgcacaatgtggagaaaagtgttttatggcaggcctgaactgctgctattcttaggaaagcctgcttgcaagattggcctttggctagcatctgaaaacttggctggcaaacagttccttacacaaatctttccctaattgataaggtggctcactgtccctctctgtttgtacaaagaatgcagaattttggtacatgctaggcagaggttgccaatgtggccaattctgaatgaagtcctcggacactgcgtttctagtgggttttcctgggtagaaacataacctagaggttgctgcatttttgttgatggggaaggatgcactatgactcttatggggagaggtagcatgagaagtctgcacatagattcctgcagacactgcctgtgtcttatgatttgactgtgtggtgaattctcattaagctcctgtaatcaatgttagctgtgaagacaaccctattgtgagtcttgagtcctggtagtaaagatttgaatgtggggttggtcttgggaactccccaacaaaacaaacacatgtaatatttttaaaagaagagatcgtattatctgcacctttttctacctttttatatagaatacatagaaaatatgtttctacatcaataaatatagacctataataatattataaattattgcatagcattatagttagtcttggacttttaagttttgcacattttaccacactgtgacttaaaaaaaaaccttgagatatagcaacacttaatcctcattcattattacttatgaggaaatttaaaaaatagtaattctgggtctacatacatttttatgatttttgatacatattgtaaactggcctttagaaagcatgagccagtgtccacataaagataatgtgtaatcaaaagtctcatttcccatttccagaatgataaaatcattaatttaaaaaaatttgattgatgcagcagagtgataattctttgttttaaattgcattacttcaattgtttacacaattggacatttttcaaaattcttctaaaaattgactatgatttcttcttttagaaatgcttatttatttatatttttcatttaaaaattatttggctttacttttatgatatttacatttctacaattaaagaagtaattatctttcataagtgtttaaaatattttttactcatttttaaattttatttaatagccacttcatattgttatataaataatttacacattcaaatcttttccttcatgcttacaacttctattttcatgtttgtagagctcgtctatatttagagaactctccaacattagatgcatttacctaaatttgctttttaactcactataattttgaaaatatggtaatatctgatctattttattttagattatattttgaacataatctaattggcatcctttagtattaaacaatgtgcatacttctatttctttaaaaaataacctttcctcactaattgaatattacttttgaatattccattctgtgctttgatttgtgtgtctagtttgctctaatatcacactggttgagtagttattttatagtacattttactttctgacagagcaaatcttatcactattctttagaagctctttgaatattaattcatgaccaatcttgcacttgaaattcaaggatatttatcatattatcaaaaataggtatttgattggaatttatttaaactttgaagtgaaataaggattaattggcatctctaaaattgagaaatcctatccttgtataatgcgtgcctgtattcattcatttttcattctgtatccccgtgaaaagtttttaatggctttactttaataggccttcactagaaattgttaaatgtgtttctggaagttttgtgagctgactaatttaaatttcctatttttcaaccactttccatctacctcacacaggtgtgttgagctccctaaaggatctcatatatctcacaccttacactgtcctcttgctttcctcatttgtaggctgttcagggatttactcttaaactatcagagttctgtggtctctagcacatccccctactttgttgaatcactgctctgtggagtttgtctcttctacaggatccttctgagctgtttccccactgatgaccagtgatgtcccctttggtctcttgccaaccacagatacaacatctgctttgtagcggtgttatcatatcctttgggaatgattactgcattatgttttaacaccagttctttcgaccttgctaataaagattaatcaagggaaatgaacaactttcctaagacatgaaagtgaagaagcaaaaaaagtggaatcaaattccaaaaggttttcatgatggaaagttaaaagtagagtggaatggttgggaaaatcatctcaggaatcacagaagttttggcggaaggacataggaagtcacctcagtgagcattgagagaggatgaaatcaatgcacggaagttgagtcagttagtgtcagataatttgctagagaatgactgtttctaaaatcttattttgccatgatttaattactctcttcccattacatgtagtcatactatattttctggttgttagagtcaacatcctattaaaaacaggtacacttgtgctacagacacattttattttatttttattttttcaggctggagtgcagtgagacaatcatagctcactgcagcctcaaactcctgaactcaagcaattctcctacctcagcctcccgagtagctgggactacagatgtgccaccatgccgagctaatttatgtatttatttactttagagatgagtggggagtcattttgttgtccaggctggtcttaaactcctggcctcaagttatcctcctgcctcagcttctccagtagctgggattacaggtgtgagccaccatgcccagccacagacacaatttaatcatgaaggattataagaagaggatagctccacacatttttaaatgtgcatgggcacttttgtgaggagttgccttttcaaatgaaagacccctactgtcctagaaaagtgtccaggactcagctagcctatgtaaaacaaaatgttttattttacaatggctgactttctaaaaatgtgtgaaactattaatatctcactaaaatcagcaggaaataaactgatttagaataattatgagttggatttttttctttgagtattactacccaatcaacctaattaactcttgatctttgtaaccgttaagactgttatatattaatgcataggaaaacaaactgtatttttaaataacctctcactaacttgtgtggaagatatagccttgatgtggaagactctttattgctaaacacaaacctactcatcttcctcggtataaatgcctctatgacagttatttacctgttgcccctcagcaataccccattcttctatattttgcaaattgccttttctttaactctcttgccttctggcttttgatacttgttaagcactgaagaaaaggatgtaagagaaggtgcttcccttctgcttccagctcctgttggcatttcctagtagcagtagatagctgtggatccagcctccagtttcctttaacactcttagtgccaaacttgctgtgttccctcagaggtaccagccacatccatgccaatcctctgggtaatcatgatactactgcatgctcccacacccactgggtatacccaatagatcacttaatcaactgtcaggccactcccactcctggaaggttctagcattggacctccaaggtttcttctctgaactcctggtttctattaacaccacattttctccatccattgcaagtctaagcagagtagccattttccttaattatacaaatgtgttgccctagcatccctttttgcttttacaaattttcacattcgtgtaagcaattccctatattaaaccagttctgtttgaaataattgccattacttttgttttattggcttacccttgagagttagagcctcggacataacagaatataacattgaatattgagtgtgggtaagcactttagacttccatgatatgtgggagaaacggaatgctcagcataagatggagtgagtaacggaagtggtactctggtagagattcaggattggcacatgttctcacaagtgggagctaaacagtgggtacgcatggacataaagatggaaatggcagacactgaggaccccaaatgagggagggtgagagggaggttagtgctgaaaaattatctatcgggtacaatgttcactgtttgggtgatgagtacactagaagcccaaacctcagcatcacacaatatatctatataacaaacctgcacatgtgcctgttgaatctattttaaaaaaagcttttggtttgaggaagagaaagtaggatcaattgaaggagtaggaggctggcaatctaggggagagatttgggaagagatatctaaaggtgatagttggagtggaatgtgattcttcgctaaatgtttttttgaaaataaaaatttacctgaggggttggatattggtttatatggggcactgaaaacagctattatgtgggttgccttctgttagtgtaaatgtgtacttgtcctggcacttggtgagagctgtcttatgatttcaattgtcatcgatctgttcaatgttaaaggatgtcttagaaaatcagtccaaagccagcctcataggctacaaatctagttttagtgctattataattttttgtggccaaggtaacagaaattccataatcctaacttggtataaccttttcttttccctataatgtattcaaaaacatatatacataatagactatgatgaatgggaattcaaaacatcaatttattattattggttgagtttatgcttaaaattcaagtgcagatttctctgtaagctaaacaggcatatgtggcctagttagaaggcctaagggactttactctcttgctaaggccaactacaaatctaaaaatcttacccctctcctgcaaattacatatgactggacaatagagatatgtgtgtatgcacatgtgtgaaaagaggaacagagagagcaagatgttcagttttaacacatgggagtgcaggccaatgtcaacttaatttttcattcacatggaaaaaataagaggaaggataaaatgccaggagagtcattttttatattcatgaccttttaacacaaagtggtcctgcccacttgtcagtgttgtggtgtcagacaccaaaataatagtatccacagctcttggaaagaaacaaggtttaaaagactatttaacatcaagagtatatcttcccacagatgacccatggcttaatcttctattgttaaaggtgaatacttacacattctactccattaattaaaaaaatcacagtcttttcaagaggtcatgtgagagcattataaaagcattaataaagaaaatatatcgtgtggactcagtattaattcttttactcattagtaggcaagtcattttatttgtctgagtgtcatttttctcatctacaaaatgtcgattataatatatacttcccagtgtgctctaaagtttagatgaagtaacattgttaaactgtagcacacaatgcagctataaataattaacacattaaacttagctcatttacttttcatgaatcaacttgatatgctagaggcctggggtgggcaatatggccaaggaaaagacaacttgggggccactgaaattaattttgcatcaatctctgattttcaacccccatcagcagcaagtttcaagcttcaaagaaatggaagaaatgcaattgacttacattcttggcagatgtcctacaataaacagggcctgtgaacaaaacattccacttccttccctcccgcttcaacccctaaaaggtatatgtcattgatgaacagactggtggaagaaaaggaaaaaacttagaaacaaaattaatcaatcaggagacttttattgacaggtggatcttgaaaagagttataatgtgagaaatcatcactattgtgaaacttcctttttgctatcactcctgcagaaaggacagagaatgaaaacatcaaattcaagttctggtctcaaatgtacatgcaaactcttgagttgcatggagaacagattgattgttccatatgcaaagctttccacttggtgaatatttagtgaaattgacttaatctttagcttgccttgcctggtcatttagggtgattttaggtcttgtcattaatccattcccctgtgaatatcttcagggtatgctgaccaaatttagatgtatgcctctgcatacaaaattagaaatgatcgtatttcattaatacactagaagcatggaaatggataacttttcataattttctaacttgaacattatttggatatatcttatgcatcaaacattgtaatgacgcttaacaagtatctcatttaacaattgtgatctgatgattttggaagactgaggtccagaaaacatctagaaaaatcctagagatagaagcttcctgagtccctagaacagagagttagggttgtgccacattttgcagggttagttttcagcctggctgagattgggtgacagtatttactacagcctaaaattctgaacatacatatgcttccccataatagccatctgttattttttaatcaattgaaaaaaaaccttcaataggcaataattcatgtgagtgtggcaagcagttgtgtttttctaaattttatatttggagatgacgggaaatgatctatgttcctgaacataatgctcaattgcttgatcatttgtgttctgggaattttttcctgtataacttgtggtttacggtttggaaagagcagaatcaagatttttttttttttttttttttgagacagagtcttactctgtcgcccaggctggagtgcagtgacacaatctcactgcaaccttctgcctcctgggttcaagtgattctcctgtctcaggctcctgagtagctgagattacaggagcccaccacagcatccggttaatttgtatattttttggtagagataggatttcactatgttggccaggctggtcttgaagtcctgacctcaggtgatccacctgtctcagccttccaaaatgctgggattacagtcgtgagaaactgcacctggccagaatcaagattaattttacaacagggaaattgttcttctcacaattttacaaagagctgatttgaactctttatttctcaagctataaatcattgggttgaacaatggagttataatagtgtaggatactgatattagagcatcctggcttgaggagtagttggactgaggccttaagtagataaaggaggcacagccatagtggacagtgacaataatgaggtgagatacacaggtagaaaaagctttgcacctacccagtgtggaaggaaactgaagtatggcagagaggatgtgaacataggacaccaagatcaacaataaggggatagccaggaccaatgtacagagcatgaagatgacaatctgactaaagtggttatggtgagatgccagcttgaggacaggagcaatgtcacagaagaagtgatgtagttgattggaggaataaaaaggcaggtgaaataccaaggatgtgatgatctgtgcaacagtgaagccacaggcacaggcagcagccactagtcccatacacaccccatgtcccattagcactgagtagcgcagtgggttacagatggctatgtaacgatcataacccatgactgccagcagaaaggagtgagagcagccaaggaagaggaaggaaaacatttggatggcacagcccaggaaagaaatggtcttcttctgggacagcaggtcaaccagcatcttgggtacaatgatgaaggtgtagcaaatctcagagcaagagaggatggcaaggaagaagtacatggggatatgaagggccctgtccaggacaatggtggaaatgatgattgcattggtgcccagagtgaacaggtagaggagcaggaagataacaaagagcagctgctgcagcctggccagggatgagaagccgaggaagatgacctctctcaccacagtctcattgacccgctccatggagcatacatcatcaggagacaattagggagagaagaggagtcagcaccaaaagaaatccctgaaaataagtagattcacagaaaattgatcatcagcatgcgttctcagattttgaattctgaactcatcttttctcctccctcttgtttttccaagacatgatgttgctaaaatcaggcaagcgttcatcaaactttgagtctccatagtagttctaacaccatagccaaaaagttcttgatacttttcagattcaaacaacccttcacctccattcccatcatgaggctgttgctcagattcttagcctttcactcttagattatcttgcaaagcttgtctttttggctttcttgtatctgtattctctcaaaaaaaaaatctttcctacagttgcaaagtaaaccatctgaatataccatagttgtctataactcaatggttcttaattgtcattctcgttacacctaaacccccactcttagttttagtctctccataacttagattcaaattctcttcaacactgttttccttacctcctggcaggaattgtcagttatacttttattatgctggctcttgtttctgtagctttatcctggtcccaaagtggaaaaaaattatcttcttctttctattcattaatttcaaggtggaatatgtctacactttctctaatacctatatcagagtctttcctctggcagggaggaccacaatctttcttcaggattctgtccattttcatcacttcccttaaatccttctgaagtcacattatggctcctttcccatggccactcttacatctctgcaacccatctttcccgtgatgacagcctaatcttttaaaatataaattgtattatattgcttcactcttcaaaaattttacttatggctatagtatagagttcaaactcttcattgtttccaaaagtctttcctttggggaaataaactatctttctgtgctcattttcagaccttaatgggatgtatcactattgcccaaatatgttctggacttccttgcctctattccttgctcatattagtttgttattcacttttatttacagatattttatggttcttctgaagcctccattaaaatctacctttttgaaagctttcttgctaccttctggtgaaaggagttgtttcttcctcagaactcacataagacattgattgccaaaattatgtgatatttacttaattttgaaatattctaatgaattgttcttatatctattttctctaccagattttgtacctttgaggaaaatatgtgaattttatgtatctttgcctgagttctcctctgcatctagaaccatattaagtacttaacaccttctcaataaataattgttatataaataagagagttaacttgccacattatctattttattcatgggcagcacagacttcttagaactcacagagtaagagaattc

SEQ ID NO: 2 - DNA, 2072 bp, Artificial Sequence
  Description of Artificial Sequence/note = Synthetic Construct
gggcagtcgtaggagactctgaaaaagcaaataaatcaatgttaaatcagaaatgtgaatgtagtaaggggctgaagagacaggggaagagaatacatgggaaaatattgaaaaggacagagtgatcaaaaaaagcagggacatgggagcattgggcagcacactgggagccatttactttatgctcttattgtatgattgagaaaaaaaatgtccttagtggttaagtggcttttcaatgccacatcagacttgttccatagcagttgaattaggggaaggtgaataagttggaggttggtgacaaggagagaagctggaacagagaggagagtcagaaccagagggaaatgagagactgagtaggcatctcagggtttttgaaggagtggattttctttgttgcagtcaggggaggtttgtctgttggctgcagaaagaagtcagaatagagatatcgtggggtaggtttgtttggaacagaaatcaaagaccaatttttctgagagaaggaaataacatctgcaaatgatatgctgtttttgctacttccattgttagctgttctcccaggtgatggcaatgcagacgggctcaaggagcctctctccttccatgtcatctggatcgcatccttttacaaccattcctggaaacaaaatctggtctcaggttggctgagtgatttgcagactcatacctgggacagcaattccagcaccatcgttttcctgtggccctggtccaggggaaacttcagcaatgaggagtggaaggaactggaaacattattccgtatacgcaccattcggtcatttgagggaattcgtagatacgcccatgaattgcagtttgaatatccttttgagatacaggtgacaggaggctgtgagctgcactctggaaaggtctcaggaagcttcttgcagttagcttatcaaggatcagactttgtgagcttccagaacaattcatggttgccatatccagtggctgggaatatggccaagcatttctgcaaagtgctcaatcagaatcagcatgaaaatgacataacacacaatcttctcagtgacacctgcccacgtttcatcttgggtcttcttgatgcaggaaaggcacatctccagcggcaagtgaagcccgaggcctggctgtcccatggccccagtcctggccctggccatctgcagcttgtgtgccatgtctcaggattctacccaaagcccgtgtgggtgatgtggatgcggggtgagcaggagcagcagggcactcagcgaggggacatcttgcccagtgctgatgggacatggtatctccgcgcaaccctggaggtggccgctggggaggcagctgacctgtcctgtcgggtgaagcacagcagtctagagggccaggacatcgtcctctactgggagcatcacagttccgtgggcttcatcatcttggcggtgatagtgcctttacttcttctgataggtcttgcgctttggttcaggaaacgctgtttctgttaagacacaccatgagcctcctcgtcacccttctccttttggggtgagagaccagcagcccaagggctccagacacacctgaacacatcgtgatgatgacgtcctctcaactctctttgtaaaaattttgttatttttgcttgtttctgattaatgattgtttgtcaatataagctcaatttaattttgcaggatttgttgttctgacctgggttctgggacttttaaattcaaattttatctccagatggaatggggtcctagcaacctccacatgttcacctattaatggatcatcaggcctgttttagatatcccttactccagagggccttccctgacttacaagtgggaagcagtctcttcctggtctgaactcccgccacattttagccgtactttgctaactgtgctcctcacttcctcttcttcattgcagttatttagatcccccctttccttctaatttttcagctccttcaatgcaaagtacatgtatttttaatatatgcatccctggtgaaggatcttgcctgcatgaaacatgttctcaataaa

SEQ ID NO: 3 - protein, 327 aa, Artificial Sequence
  Description of Artificial Sequence/note = Synthetic Construct
MLFLLLPLLAVLPGDGNADGLKEPLSFHVIWIASFYNHSWKQNLVSGWLSDLQTHTWDSNSSTIVFLWPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 4 - DNA, 1295 bp, Artificial Sequence
  Description of Artificial Sequence/note = Synthetic Construct
atcaaataccagctctgccagtaagaagttgcatctcccagtgaaatgctgctgctgccatttcaactgttagctgttctctttcctggtggtaacagtgaacatgccttccaggggccgacctcctttcatgttatccagacctcgtcctttaccaatagtacctgggcacaaactcaaggctcaggctggttggatgatttgcagattcatggctgggatagcgactcaggcactgccatattcctgaagccttggtctaaaggtaactttagtgataaggaggttgctgagttagaggagatattccgagtctacatctttggattcgctcgagaagtacaagactttgccggtgatttccagatgaaatacccctttgagatccagggcatagcaggctgtgagctacattctggaggtgccatagtaagcttcctgaggggagctctaggaggattggatttcctgagtgtcaagaatgcttcatgtgtgccttccccagaaggtggcagcagggcacagaaattctgtgcactaatcatacaatatcaaggtatcatggaaactgtgagaattctcctctatgaaacctgcccccgatatctcttgggcgtcctcaatgcaggaaaagcagatctgcaaagacaagtgaagcctgaggcctggctgtccagtggccccagtcctggacctggccgtctgcagcttgtgtgccatgtctcaggattctacccaaagcccgtgtgggtgatgtggatgcggggtgagcaggagcagcagggcactcagctaggggacatcctgcccaatgctaactggacatggtatctccgagcaaccctggatgtggcagatggggaggcggctggcctgtcctgtcgggtgaagcacagcagtttagagggccaggacatcatcctctactggagaaaccccacctccattggctcaattgttttggcaataatagtgccttccttgctccttttgctatgccttgcattatggtatatgaggcgccggtcatatcagaatatcccatgagccatcatcatgtctcctctcccattcgcaataagctaccaagaagcccaagatatcagcccaaaaatcaatcttatcatatttcaaatgattttcaaatttgatgaaatcagagttttcatgtattttaaaataaattattatttaaacatcagcaaaaaagtacttaaaactgtaaatttattatgagactgtactaacagtgtgattcaccctgattttacacacattaaaatgttagaaaaaat

SEQ ID NO: 5 - protein, 333 aa, Artificial Sequence
  Description of Artificial Sequence/note = Synthetic Construct
MLLLPFQLLAVLFPGGNSEHAFQGPTSFHVIQTSSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRNPTSIGSIVLAIIVPSLLLLLCLALWYMRRRSYQNIP

SEQ ID NO: 6 - DNA, 1207 bp, Artificial Sequence
  Description of Artificial Sequence/note = Synthetic Construct
acagagatcagcaaacagcttttctgagagaaagaaacatctgcaaatgacatgctgtttctgcagtttctgctgctagctcttcttctcccaggtggtgacaatgcagacgcatcccaggaacacgtctccttccatgtcatccagatcttctcatttgtcaaccaatcctgggcacgaggtcagggctcaggatggctggacgagttgcagactcatggctgggacagtgaatcaggcacaataattttcctgcataactggtccaagggcaacttcagcaatgaagagttgtcagacctagagttgttatttcgtttctacctctttggattaactcgggagattcaagaccatgcaagtcaagattactcgaaatatccctttgaagtacaggtgaaagcgggctgtgagctgcattctggaaagagcccagaaggcttctttcaggtagctttcaacggattagatttactgagtttccagaatacaacatgggtgccatctccaggctgtggaagtttggcccaaagtgtctgtcatctactcaatcatcagtatgaaggcgtcacagaaacagtgtataatctcataagaagcacttgcccccgatttctcttgggtctcctggatgcagggaagatgtatgtacacaggcaagtgaggccagaagcctggctgtccagtcgccccagccttgggtctggccagctgttgctggtttgtcatgcctccggcttctacccaaagcctgtttgggtgacatggatgcggaatgaacaggagcaactgggcactaaacatggtgatattcttcctaatgctgatgggacatggtatcttcaggtgatcctggaggtggcatctgaggagcctgctggcctgtcttgtcgagtgagacacagcagtctaggaggccaggacatcatcctctactggggacaccactcttccatgaattggattgccttggtagtgatagtgcccttggtgattctaatagtccttgtgttatggtttaagaagcactgctcatatcaggacatcctgtgagactcttccccctgactcccccattgtgttaagaacccagcaacccaggagcctagtacaatatagtgatgccatcccgtcgactctccatttaaattgtttctctttctgcataataaacatttgttaataaaaaccaaaaaaaaaaaaaaaa

SEQ ID NO: 7 - protein, 333 aa, Artificial Sequence; identical to SEQ ID NO: 5
  Description of Artificial Sequence/note = Synthetic Construct

(19) United States
(12) Patent Application Publication
(10) Pub. No.: US 2006/0021075 A1
(43) Pub. Date: Jan. 26, 2006

(54) Group 1 CD1 transgenic mice and their uses

(76) Inventor: Chyung-Ru Wang, Chicago, IL (US)

Correspondence Address: NEEDLE & ROSENBERG, P.C., SUITE 1000, 999 PEACHTREE STREET, ATLANTA, GA, 30309-3915, US

(21) Appl. No.: 11/181,234

(22) Filed: Jul. 14, 2005

Related U.S. Application Data
(60) Provisional application No. 60/588,192, filed on Jul. 15, 2004.

(51) Int. Cl. A01K 67/027

(52) U.S. Cl. 800/18

(57) ABSTRACT

Disclosed are compositions and methods relating to a human group 1 CD1 transgenic mouse.

This application claims priority to U.S. Provisional Application No. 60/588,192, filed on Jul. 15, 2004, for Group 1 CD1 Transgenic mice and their use,” and is herein incorporated by reference in its entirety.

This invention was made with government support under NIH Grant R01-40301. The government may have certain rights in the invention.

### I. BACKGROUND OF THE INVENTION

The CD1 family comprises a third lineage of antigen presenting molecules which are specialized in presenting lipid antigens to T cells. In humans, products of four of the five CD1 genes, designated CD1a, CD1b, CD1c, and CD1d, are known to be expressed on the cell surface and mediate T cell recognition. Based on their sequence similarities, these human CD1 isoforms can be classified into group 1 (CD1a, b, c) and group 2 (CD1d). Homologues of human group 2 proteins are conserved in all mammals studied to date, but group 1 proteins are not found in rats or mice. Thus, characterization of the function of group 1 CD1 has been limited to human-derived cells. The group 1 CD1 has been shown to present lipid/glycolipid antigens derived from mycobacterial cell wall to various human T cell subsets. In addition to their role in the adaptive immune response, some group 1 CD1-restricted T cells exhibit autoreactivity and may have an analogous functional role to group 2 CD1-restricted NKT cells in activation of innate immunity. To address these questions, transgenic mice were developed that express human group 1 CD1 proteins (hCD1Tg mice). The studies disclosed herein show that the expression pattern of CD1a, CD1b, and CD1c in hCD1Tg mice mimics that seen in human tissues, and that group 1 CD1 molecules act as target antigens for T cell recognition in the hCD1 transgenic mice.

### II. SUMMARY OF THE INVENTION

In accordance with the purposes of this invention, as embodied and broadly described herein, this invention, in one aspect, relates to mice transgenic for the human CD1 gene (hCD1Tg mice) and uses thereof.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the genetic map of the human CD1 gene complex. RP11-101J8, a BAC clone that spanned 169 kb of the human CD1 locus was obtained from BACPAC resources (Roswell Park Cancer Institute) and used to generate hCD1 transgenic mice. Open boxes indicate CD1 genes. The direction of transcription of each gene is shown by the arrows. The integrity of the clone was confirmed by pulse-field gel electrophoresis and Southern blot analysis.

FIG. 2 shows expression of CD1a, CD1b, and CD1c in Tg64, Tg78, Tg55 mice. Thymocytes and splenocytes of indicated mice were stained with FITC conjugated anti-CD1a, CD1b, or CD1c and analyzed by flow cytometry. Specific fluorescence profiles from Tg mice (solid line) were overlayed onto profiles obtained from wild type (Tg⁻) mice (dotted line).

FIG. 3 shows flow cytometric analysis of CD1c expression from various splenic subpopulations from hCD1Tg mice. Splenocytes from hCD1Tg mice were stained with antibodies against CD1c, B220, F4/80, and CD11c. Specific fluorescence profiles in gated population from Tg mice (solid line) were overlayed onto profiles obtained from wild type (Tg⁻) mice (dotted line).

FIG. 4 shows the expression of human CD1 proteins on freshly-isolated bone marrow cells (a), and bone marrow-derived dendritic cells (BM-DCs) (b). BM-DC were obtained by culturing bone marrow cells in medium containing GM-CSF and IL-4 for 6-7 days.

FIG. 5 shows the expression of human CD1 proteins on Langerhans cells. Single cell suspensions from epidermis were stained with antibodies against CD11c, I-A^{b}, and CD11a, b, or c. Specific fluorescence profiles in CD11c⁺/1-A⁺ gated population from Tg mice (solid line) were overlayed onto profiles obtained from wild type (Tg⁻) mice (dotted line).

FIG. 6 shows the screening of short-term T cell cultures for group 1 CD1-reactive T cells by cytotoxicity assays. BM cells from Tg⁺ and Tg⁻ mice were cultured with GM-CSF+IL4. After 7 days, Mtb antigens were added to some of the BM cultures and incubated for an additional 12 hours. Cells were harvested, labeled with 51 Cr, and used as targets in a standard cytotoxicity assay. The cytotoxic response of T cells harvested from individual wells against the targets was measured in triplicate in a ⁵¹Cr-release assay.

FIG. 7 shows the target specificity of HN2 T cell line. The cytotoxic response of HN2 against the targets was measured in triplicate in a ⁵¹Cr-release assay. Target cells were CD11c⁺DCs from Tg⁺ and Tg⁻ mice, C1R, and various CD1-transfected C1R cells in the presence or absence of Mtb antigens. CD11c⁺DCs were isolated from GM-CSF+IL4 treated BM cells by magnetic sorting (purity>95%). (b) The proliferative responses of HN2 to Mtb lipid extracts. Target cells were CD11c+ DCs from Tg+ and Tg− mice pulsed with Mtb lipid extract overnight. The results shown are representative of one out of two independent experiments.

FIG. 8 shows the target specificity of HN1-4 CTL clone. (a) HN1-4 CTLs were incubated with ⁵¹Cr-labeled target cells for 4 h at an effector to target (E:T) ratio of 30:1. Target cells were CD11c⁺DCs from Tg⁺ and Tg⁻ mice, C1R, and various CD1-transfected C1R cells in the presence or absence of Mtb antigens. (b) HN1-4 CTLs were incubated with ⁵¹Cr-labeled C1R transfectants expressing the CD1a, CD1b, and CD1c at indicated E:T ratio. The results are representative of three experiments.

FIG. 9 shows that anti-CD1c antibody inhibits the response of HN1-4 T cells to human T cell lines. Jurkat and MOLT-4 human cell lines were labeled with 51 Cr, and used as targets in a standard cytotoxicity assay. The HN1-4 CTLs were incubated with target cells in the presence of mAbs against CD1a, CD1b, and CD1c or medium alone. The E:T ratio was 10:1.

FIG. 10 Characterization of long-term T cell cultures for group 1 CD1-restricted ganglioside 1 (GM1)-specific T cells. A. Antigen-dependent IFN-γ production by GM 1-specific CTLs. BM cells from Tg⁺ and Tg′ mice were cultured with GMCSF+IL4. After 7 days, GM1 was added to some of the BM cultures and used as stimulators. CTLs derived from mice immunized with GM1-pulsed hCD1Tg DC were stimulated with various BM cultures for 48 hours. The amounts of IFN-γ in the culture supernatants were measured by ELISA. Results shown are the means from triplicate wells and the standard errors are shown. CTLs cultured with Tg− DC did not secrete detectable amounts of IFN-≡ (<1 unit/ml). B. Target specificity of HJ1 CTL clone. HJ1 CTLs were incubated with ⁵¹Cr-labeled C1R transfectants expressing the CD1a, CD1b, and CD1c in a 4 h ⁵¹Cr-release assay. The effector to target ratios (E:T) are shown in the figures. The results are representative of three experiments.

FIG. 11 Reactivity of CD1b-restricted GM-1 specific CTLs to lipooligosaccharides (LOS) from various isolates of Campyolobacter jejuni. CTLs were stimulated with Tg+ or Tg− DC pulsed with LOS from various Campylobacter. After 2 days, the levels of IFN-γ in the culture supernatants were quantitated by ELISA.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

23. “Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, or cultures derived from such cells. A “culture” refers to a composition comprising isolated cells of the same or a different type.

The term “pro-drug” is intended to encompass compounds which, under physiologic conditions, are converted into therapeutically active agents. A common method for making a prodrug is to include selected moieties which are hydrolyzed under physiologic conditions to reveal the desired molecule. In other embodiments, the prodrug is converted by an enzymatic activity of the host animal.

The term “metabolite” refers to active derivatives produced upon introduction of a compound into a biological milieu, such as a patient.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular human group 1 CD1 molecule (e.g., human CD1a, CD1b, or CD1c) is disclosed and discussed and a number of modifications that can be made to a number of molecules including the human group 1 CD1 nolecule are discussed, specifically contemplated is each and every combination and permutation of a human group 1 CD1 molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Investigation of the role of group 1 CD1-specific T cells in the immune system has been hampered by the absence of a suitable animal model. A recent study has suggested the use of the guinea pig as an animal model to examine the in vivo function of group 1 CD1-restricted immune responses (Hiromatsu, K., et al. 2002. J. Immunol. 169:330-339). However, the CD1 system in guinea pig is far more complex than that in humans. More than ten CD1 genes have been identified in the guinea pig, of which four genes are homologous to human CD1B, three genes are homologous to CD1C, but none of them are homologous to CD1A (Dascher, C. C., et al. 1999. J. Immunol. 163:5478-5488), (Hiromatsu, K., et al. 2002. Immunology 106:159-172). Furthermore, the lack of serological reagents to various cell surface markers and cytokines pose serious constraints on the use of this animal model. These problems are addressed through the development of transgenic animals, such as mammals, such as mice, which express human group 1 CD1 molecules and can be used as a model to examine the importance of group 1 CD1-restricted T cells to specific microbial infections, and to characterize the requirements for the development of group 1 CD1-restricted T cells as well as in the development and testing of vaccines for various microbial infections.

The present application relates in one part to transgenic animals comprising a human group 1 CD1 gene (hTgCD1). It is understood and herein contemplated that the disclosed transgenic animals can comprise the gene for one or more of CD1a, CD1b, CD1c, and CD1e, or any combination thereof. It is also understood that the use of the word gene indicates control regulatory regions are associated with the coding sequence. Also disclosed are transgenic animals that comprise one or more coding regions for a CD1 gene. The coding region is defined as the region of the gene encoding protein. It is understood that this can occur as a transgenic using just a cDNA for the coding region or a region containing the exons and introns for the gene, prior to splicing into the final mRNA. It is also understood that fragments of any of the disclosed CD1 genes can be used to make the disclosed transgenic animals. It is also understood that unless specifically indicated to the contrary or where a skilled artisan would understand it to be so limited, where disclosure of the word gene, cDNA, nucleic acid, etc it is also disclosure for all of the other types of nucleic acids, such as fragments of the gene, the coding regions, fragments of the coding regions, exons and introns, and a coding region cDNA, for example.

It is also understood that the animal can comprise any mammal. For example, the animal can be a mouse, vole, rat, guinea pig, cat, dog, cow, pig, monkey, or human. Thus, for example, disclosed are transgenic mice comprising the human group 1 CD1 genes CD1a, CD1b, CD1c, and CD1e. Also disclosed, for example, are transgenic mice comprising the human group 1 CD1 gene CD1a.

It is well understood in the art that gene expression is dependent on promoters to drive the transcription process. Specifically, it is understood that herein disclosed are transgenic mice whose genome comprise a transgene comprising a transcriptional control region operably linked to a nucleic acid, such as a cDNA, encoding a human CD1, whrein said control region comprises a human CD1 promoter. Thus, also disclosed are hTgCD1 animals further comprising a CD1 promoter operatively linked to a human group 1 CD1 gene or group 1 CD1 locus. For example, disclosed is a transgenic mouse that produces human CD1a, wherein said transgenic mouse has stably intergrated into its genome an exogenous expression vector that comprises a nucleotide sequence comprising a CD1 promoter operably linked with a nucleotide sequence encoding said CD1.

It is understood that many methods are known in the art can be used to derived the disclosed transgenic animals. For example, many methods can be used to generate a transgenic mouse having somatic and germ cells containing a transgene, said transgene encoding and expressing human CD1 polypeptide. Therfore, specifically disclosed is each and every method to make the presently disclosed transgenic animals. For example, specifically disclosed and herein contemplated is a method for producing the disclosed transgenic mouse comprising (a) providing a vector that comprises a nucleotide sequence comprising a non-mouse CD1 promoter in operable linkage with a nucleotide sequence encoding said CD1; (b) introducing the expression vector of step (a) into a fertilized mouse oocyte; (c) allowing said fertilized mouse oocyte to develop to term; and (d) identifying a transgenic mouse whose genome comprises the CD1 nucleotide sequence, wherein expression of said CD1 results in an increase in CD1 restricted T-cells. The disclosed transgenic mice can also be produced by (a) injecting into a fertilized mouse egg: a transgene comprising a transcriptional control region operably linked to a nucleic acid, such as cDNA encoding a human CD1, wherein said control region comprises the human CD1 promoter; (b) transplanting the injected egg in a foster parent female mouse; (c)selecting a mouse derived from an injected egg whose genome comprises a human CD1. A third example of a method for producing a transgenic mouse comprising a human CD1 gene, comprises (a) introducing a CD1 gene targeting construct into a murine embryonic stem cell; (b) introducing the murine embryonic stem cell into a blastocyst; (c) implanting the resulting blastocyst into a pseudopregnant mouse, wherein the pseudopregnant mouse gives birth to a chimeric mouse; and (d) breeding the chimeric mouse to produce the transgenic mouse, wherein where the disruption is homozygous, and the transgenic mouse produces human CD1, and produces CD1 restricted T-cells. It is understood that the injection can occur at later stage embryos, and can occur through, for example, ES technology. Thus, the nucleic acids can be injected into Blastocysts having less than or equal to 200 cells, 150 cells, 100 cells, 64 cells, 32 cells, 16 cells, 8 cells, 4 cells, or 2 cells.

As it is understood and herein contemplated that each of the disclosed methods of producing a transgenic mouse comprising the human CD1 gene are effective for what they disclose, specifically contemplated are mice produced by the disclosed methods.

Disclosed herein, are vectors comprising the genes, coding regions, or fragments for human group I CD1.

Vectors are understood in the art to be vehicles for the delivery of genetic information to a cell. Thus, such vehicles can comprise virus, plasmids, bacterium, as well as other means discussed below. Understood and herein disclosed are vectors comprising the genes for human group 1 CD1, wherein the vector is a bacterial artificial chromosome (BAC) clone.

Disclosed are cells produced by the process of transforming the cell with any of the disclosed nucleic acids. Disclosed are cells produced by the process of transforming the cell with any of the non-naturally occurring disclosed nucleic acids.

Also disclosed is a cell comprising a the disclosed nucleic acids and vectors comprising the genes for human group I CD1. It is understood and herein contemplated that the cell can be of any species and any tissue origin including but not limited to ES cells. For example, the cell can be an HN1 cell, HN2 cell, HN3 cell, or HN4 cell.

Disclosed are animals produced by the process of transfecting a cell within the animal with any of the nucleic acid molecules disclosed herein. Disclosed are animals produced by the process of transfecting a cell within the animal with any of the nucleic acid molecules disclosed herein, wherein the animal is a mammal. Also disclosed are animals produced by the process of transfecting a cell within the animal with any of the nucleic acid molecules disclosed herein, wherein the mammal is mouse, rat, rabbit, cow, sheep, pig, or primate, for example.

It is understood that the cells disclosed herein can be used to make a transgenic animal. Such animals are well-known in the art and can be made to express a variety of genes including genes engineered by man or genes of a different species. Typically, mice are used for this purpose, but other animals can be used including but not limited to voles, rats, guinea pigs, pig, cow, rabbits, dogs, cats, and primates (e.g., monkeys, and chimpanzees). Thus, for example, disclosed are mice comprising the cells disclosed herein.

Also disclosed is a transgenic mouse, wherein the mouse is a human TgCD1 (hTgCD1) mouse. As noted above, the human group 1 CD1 locus comprises multiple genes including CD1a, CD1b, CD1c, and CD1e. Therefore, specifically disclosed are mice transgenic for the group 1 CD1 locus and transgenic mice for the Group 1 CD1 genes. For example, specifically disclosed is a hTgCD1 mouse, wherein the mouse expresses human CD1a, CD1b, CD1c, and CD1e. Also disclosed is a hTgCD1 mouse, wherein the mouse expresses human CD1b, and CD1c. It is understood that other CD1 gene combinations can be found in hTgCD1 mice including but not limited to CD1a and CD1c, CD1a and CD1b, as well as the expression of only one gene from the group consisting of CD1a, CD1b, CD1c, and CD1e.

1. The CD1 Gene Family

The CD1 gene family comprises a group of nonpolymorphic genes located outside the MHC that encode proteins structurally related to MHC class I molecules. Every mammalian species examined thus far, including mouse (Balk, S. P., et al. 1991. J. Immunol. 146:768-774), rat (Ichimiya, S., et al. 1994. J. Immunol. 153:1112-1123), rabbit (Calabi, F., et al. 1989. Immunogenetics 30:370-377), sheep (Ferguson, E. D., et al. 1996. Immunogenetics 44:86-96), pig (Chun, T., et al. 1999. J. Immunol. 162:6562-6571), guinea pig (Dascher, C. C., et al. 1999. J. Immunol. 163:5478-5488), and human (Calabi, F., and C. Milstein. 1986. Nature 323:540-543), (Martin, L. H., et al. 1986. Proc. Natl. Acad. Sci. U.S.A. 83:9154-9158) possesses at least one or more CD1 genes. The best-characterized CD1 genes are those of the human CD1 family. It is understood that a Group 1 CD1 locus comprises more than one of the CD1 genes, and can include all of the CD1 genes, including upstream and down stream sequence. Five CD1 genes, CD1a, CD1b, CD1c, CD1d and CD1e, are known in humans. Four of the five CD1 gene products have been defined serologically, are referred to as CD1a, CD1b, CD1c and CD1d and are distinguished by unique heavy chains with approximate molecular weights of 49 kDa, 45 kDa, 43 kDa and 48 kDa respectively (Amiot, M., et al., J. Immunol. 136:1752-1758 (1986); Porcelli, S., et al., Immunol. Rev. 120:137-183 (1991); Bleicher, P. A., et al., Science 250:679-682 (1990)). Based on sequence similarities, CD1 genes can be separated into two distinct groups, with CD1A, -B, and —C in group 1 and CD1D in group 2. The human CD1E gene is intermediate between these two groups (Calabi, F., et al. 1989. Eur. J. Immunol. 19:285-292), (Porcelli, S. A. 1995. Adv. Immunol. 59:1-98). Mice have a much simpler CD1 system, which contains only genes encoding CD1d-like proteins (CD1 d1, and CD1d2, group 2 CD1). CD1 proteins are displayed on a number of antigen presenting cells (APCs) including Langerhans cells, activated B-cells, dendritic cells in lymph nodes, and on activated blood monocytes (Porcelli, S., et al., Nature 360:593-597 (1992); Leukocyte Typing IV, Knapp, W., ed., Oxford University Press, Oxford, U.K., pp. 251-269, 1989; Tissue Antigens, Kissmeyer-Nielsen, F., ed., Munksgard, Copenhagen, Denmark, pp. 65-72, 1989. It is understood that Group I CD1 proteins are presented primarily on “professional APCs,” while group II CD1 proteins are presented are a larger pool of cells.

Because of the structural resemblance of CD1 moleculesto MHC molecules (Calabi, F. and Milstein, C., Nature 323:540-543 (1986); Balk, S. P., et al., Proc. Natl. Acad. Sci. USA 86:252-256 (1989)), it has been understood that CD1 can represent a family of antigen presenting molecules separate from those encoded by the MHC genes. Porcelli, S., et al., Nature 341:447-450 (1989); Strominger, J. L., Cell 57:895-898 (1989); Porcelli, S., et al., Immun. Rev. 120:137-183 (1991). The five CD1 genes reveal exon and domain structure (α α1, α2, α α3) that is similar to that of MHC class I genes, including the association with β₂-microglobulin (β₂M) light chain for proper folding and presentation on the cell surface, yet the proteins are only distantly related in sequence. All CD1 family members share a conserved α3 domain; however, even this domain shows only 32% homology in amino acid sequence with consensus residues of class I MHC α α3 domains and there is no detectable homology with α1 domains. A major difference between MHC and CD1 molecules is presence of polymorphism in the gene transcript. Human MHC genes are extremely polymorphic: multiple alleles have been described at each known MHC locus. However, CD1 genes are typically nonpolymorphic. Despite these differences, the CD1 proteins, like MHC Class I molecules, are expressed as large subunits (heavy chains) non-covalently associated with ββ₂-microglobulin. Van Agthoven, A., and Terhorst, C., J. Immunol. 128:426-432 (1982); Terhorst, C., et al., Cell 23:771-780 (1981)).

Recent studies suggest that both group 1 and group 2 CD1 molecules may participate in innate and adaptive immune responses through presentation of self and foreign lipid/glycolipid antigens to T cells (Sugita, M., and M. B. Brenner. 2000. Semin. Immunol. 12:511-516). Group 1 CD1 can present microbial and self-glycolipid antigens to multiple subsets of T cells, including CD4⁺, CD8⁺, and CD4⁻CD8⁻ (DN) TCRαβ T cells (Beckman, E. M., et al. 1994. Nature 372:691-694), (Sieling, P. A., et al. 1995. Science 269:227-230), (Sieling, P. A., et al. 2000. J. Immunol. 164:4790-4796), (Rosat, J. P., et al. 1999. J. Immunol. 162:366-371), and TCRγδ T cells (Faure, F., S. et al. 1990. Eur. J. Immunol. 20:703-706), (Spada, F. M., et al. 2000. J. Exp. Med. 191:937-948). Group 2 CD1 present α-galactosylceramide (α-GalCer) and possibly self-glycolipids to a unique subset of T cells, the NKT cells, which express an invariant TCR α chain encoded by the Vα14-Jα18 gene segment in the mouse, and Vα24-Jα15 gene segment in the human (18), (Spada, F. M., et al. 1998. J. Exp. Med. 188:1529-1534), (Gumperz, J. E., et al. 2000. Immunity 12:211-221), (Lantz, O., and A. Bendelac. 1994. J. Exp. Med. 180:1097-1106), (Dellabona, P., et al. 1994. J. Exp. Med. 180:1171-1176).

The sequence contig found at NT_{—}079484 contains 1 the Homo sapiens CD1A genomic gene sequence. The symbol is HGNC. The locus ID is 909. This is the CD1A antigen precursor. It is also known as the hTal thymocyte antigen, thymocyte antigen CD1A, T-cell surface antigen T6/Leu-6, or T-cell surface glycoprotein CD11a precursor. CD1a precursor is located on Chromosome: 1. A description of the mapping of CD11a precursor is shown in Table 6,

TABLE 6

| | | | | |
|---|---|---|---|---|
| Chromosome: | 1 | | | mv |
| Cytogenetic: | 1q22-q23 | | HUGO | |
| Markers: | Chr. 1 | D1S3258 | D1S3258 | mv |
| | Chr. 1 | RH11438 | | mv |
| | Chr. — | RH120517 | | |

The mRNA sequence of the CD1a gene is set forth in SEQ ID NO:2 and can be found at Genbank Acc. No. NM 001763. The protein sequence can be found at Genbank Acc. No. NP 001754, SEQ ID NO:3, and has domains of cd00098: Immunoglobulin domain constant region subfamily and pfam00129: Class I Histocompatibility antigen, domains alpha 1 and 2.

The genomic contig can be found at NT 079484. Related sequences can be found in Table 7 in which Genbank Acc. Nos. of the nucleotides and the proteins are shown. These sequences are specifically incorporated herein by reference to there accession numbers. Other reference numbers are OMIM: 188370 and UniGene: Hs.1309.

TABLE 7

| Nucleotide | Type | Protein | |
|---|---|---|---|
| AF142665 | g | AAD37578 | BL |
| M14663 | g | AAA51934 | BL |
| M22167 | g | AAA51932 | BL |
| BC031645 | m | AAH31645 | BL |
| M27735 | m | AAA51933 | BL |
| M28825 | m | AAA51931 | BL |
| X04450 | m | CAA28049 | BL |
| None | p | P06126 | BL |

The sequence contig found at NT_{—}079484 contains the Homo sapiens CD1B genomic gene sequence. The symbol is HGNC. The locus ID is 910. This is the CD1B antigen precursor. It is also known as the thymocyte antigen CD1B. CD1B precursor is located on Chromosome: 1. A description of the mapping of CD1B precursor is shown in Table 8,

TABLE 8

| | | | | |
|---|---|---|---|---|
| Chromosome: | 1 | | | mv |
| Cytogenetic: | 1q22-q23 | | HUGO | |
| Markers: | Chr. 1 | SHGC-12745 | | mv |
| | Chr. - | PMC105497P1 | | |

The mRNA sequence of the CD1B gene is set forth in SEQ ID NO:4 and can be found at Genbank Acc. No. NM 001764. The protein sequence can be found at Genbank Acc. No. NP 001755, SEQ ID NO:5, and has a domain of smart00407: Immunoglobulin C-Type.

The genomic contig can be found at NT 079484. Related sequences can be found in Table 9 in which Genbank Acc. Nos. of the nucleotides and the proteins are shown. These sequences are specifically incorporated herein by reference to there accession numbers. Other reference numbers are OMIM: 188360 and UniGene: Hs.1310.

TABLE 9

| Nucleotide | Type | Protein | |
|---|---|---|---|
| M14665 | g | AAA51936 | BL |
| M22173 | g | AAA51940 | BL |
| BC069481 | m | AAH69481 | BL |
| M28826 | m | AAA51939 | BL |
| None | p | P29016 | BL |

The sequence contig found at NT_{—}079484 contains the Homo sapiens CD1C genomic gene sequence. The symbol is HGNC. The locus ID is 911. This is the CD1C antigen precursor. It is also known as the thymocyte antigen CD1C. CD1B precursor is located on Chromosome: 1. A description of the mapping of CD1C precursor is shown in Table 10,

TABLE 10

| | | | | |
|---|---|---|---|---|
| Chromosome: | 1 | | | mv |
| Cytogenetic: | 1q22-q23 | | HUGO | |
| Markers: | Chr. 1 | D1S3359 | D1S3359 | mv |

The mRNA sequence of the CD1C gene is set forth in SEQ ID NO:6 and can be found at Genbank Acc. No. NM 001765. The protein sequence can be found at Genbank Acc. No. NP 001756, SEQ ID NO:7, and has a domain of cd00098: Immunoglobulin domain constant region subfamily.

The genomic contig can be found at NT 079484. Related sequences can be found in Table 11 in which Genbank Acc. Nos. of the nucleotides and the proteins are shown. These sequences are specifically incorporated herein by reference to there accession numbers. Other reference numbers are OMIM: 188340 and UniGene: Hs.1311.

TABLE 11

| Nucleotide | Type | Protein | |
|---|---|---|---|
| M14667 | g | AAA51938 | BL |
| M22178 | g | AAA51942 | BL |
| CR457080 | m | CAG33361 | BL |
| M28827 | m | AAA51941 | BL |
| None | p | P29017 | BL |

In one embodiment, the CD1 genes can be contained in a nucleic acid which can be used as a vector, such as a BAC, containing the CD1 genes. Such an example can be found in SEQ ID NO:1. This clone was used to generate the transgenic mice disclosed in the Examples. In SEQ ID NO:1 the order of the genes is CD1A, CD1C, CD1B, and CD1E. In humans, T cell response specific to CD11a, b, and c has been described.

In SEQ ID NO:1, the exons of CD1A occur in the following positions: exon 1—nt 552-1142 (translational start site is at nt #1085), exon 2—nt 1495-1762, exon 3—nt 2415-2694, exon 4—nt 3197-3476, exon 5—nt 3832-3923, and exon 6—nt 4092-4657. In SEQ ID NO:1, the exons of CD1B occur in the following positions: exon 1—nt 77772-77878, exon 2—nt 77205-77472, exon 3—nt 76261-76540, exon 4—nt 75779-76058, exon 5—nt 75329-75424. In SEQ ID NO:1, the exons of CD1C occur in the following positions: exon 1—nt 36426-36538, exon 2—nt 37546-37813, exon 3—nt 38496-38778, exon 4—nt 39008-39287, exon 5—nt 39624-39715, and exon 6—nt 39867-40030. In SEQ ID NO:2, the exons of CD1E occur in the following positions: exon 1—nt 100400-100458, exon 2—nt 100818-101085, exon 3—nt 101711-101981, exon 4—nt 102238-102517, amd exon 5—nt 102909-103004.

2. Immune System

The immune system of animals, such as mammals, includes both molecular and cellular components. These components both recognize and attack potentially harmful foreign or endogenous but abnormal cells (for example, pathogens such as bacteria or viruses, and cancerous or pathogen-infected cells), but do not attach endogenous normal cells. When stimulated by foreign or abnormal biomolecules, the immune system undergoes a series of activities designed to neutralize and destroy the pathogens, or cancerous or pathogen-infected cells, with which the foreign or abnormal biomolecules are associated. These activities, collectively known as an addaptive immune response, may consist of a cell-mediated immune response, a humoral (antibody-mediated) immune response, or an immune response that includes elements of cell-mediated and humoral responses.

Humoral immune responses are mediated by antibodies that bind specific foreign or abnormal biomolecules. Antibodies are immunoglobulin (Ig) molecules produced by B-cells, as lymphocytes which originate in avian bursa or in mammalian bone marrow, but migrate to and mature in other organs, particularly the spleen. Robertson, M., Nature 301:114 (1983). Cell-mediated immune responses are the result of activities of T-cells, lymphocytes that undergo maturation within the thymus of an animal. Tizard, I. R., Immunology: An Introduction, 2d Ed., Saunders, Philadelphia (hereafter “Tizard”), p. 163, 1988. Both T and B-cells migrate between various organs and/or tissues within an animal's body. Lydyard, P., and Grossi, C., Chapter 3 in Immunology, 2d Ed., Roitt, I., et al., eds., Gower Medical Publishing, London, New York, 1989.

T-cells mediate at least two general types of immunologic functions, effector and regulatory, reflecting the fact that T-cell activities vary considerably among different subpopulations of T-cells within an animal. Rook, G., Chapter 9 in Immunology, 2d Ed., Roitt, I., et al., eds., Gower Medical Publishing, London, New York, 1989. Effector functions include delayed hypersensitivity, allograft rejection, tumor immunity, and graft-versus-host reactivity. Effector functions reflect the ability of some T-cells to secrete proteins called lymphokines, and the ability of other T-cells (“cytotoxic” or “killer” T-cells) to kill other cells. The regulatory functions of T-cells are represented by the ability of “helper” T-cells. Helper T-cells interact with, and produce biomolecules that influence the behavior of, both B-cells and cytotoxic T-cells, in order to promote and direct antibody production and cytotoxic activities, respectively. Mosier, D. E., Science 158:1573-1575 (1967). Other classes of T-cells, including suppressor T-cells and memory T-cells, also exist. Miedema, F., and Melief, C. J. M., Immunol. Today 6:258-259 (1983); Tizard, pp. 225-228.

3. Antigen Recognition

In order to function properly, the T- and B-cells of an animal's immune system must accurately and reliably identify the enormous number of molecular compositions derived from foreign (“non-self”), or endogenous (“self”) but abnormally expressed, compositions that are encountered. Recognition and identification by the immune system occurs at the molecular level. An antigen, a molecular composition having the potential to generate an immune response, is composed of one or more molecular-sized identifying features known as epitopes. A polypeptide antigen which has an amino acid sequence which comprises, e.g., a hundred amino acids might comprise dozens of epitopes, wherein each epitope is defined by a portion of the polypeptide comprising from about 3 to about 25 amino acids. The number of eptitopes derivable from polypeptides alone is estimated to be about ten million. Tizard, p. 25.

An antigen encountered by a T or B-cell of an animal must be identified as either being associated with normal endogenous (i.e., self) antigens, an immune response to which would be injurious to the animal, or with foreign or abnormal (i.e., non-self) antigens, to which an immune response should be mounted. As part of the immune system's means of identifying antigens, individual T and B-cells produce antigen receptors which are displayed on the T or B-cell's surface and which bind specific antigens. Turner, M., Chapter 5 in Immunology, 2d Ed., Roitt, I., et al., eds., Gower Medical Publishing, London, New York, 1989. B-cells produce and display antigen receptors that comprise Ig molecules which have unique antigen-binding portions due to unique amino acid sequences in the variable regions of each of the two antibody subunits, known as the Ig heavy and Ig light chains. Each B-cell membrane comprises from 20,000 to 200,000 identical Ig molecules. Tizard, pp. 78-80 and 202.

The T-cell antigen receptors (TCRs) produced by and displayed on individual T-cells comprise heavy (TCR.β.) and light (TCRα) chains (polypeptide subunits) which are linked by a disulfide bond on the T-cell surface. Each TCRα and β subunit has a carboxy-terminal constant region, the amino acid sequence of which does not vary from T-cell to T-cell, and an amino-terminal variable region, the amino acid sequence of which does vary from T-cell to T-cell. When TCRα and TCRβ subunits associate with each other, the variable regions of the TCRα and TCRβ polypeptide subunits combine to form the unique antigen-binding portion of an α:β TCR. A second type of TCR heterodimer, γ:δ, has been described but its function, if any, is unknown. Davis, M. M., and Bjorkman, P. J., Nature 334:395-404 (1988). Although at least one mixed TCR heterodimer of unknown function, β:δ TCR, has been described, T-cells bearing α:β TCR molecules are numerically dominant in mature animals. Hochstenbach, F., and Brenner, M. B., Nature 340:562-565 (1989).

Although each individual T- or B-cell displays identical antigen receptors, the receptor displayed varies from cell to cell; an animal's collection of different antigen receptors is thus quite diverse. The genetic basis of this diversity is as follows. The variable region of an Ig heavy chain, or that of a TCRβ chain, is encoded by three gene segments, the variable (V), diversity (D) and joining (J) segments. The variable region of an Ig light chain, or that of a TCRα chain, is encoded by V and J gene segments. Multiple DNA sequences encoding many different V, D and J gene segments are present as unexpressed copies in germline DNA; an analogous but different collection of variable gene segments for TCR subunits is also present. During development of an animal, genes encoding diverse variable regions are generated in individual cells of the immune system by the random joining of V, D and J, or V and J, gene segments. The process of DNA rearrangements that generates a randomly assembled variable region of an Ig heavy or TCRβ subunit is called V-D-J joining; the analogous process that generates a rearranged variable region of an Ig light or TCRα subunit is called V-J joining. Sakano, H., et al., Nature 280:288-294 (1979); Early, P., et al., Cell 19:981-992 (1980); Alt, F. W., et al., Science 238:1079-1087 (1987); Harlow, E., and Lane, D., Antibodies: A Laboratory Manual, Cold Spring Harbor Laboratory Press, Cold Spring Harbor, pages 10-18, 1988; Davis, M. M., and Bjorkman, P. J., Nature 334:395-404 (1988).

A functionally rearranged Ig or TCR subunit gene is one in which the DNA rearrangements of V-D-J or V-J joining have not resulted in a reading frame that is prematurely terminated because of the introduction of stop codons or frameshifting mutations. Because each T or B-cell of the immune system expresses genes encoding their respective antigen receptors in which a unique functionally rearranged variable region is present, many different T or B-cells, each producing a receptor that has a unique antigen-recognizing region, are generated. Hay, F., Chapter 6 in Immunology, 2d Ed., Roitt, I., et al., eds., Gower Medical Publishing, London, New York, 1989. The total catalog of different antigen receptors displayed on the T-cells of an animal is referred to as the animal's TCR repertoire. Bevan, M. J., et al., Science 264:796-797 (1994).

For mature T- or B-cells, binding of antigen to a cell's antigen receptor activates the cell, i.e., stimulates the cell to undertake activities related to generating a cell-mediated or humoral immune response. Typically, activated mature T or B-cells proliferate in response to antigen. In contrast, for immature T or B-cells, binding of antigen to a displayed TCR or B-cell antigen receptor, respectively, results in elimination of the cell by a process called negative selection or clonal deletion. Clonal deletion occurs during normal development of a healthy wildtype animal, and is a mechanism by which the immune system learns to tolerate the animal's normal endogenous (self) antigens, i.e., to treat the animal's self antigens as non-immunogenic antigens. Failure of the immune system to achieve or maintain tolerance of self antigens may result in autoimmune responses (i.e., autoimmune response to self antigens) that can culminate in autoimmune disease in animals including humans. Autoimmune disease can occur when an appropriate immune response to a non-self antigen results in the production of immune effector biomolecules (e.g., autoantibodies) or cells that cross-react with self antigens. Human autoimmune diseases include such crippling conditions as Multiple Sclerosis (MS) and Systemic Lupus Erythematosus (SLE). Roitt, I., Chapter 23 in Immunology, 2d Ed., Roitt, I., et al., eds., Gower Medical Publishing, London, New York, 1989; Steinman, L., Sci. American 269:107-114 (1993).

4. Antigen Presentation

Although the antigen receptors of B-cells can directly bind soluble antigen, T-cells typically respond to antigen only when it is in the context of MHC displayed on specific classes of other cells known generically as an antigen-presenting cells (APCs). Feldmann, M., and Male, D., Chapter 8 in Immunology, 2d Ed., Roitt, I., et al., eds., Gower Medical Publishing, London, New York, 1989. The nomenclature for MHC gene products varies from species to species. For example, human MHC proteins are also referred to as human lymphocyte antigens (HLA), murine MHC proteins are also referred to as H-2 antigens, and rat MHC proteins are also called RT1 antigens. Tizard, p. 181. Particular MHC proteins bind selected classes of antigens with limited specificity. For the most part, the specificity determinants in a TCR:Ag:MHC complex are (1) the unique polypeptide sequences of the variable portion of the TCR and (2) the unique polypeptide sequences of antigen. However, to some degree, MHC-presented oligopeptide antigens are embedded within an MHC molecule and TCR recognition of antigen only occurs within the context of an appropriate class of MHC molecule. Janeway, C. A., Sci. American 269:73-79 (1993). This phenomenon, called MHC restriction, is of fundamental importance to T-cell antigen recognition and physiology. Zinkemagel, R. M., and Doherty, P. C., Nature 248:701-702 (1974).

In MHC-mediated presentation of antigens, the αβαβT-cell antigen receptor recognizes peptide antigens in conjunction with products of MHC genes. In the case of soluble antigens, recognition occurs in conjunction with Class II molecules. For viral antigens, recognition is in conjunction with Class I molecules. Furthermore, large soluble antigens are processed from polypeptides by an appropriate accessory cell, such as a macrophage or dendritic cell.

The general sequence of events involved in T-cell recognition of polypeptide antigens in MHC restriction is as follows. A polypeptide antigen is phagocytosed by an antigen-presenting cell, internalized, processed, and then a peptide derived from the polypeptide is displayed on the cell surface in conjunction with Class I or Class II MHC molecules. In order to present antigen, MHC Class I molecules require an additional protein, ββ₂-microglobulin. Tizard, pp. 181-183. A T-cell antigen receptor a: heterodimer then recognizes the peptide antigen plus the MHC gene product. Recognition of peptide antigen alone or MHC gene product alone is not sufficient to signal T-cell activation. Only the MHC:Ag complex can be appropriately recognized by a TCR molecule. Steward, M., Chapter 7 in Immunology, 2d Ed., Roitt, I., et al., eds., Gower Medical Publishing, London, New York, 1989.

The genes encoding MHC proteins are diverse; however, unlike Ig and TCR molecules, which vary from cell to cell in an individual animal, MHC antigens vary from individual animal to individual animal or from one group of related individual animals to another group. Members of familial groups, represented in the mouse by inbred strains of mice, share similar MHC antigens with each other, but not with individuals from other strains of mice. Snell, G. D., Science 213:172-178 (1981); Owen, M., Chapter 4 in Immunology, 2d Ed., Roitt, I., et al., eds., Gower Medical Publishing, London, New York, 1989. Because variant MHC molecules will be capable of binding different antigens, the antigens that T-cells will be able to recognize (i.e., specifically bind in the MHC context) and respond to varies among different strains of mice. Cooke, A., Chapter 11 in Immunology, 2d Ed., Roitt, I., et al., eds., Gower Medical Publishing, London, New York, 1989. In humans, particular genetic alleles encoding MHC (HLA) molecules are more highly associated with autoimmune diseases, presumably because these MHC molecules are more competent at binding (and thus presenting to T-cells) self antigens. Vaughan, in Immunological Diseases, 3rd Ed., Vol. II, Samter, M., ed., pp. 1029-1037 (1978); Steinman, L., Sci. American 269:107-114 (1993).

5. T-Cell Subsets

Classes of T-cells are to some extent distinguished on the basis that different T-cells display different CD proteins on their surfaces. Immature T-cells display both CD4 and CD8 proteins (i.e., immature T-cells are CD4+ 8⁺), mature helper T-cells are CD4+ CD8− (i.e., display CD4 protein but not CD8 protein) and mature cytotoxic T-cells are CD8-8.sup.+ (i.e., display CD8 protein but not CD4 protein). Smith, L., Nature 326:798-800 (1987); Weissman, I. L., and Cooper, M. D., Sci. American 269:65-71 (1993).

In most cases so far examined, CD8+ T lymphocytes recognize MHC class I complexes, while CD4+ cells recognize MHC class II complexes on antigen presenting cells. The involvement of CD8 and CD4 in antigen recognition by αβ TCRs is significant. CD4 and CD8 molecules increase the avidity of the TCR interaction Ag:MHC complexes and are sometimes referred to as co-receptors (Bierer, B. E., et al., Ann. Rev. Immunol. 7:579-599 (1989); Steward, M., Chapter 7 in Immunology, 2d Ed., Roitt, I., et al., eds., Gower Medical Publishing, London, New York, 1989). Because of the importance of CD4 and CD8 in antigen recognition in the MHC context, CD8− CD8− (double negative; DN) T-cells have classically been considered to be immature thymic T-cell precursors. Lydyard, L., and Grossi, C., Chapters 2 and 14 in Immunology, 2d Ed., Roitt, I., et al., eds., Gower Medical Publishing, London, New York, 1989; Smith, L., Nature 326:798-800 (1987); Strominger, J. L., et al., Int. J. Cancer Suppl. 4:43-47 (1989); Shirai, T., et al., J. Immunology 144:3756-3761 (1990); Weissman, I. L. and Cooper, M. D., Sci. American 269:65-71 (1993).

The DN subpopulation of T-cells is distinctive in regard to the TCRs that they display. The majority of human DN T-cells isolated from peripheral blood express δγγδ TCRs. Porcelli, S., et al., Immunological Reviews 120:137-183 (1991). A large proportion (approximately 60%) of murine DN αβαβ TCR T-cells express Vββ8 gene products (Fowlkes, B. J., et al., Nature 329:251-254 (1987); Bix, M., et al., J. Exp. Med. 178:901-908 (1993)). Several analyses in mice point to a striking lack of junctional (V-J or V-D-J) diversity and restricted use of germline V and J gene elements, especially for TCRα subunits. Koseki, H., et al., Proc. Natl. Acad. Sci. USA 87:5248-5252 (1990); Kubota, H., et al., J. Immunol. 149:1143-1150 (1992).

6. T Cell Recognition of Group 1 CD1-Presented Antigens

a) Recognition of Group 1 CD1 in the Absence of Foreign Antigens

The first report linking T cell recognition to CD1 molecules suggested that CD1-restricted T cells were “autoreactive”, since the expression of CD1a or CD1c on the transfected cells in the absence of foreign exogenous antigens was sufficient for specific target recognition (Porcelli, S., et al. (1989) Nature 341:447-450). Direct recognition of CD1c has also been demonstrated in several γδTCR-bearing T cell lines (Faure, F., S. et al. 1990. Eur. J. Immunol. 20:703-706), (Spada, F. M., et al. 2000. J. Exp. Med. 191:937-948). Although the frequency of group 1 CD1-restricted autoreactive T cell is unknown, a recent analysis of approximately five hundred T cell clones from human peripheral blood lymphocytes (PBLs) yield fourteen that were self-reactive to CD1a, CD1b, or CD1c, suggesting that as many as two to three such precursors could be present per 100 circulating T cells in normal individuals (Vincent, M. S., et al. (2002) Nat. Immunol. 3:1163-1168). Most of the autoreactive group 1 CD1-restricted T cells lyse CD1-bearing target cells, and secrete Th1 cytokines, such as IFN-γ and TNF-αα Recent studies implicated that interactions between autoreactive group 1 CD1-restricted T cells and CD1-expressing immature DCs could lead to maturation of DCs (Vincent, M. S., et al. (2002) Nat. Immunol. 3:1163-1168), (Leslie, D. S., et al. (2002) J. Exp. Med. 196:1575-1584), which could potentially polarize the subsequent adaptive immunity toward a Th1 response. However, it is unclear whether these group 1 CD1-restricted autoreactive T cells can indeed be activated during infection and how these T cells are selected and maintained to avoid causing an autoimmune response under normal circumstances. To address these questions, a CD1c-specific autoreactive T cell clone from hCD1Tg mice was derived and characterized. Transgenic mice which express the TCR from this CD1 c-reactive T cell clone can be derived and used to study the requirements for the development and maintenance of group 1 CD1-restricted autoreactive T cells.

7. Transgenic Animals

Disclosed are transgenic animals comprising the disclosed nucleic acids. For example, disclosed are non-human transgenic animals comprising a human group 1 CD1 gene. Also disclosed are transgenic animals wherein the human group 1 CD1 gene is controlled by a promoter which is not endogenous to the transgenic animal, such as a human CD1 promoter.

Also disclosed are animals, wherein the human group 1 CD1 gene is selected from the group of CD1 genes consisting of CD1a, CD1b, and CD1 c, and animals wherein the CD1a, CD1b, CD1c genes are set forth in SEQ ID NOs:2, 4, and 6 respectively.

Also disclosed are transgenic animals, wherein the transgenic animal comprises one or more of the CD1 genes consisting of CD1a, CD1b, and CD1c, or any combination thereof.

The animal can be any animal, including mouse, rat, ovine, bovine, primate, chimpanzee, gorilla, or monkey.

Also disclosed are animals, wherein the animal comprises each of CD1a, CD1b, and CD1c.

Disclosed are animals, wherein the animal comprises the sequence set forth in SEQ ID NO:1.

8. Method of Producing Transgenic Animals

Disclosed are methods for producing the disclosed transgenic animals, such as a mouse comprising: a) providing a vector that comprises a nucleotide sequence comprising a non-mouse CD1 promoter in operable linkage with a nucleotide sequence encoding said CD1; b) introducing the expression vector of step (a) into a fertilized mouse oocyte; c) allowing said fertilized mouse oocyte to develop to term; and d) identifying a transgenic mouse whose genome comprises the CD1 nucleotide sequence, wherein expression of said CD1 results in an increase in CD1 restricted T-cells.

Disclosed are methods for producing a transgenic animal, such as a mouse, whose genome comprises a human CD1 comprising: (a) injecting into a fertilized animal egg: a transgene comprising a transcriptional control region operably linked to cDNA encoding a human CD1, wherein said control region comprises the human CD1 promoter; (b) transplanting the injected egg in a foster parent female animal; (c) selecting an animal derived from an injected egg whose genome comprises a human CD1.

Disclosed are methods of producing a transgenic animal, such as a mouse, comprising a human CD1 gene, comprising: introducing a CD1 gene targeting construct into an embryonic stem cell; introducing the embryonic stem cell into a blastocyst; implanting the resulting blastocyst into a pseudopregnant animal, wherein the pseudopregnant animal gives birth to a chimeric animal; and breeding the chimeric animal to produce the transgenic animal, wherein where the disruption is homozygous, and the transgenic mouse produces human CD1, and produces CD1 restricted T-cells.

Also disclosed are the animals produced by any of the methods disclosed herein and their prodgeny.

Also disclosed are transgenic cells, wherein the cell comprises a transgenic human group I CD1 gene, such as a mouse cell. Also disclosed cells expressing a group 1 CD1 gene, wherein the cells are antigen producing cells, and antigen producing cells (APC) that are expressing a human group 1 CD1 gene. Also disclosed are cells, wherein the APC is a dendetric cell or a Langerhans cell, or a bone marrow cell.

Disclosed are methods of using the disclosed cells, comprising administering the cell to an animal to elicit an immune response, or assaying materials to see if the materials bind the cell expressing the human group 1 CD1 gene. It is also understood that animals, such as a mouse, comprising the disclosed cells, and their use, are also disclosed.

Disclosed are animals, such as a mouse, wherein the mouse is a human TgCD1 (hTgCD1) mouse, as discussed herein, such as a mouse that expresses human CD1a, CD1b, and CD1c, or any sub-combination of these.

9. Methods of Using the Compositions

The disclosed compositions can be used in a variety of ways as research tools. For example, the disclosed compositions, such as SEQ ID NOs:1-7 can be used to study the interactions between human group 1 CD1 and T cells.

The disclosed compositions can also be used as diagnostic tools related to bacterial infections and autoimmune diseases, such as Mycobacteria tuberulosis infections and Multiple Sclerosis.

Disclosed are methods for screening compounds that promote the activation of CD1 restricted T-cells in a transgenic animal producing non-mouse CD1 cells, comprising: providing the transgenic animals disclosed herein; providing a compound to said animal; and assaying the animal for activation of CD1 restricted T-cells.

Also disclosed are methods of screening for an antigen involved in an immune response comprising: administering a pathogen to or inducing a cancer in an animal transgenic for human group I CD1; isolating an antigen presented on CD1 restricted T cells; wherein the antigen is a lipid, glycolipid, lipopeptide, glycopeptide, or polypeptide of the pathogen or cancer.

Also disclosed are antigens isolated by the disclosed methods, as well as vaccines comprising the antigens isolated by the disclosed methods.

Disclosed are methods of immunizing a subject for a condition or disease comprising administering the antigen isolated by the disclosed methods.

Disclosed are methods of investigating the role of CD1 restricted T cells in immune responses comprising introducing an antigen to a human TgCD1 animal, such as a mouse, and measuring the number of CD1-specific T cells, wherein an increase in the number of CD1 T cells relative to a control TgCD1 animal indicates that CD1-specific T cells are involved in the immune response to the antigen.

Disclosed are methods, wherein the antigen is a lipid or glycolipid, protein, lipoprotein, lipopeptide, glycoprotein, or glycopeptide.

Disclosed are methods, wherein the antigen is introduced by microbial infection.

Disclosed are methods, wherein the microbial infection is caused by a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella bumetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Disclosed are methods, wherein the microbial infection is a Mycobacterial infection.

Disclosed are methods, wherein the mycobacterial infection is Mycobacterium tuberculosis.

Disclosed are methods, wherein the antigen is an antigen from an autoimmune disease or inflammatory condition.

Also disclosed are methods, wherein the antigen is an antigen selected from the group of autoimmune disease or inflammatory conditions consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.

Disclosed are methods of screening for a vaccine to inhibit a microbial infection comprising administering an agent to a disclosed transgenic animal, removing a tissue sample from the animal, measuring the specificity and activitation level of T cells and comparing the level and specificity of T cells.

Disclosed are vaccines for treating a microbial infection identified by the disclosed methods.

Disclosed are methods of treating a subject with a microbial infection comprising administering the disclosed vacinnes.

Disclosed are methods of vacinnating a subject with the potential of obtaining a microbial infection comprising administering the disclosed vacinnes.

It is understood that the subject can be any animal, including any animal specifically disclosed herein, such as a mouse or human.

Disclosed are methods, wherein the microbial infection is a bacterial infection.

Also disclosed are methods, wherein the bacteria causing the bacterial infection can be selected from the group of bacterial consisting of M. tuberculosis, M. bovis, M. bovis strain BCG, BCG substrains, M. avium, M intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Disclosed are methods of treating a subject with autoimmune disease or inflammatory condition comprising administering any of the disclosed vacinnes.

Disclosed are methods of treating, wherein the autoimmune disease or inflammatory condition can be selected from the group of autoimmune disease or inflammatory conditions consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.

Disclosed are vaccines comprising an antigen identified by any of the methods disclosed herein, such as a vaccine directed to a bacterial infection, such as, wherein the bacteria causing the bacterial infection can be selected from the group of bacterial consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

A method of investigating the role of CD1 restricted T cells in an immune response comprising introducing an antigen to a human TgCD1 mouse and measuring the number of CD1-specific T cells, wherein an increase in the number of CD1 T cells relative to a control TgCD1 mouse indicates that CD1-specific T cells are involved in the immune response to the antigen. Typically antigens can refer any native or exogenous peptide, polypeptide, protein, lipid, or glycoliped to which an immune response can occur. Thus the disclosed methods can comprise administering a lipid antigen to a human TgCD1 mouse. Also disclosed are methods of investigating the role of CD1 restricted T cells in immune responses, wherein the antigen is a glycolipid.

The term “increase” is understood herein to mean any change in a number or quantity that results in a larger number or quantity existing relative to a control or the number or quantity prior to the change. Thus, for example, a change in the number of T cells in the spleen of a mouse from 70×10⁶ to 80×10⁶ would be an increase. Similarly, a comparison of a mouse being used in the disclosed screening methods and a control mouse can show, for example, a difference in the number of T cells in a given volume of blood. Where the difference is such that the mouse being used in the disclosed screening methods has more T cells than the control mouse, this is understood to be an increase.

a) Recognition of Microbial Lipid Antigens by CD1-Restricted T Cells

A breakthrough in understanding the function of group 1 CD1 molecules came with the finding that CD1b can present mycobacterial lipid antigens to T cells (Beckman, E. M., et al. 1994. Nature 372:691-694), (Porcelli, S., et al. (1992) Nature 360:593-597). CD1b has now been shown to present three distinct components of mycobacterial cell walls, namely mycolic acid, lipoarabinomannan (LAM), and glucose monomycolate (GMM), to CD1b-restricted T cells (Beckman, E. M., et al. 1994. Nature 372:691-694), (Sieling, P. A., et al. 1995. Science 269:227-230), (Moody, D. B., et al. (1997) Science 278:283-286). These CD1b-restricted lipid antigens share a general motif in which two hydrophobic acyl chains are capped with a hydrophilic moiety. In addition to CD1b, CD1a and CD1c could also present protease-resistant, mycobacterial antigen to T cells (Rosat, J. P., et al. 1999. J. Immunol.162:366-371), (Beckman, E. M., et al. (1996) J. Immunol. 157:2795-2803). In contrast to CD1b antigens, the CD1c-presented lipids have a single alkyl chain tail, suggesting different CD1 isoforms might be specialized to present different types of lipid antigens. The only characterized CD1c-presented antigen from Mycobacteria has been identified as a mannosyl-β1-phophoisoprenoid (MPI) (Moody, D. B., et al. (2000) Nature 404:884-888). Because MPI is similar in structure to eukaryotic polyisoprenylglycolipids, it is understood that human CD1c-restricted T cells can recognize both mammalian and microbial versions of these antigens.

Based on the putative motif for CD1b-presented antigens (dual alkyl chains linked to a hydrophilic cap), several potential targets for group 1 CD1-restricted T cells can exist. These include the lipoteichoic acid (LTA) of gram-positive bacteria, capsular polysaccharides of virulent gram-negative bacteria, and also components or precursors of the lipopolysaccharides of gram-negative bacteria (Porcelli, S. A., et al. (1998) Immunol. Today 19:362-368).

There are a variety of methods that may be used to introduce an antigen to a subject. For example, the antigen may be injected intraveneously when the subject is an animal or added to a media where the subject is a cell or cell line. Specifically disclosed are methods of introducing an antigen to a subject including, but not limited to injection (subcutaneous, intravenous, intradermal, intracranial), inhalation, and contact (including, for example, infection). Thus also disclosed are methods of investigating the role of CD1 restricted T cells in an immune response, wherein the antigen is introduceded by microbial infection.

There are many known bacteria which may be used with the disclosed methods. Thus, specifically disclosed are methods of investigating the role of CD1 restricted T cells in an immune response, wherein the bacterial infection is caused by a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella bumetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Thus one embodiment of the disclosed methods are methods, wherein the bacterial infection is a Mycobacterial infection. Another embodiment of the disclosed methods are methods, wherein the mycobacterial infection is Mycobacterium tuberculosis.

Also disclosed are methods of investigating the role of CD1 restricted T cells in an immune response, wherein the antigen is an antigen from an autoimmune disease or inflammatory condition. Thus, for example, disclosed are methods of investigating the role of CD1 restricted T cells in an immune response, wherein the antigen is an antigen selected from the group of autoimmune disease or inflammatory conditions consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.

b) Functions of Group 1 CD1-Restricted T Cells in Mycobacteria Infection

Mycobacterium tuberculosis (Mtb) have evolved many specific adaptations that enable it to infect and survive within specific host cells, such as a lipid-rich cell wall and the ability to inhibit phagosome maturation (Sturgill-Koszycki, S., et al. (1994) Science 263:678-681). The immune response to Mtb is T cell-dependent. The protective immune response comprises both CD4 and CD8 T cells and perhaps unconventional T cell population such as γδ T cells and CD1-restricted T cells (Orme, I. M. (1987) J. Immunol. 138:293-298), (Caruso, A. M., et al. (1999) J. Immunol. 162:5407-5416), (Flynn, J. L., et al. (1992) Proc. Natl. Acad. Sci U.S.A. 89:12013-12017), (Sousa, A. O., et al. (2000) Proc. Natl. Acad. Sci. U.S.A. 97:4204-4208), (Shen, Y., et al. (2002) Science 295:2255-2258), (Flynn, J. L., and J. Chan (2001) Annu Rev Immunol 19:93-129). Presentation of lipid antigens by group 1 CD1 helps to detect Mycobacteria that persist within deacidified endosomes and which avoid presentation by the MHC molecules. Mycobacteria-specific, CD1-restricted T cell responses have been detected in human PPD⁺ subjects recently infected with Mtb, but not in naïve healthy subjects, indicating that group 1 CD1-restricted T cells are indeed activated following infection with Mycobacteria (Moody, D. B., et al. (2000) Nature 404:884-888). Most of the Mycobacteria-specific group 1 CD1-restricted T cells secrete high levels of proinflammatory cytokines, including IFN-γ, TNF-α, and GM-CSF, upon antigen stimulation, which may facilitate the development of cell-mediated immunity against intracellular pathogens. These T cells also possess other effector mechanisms, such as cytolysis and granulysin delivery, that may directly contribute to bacteria clearance (Stenger, S., et al. (1998) Science 282:121-125). Taken together, these observations strongly indicate that group 1 CD1-restricted Mycobacteria specific T cells may contribute to host defense against intracellular infection. hCD1Tg mice can be infected with Mtb and assess the in vivo function of group 1 CD1-restricted T cells in protective immunity against Mtb infection.

Disclosed herein are methods of screening for agents to inhibit a microbial infection or autoimmune condition comprising introducing the agent to a human group 1 CD1 transgenic mouse, removing a tissue sample from the mouse, measuring the specificity and activitation level of T cells and comparing the level and specificity of T cells. It is understood, that many agents can inhibit a microbial infection or autoimmune condition, such agents can include chemical agents as well as biological agents such as chemokines; cytokines; or peptides, lipopetides, lipoproteins, glycopeptides and glycoproteins, polypeptides, proteins, lipids, and glycolipids which may be used to generate an immune response. The disclosed methods of screening can also be used to identify antigens to which the CD1 restricted peptides are specific. For example, the disclosed methods can be used to screen for an antigen involved in an immune response comprising administering a pathogen to or inducing a cancer in an animal transgenic for human group I CD1; isolating an antigen presented on CD1 restricted T cells; wherein the antigen is a lipid, glycolipid, or polypeptide of the pathogen or cancer. Agents (including, for example, antigens) identified by the disclosed methods can be used for a variety of activities, including but not limited to vaccination to inhibit a microbial infection or autoimmune disease and treatment of a subject with an autoimmune condition or microbial infection. Thus, specifically disclosed and herein contemplated are agents identified by the disclosed methods used for treating a microbial infection or autoimmune condtions. It is understood that such agents, including antigens identified by the disclosed screening methods can be used in a vaccine. Thus, also disclosed are methods of treating a subject with a microbial infection comprising administering the agent identified by the disclosed methods. Additionally, disclosed are methods of immunizing a subject for a condition or disease comprising administering the antigen isolated by the methods.

The disclosed methods can also be used to investigate the role of CD1 restricted T cells in immune responses comprising introducing an antigen to a human TgCD1 mouse, removing a tissue sample (e.g., blood, organ tissue biopsy, etc) for the mouse, and measuring the number of CD1-specific T cells, wherein an increase in the number of CD1 T cells relative to a control TgCD1 mouse indicates that CD1-specific T cells are involved in the immune response to the antigen. It is understood that many methods for assessing the specificity, number, and/or activation state of a T cell are known in the art and can be used with the disclosed methods. For example, the methods can comprise injecting and antigen into a hTgCD1 mouse, removing blood and isolating the peripheral blood lymphocytes. The assessing the number of CD1 restricted T cells specific for the antigen via a fluorescent multimeric antigen-CD1 molecule (tetramer) and comparing the number of antigen-specifc CD1 restricted T cells to a control. Other methods of measuring T cells include but are not limited to cytokine ELISpot, Fluorescence Aquired Cell Sorting (FACS), Proliferation assays, Intracellular Cytokine Assays, and CFSE staining.

Also disclosed are methods for screening compounds that promote the activation of CD1 restricted T-cells in a transgenic mouse producing non-mouse CD1 cells, comprising providing the transgenic mouse; providing a compound to said mouse; and assaying the mouse for activation of CD1 restricted T-cells.

Herein, “inhibit” or “inhibition” refers to an interruption of a condition or microbial infection. This can include, but is not limited to the the maintenance of the present state of a condition or infection therefore preventing an increase in said condition or infection. Inhibit can also mean a reduction in the rate of an increasing infection. For example, an infection that when treated with an agent reveals the number of bacteria in a subject to increase from 100 to 150, but without the treatment increases from 100 to 1000, that agent is said to “inhibit” the infection. Furthermore, inhibition can result in a reduction in the disease or condition. Such a reduction can comprise as little as a 5% reduction in the symptoms or infecting units associated with the disease or condition. The reduction can also comprise a 10%, 15%, 20% 30%, 40% 50% 60%, 70%, 80%, 90% reduction or the complete ablation of the disease or condition. The term “inhibit” can also be used to refer to preventative measures such as prophylactic vaccines. Such inhibition can include but is not limited to the prevention of the disease or condition being vaccinated against or a lessening of the potential severity of the disease or condition. For example, an agent that upon beig administered prevents a microbial infection is said to “inhibit” the infection.

The use of vaccines for the prophylactic or therapeutic treatment of a microbial infection or condition is well-known in the art. Typically, a vaccine comprises an antigen to generate an immune response. Thus, specifically disclosed herein is a vaccine comprising an agent identified by the disclosed screening methods. Also disclosed herein is a vaccine comprising an agent identified by the disclosed screening methods, wherein the agent is an antigen.

Therefore, disclosed herein are vaccines, wherein the vaccine is directed to a bacterial infection. Also disclosed are vaccines, wherein the bacteria causing the bacterial infection can be selected from the group of bacterial consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

It is understood and herein contemplated, that the term “treatment” refers to any method that can be used to decrease the severity or symptoms of a disease (ie., microbial infection) or condition. Thus, it is understood that a “treatment” can result in as little as a 5% reduction in a disease or condition or as much as the complete ablation of the condition. Treatment can also refer to methods that reduce the potential severity of a disease or condtion.

Also disclosed are methods of treating a subject with an autoimmune disease or condition comprising administering an agent identified by the disclosed screening methods. For example, herein disclosed are methods of treating a subject with an autoimmune disease or inflammatory condition, wherein the autoimmune disease or inflammatory condition is selected from the group consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.
c) Expression and Tissue Distribution of group 1 CD1 Molecules

Numerous monoclonal antibodies (mAbs) against the human CD1 molecules have been generated as a result of the initial interest in CD1 as a differentiation marker for thymocytes. In contrast to group 2 CD1, which are widely expressed on hematopoietic cells (Brossay, L., et al. 1997. J. Immunol. 159:1216-1224), (Mandal, M., et al. 1998. Mol. Immunol. 35:525-536), (Exley, M., et al. 2000. Immunology 100:37-47), the distribution of group 1 CD1 molecules is relatively restricted. CD1a, CD1b, and CD1c are mainly expressed on immature cortical thymocytes, and on some antigen presenting cells, such as the myeloid lineage of dendritic cells (DCs) and Langerhans cells in skin (Porcelli, S. A. 1995. Adv. Immunol. 59:1-98), (Cattoretti, G., et al. 1987. in Leukocyte typing III: White cell differentiation antigens. Oxford University Press, Oxford. 89-91 pp). Only CD1c is expressed on a subpopulation of peripheral B cells that also express CD19, CD20, and surface Ig (Small, T. N., et al. (1987). J. Immunol. 138:2864-2868). The expression of CD1a, CD1b, and CD1c cannot be detected on monocytes or peripheral T lymphocytes. The expression of all three group 1 CD1 molecules can be induced in vitro on peripheral blood monocytes as they differentiate into DCs by exposure to GM-CSF and IL-4 (Kasinrerk, W., et al. (1993) J. Immunol. 150:579-584). This raises the possibility that group 1 CD1 molecules can be upregulated on tissue macrophages or DCs in response to agents such as bacteria or inflammatory products that induce the secretion of GM-CSF. Indeed, the expression of CD1 has been shown to be strongly upregulated in dermal DCs in tuberculoid skin lesions from patients infected with Mycobacterium leprae (Sieling, P. A., et al. (1999) J. Immunol. 162:1851-1858).

d) Intracellular Localization and Trafficking of Group 1 CD1 Molecules in Normal and Infected Antigen Presenting Cells

Human bone marrow-derived DCs (BM-DCs) express high levels of CD1a, b, and c at their surface, but significant amounts of CD1 molecules, especially CD1b, are present intracellularly. Recent studies reveal that different CD1 isoforms accumulate in distinct intracellular compartments, which might be important for these molecules to sample microbial antigens from different subcellular compartments of infected cells. CD11a molecules are primarily found in early/recycling endosomes (Sugita, M., et al. (1999) Immunity 11:743-752), (Salamero, J., et al. (2001) J. Invest. Dermatol. 116:401-408) while CD1b molecules accumulate in late endosomes, lysosomes and a subpopulation of lysosomes containing MHC class II molecules (MIIC compartments) (Sugita, M., et al. (1996) Science 273:349-352), (Briken, V., et al. (2000) Semin. Immunol. 12:517-525). The distribution of CD1c partially overlaps with CD1a and CD1b molecules, as it is found in both early and late endosomes (Briken, V., et al. (2000) Semin. Immunol. 12:517-525), (Sugita, M., et al. (2000) Proc. Natl. Acad. Sci. U.S.A. 97:8445-8450).

Most of the group 1 CD1-presented lipid antigens identified to date are components of the mycobacterial cell wall. How do these mycobacterial lipid antigens reach CD1⁺ compartments? A recent study by Schaible et al. (Schaible, U. E., et al. (2000) J. immunol. 164:4843-4852) showed that all three group 1 CD1 molecules intersect with mycobacterial phagosomes at different stage of maturity in Mycobacteria-infected DCs. CD1a and CD1c are present in phagosomes arrested at the early endosomal stage, whereas CD1b is located in phagolysosomes. Thus, it is possible that the loading of CD1 with antigen can take place directly in the different maturation stages of mycobacterial phagosomes, which contain different CD1 molecules. In addition, it is now clear that mycobacterial lipids can be released from the phagosomes into various endocytic compartments which could then be sampled by different CD1 molecules (Mukherjee, S., et al. (1999) J. Cell Biol. 144:1271-1284). Although direct evidence for the loading of a microbial lipid antigen onto CD1 in a specific compartment is currently lacking, analysis of the effect of drugs on the presentation of lipid antigens by CD1 provide indirect evidence for the nature of the loading compartment. It has been shown that the presentation of exogenous microbial lipid antigens by CD1b is sensitive to inhibition of endosomal acidification (Sugita, M., et al. (1999) Immunity 11:743-752), (Briken, V., et al. (2000) Semin. Immunol. 12:517-525). In contrast, antigen presentation mediated by CD1a and CD1c in the few examples evaluated so far appears to be independent of endosomal acidification (Sugita, M., et al. (1999) Immunity 11:743-752). These results indicate that antigen loading of CD1b occurs in late endosomes or lysosomes, whereas CD1a and CD1c associate with lipids in early endosomes or at the plasma membrane. However, a more complex picture emerged from recent analysis of CD1b-mediated lipid antigen presentation, in that certain CD1b-presented antigens require processing in endosomes, whereas other antigens do not. The requirement for endosomal processing appears to correlate to the overall lipid length of the antigens. In general, antigens with longer alkyl chain require endosomal processing for presentation, whereas antigens with shorter alkyl chain do not (Moody, D. B., et al. (2002) Nat. Immunol. 3:435-442). The lipid antigen recognized by a newly derived CD1a-restricted mycobacterial lipid-specific T cell line can be characterized and the cellular requirements for presentation of mycobacterial lipid antigens by CD11a can be examined.

e) Interactions Between TCR and CD1/Lipid Antigen Complexes

The structural requirements for glycolipid antigen recognition by group 1 CD1 shows a high level of specificity. Group 1 CD1-restricted T cell clones can discriminate between CD1 isoforms, as well as the precise structure of lipids that are bound in the CD1 groove. More detailed studies of the CD1b-restricted and CD1c-restricted T cell lines reveal that the recognition of glycolipid antigen by these T cells was extremely sensitive to alterations in its carbohydrate moiety, but not affected by substantial variations in its lipid tails (Moody, D. B., et al. (1997) Science 278:283-286), (Moody, D. B., et al. (2000) Nature 404:884-888), (Moody, D. B., et al. (1999) Immunol. Rev. 172:285-296). These studies, along with the solved crystal structures of mouse CD1d1 (Zeng, Z., et al. (1997) Science 277:339-345) and human CD1b (52), suggest a model of recognition whereby T cells recognize the epitope formed by the α and α2 helices of CD1 and the hydrophilic portions of the lipid antigen. The acyl chains of lipid antigen are most likely buried within hydrophobic pockets of the CD1 antigen binding groove.

Sequence analysis of T cell clones restricted by human group 1 CD1 molecules showed heterogeneous usage of TCR Vα and Vβ segments, and extensive junctional diversity of CDR3-encoded residues (Grant, E. P., et al. (1999) J. Exp. Med. 189:195-205). This data indicates that, unlike group 2 CD1-restricted NKT cells, group 1 CD1-restricted T cells appear to use diverse TCRs for antigen recognition. It is noteworthy that CDR3 regions of TCRs from mycolic acid and MPI-specific clones show a high frequency of basic-charge residues, while a GMM-specific clone lacks basic-charge residues in CDR3 region. Because mycolic acid and MPI have negatively charged head groups, and GMM does not, CDR3 regions may play an important role in the specific recognition of the hydrophilic cap of lipid/glycolipid antigens presented by CD1. A panel of hCD1-restricted T cell clones specific to mycolic acid, GMM, and MPI from Mycobacteria-infected hCD1Tg mice can be generated and their TCR sequences can be determined to better understand the mechanisms governing specific interactions between TCR and CD1/lipid complexes.

10. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

11. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

12. Nucleic Acids

There are a variety of molecules disclosed herein that are nucleic acid based, including for example the nucleic acids that encode, for example, SEQ ID NOs; 3, 5, and 7 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

13. Sequences

There are a variety of sequences related to the human group 1 CD1 genes set forth in SEQ ID NOs: 1-7, these sequences and others are herein incorporated by reference in their entireties as well as for individual subsequences contained therein.

One particular sequence set forth in SEQ ID NO: 1 and is used herein, as an example, to exemplify the disclosed compositions and methods. It is understood that the description related to this sequence is applicable to any sequence related to group 1 CD1 genes unless specifically indicated otherwise. Those of skill in the art understand how to resolve sequence discrepancies and differences and to adjust the compositions and methods relating to a particular sequence to other related sequences.

14. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as human group 1 CD1a into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRS) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

15. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. β actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR cells and mouse LTK cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

16. Peptides

a) Protein Variants

As discussed herein there are numerous variants of the CD1a protein, CD1b protein, and CD1c protein that are known and herein contemplated. In addition, to the known functional group 1 CD1 (CD1a, CD1b, and CD1c) strain variants there are derivatives of the group 1 CD1 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables IV and V and are referred to as conservative substitutions.

TABLE IV
Amino Acid Abbreviations

| Amino Acid | Abbreviations |
|---|---|
| alanine | Ala A |
| allosoleucine | AIle |
| arginine | Arg R |
| asparagine | Asn N |
| aspartic acid | Asp D |
| cysteine | Cys C |
| glutamic acid | Glu E |
| glutamine | Gln Q |
| glycine | Gly G |
| histidine | His H |
| isolelucine | Ile I |
| leucine | Leu L |
| lysine | Lys K |
| phenylalanine | Phe F |
| proline | Pro P |
| pyroglutamic | pGlu |
| acidp | |
| serine | Ser S |
| threonine | Thr T |
| tyrosine | Tyr Y |
| tryptophan | Trp W |
| valine | Val V |

TABLE V
Amino Acid Substitutions
Original Residue Exemplary Conservative
Substitutions, others are known in the art.
Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table V, i.e., selecting residues that differ more significantly in their effect on maintaining (a) the structure of the polypeptide backbone in the area of the substitution, for example as a sheet or helical conformation, (b) the charge or hydrophobi city of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 3 sets forth a particular sequence of CD11a and SEQ ID NO: 5 sets forth a particular sequence of a CD1b protein and SEQ ID NO:7 sets forth a particular sequence of a CD1c protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

17. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antinflammatory agents, anesthetics, and the like. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

18. Compositions with Similar Functions

It is understood that the compositions disclosed herein have certain functions, such as presenting lipid antigens to T cells and presenting self-antigens associated with autoimmunity to T cells. Disclosed herein are certain structural requirements for performing the disclosed functions, and it is understood that there are a variety of structures which can perform the same function which are related to the disclosed structures, and that these structures will ultimately achieve the same result, for example presentation of lipid antigens on T cells.

19. Method of Presenting Cancer Antigens

It is understood that cancers express high concentrations of glycolipids. It is therefore, contemplated that lipids associated with a variety of cancers can be expressed by the CD1 genes. For example, melanoma has a high concentration of GD3. The disclosed compositions can be used to present any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to present antigens is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for presenting antigens of precancer conditions such as cervical and anal dysplasias, other dysplasias, severe dysplasias, hyperplasias, atypical hyperplasias, and neoplasias.

It is contemplated that vacinnes and treatments related to these cancers are also disclosed.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Unlike MHC class I and class II molecules that present peptide antigens, CD1 molecules enable the recognition of lipid-containing antigens by T cells. While firmly established as a family of lipid antigen presenting molecules, the in vivo function of group 1 CD1-restricted T cells in immune responses remains to be defined. Use of human group 1 CD1 transgenic mice can provide the ability to address the significance of group 1 CD1-restricted T cells in protective immune responses to various microbial pathogens, including Mycobacteria. Even with the arsenal of drugs in modern day medicine, tuberculosis remains a significant worldwide health problem. Therefore, vaccines that activate multiple effector arms of the immune system may be necessary to achieve protective immunity against Mtb. Peptide-based vaccines targeted to MHC molecules have to be tailored according to the MHC haplotype of the individual. In contrast, CD1 molecules are nonpolymorphic. Thus, the microbial lipid antigens presented by CD1 may offer particular advantages as subunit vaccines. The hCD1Tg mouse model can be used to determine the immunogenecity of individual microbial lipid antigens and to evaluate the potential use of these antigens for a novel antituberculosis vaccine. The frequency of group 1 CD1-restricted autoreactive T cells in normal individuals implies that these cells must serve an important, foreign antigen-independent function in the periphery. These group 1 CD1-restricted autoreactive T cells can be involved in shaping the adaptive immune response to microbial pathogens by virtue of their distinct capacity to recognize CD1-expressing DCs at early stages of infection.

The lack of a suitable small animal model for the study of group 1 CD1 leaves open an important question concerning the significance of CD1-restricted T cells in response to specific bacterial challenges. Because group 1 CD1 molecules are absent in rodents, the powerful genetics and ever expanding knowledge of the mouse immune system cannot be applied toward this problem. Therefore, the immune function of group 1 CD1 was addressed in transgenic mice. Toward this end, transgenic mice were generated using a bacterial artificial chromosome (BAC) clone containing the entire coding sequences for human CD1A, B, and C. In addition, it was demonstrated that antigen-specific, group 1 CD1-restricted T cells can be isolated from the human group 1 CD1 transgenic mice (hCD1Tg). These data support using hCD1Tg mice as an animal model to address the in vivo role of the group 1 CD1 in the protective immunity against pathogens and in T cell development. This approach provides the advantage of the well-characterized serologic definition of human CD1, in addition to the many advantages of using mice (e.g. well defined genetics, gene knockouts, numerous reagents) as opposed to developing other experimental animals such as rabbits or guinea pigs, which express homologues of the group 1 CD1.

a) Generation of Transgenic Mice Expressing Human Group 1 CD1 Molecules.

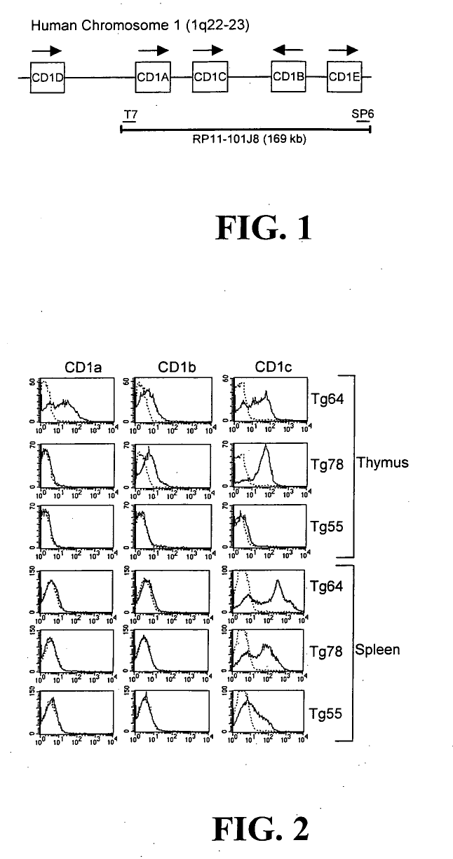

The group 1 CD1 molecules have a unique expression pattern. Unlike MHC class I molecules, they are not ubiquitously expressed. The CD1a, b, and c proteins are expressed predominantly on cortical thymocytes but not mature T cells. Peripheral blood lymphocytes (PBL)-derived monocytes can also be induced to express all three group 1 CD1 molecules by culture in GM-CSF and IL-4. CD1c, unlike CD1a and CD1b, is also expressed on B cells. No promoters for any CD1 genes have yet been identified, and no suitable surrogate promoters were known which would express the group 1 CD1 molecules in a physiologically relevant manner. Therefore the entire human group 1 CD1 locus was introduced into the mouse with the aim that endogenous promoters/regulatory sequences would replicate human expression patterns in the mouse. The R1 ES cells (129 SVJ) were transfected with a 169-kb BAC that contained the entire coding region of human group 1 CD1 (CD1A, -B, and -C), CD1E and extensive 5′- and 3′-flanking sequences (FIG. 1). Because the intragenic distance between CD1B and CD1E is the shortest among all the CD1 genes, the CD1E gene was not deleted from the construct to preserve the integrity of transcriptional regulation. Furthermore, a recent study shows that although CD1e molecules can be detected intracellularly, they never seem to reach the cell surface, which would exclude their direct role in T cell-recognition (Angenieux, C., et al. (2000) J. Biol. Chem. 275:37757-37764). The transfected ES cells were screened by PCR primers specific to CD1 genes and both ends of the BAC clone to ensure the presence of intact transgene.

Ten ES clones were injected into B6 blastocysts to generate chimeric mice. Chimeric mice which expressed CD1c in PBL were chosen as founders and bred with C57BL/6 mice to produce transgene-positive offspring. Three lines of transgenic mice were established and chosen for further characterization. The Tg64 line expresses all three group 1 CD1 molecules on immature thymocytes (FIG. 2). A second line expresses only CD1b and CD1c (Tg78). The expression of CD1b and CD1c is very similar between line 64 and 78. The third Tg line (Tg55) expresses only CD1c, albeit at much lower levels than lines 64 and 78 in both thymus and spleen (FIG. 2). The present study establishes the Tg64 line as a functional model for the study of the group 1 CD1-restricted T cells, since the Tg64 line expresses all three group 1 CD1 molecules and displays a tissue distribution pattern very similar to that of humans (see below). In addition, the Tg64 line, hereon referred to as hCD1Tg, will be introduced onto class Ia-deficient (K^{bo}D^{bo}), class II-deficient (MHC II^{o}), and CD1-deficient (CD1^{o}) background to assess the overall contribution of group 1 CD1 in microbial immunity and T cell development.

b) Characterization of Group 1 CD1Expression in hCD1Tg Mice.

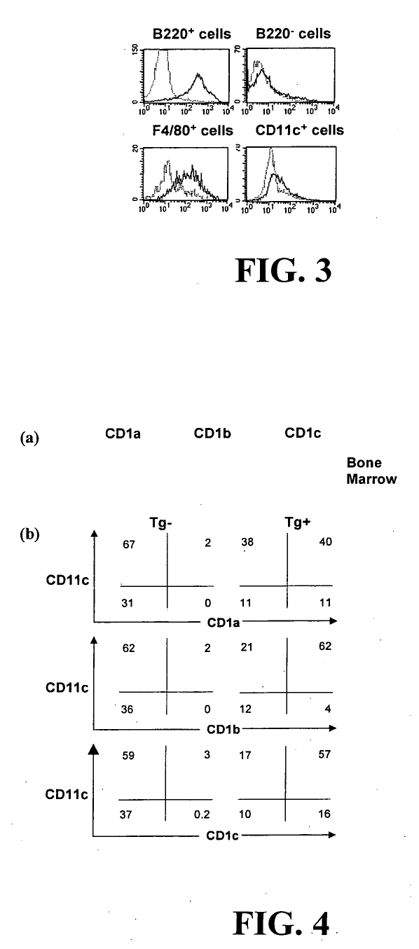

The expression of the human CD1 molecules on cells and tissues from thymus, spleen, lymph nodes, bone marrow (BM), and PBL from hCD1Tg mice was examined by flow cytometric analysis. The expression pattern of CD1a, b, and c in hCD1Tg is summarized in Table I. Similar to humans, CD1a, b, and c can be detected on cortical thymocytes (CD4⁺ CD8⁺ thymocytes) of hCD1Tg, but not mature T cells. In PBL and splenocytes, only CD1c is expressed. Two-color immunofluorescence analysis showed that greater than 90% of B220⁺ splenocytes express high levels of CD1c (FIG. 3). Lower levels of CD1c expression can be detected on splenic macrophages (F4/80⁺) and splenic DC (CD11c⁺).

TABLE I
The expression levels of CD1a, b, and c in hCD1Tg

| Cell Type | CD1a | CD1b | CD1c |
|---|---|---|---|
| Cortical thymocytes | ++ | + | ++ |
| Medullary thymocytes | − | − | − |
| Thymic epithelial cells | + | − | + |
| Splenic B cells | − | − | +++ |
| Splenic T cells | − | − | − |
| Splenic macrophages | − | − | ++ |
| Splenic DCs | ± | ± | + |
| BM-derived macrophages | − | − | + |
| BM-derived DCs | ++ | +++ | +++ |
| Langerhans cells | +++ | + | ++ |

CD1a, b, and c expression levels were determined by flow cytometric analysis. The expression levels are indicated as follows: −, negative staining; ±. <50% reactivity above background; the number of “+” (+, ++, +++) correlates with the staining intensity of cells.

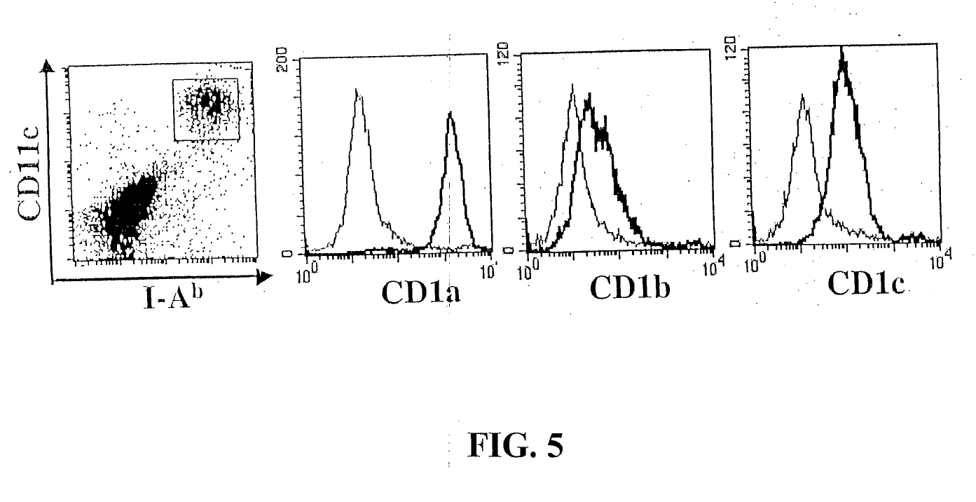

Freshly isolated BM cells also have a small subpopulation of B220⁺ cells which expresses only CD1c (FIG. 4A). The majority of BM-derived DCs (BM-DCs), obtained by culturing BM cells in GM-CSF and IL-4, express all three group 1 CD1 molecules (FIG. 4B). Unlike MHC class II, the expression of CD1a, b, and c cannot be upregulated by IFN-γ, TNF-α, LPS, or Mtb antigens on BM-DCs. In addition to the expression on BM-DCs, all three group 1 CD1 molecules can be detected on CD11c⁺/MHC class II⁺ cells isolated from skin, presumably Langerhans cells (FIG. 5). Taken together, the expression pattern of CD1a, CD1b, and CD1c in hCD1Tg mimics that seen in humans.

c) T Cell Development in hCD1Tg Mice.

Since CD1 was first discovered as a thymocyte differentiation antigen, it has been indicateed that CD1 might be involved in selection/maturation of thymocytes either through direct TCR/CD1 interactions or through a TCR-independent signaling pathways. Therefore the development of various T cell subsets in hCD1Tg mice was examined. Thymocytes, splenocytes, hepatic lymphocytes, and intraepithelial lymphocytes isolated from hCD1Tg and Tg− littermate controls were stained with antibodies specific for CD4, CD8, TCRαβ, TCRγδ, and NK1.1. No significant difference in the number of CD4+/TCRαβ, CD8+/TCRαβ, TCRγδ, and NK1.1+ T cells was detected between hCD1Tg and control mice. It is possible that the presence of murine MHC class I and class II molecules in hCD1Tg mice may limit the contribution of hCD1 in T cell development. To uncover the potential role of group 1 CD1 on the development of CD4+ and CD8+ T cells, hCD1Tg+IIo and hCD1Tg+KboDbo mice can be generated and the generation of T cells in these mice compared to that in IIo or KboDbo mice.

d) Derivation of Group 1 CD1-Restricted T Cells from hCD1Tg Mice Immunized with Mycobacterial Antigens

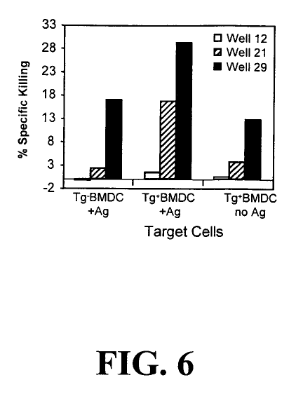

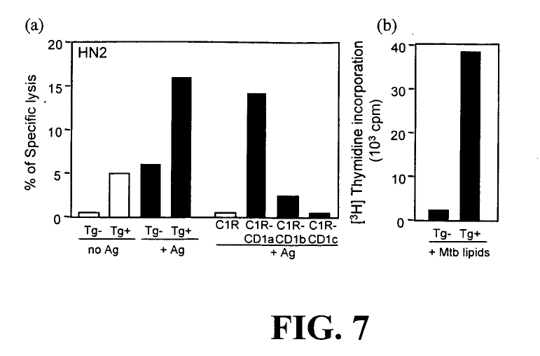

As an initial step to see whether hCD1 molecules can function as restriction elements to murine T cells, hCD1Tg mice were immunized with heat killed Mtb in incomplete Freund's adjuvant and boosted a week later with BM-DCs pulsed with Mtb antigens (prepared by sonication of lyophilized Mtb in PBS as described by Roast et al. (Rosat, J. P., et al. 1999. J. Immunol. 162:366-371). Crude sonicate of Mtb, rather than selected CD1 lipid antigens, was used to increase the potential repertoire of hCD1-restricted T cells stimulated. Splenocytes and lymph node cells from immunized mice were then restimulated weekly in vitro with BM-DCs pulsed with Mtb antigens. After one month of in vitro restimulation, cytotoxic T cell assays and cytokine release assays were performed to examine the reactivity of each T cell culture. FIG. 6 showed a representative screening for hCD1Tg-reactive T cells from three independently-derived T cell cultures by standard ⁵¹Cr-release assays, using BM-DCs from Tg⁺ and wild type control mice as targets. Wells that showed hCD1Tg-dependent reactivity (e.g. wells 21 and 29) were chosen for further restimulation and characterization. It is worth noting that a number of wells that were screened showed allo-reactivity possibly due to heterogeneity in the genetic background of hCD1Tg mice used in immunization and restimulation (3 to 4 generation backcrossed onto B6 background). Four long-term hCD1Tg-specific T cell lines, designated HN1, HN2, HN3, and HN4, have been established after 3-4 months of in vitro restimulation and expansion. The specificity of each T cell line was determined by CTL assays using BM-DCs from Tg⁺ and Tg⁻ animals in the presence or absence of Mtb antigens, as well as using C1R, a human B lymphoblastoid cell line transfected with a single CD1 gene as targets. The characteristics and specificity of these T cell lines are summarized in Table II. All four hCD1-restricted T cell lines express TCRαβ. Although Mtb antigen was used in both in vivo immunization and in vitro restimulation, three of the T cell lines appear to recognize hCD1-expressing targets in the absence of any exogenously added antigen, indicating these T cells lines might recognize hCD1 with self-glycolipid antigen(s). Only one of the T cell lines, HN2, preferentially recognizes hCD1Tg+BM-DCs in the presence of Mtb antigens (FIG. 7A). HN2 cells reacts specifically to Mtb antigen-pulsed CD1a-transfected C1R cells (C1R/CD1a), but not antigen-pulsed mock-transfected C1R cells (FIG. 7A), indicating HN2 recognizes Mtb

TABLE II
Properties of T cell lines derived from hCD1Tg

| Line | Co-receptor | Specificity | Cytokine |
|---|---|---|---|
| HN1 | CD8αα | CD1c autoreactive | IFN-γ |
| HN2 | CD4 | CD1a + Mtb lipid Ag | IFN-γ |
| HN3 | CD4 | CD1a autoreactive | IFN-γ, IL-4 |
| HN4 | CD8αβ | CD1a autoreactive | IFN-γ |
| HN5 | CD4 | MHC II + Mtb Ag | IFN-γ, IL-4 |

antigens in the context of CD1a. Furthermore, HN2 cells proliferate vigorously in response to Mtb lipid extract in a Tg-dependent manner (FIG. 7B), indicating the recognition of HN2 cells may involve a mycobacterial lipid antigen. Low reactivity of HN2 detected against antigen-pulsed Tg− cells in CTL assays can reflect the heterogeneic nature of this T cell line.

e) Characterization of an Anti-CD1c Autoreactive T Cell Clone

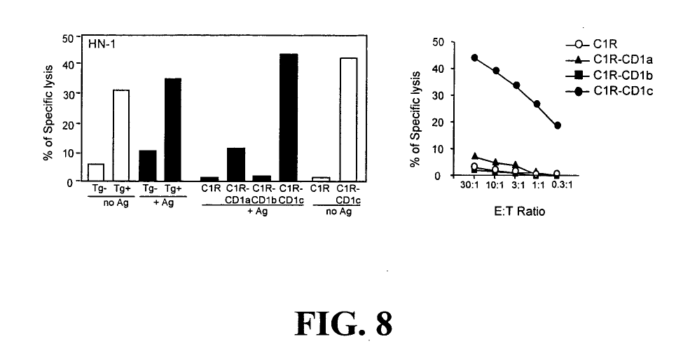

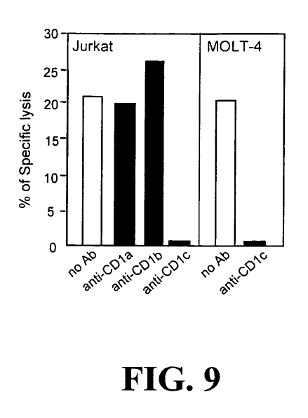

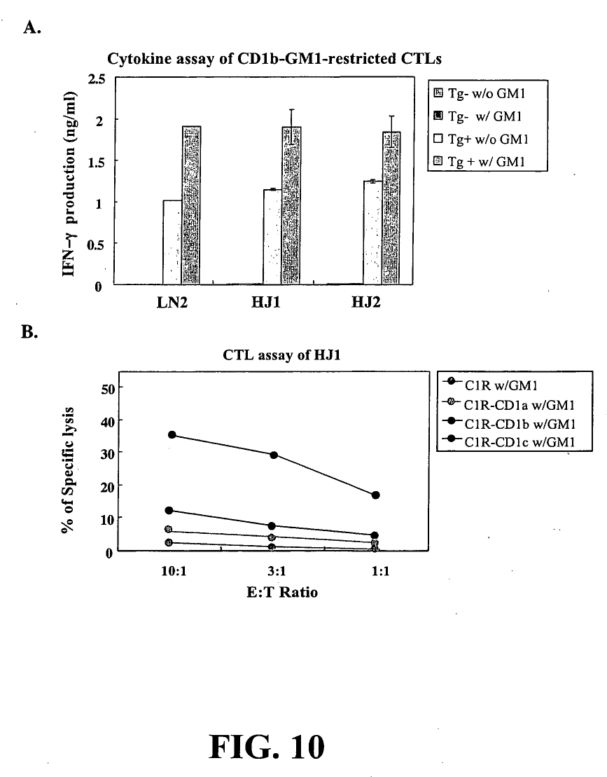

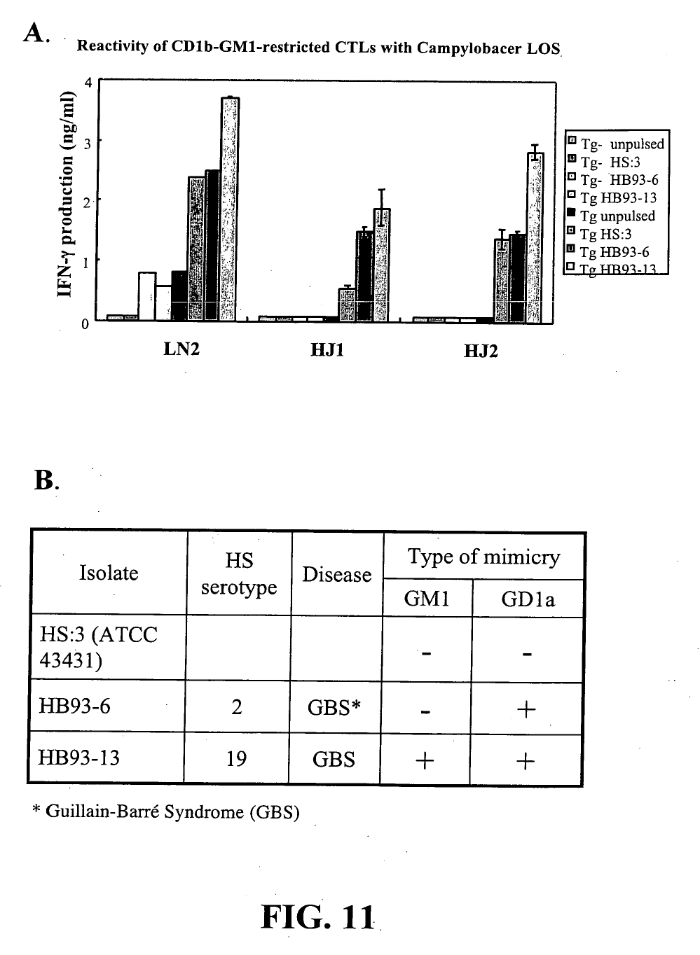

HN1, a fast-growing CD1c-autoreactive T cell line, has been subcloned by limiting dilution. The specificity of one of the HN1 T cell clones, HN1-4 is shown in FIG. 8A. Similar to the parental line, it can kill Tg⁺ cells in the absence of exogenous antigens, and its reactivity cannot be enhanced by the addition of Mtb antigens. HN1-4 T cells showed preferential reactivity toward CD1c transfectant, although some low levels of cross-reactivity to a CD1a transfectant can be detected at the high effector to target ratio (FIG. 8B). To test whether HN1-4 cells can recognize CD1c constitutively expressed on human cells, the reactivity of HN1-4 was examined with Jurkat and MOLT-4, two human T cell lines which express lower levels of CD1a, b, and c molecules compared to Tg⁺ DC and C1R transfectants. FIG. 9 showed that HN1-4 cells readily lysed Jurkat and MOLT-4 cells, and the lysis can be specifically blocked by antibody to CD1c. This data further confirmed the CD1c-specific, and autoreactive nature of HN1-4 T cells.

It is not clear if HN1-4 cells recognize CD1c molecules alone or CD1c molecules containing a self-lipid molecule. Several recent findings on the recognition of CD1d (group 2 CD1) by autoreactive NKT cells indicate that the recognition involves CD1d-bound lipid (Gumperz, J. E., et al. 2000. Immunity 12:211-221). Thus, it is disclosed herein that HN1-4 can also recognize a self-lipid presented by CD1c. The fact that HN1-4 can recognize various CD1c-expressing cells, including DCs, B cells, T cells, and fibroblasts (L929-CD1c transfectant) of either mouse and human origin, indicated that such a lipid must be broadly distributed.

Surface phenotypes and TCR usage of HN1-4 cells were determined by flow cytometric analysis. HN1-4 expresses CD8α, but not CD8β, which can indicate a unique requirement for its development. Addition of anti-CD8α antibody did not block the CTL response of HN1-4 cells, indicating the affinity of the TCRs and CD1c is sufficient to trigger the CTL response. Unlike most of the CD1d-specific autoreactive T cells which express NK cell surface markers, HN1-4 does not express any of the NK cell surface markers tested, such as NK1.1 and Ly49A. HN1-4 cells react positively with anti-Vβ9 antibody, but negatively with all four commercially available anti-Vα antibodies. The Vα usage of HN1-4 by RT-PCR was determined using primer sets specific for each Vα segment and found that Vα5 was expressed by HN1-4.

A large panel of autoreactive T cell clones specific to CD1a, CD1b, or CD1c have been isolated from humans. It has been postulated that these group 1 CD1-restricted autoreactive T cells may participate in activation of innate immunity, such as DC maturation (Vincent, M. S., et al. (2002) Nat. Immunol. 3:1163-1168), thereby modulating the upcoming acquired immune response. To investigate the requirement for the selection and development of CD1 c-specific autoreactive T cells TCR transgenic mice which express HN1-4 TCR can be generated.

Transgenic mice expressing HLA class I and class II molecules have been used to provide a suitable animal model to study the functions of HLA molecules. The studies disclosed herein demonstrated that group 1 CD1 molecules can serve as restriction elements to present either self-antigens or microbial antigens to murine CD4+ and CD8+ T cells in hCD1Tg mice. In addition, the group 1 CD1-specific T cells analyzed thus far recognized hCD1Tg⁺ mouse cells as efficiently as group 1 CD1⁺ human cells, indicating that the antigen processing/presentation machinery in the mouse is sufficient to support the human group 1 CD1-dependent antigen recognition. Together, these data indicate that the hCD1Tg mouse can be used as an animal model to examine the immune function of human group 1 CD1 molecules. Currently, hCD1Tg have been backcrossed eight generations onto B6 background. hCD1Tg can be further crossed with MHC class Ia-deficient (Kb^{bo}D^{bo}), MHC class II-deficient (II^{o}) and CD1-deficient mice (CD1^{o}) to generate hCD1TgK^{bo}D^{bo}, hCD1TgII^{oo}, and hCD1TgCD1^{o} mice. These mice allow the relative contribution of group 1 CD1-restricted T cells and MHC-restricted T cells in the immune response against microbial infection to be compared, and evaluate the protective role of group 1 CD1-restricted response in the absence of MHC class Ia or class II-restricted responses. In addition, these mice can be used to examine the function of group 1 CD1 in the development of CD8, CD4, and NKT cell compartments.

### 2. Example 2

### Examine the Contribution of Group 1 CD1-Restricted T Cells in Mycobacterial Infection

Group 1 CD1 presents mycobacterial lipid/glycolipid antigens to T cells, which extends the spectrum of immune recognition of microbial pathogens beyond the peptide antigens presented by MHC-class I and class II molecules. The unique nature of the CD1-presented antigens and the distinct pattern of CD1 expression indicate that the group 1 CD1-restricted response may play a complementary role to MHC class I-restricted response in host defense against Mycobacteria. Due to the lack of a suitable animal model, analysis of group 1 CD1-restricted responses to Mycobacteria has been limited to the use of human T cell lines or clones derived from naturally primed individuals. The in vivo function of these Mycobacteria-specific human group 1 CD1 (hCD1)-restricted T cells is currently unknown. In addition, little is known about the frequency and the kinetics of group 1 CD1-restricted T cells during the course of infection, or whether group 1 CD1-restricted T cells can mount a vigorous memory response upon rechallenge. This information is critical with respect to targeting group 1 CD1-restricted T cells for the development of a Mycobacterium tuberculosis (Mtb) vaccine. The kinetics of group 1 CD1-restricted T cell responses in hCD1Tg mice can be determined during primary and secondary mycobacterial infection. The kinetics of MHC class I-restricted and class 11-restricted T cells can be determined concurrently for comparison. In addition, the effect of the CD1-restricted responses during infection with Mtb can be evaluated by comparing the course of and susceptibility to infection between hCD1Tg mice and control mice in a B6, MHC class Ia-deficient (KboDbo), and MHC class II Aβ-deficient (MHC II) background.

a) Kinetic Analysis of Group 1 CD1, MHC Class Ia, and class II-Restricted T Cell Responses Following Mycobacteria Infection

To compare the emergence of group 1 CD1, MHC class Ia and class II-restricted T cells following Mycobacteria infection, hCD1 Tg+ and non-Tg mice can be infected with Mycobacterium bovis bacillus Calmette-Guérin (BCG, strain Aventis Pasteur, a kind gift from Dr. JoAnne Flynn, University of Pittsburgh School of Medicine). BCG is chosen for the kinetics study so that this extensive analysis can be performed in the biosafety level 2 facility; however it is understood that any Mycobacteria strain may be used. BCG shares not only numerous MHC-restricted antigens with virulent Mtb; it also shares the same basic structure with several known group 1 CD1-presented Mtb antigens, such as lipoarabinomannan (LAM) and mycolic acid (Gilleron, M., L. et al. (2000) J. Biol. Chem. 275:677-684), (Venisse, A., et al. (1993) J. Biol. Chem. 268:12401-12411). Studying the hCD1-restricted T cell response in BCG-immunized mice may reveal the contribution of hCD1-restricted T cells in BCG-mediated protection against mycobacterial infection in vaccinated subjects, as well as provide insight into the potential role of hCD1-restricted T cells in Mtb infection.

(1) Primary Infection

To compare the relative contribution of hCD1-restricted and MHC-restricted T cells during the course of mycobacterial infection, hCD1Tg+ and non-Tg mice can be infected with BCG (1×106 CFU) intravenously. Mice can be sacrificed at various times post-infection: days 6, 8, 10, 12, 14, 20, and 30. Lymphocytes isolated from spleen, lungs, and lung-draining lymph nodes can be subjected to surface phenotype and functional analysis. Briefly, flow cytometric analysis can be performed to compare the proportion of various T cells subsets (i.e. CD4+, CD8+, DN, and γδT cells) in infected hCD1Tg to that of control mice. The activation status of various T cell subsets can also be determined by their reactivity to antibodies specific for CD44, CD62L and CD69. The newly activated T cells are expected to be CD44hiCD62LloCD69+. This analysis can reveal whether any T cell subsets are preferentially activated in the infected hCD1 Tg mice. To analyze the function of these cells, T cells from infected hCD1 Tg mice can be enriched by magnetic sorting and stimulated in vitro to compare the frequency of class Ia, class II and hCD1-restricted T cells in the spleen, lungs, and lung-draining lymph nodes. BCG-specific T cells can be analyzed by ELISPOT assay and intracellular cytokine staining for IFN-γ using BCG-infected BM-DCs as stimulators. BM-DCs from wild type (Tg−), hCD1Tg+, hCD1Tg+KboDbo, and hCD1Tg+IIo mice can be used as stimulators to assess the relative frequency and peak appearance of hCD1-restricted, class Ia-restricted, and class II-restricted responses to BCG. All the mice used in this study can be backcrossed at least 10 generations onto B6 background to eliminate the alloreactive response against DC isolated from various strains of mice. The expression of CD4 and CD8 on IFN-γ secreting cells can also be examined to see whether hCD1-restricted BCG-specific T cells are enriched in CD4+, CD8+, or DN T cell subsets. These studies can provide information about the presence and rate of appearance of hCD1-restricted T cells in the relevant organs following BCG infection.

In addition to measuring IFN-γ production, the ability of hCD1-restricted T cells to function as cytotoxic T cells can also be examined. T cells isolated from lungs and spleen of BCG-infected hCD1Tg mice can be cultured for 5 days in vitro with BM-DCs from hCD1Tg in the presence of BCG antigens. BCG-infected wild type or hCD1Tg+ DCs can be used as targets in 51Cr-release CTL assays. Specific lysis of the infected DC that can be blocked by anti-hCD1 antibodies can indicate that hCD1-restricted CTLs exist in vivo and are capable of lysing the infected hCD1-expressing targets (e.g. DCs and macrophages) that would be encountered in the lungs.

(2) Secondary Infection

A similar kinetic analysis can be performed to examine the expansion and contraction of hCD1, class Ia and class II-restricted T cells following a secondary BCG infection. hCD1Tg+ mice can be infected with BCG and treated with antimycobacterial drugs (isoniazid and pyrazinamide) for four weeks as described by Scanga et al. (Scanga, C. A., et al. (1999) Infect Immun. 67:4531-4538) to eliminate the majority of the bacteria. The mice can then be rechallenged with BCG and at day 4, 6, 8, 10, 12, 14 and 21 post-infection, lung and spleen cells can be analyzed as above. The presence of BCG-specific hCD1, class Ia and class II restricted T cells can be assessed as described above.

(a) Expected Results, Potential Pitfalls and Alternative Approaches

Group 1 CD1-restricted responses can be observed in the BCG-infected hCD1Tg mice. Additionally, it was shown herein that T cells recognizing Mtb antigens in the context of group 1 CD1 can be derived from hCD1Tg mice immunized with Mtb antigens. However, the kinetics of appearance may be distinct from that of MHC class Ia and class II-restricted responses. Prior studies have shown that the MHC class Ia and class II-restricted responses peak around 2 weeks after primary infection with Mycobacteria (Kamath, A. T., et al. (2000) Clin. Exp. Immunol. 120:476-482), (Kamath, A. T., et al. (2000) Clin. Exp. Immunol. 120:476-482). Group 1 CD1-restricted T cells can but are not limited to function similar to NKT cells or other MHC class Ib-restricted T cells (i.e. H2-M3-restricted T cells) (Kerksiek, K. M., et al. (1999) J. Exp. Med. 190:195-204). Thus, one would expect the group 1 CD1-restricted T cells can appear earlier than MHC class Ia and class II-restricted T cells during the course of infection. Group 1 CD1-restricted T cells actively participating in the recall response to BCG challenge can be faster and stronger upon secondary infection. Alternatively, hCD1Tg+KboDbo and hCD1Tg+IIo mice can be infected to screen for group 1 CD1 restricted T cells in the residual CD8+ and CD4+ populations, respectively. Briefly, hCD1Tg+KboDbo mice can be infected with BCG; spleen, lungs, and lymph nodes can be obtained from sacrificed mice at the time points indicated above. CD8+ T cells can be purified by magnetic sorting and stimulated in vitro with DCs from hCD1Tg+KboDbo and KboDbo mice. Additionally, similar analyses can be performed in hCD1Tg+IIo mice, using DCs from hCD1Tg+IIo and IIo mice as stimulators to determine the kinetics of appearance of CD4+ hCD1-restricted cells following BCG infection.

(b) Examine the Overall Contribution of Group I CD1-Restricted Responses in the Protective Immunity Against Mycobacterium tuberculosis

Mycobacteria-specific, group 1 CD1-restricted T cells isolated from humans can produce IFN-γ and lyse infected macrophages, effector mechanisms important in control of tuberculosis (Flynn, J. L., et al. (1993) J. Exp. Med. 178:2249-2254), (Cooper, A. M., et al. (1993) J. Exp. Med. 178:2243-2247). However, it is currently unknown whether these T cells mediate protection against mycobacterial infection. To address this question, hCD1Tg+ and wild type mice can be infected with a virulent strain of Mtb (Erdman strain) and can compare the relative susceptibility to Mtb infection between these two groups of mice. Groups of mice can be infected either via the aerosol (100 CFU/mouse) or intravenous (2×105 CFU/mouse) routes because these two routes of infection result in differential distribution of bacilli in various organs. Mice inoculated intravenously with Mtb develop a systemic infection while mice challenged aerogenically with Mtb develop an actively growing infection in the lung and then disseminate to the spleen and liver at later time points. It has been shown that the intravenous model of murine tuberculosis is less pathogenic than aerogenic model owing to a more rapid induction of systemic immunity (Cardona, P. J., et al. (1999) Scand. J. Immunol. 49:362-366). The course of infection can be monitored by determining bacterial loads in the lungs, liver and spleen between day 15 and day 120 post-infection. Briefly, organs can be aseptically removed from infected animals and homogenized in phosphate-buffered saline containing 0.05% Tween-80. Ten-fold serial dilution can be plated onto 7H10 agar plates and colonies can be counted after incubation for three weeks at 37° C. Because IFN-γ is central to defense against Mycobacteria, IFN-γ production can be assessed by spleen cells from various infected mice after in vitro restimulation with Mtb sonicate. The observed phenotype may not be solely due to the presence of antigen-specific group 1 CD1-restricted T cells. Activation of “autoreactive” group 1 CD1-restricted T cells during Mtb infection, possibly by up-regulation of CD1 expression, may modulate Th1 development which is required for effective protection.

It has been shown that group 1 CD1 proteins are highly expressed in the skin lesions of human patients with the self-healing (tuberculoid granulomas) form of leprosy (Sieling, P. A., et al. (1999) J. Immunol. 162:1851-1858). The positive correlation between the level of group 1 CD1 expression and the degree of effective cellular immunity to Mycobacteria leprae leads to the hypothesis that upregulation of group 1 CD1 may significantly enhance the ability of the T cells to detect intracellular parasites such as Mycobacteria. Therefore, the levels of group 1 CD1 expression can be examined by immunohistochemical staining of tissue sections from the spleen and lungs of Mtb infected mice. In addition, two-color immunofluorescence staining of cryostat sections with anti-hCD1 antibodies and antibodies specific to CD11c (DC-specific), F4/80 (macrophage-specific), and B220 (B cell-specific) can be performed to identify the cell type(s) that express group 1 CD1 proteins.

Similar to humans, the group 1 CD1-restricted Mtb antigen-specific T cells lines derived from hCD1Tg mice can also secrete IFN—Y and have cytolytic activity. Thus, if the frequency of Mtb-specific group 1 CD1-restricted T cells are induced substantially in hCD1Tg mice during Mtb infection, as implicated from the study of Mtb patients (Moody, D. B., et al. (2000) Nature 404:884-888), hCD1Tg⁺ mice can control infection better. For example, they may have decreased bacterial burden compared to wild type controls.

It has been shown that K^{bo}D^{bo}, mice can control the acute phase of Mtb infection but are less efficient at maintaining the stationary phase of Mtb infection, while MHC II^{o} mice are incapable of controlling the acute phase Mtb infection and succumb to infection much earlier than K^{bo}D^{bo} and wild type mice (Mogues, T., et al. (2001) J. Exp. Med. 193:271-280), (Turner, J., et al. (2001) Am. J. Respi. Cell. Mol. Biol. 24:203-209), (Rolph, M. S., et al. (2001) Eur. J. Immunol. 31:1944-1949). Therefore, the data presented herein of the protective effects of hCD1-restricted response in hCD1Tg+KboDbo and hCD1Tg+IIo mice can also provide information on whether hCD1-restricted T cells contribute to acute and/or chronic phase immunity to Mtb infection. In addition, adoptive transfer experiments allow for determining that group 1 CD1-restricted Mtb lipid antigen-specific T cells are protective in vivo, and this would provide evidence for a role for group 1 CD1-restricted cells in the protective immune response to Mtb.

### Example 3

### Study the Immune Response to Known hCD1-Restricted Mycobacterial Lipid Antigens in hCD1Tg Mice

Several mycobacterial lipid antigens presented by human group 1 CD1 molecules have been identified and characterized. These include mycolic acid, lipoarabinomannan (LAM), and glucose monomycolate (GMM) for CD1b (Beckman, E. M., et al. 1994. Nature 372:691-694), (Sieling, P. A., et al. 1995. Science 269:227-230), (Moody, D. B., et al. (1997) Science 278:283-286), (Crich, D., and V. Dudkin (2002) J. Am. Chem. Soc. 124:2263-2266), and β-mannosyl phosphoisoprenoid (MPI) for CD1c (Moody, D. B., et al. (2000) Nature 404:884-888). Such conserved microbial lipid antigens may very well serve as candidate antigens for a Mtb vaccine. However, the immunogenecity and potential efficacy of these mycobacterial lipid antigens are still unknown. Whether the Mycobacteria-infected hCD1Tg mice can generate immune responses to these known CD1-presented mycobacterial lipid antigens as seen in humans can be examined. Similar specificity of CD1-restricted responses can be observed in hCD1Tg mice, thus the relative frequency of CD1-restricted T cells to each individual lipid antigen in infected mice can be determined. In addition, T cell lines specific to these CD1-restricted mycobacterial, lipid antigens can be established and examined for their functional properties and TCR diversity, and evaluate the protective role of these CD1-restricted, mycobacterial lipid-specific T cells in host defense against mycobacterial infection. Determine the frequency of GMM, mycolic acid, and MPI-specific CD1-restricted T cell in the BCG-infected hCD1Tg⁺ mice To see whether Mycobacteria-infected hCD1Tg mice can generate T cell responses to the same mycobacterial lipid antigens as human CD1-restricted CTLs, the emergence of hCD1-restricted T cells specific to GMM, mycolic acid, and MPI can be monitored following BCG infection of hCD1Tg mice can be monitored. Mycolic acid purified from a human strain of Mtb is commercially available. Although these cell wall components may have some distinct features in various strains of Mycobacteria, many general structures are conserved. Briefly, mice can be sacrificed at selected times post-immunization. The appearance of T cells specific for mycobacterial lipid antigens in the spleen, lungs, and lung-draining lymph nodes can be determined by ELISPOT and intracellular cytokine staining for IFN-γ, using BM-DCs from hCD1Tg and non-Tg mice pulsed with either GMM, mycolic acid, MPI or Mtb lipid extracts as stimulators. The restriction by hCD1 can also be confirmed by specific anti-hCD1 mAb blocking. The identification of GMM, mycolic acid, and MPI-specific hCD1-restricted T cells from infected mice indicates these lipid antigens are actually presented in vivo. In addition, these data allow for the assessment of the relative frequency of hCD1-restricted responses to individual lipid antigens.

A kinetic analysis can be performed on hCD1Tg mice following a secondary BCG infection to examine the expansion and contraction of these mycobacterial lipid-specific hCD1-restricted T cells. hCD1Tg mice can be infected and rechallenged with BCG as described. The presence of GMM, mycolic acid, and MPI-specific hCD1-restricted T cells can be assessed as described above. This study can reveal that these hCD1-restricted Mtb-lipid antigen specific T cells have the capacity to generate memory T cell responses upon reinfection, and that the strength of the primary response to a given lipid antigen correlates with that of the secondary response.

a) Generation and Characterization of Mycolic Acid, GMM, and MPI-Specific hCD1-Restricted T Cell Lines

Mycolic acid, GMM, MPI-specific hCD1-restricted T cell lines can be generated from infected hCD1Tg mice for both in vivo and in vitro functional studies. Briefly, spleen, lungs, and lymph node cells from infected mice can be obtained at the time points found to be the peak of the hCD1 response as indicated above. T cells can be expanded in vitro for several rounds by weekly restimulation, alternating between with lipid antigen-pulsed DC derived from hCD1Tg⁺K^{bo}D^{bo} and hCD1Tg⁺II^{o} mice. Additionally, the use of class Ia and class II-deficient hCD1-expressing DCs as stimulators can greatly reduce the possibility of generating all-reactive T cells specific to minor histocompatibility antigens. Mycolic acid, GMM, or MPI-specific CTLs can be screened for by ⁵¹Cr release assays and cytokine secretion assays with antigen-pulsed hCD1Tg+DC first, and subsequently with C1R-CD1A, B, or C transfected cells pulsed with and without antigen (as described in the preliminary studies). Antibody blocking can also be used to demonstrate group 1 CD1 specificity. Once the specificity of T cell lines is determined, T cell clones can be generated by limiting dilution. To compare the affinity among these T cell clones to their corresponding ligands, equal numbers of T cells from each clone can be incubated with hCD1 Tg+BM-DCs in the presence of various concentrations of the target antigen for 48 hr. The amount of cytokine released can be quantitated by ELISA and used as an indicator of the strength of reactivity. T cell clones with highest affinity to GMM, mycolic acid or MPI can be chosen for the adoptive transfer experiments.

Surface phenotypes and Vα and Vβ usage in these clones can be determined by FACS analysis. If these T cell clones use a restricted set of Vα and Vβ, the sequence of the TCR expressed by these clones can be determined by 5′-RACE and PCR using Cα-specific and Cβ-specific primers as described (Wang, B., et al. (2001) J. Immunol. 166:3829-3836), (Bradbury, A., et al. (1988) EMBO J. 7:3081-3086). Previous studies using five human T cell clones with diverse specificity for lipid antigens and CD1 isoform did not show any clear bias in V gene usage by these TCRs (Grant, E. P., et al. (1999) J. Exp. Med. 189:195-205), (Grant, E. P., et al. (2002) J. Immunol. 168:3933-3940). Analysis can be performed on T cells with specificity for a single lipid antigen in the context of a common CD1 isoform, which may reveal antigen-specific, or isoform-specific bias in V gene usage. Results from these experiments provide information about the diversity of the T cell receptor repertoire among group 1 CD1-restricted, lipid antigen-specific T cells. Several structural variants of GMM have been synthesized, including two stereoisomers of GMM (mannose monomycolate and galactosemonomycolate) and GMMs with different alkyl chain lengths (Moody, D. B., et al. (1999) Immunol. Rev. 172:285-296). To determine the fine specificity of TCR recognition, the reactivity of the GMM-specific CD1-restricted T cells with these GMM analogs can be examined.

b) Adoptive Transfer of Mycobacterial Lipid-Specific CD1-Restricted CTLs

Human CTLs specific to Mtb lipid antigens are capable of secreting IFN-[□] and lysing Mtb-infected target cells which raises the possibility that these Mtb lipid antigens can be used as vaccines against Mtb. Described above is a method for generating Mtb lipid-specific hCD1-restricted T cell lines from infected mice. To evaluate the protective effect of Mtb lipid specific CD1-restricted CTLs, T cell lines and clones (5×106 cells) can be harvested at day 7 after restimulation and adoptively transferred into hCD1Tg+ mice. Animals can be challenged immediately with Mtb by i.v. injection of Mtb (1×105 CFU). Using this type of protocol, other investigators have shown that adoptive transfer of CD4+ or CD8+ T cell clones specific to mycobacterial heat-shock protein 65 can significantly reduce the bacterial burden in recipient mice (Silva, C. L., et al. (1994) Immunology 83:341-346). As a negative control, a non-relevant CTL clone specific to SIINFEKL-peptide in the context of H-2 Kb can be adoptively transferred into hCD1Tg+ mice, which should have no effect on the control of Mtb infection. At 1, 2, 3, and 4 weeks post-infection, the number of viable Mtb organisms in spleen, liver, and lungs from each group can be determined. In addition, IFN-γ production can be followed by RNA analysis and intracellular cytokine staining of the spleen and lung cells at each time points.

4. Example 4

### Identify Novel Microbial Antigens Presented by Human Group 1 CD1 Molecules

Relatively few mycobacterial CD1-restricted lipid antigens have been identified. The hCD1Tg mice provide a valuable tool to identify new mycobacterial lipid antigens for the potential use as subunit vaccines for Mtb infection. Previously unknown CD1a-restricted Mtb lipid antigen can be identified, using the newly derived T cell line, HN2, which recognizes lipid extracts from Mtb in the context of CD1a. This CTL can also be used to characterize the processing and presentation of Mtb lipid antigen by CD11a.

Group 1 CD1 molecules can be involved in the presentation of lipid antigens from a broad range of microbial pathogens. To see whether group 1 CD1 can present antigen derived from Gram-positive bacteria, Tg mice can be infected with Listeria monocytogenes to derive group 1 CD1-restricted anti-listerial T cells, and determine what listerial antigens, are presented. Aside from being a well-characterized intracellular bacterial infectious model, Listeria is of interest because it contains a variety of glycosylated lipoteichoic acids (LTA) (Fischer, W., et al. (1990) Biochem. Cell. Biol. 68:33-43), which share several common features with other CD1-binding antigens. It is possible that some of these glycolipid antigens derived from the listerial cell wall can bind the CD1 antigen-binding pocket and be available for TCR recognition. The demonstration of the presence of T cells which recognize listerial lipid/glycolipid antigens in the context of human group 1 CD1 molecules can support a general role of group 1 CD1-mediated lipid antigen presentation in host defense against microbial pathogens.

a) Determine the Molecular Nature and Presentation Requirements of a CD1a-Restricted Mycobacterial Lipid Antigen

Disclosed herein is a CTL line, HN2, which recognized lipid extracts from Mtb in the context of CD1a. As the initial step to characterize the Mtb lipid antigen recognized by HN2, proliferation assays can be used to test the reactivity of HN2 with various lipid fractions from Mtb, including neutral lipid, glycolipid, and phospholipid fractions (kindly provided by Dr. Michael Brenner, Harvard Medical school (Kenji Hiromatsu et al., The Journal of Immunology, 2002 169:330-339). Active fractions can be further fractionated on silica columns, using a step gradient of increasing methanol percentage in chloroform for elution (Rosat, J. P., et al. 1999. J. Immunol. 162:366-371). The partially purified lipid fraction can be separated by thin layer chromatography (TLC) on silica plates, and individual spots corresponding to the separated lipids can be scraped from the TLC plates. After re-extraction of the lipids, each can be tested for antigenic activity.

The antigen presentation/processing requirements for various CD1b-presented antigens have been studied extensively, and show that the molecular structure of antigens may affect their processing requirements (Moody, D. B., et al. (2002) Nat. Immunol. 3:435-442). In contrast, information on the presentation of mycobacterial lipid antigen by CD11a has been limited to only one human T cell line, CD8-2. The recognition of Mtb-derived antigen by CD8-2 requires intracellular delivery of lipid antigen, but does not depend on vesicular acidification (Rosat, J. P., et al. 1999. J. Immunol. 162:366-371). To further understanding on the antigen presentation pathway mediated by CD11a, it can be determined if Mtb lipid antigens recognized by HN2 requires intracellular processing. Briefly, glutaraldehyde fixation of hCD1Tg+DCs before or at different time points after addition of Mtb lipid extracts can be performed to determine if antigen processing is required. When antigen processing is required, the effects of various inhibitors such as chloroquine, Concanamycin A, Balifomycin A, and Wortmannin A, which block various steps in antigen presentation pathways, can be used. The effects of these inhibitors are as follows: chloroquine is a weak base which elevates the pH in endocytic compartments and thereby inhibits the action of acid-optimal proteinases; Concanamycin A blocks transport from early to late endosomes; Balifomycin A is known to block transport from late endosomes to lysosomes (Benaroch, P., et al. (1995) EMBO J. 14:37-49), (Valitutti, S., et al. (1997) J. Exp. Med. 185:1859-1864); and Wortmannin A has been shown to inhibit endosomal recycling pathways, such as internalization of molecules from the cell surface, without effecting endosomal acidification (Mayor, S., et al. (1993) J. Cell. Biol. 121:1257-1269), (Shepherd, P. R., et al. (1995) Biochem. Biophys. Res. Commun. 211:535-539). The ability of these reagents to block the reactivity of HN2 T cells can reveal whether endosomal acidification is required for the presentation of HN2 antigen and which compartment is involved in antigen presentation by CD1a molecules.

b) Generation and Characterization of Group 1 CD1-Restricted Listerial Antigen-Specific T Cells from Listeria-Infected hCD1Tg Mice

Listeria monocytogenes is a Gram-positive, facultative intracellular pathogen that is widespread in the environment and capable of causing infections in humans and animals. Listeria infection is known to induce both CD4+ and CD8+ T cells (Kaufmann, S. H. (1993) Annu Rev Immunol 11: 129-163), indicating that listerial antigens can be processed and presented by both MHC class I and class II antigen-presentation pathways. To derive group 1 CD1-restricted listerial antigen-specific T cells, hCD1Tg mice can be infected with Listeria monocytogenes (5×103CFU/mouse) intravenously. T cells isolated from the spleen and lymph nodes of infected mice can be subjected to weekly restimulation with hCD1-expressing BM-DCs pulsed with lipid extracts from Listeria. Crude lipid extracts from Listeria can be prepared as described by Folch et al. (Folch, J., et al. (1957) J. Bio. Chem. 226:497). Irradiated hCD1Tg+KboDbo DCs and hCD1Tg+IIo DCs can be used as stimulators at alternating weekly intervals to minimize the expansion of MHC class Ia and class II-restricted Listeria-specific T cells in vitro. hCD1-restricted Listeria-specific T cells can be screened for by 51Cr release assays and cytokine secretion assays using hCD1Tg+ and hCD1Tg− DC with and without listerial antigens as targets. Listeria-specific hCD1-restricted T cells can react with cells from hCD1Tg+mice in an antigen-dependent manner but not with cells from Tg− mice. The restriction by CD1a, b, or c can be established by specific anti-hCD1 mAb blocking and by analysis of reactivity against CD1A, B, and C-transfected C1R cells.

c) Determine the Specificity and Type of Listerial Antigens Recognized by Group 1 CD1-Restricted T Cells

Once the hCD1-restricted Listeria-specific CTLs are established, the following experiments can be performed to investigate the chemical nature of the microbial antigens recognized by these T cells. The approach can abrogate killing of antigen pulsed targets by subjecting the Listeria lipid extracts to various treatments, including digestion with various proteases (e.g. proteinase K), digestion with glycanase (e.g. α-exomannosidase), mild alkali hydrolysis (de-acylation), and extraction with detergent or chloroform (de-lipidation). When these experiments indicate that the antigen is unlikely to be peptides, then various lipid fractions from Listeria can be tested for reactivity with these hCD1-restricted Listeria-specific CTLs. LTA can be the first candidate to test. LTA from Listeria can be isolated by phenol/water extraction, followed by hydrophobic chromatography as described by Geyer et al. (Morath, S., et al. (2002) Infect. Immun. 70:938-944).

As listerial antigens have access to both cytosolic and lysosomal antigen processing pathways, can be accessible to group 1 CD1 molecules in the antigen presenting cells. Due to the structural similarity between listerial lipid/glycolipid antigens (i.e. LTA) with known hCD1-binding antigens (i.e. LAM), hCD1-restricted responses can be observed in the Listeria-infected hCD1Tg mice.

### Example 5

### The Role of Group 1 CD1 in T Cell Development and Maintenance

CD1 represents a third lineage of antigen presenting molecules. While the role of group 2 CD1 in T cell development is well established, little is known about the contribution of group 1 CD1 to the overall T cell repertoire. Herein, the contribution of group 1 CD1 in the development of various T cell subsets in homeostatic conditions can be examined. To rule out the possibility that the effects of the hCD1 transgene may be obscured by the presence of endogenous MHC class Ia, class II, and mouse CD1d molecule, the function of group 1 CD1 in the development of CD8, CD4, and NK T cell compartments can be examined in hCD1Tg⁺K^{bo}D^{bo}, hCD1Tg⁺II^{o}, and hCD1Tg+CD1^{o} mice, respectively.

Two different types of T cell populations reactive to human group 1 CD1 have been reported. One type of hCD1-restricted T cell recognizes microbial antigens in the context of group 1 CD1 molecules, and presumably function similar to conventional antigen-specific CD4+ and CD8+ T cells in adaptive immunity. The other type of hCD1-restricted T cell recognizes group 1 CD1 in the absence of foreign antigens. This feature is shared with NKT cells, which recognize group 2 CD1 independent of foreign lipid antigen. Recent studies showed the requirement of thymic selection and peripheral survival of NKT cells are distinct from those of conventional T cells (Matsuda, J. L., et al. (2002) Nat. Immunol. 3:966-974). Disclosed herein a CD1c-restricted autoreactive T cell clone, HN1-4 was generated and characterized. transgenic mice expressing HN1-4 TCR can be generated and crossed onto hCD1Tg background to study the requirements for selection and maintenance of group 1 CD1-restricted T cells.

a) Determine the Role of Group 1 CD1 in the Development of CD8, CD4, and NKT Cells in hCD1Tg⁺K^{bo}D^{bo}, hCD1Tg⁺II^{o}, and hCD1Tg⁺CD1^{o} mice

(1) Determine the Contribution of Group 1 CD1 in the Development of Various T Cells Subsets

Group 1 CD1-restricted T cells have been identified from multiple T cells subsets in humans, including CD4⁻CD8⁻ (DN)/TCRαβ, CD8αα⁺/TCRαβ, CD8αβ⁺/TCRαβ, CD4⁺/TCRαβ, and TCRγδT cells. Therefore, hCD1Tg⁺K^{bo}D^{bo} and hCD1Tg⁺II^{o} mice can be used to directly examine the role of group 1 CD1 in the development of CD8 and CD4 T cell compartments in the thymus and periphery. The number of the CD4⁺, CD8⁺ (CD8αα⁺ and CD8αβ), and DN T cells can be determined from various lymphoid organs, including thymus, spleen, lymph nodes, liver, and intestine, from hCD1Tg⁺K^{bo}D^{bo} and hCD1Tg⁺II^{o} mice, and compare these populations to that of K^{bo}D^{bo} and MHC 11 mice, respectively. In addition, VP usage of residual CD8⁺ T cells in K^{bo}D^{bo} and hCD1Tg+K^{b}D^{bo} mice can be compared to determine whether a particular set of T cells is preferentially expanded by hCD1 transgene. Similar analysis can be performed for the residual CD4⁺ T cells in MHC II^{o} and hCD1Tg⁺II^{o} mice.

(2) To Examine Whether there is a Functional Overlap Between Group 1 and Group 2 CD1 in the Selection of NKT Cells.

The sequence homology and tissue distribution of two CD1 classes indicates they can serve different functions in the immune system. However, the overall homology between the two groups is still high, and similar ligands, such as α-galactosylceramide (α-GalCer) and GM1, can bind both group 1 (CD1b) and group 2 CD1 (Naidenko, O. V., et al. (1999) J. Exp. Med. 190:1069-1080). Unlike mouse CD1d, human CD1d (hCD1d) shows low levels of expression on thymocytes, the cell type that mediates positive selection of NKT cells (Bendelac, A., et al. (1995) Science 268:863-865), (Coles, M. C., and D. H. Raulet (2000) J. Immunol. 164:2412-2418), and therefore hCD1d may not be as an efficient selecting element as mouse CD1d1 for NKT cells. Since group 1 CD1 molecules are primarily expressed on thymocytes, this raises the question as to whether group 1 CD1 can at least in part compensate for the function of group 2 CD1 in selecting this special subset of T cells. To address this, hCD1Tg mice can be crossed into a CD1-deficient background, and FACS analysis performed to determine if this NKT cell subset is reconstituted in hCD1Tg+CD1o mice. Although CD1d/αGalCer tetramers react with the majority of invariant Vα4+NKT cells, there are some NKT cells that do not recognize α-GalCer (Matsuda, J. L., et al. (2000) J. Exp. Med. 192:741-754), (Park, S. H., et al. (2001) J. Exp. Med. 193:893-904), (Benlagha, K., et al. (2000) J. Exp. Med. 191:1895-1903). Therefore, both TCR-β/NK1.1 and CD1 d/αGalCer tetramer staining can be used to examine NKT cell development in the hCD1Tg+CD1o mice and compare them to that in CD1o mice.

(3) Examine the Requirements for the Development and Maintenance of Group 1 CD1-Restricted T Cells

The group 2 CD1-reactive NKT cells represent a unique lineage of T cells, which express several phenotypic markers usually associated with NK cells (e.g. NK1.1 and Ly49) and activated/memory T cells (e.g. CD62Llo and CD44hi) (Bendelac, A. (1995) Curr. Opin. Immunol. 7:367-374), (Bix, M., and R. M. Locksley (1995) J. Immunol. 155:1020-1022). Most of the NKT cells recognize group 2 CD1 in the absence of exogenous antigen. The autoreactive nature of NKT cells may partly contribute to their unique surface phenotype and functional properties. A large proportion of group 1 CD1-restricted T cell lines isolated so far (from humans and the Tg mice disclosed herein) are also autoreactive. However, unlike NKT cells, the surface phenotype and TCR repertoire for group 1 CD1-reactive T cells appears to be quite heterogeneous. It is unclear whether these group 1 CD1-restricted autoreactive T cells have functional properties similar to NKT cells (e.g. promptly acquire effector functions upon primary stimulation), and whether the mechanism described for the development and selection of NKT cells also applies to these group 1 CD1-restricted autoreactive T cells. To address these questions, transgenic mice can be generated bearing TCR specific to CD1c, the CD1 isoform recognized by most of the autoreactive CD1-restricted T cells, and study the development and functional properties of the TCRTg+ T cells.

(a) Generation of Transgenic Mice that Express TCR Specific for CD1c

Clone HN1-4, which expresses Vα5/Vβ9 and recognizes CD1c-expressing targets in the absence of exogenous antigens (preliminary studies), can be used as the source for rearranged TCR genes to construct TCR transgenic mice. Rearranged VαJα and VβDJβ fragments can be amplified by PCR from genomic DNA of HN1-4 and cloned into the TCR cassette vectors, pTαcass and pTβcass (provided by Dr. D. Mathis), derived from HY-TCR α and β cosmid clones, respectively (Kouskoff, V., et al. (1995) J. Immunol. Meth. 180:273-280). Identification of founders can be performed by Southern blot analysis with single copy probes for the Cα and Cβ regions as described previously (Chiu, N. M., et al. (1999) J. Exp. Med. 190:1869-1878).

(b) Investigation of the Development of HN1Tg⁺ T Cells in hCD1Tg Background

HN1Tg founder mice can be crossed onto hCD1Tg background and used to determine whether the expression of human group 1 CD1 transgene is required for the development of HN1Tg+ T cells. Since HN1-4 T cells showed no cross-reactivity to mouse CD1d, it is unlikely that mouse CD1d can mediate positive selection of HN1 Tg+ T cells effectively. Therefore, transgenic Vβ9+ T cells can be highly enriched in HN1Tg+hCD1Tg+but not in HN1Tg+ hCD1Tg− mice. TCRα-deficient background can be used instead of RAG-deficient background for this study in order to preserve the B cell populations, which express CD1c molecules in hCD1 Tg mice.

(i) Analysis of Tissue Distribution and Surface Phenotype of HN1Tg⁺ T cells.

Similar to several autoreactive group 1 CD1-restricted T cells identified in humans, clone HN1-4 express CD8αβ but not CD8αβ. The expression of CD8αα on HN1-4 T cells can simply be due to downregulation of CD8β expression as a consequence of long-term in vitro culture and antigen stimulation. Alternatively, it can reflect a distinct developmental/selection condition in the generation of these T cells. A recent study using TCR transgenic mice specific for MHC class I or class II-restricted antigens revealed that CD8αα/TCRαβ T cells derived from the thymus after positive selection by agonist self-peptides and that they exhibit distinct functions from conventional CD8αβ/TCRαβ T cells (Leishman, A. J., et al. (2002) Immunity 16:355-364). In addition, they are most abundant in intestinal intraepithelial lymphocyte (IEL) population. To examine the relative abundance of HNTg+ T cells in various lymphoid organs and see if CD8αα is expressed by HN1Tg+ T cells, lymphocytes can be isolated from various organs from HN1Tg+hCD1Tg+ mice, including thymus, spleen, lymph nodes, liver and intestine, and perform flow cytometric analysis using antibodies specific to Vβ9, CD8α, CD8β, and CD4. In addition, the expression of various activation/memory markers (e.g.CD44, CD62, Ly6C, and CD122) on HN1Tg+ T cells can also be examined.

(ii) Determine the Functional Properties of HN1Tg⁺ T Cells.

HN1-4 T cell clone secretes IFN-γ, but not IL-4 when stimulated with hCD1Tg+DCs. In addition, HN1-4 T cells can lyse CD1c-bearing targets. To examine if effector functions of clone HN1-4 are preserved in HN1Tg+ T cells, cytokine secretion and CTL activity of HN1Tg+ T cells isolated from various organs can be examined. If HN1Tg+ T cell can secrete IFN-γ in response to hCD1Tg+DCs, the kinetics of IFN-γ secretion by HN1Tg+ T cells to naïve and memory OT-1 TCR Tg+ T cells (specific for SIINFEKL peptide of OVA in the context of H2-Kb) can be compared. Because OT-1 TCR Tg+RAGo mice (kindly provided by Dr. Averil Ma, University of Chicago, which is available commercially have an endogenous population of TCR Tg+memory T cells (CD44hi, Ly6C+) (Curtsinger, J. M., et al. (1998) J. Immunol. 160:3236-3243), naïve (CD44 lo) and memory (CD44hi) OT-1 TCR Tg+ T cells can be isolated by FACS. Naïve and memory OT-1 TCR Tg+ T cells can be cultured with B6 DCs preloaded with SIINFEKL peptide, cells can be harvested at various time points (e.g. 4, 8, 12, 24, and 48 hr) and the number of IFN-γ producing cells can be determined by intracellular staining and compared to that of HNTg+ T cells in response to hCD1 Tg+ DCs stimulation. HN1Tg+ T cells that undergo agonist driven selection (i.e. selected by CD1c/self lipid antigen in the thymus), can confer the activation phenotype and rapid cytokine response to HN1Tg+ T cells.

(c) Determine the Requirements for Selection, Survival, and Expansion of HN1Tg⁺ T Cells

(i) What are the Cell Types that Mediate Positive Selection of HN1Tg⁺ T Cells?

Recent studies revealed that unlike conventional T cell positive selection, which is mediated by MHC molecules on thymic epithelial cells, NKT cells are positively selected by CD1d1 expressed on thymocytes (Coles, M. C., and D. H. Raulet (2000) J. Immunol. 164:2412-2418), (Coles, M. C., and D. H. Raulet (1994) J. Exp. Med. 180:395-399), (Bendelac, A. (1995) J. Exp. Med. 182:2091-2096). Although hCD1a, b, and c are expressed predominantly on thymocytes, low levels of hCD1a, b, and c expression can be detected on thymic epithelial cells in hCD1Tg mice. To investigate the contribution of hCD1expression by hematopoietic cells or non-hematopoietic cells (e.g. thymic epithelial) in the selection of HN1Tg+ T cells, BM chimera experiments can be performed. Table III outlines an example of the combination of donor and recipient mice being used in the study as well as the predicted results.

TABLE III

| | | Presence of Vβ9⁺ Cells | |
|---|---|---|---|
| BM Donor | Irradiated | If Selected | If Selected by |
| (TCRα° background) | Recipient | by TEC | Thymocytes |
| HN1Tg⁺hCD1Tg⁺ | hCD1Tg⁺ | + | + |
| HN1Tg⁺hCD1Tg⁺ | hCD1Tg⁻ | − | + |
| HN1Tg⁺hCD1Tg⁻ | hCD1Tg⁺ | + | − |
| HN1Tg⁺hCD1Tg⁻ | hCD1Tg⁻ | − | − |

(ii) (Ichimiya, S., et al. 1994. J. Immunol. 153:1112-1123) Is the Expression of CD1c-Required for Homeostatic Proliferation of HN1Tg⁺ T Cells?

Survival and homeostatic expansion of naïve conventional T cells requires an interaction with cognate self-MHC molecules whereas maintenance of memory cells is independent of self-MHC stimulation (Tanchot, C., et al. (1997) Science 276:2057-2062), (Murali-Krishna, K., et al. (1999) Science 286:1377-1381). A recent study showed that group 2 CD1-restricted NKT cells do not depend on TCR/CD1 d interactions to undergo expansion in a lymphopenic environment, consistent with their memory phenotype (Matsuda, J. L., et al. (2002) Nat. Immunol. 3:966-974). HN1Tg⁺ T cells can be adoptively transferred into either hCD1Tg⁺ or hCD1Tg⁻ mice to examine whether interaction with CD1c is essential for the homeostatic expansion of CD1c-restricted T cells.

To avoid activation of HN1Tg⁺ T cells during purification, HN1Tg⁺ T cells can be enriched by depletion of other cell populations with a mixture of magnetic beads specific for MHC class II, DX5, CD11b, and CD19. HN1Tg⁺ T cells can be isolated from spleen and lymph nodes of HN1Tg⁺hCD1Tg⁺TCRα^{o} mice and labeled with the carboxyfluorescein diacetate succinimidyl diester (CFSE). 2×106 CFSE-labeled HN1Tg+ T cells (Ly5.2⁺) can be transferred into irradiated B6-Ly-5.1 congenic and hCD1Tg (on Ly5.1⁺ background) mice. The number of cell divisions can be determined by analyzing the CFSE intensity of Vβ9⁺ cells 3, 7, 10, and 14 days after transfer. If HN1Tg⁺ T cells proliferate in hCD1Tg⁺ mice but not in control mice, that would indicate that CD1c is essential for the maintenance of CD1c-restricted T cells.

(iii) (Calabi, F., et al. 1989. Immunogenetics 30:370-377) Can Infection Induce Activation and Proliferation of HN1Tg⁺ T Cells?

To investigate whether HN1Tg+ T cells can be activated and expanded during infection, 5-10×106 CFSE-labeled HN1Tg+ T cells (Ly5.2+) can be adoptively transferred into intact hCD1Tg (on Ly5.1+ background) mice. The mice can be immediately infected with BCG intravenously. The proliferative ability of HN1Tg+ T cells from spleen and lungs of infected mice can be determined by analyzing the CFSE intensity of Vβ9+cells 3, 7, 10, and 14 days after transfer. In addition, the surface phenotype and cytokine secretion capacity of HN1Tg+ T cells harvested from infected mice can be examined. When HN1Tg+ T cells undergo activation and proliferation in infected mice but not uninfected mice can indicate that autoreactive CD1c-restricted T cells can participate in the immune response against infectious disease. Adoptive transfer of OT-1 T cells can be used as an additional control.

(Balk, S. P., et al. 1991. J. Immunol. 146:768-774) Mice can be used as a model to study the function of group 1 CD1 in T cell development and host defense against intracellular bacterial infection. Toward these aims, transgenic mice have been generated that express human group 1 CD1 molecules (hCD1Tg). The hCD1Tg mice can be backcrossed onto C57BL/6 background, and then crossed with several mutant mice (i.e., K^{bo}D^{bo}, MHCII^{o}, and CD1^{o}) to dissect the relative contribution of group 1 CD1 and MHC molecules to microbial immunity and the development of various T cell subsets. In addition, transgenic mice (HN1Tg) can be generated which bear T cell receptor specific for CD1c. The HN1Tg can be crossed onto hCD1Tg and TCRα^{o} background to study the requirement for the selection and maintenance of group 1 CD1-restrcited T cells.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### D. REFERENCES

Altmann, D. M., D. C. Douek, A. J. Frater, C. M. Hetherington, H. Inoko, and J. I. Elliott. 1995. The T cell response of HLA-DR transgenic mice to human myelin basic protein and other antigens in the presence and absence of human CD4. J. Exp. Med. 181:867-875.
Angenieux, C., J. Salamero, D. Fricker, J. P. Cazenave, B. Goud, D. Hanau, and H. de La Salle. 2000 Characterization of CD1e, a third type of CD1 molecule expressed in dendritic cells. J. Biol. Chem. 275:37757-37764.
Balk, S. P., P. A. Bleicher, and C. Terhorst. 1991. Isolation and expression of cDNA encoding the murine homologues of CD1. J. Immunol. 146:768-774.
Beckman, E. M., A. Melian, S. M. Behar, P. A. Sieling, D. Chatterjee, S. T. Furlong, R. Matsumoto, J. P. Rosat, R. L. Modlin, and S. A. Porcelli. 1996. CD1c restricts responses of mycobacteria-specific T cells. Evidence for antigen presentation by a second member of the human CD1 family. J. Immunol. 157:2795-2803.
Beckman, E. M., S. A. Porcelli, C. T. Morita, S. M. Behar, S. T. Furlong, and M. B. Brenner. 1994. Recognition of a lipid antigen by CD1-restricted αβ⁺ T cells. Nature 372:691-694.
Behar, S. M., Podrebarac, T. A., Roy, C., Wang, C.-R., & Brenner, M. B. (1999) Diverse T cell receptors recognize murine CD1. J. Immunol. 162: 161-167.
Behar, S. M., C. C. Dascher, M. J. Grusby, C. R. Wang, and M. B. Brenner. 1999. Susceptibility of mice deficient in CD1D or TAP1 to infection with Mycobacterium tuberculosis. J. Exp. Med. 189:1973-1980.
Benaroch, P., M. Yilla, G. Raposo, K. Ito, K. Miwa, H. J. Geuze, and H. L. Ploegh. 1995. How MHC class II molecules reach the endocytic pathway. EMBO J. 14:37-49.
Bendelac, A. 1995. Mouse NK1⁺T cells. Curr. Opin. Immunol. 7:367-374.
Bendelac, A. 1995. Positive selection of mouse NK1⁺ T cells by CD1-expressing cortical thymocytes. J. Exp. Med. 182:2091-2096.
Bendelac, A., O. Lantz, M. E. Quimby, J. W. Yewdell, J. R. Bennink, and R. R. Brutkiewicz. 1995. CD1 recognition by mouse NK1⁺ T lymphocytes. Science 268:863-865.
Benlagha, K., A. Weiss, A. Beavis, L. Teyton, and A. Bendelac. 2000. In vivo identification of glycolipid antigen-specific T cells using fluorescent CD1d tetramers. J. Exp. Med. 191:1895-1903.
Bix, M., and R. M. Locksley. 1995. Natural T cells: Cells that co-express NKRP-1 and TCR. J. Immunol. 155:1020-1022.
Bradbury, A., K. T. Belt, T. M. Neri, C. Milstein, and F. Calabi. 1988. Mouse CD1 is distinct from and co-exists with TL in the same thymus. EMBO J. 7:3081-3086.
Briken, V., D. B. Moody, and S. A. Porcelli. 2000. Diversification of CD1 proteins: sampling the lipid content of different cellular compartments. Semin. Immunol. 12:517-525.
Brossay, L., D. Jullien, S. Cardell, B. C. Sydora, N. Burdin, R. L. Modlin, and M. Kronenberg. Mouse CD1 is mainly expressed on hemopoietic-derived cells. J. Immunol. 159:1216-1224.
Calabi, F., and C. Milstein. 1986. A novel family of human major histocompatibility complex-related genes not mapping to chromosome 6. Nature 323:540-543.
Calabi, F., J. M. Jarvis, L. Martin, and C. Milstein. 1989. Two classes of CD1 genes. Eur. J. Immunol. 19:285-292.
Calabi, F., T. K. Belt, C.-Y. Yu, A. Bradbury, W. J. Mandy, and C. Milstein. 1989. The rabbit CD1 and the evolutionary conservation of the CD1 gene family. Immunogenetics 30:370-377.
Cardona, P. J., A. Cooper, M. Luquin, A. Ariza, F. Filipo, I. M. Orme, and V. Ausina. 1999. The intravenous model of murine tuberculosis is less pathogenic than the aerogenic model owing to a more rapid induction of systemic immunity. Scand. J. Immunol. 49:362-366.
Caruso, A. M., N. Serbina, E. Klein, K. Triebold, B. R. Bloom, and J. L. Flynn. 1999. Mice deficient in CD4 T cells have only transiently diminished levels of IFN-γ, yet succumb to tuberculosis. J. Immunol. 162:5407-5416.
Cattoretti, G., E. Berti, A. Mancuso, L. D'Amato, R. Schiro, D. Soglio, and D. Delia. 1987. An MHC class I related family of antigens with widespread distribution on resting and avtivated cells, in “Leukocyte typing III: White cell differentiation antigens”. Oxford University Press, Oxford. 89-91 pp.
Chen, Y. H., N. M. Chiu, M. Mandal, N. Wang, and C. R. Wang. 1997. Impaired NK1⁺ T cell development and early IL-4 production in CD1-deficient mice. Immunity 6:459-467.
Chen, Y.-H., Wang, B., Zhao, L., Cardell, S., Wang, N. & Wang, C.-R. (1999) The expression of CD1.2 on thymocytes is not sufficient for the development of NK T cells in CD1d1-deficient mice. J. Immunol. 162: 4560-4566.
Chen, Z., N. Dudek, O. Wijburg, R. Strugnell, L. Brown, G. Deliyannis, D. Jackson, F. Koentgen, T. Gordon, and J. McCluskey. 2002. A 320-kilobase artificial chromosome encoding the human HLA DR3-DQ2 MHC haplotype confers HLA restriction in transgenic mice. J. Immunol. 168:3050-3056.
Cheuk, E., C. D'Souza, N. Hu, Y. Liu, H. Lang, and J. W. Chamberlain. 2002. Human MHC class I transgenic mice deficient for H2 class I expression facilitate identification and characterization of new HLA class I-restricted viral T cell epitopes. J. Immunol. 169:5571-5580.
Chiu, N., Chun, T., Fay, M., Mandal, M., & Wang, C.-R. (1999) The majority of H2-M3 is retained intracellularly in a peptide-receptive state and traffics to the cell surface in the presence of N-formylated peptides. J. Exp. Med. 190: 423-434.
Chiu, N. M., B. Wang, K. M. Kerksiek, R. Kurlander, E. G. Pamer, and C. R. Wang. 1999. The selection of M3-restricted T cells is dependent on M3 expression and presentation of N-formylated peptides in the thymus. J. Exp. Med. 190:1869-1878.
Chun, T., K. Wang, F. A. Zuckermann, and H. R. Gaskins. 1999. Molecular cloning and characterization of a novel CD1 gene from the pig. J. Immunol. 162:6562-6571.
Chun, T., Page, M. J., Gapin, L; Matsuda J. L. Xu. H. Nguyen, H., Kang, H.-S., Stanic, A., Joyce, S., Koltun, W. A., Kronenberg, M., & Wang, C.-R. (2003) CD1d-expressing dendritic cells but not thymic epithelial cells can mediate negative selection of NKT cells. J. Exp. Med. 197: 907-918.
Chun, T., Serbina, N. V., Dawn N., Wang, B., Chiu, N. M., Flynn, J. L., & Wang, C.-R. (2001) Induction of M3-restricted CTL responses by N-formylated peptides derived from Mycobacterium tuberculosis. J. Exp. Med. 193: 1213-1220.
Coles, M. C., and D. H. Raulet. 1994. Class I dependence of the development of CD4+ CD8-NK1.1+thymocytes. J. Exp. Med. 180:395-399.
Coles, M. C., and D. H. Raulet. 2000. NK1.1+T cells in the liver arise in the thymus and are selected by interactions with class I molecules on CD4⁺ CD8⁺ cells. J. Immunol. 164:2412-2418.
Cooper, A. M., D. K. Dalton, T. A. Stewart, J. P. Griffin, D. G. Russell, and I. M. Orme. 1993. Disseminated tuberculosis in interferon gamma gene-disrupted mice. J. Exp. Med. 178:2243-2247.
Crich, D., and V. Dudkin. 2002. Confirmation of the connectivity of 4,8,12,16,20-pentamethylpentacosylphoshoryl β-D-mannopyranoside, an unusual β-mannosyl phosphoisoprenoid from Mycobacterium avium, through synthesis. J. Am. Chem. Soc. 124:2263-2266.
Curtsinger, J. M., D. C. Lins, and M. F. Mescher. 1998. CD8⁺ memory T cells (CD44^{high}, Ly-6C⁺) are more sensitive than naïve cells to (CD44^{low}, Ly-6C⁻) to TCR/CD8 signaling in response to antigen. J. Immunol. 160:3236-3243.
Dascher, C. C., K. Hiromatsu, J. W. Naylor, P. P. Brauer, K. A. Brown, J. R. Storey, S. M. Behar, E. S. Kawasaki, S. A. Porcelli, M. B. Brenner, and K. P. LeClair. 1999. Conservation of a CD1 multigene family in the guinea pig. J. Immunol. 163:5478-5488.
Dellabona, P., E. Padovan, G. Casorati, M. Brockhaus, and A. Lanzavecchia. 1994. An invariant V alpha 24-JαQ/V β 11 T cell receptor is expressed in all individuals by clonally expanded CD4−8− T cells. J. Exp. Med. 180:1171-1176.
Dutronc, Y., and S. A. Porcelli. 2002. The CD1 family and T cell recognition of lipid antigens. Tissue Antigens 60:337-353.
Exley, M., J. Garcia, S. B. Wilson, F. Spada, D. Gerdes, S. M. Tahir, K. T. Patton, R. S. Blumberg, S. Porcelli, A. Chott, and S. P. Balk. 2000. CD1d structure and regulation on human thymocytes, peripheral blood T cells, B cells and monocytes. Immunology 100:37-47.
Fairhurst, R. M., C. X. Wang, P. A. Sieling, R. L. Modlin, and J. Braun. 1998. CD1 presents antigens from a gram-negative bacterium, Haemophilus influenzae type B. Infect. Immun. 66:3523-3526.
Faure, F., S. Jitsukawa, C. Miossec, and T. Hercend. 1990. CD1c as a target recognition structure for human T lymphocytes: analysis with peripheral blood γ/δ cells. Eur. J. Immunol. 20:703-706.
Ferguson, E. D., B. M. Dutia, W. R. Hein, and J. Hopkins. 1996. The sheep CD1 gene family contains at least four CD1B homologues. Immunogenetics 44:86-96.
Fischer, W., T. Mannsfeld, and G. Hagen. 1990. On the basic structure of poly-(glycerophosphate) lipoteichoic acids. Biochem. Cell. Biol. 68:33-43.
Flynn, J. L., and J. Chan. 2001. Immunology of tuberculosis. Annu Rev Immunol 19:93-129.
Flynn, J. L., J. Chan, K. J. Triebold, D. K. Dalton, T. A. Stewart, and B. R. Bloom. 1993. An essential role for interferon gamma in resistance to Mycobacterium tuberculosis infection. J. Exp. Med. 178:2249-2254.
Flynn, J. L., M. M. Goldstein, K. J. Triebold, B. Koller, and B. R. Bloom. 1992. Major histocompatibility complex class I-restricted T cells are required for resistance to Mycobacterium tuberculosis infection. Proc. Natl. Acad. Sci U.S.A. 89:12013-12017.
Folch, J., M. Lees, and G. H. Sloan-Stanley. 1957. A simple method for the isolation and purification of total lipids from animal tissues. J. Bio. Chem. 226:497.
Gadola, S. D., N. R. Zaccai, K. Harlos, D. Shepherd, J. C. Castro-Palomino, G. Ritter, R. R.
Schmidt, E. Y. Jones, and V. Cerundolo. 2002. Structure of human CD1b with bound ligands at 2.3 A, a maze for alkyl chains. Nat. Immunol. 3:721-726.
Gilleron, M., L. Bala, T. Brando, A. Vercellone, and G. Puzo. 2000. Mycobacterium tuberculosis H37Rv parietal and cellular lipoarabinomannans. Characterization of the acyl- and glyco-forms. J. Biol. Chem. 275:677-684.
Grant, E. P., E. M. Beckman, S. M. Behar, M. Degano, D. Frederique, G. S. Besra, I. A. Wilson, S. A. Porcelli, S. T. Furlong, and M. B. Brenner. 2002. Fine specificity of TCR complementarity-determining region residues and lipid antigen hydrophilic moieties in the recognition of a CD1-lipid complex. J. Immunol. 168:3933-3940.
Grant, E. P., M. Degano, J. P. Rosat, S. Stenger, R. L. Modlin, I. A. Wilson, S. A. Porcelli, and M. B. Brenner. 1999. Molecular recognition of lipid antigens by T cell receptors. J. Exp. Med. 189:195-205.
Gumperz, J. E., C. Roy, A. Makowska, D. Lum, M. Sugita, T. Podrebarac, Y. Koezuka, S. A. Porcelli, S. Cardell, M. B. Brenner, and S. M. Behar. 2000. Murine CD1d-restricted T cell recognition of cellular lipids. Immunity 12:211-221.
Hiromatsu, K., C. C. Dascher, K. P. LeClair, M. Sugita, S. T. Furlong, M. B. Brenner, and S. A. Porcelli. 2002. Induction of CD1-restricted immune responses in guinea pigs by immunization with mycobacterial lipid antigens. J. Immunol. 169:330-339.
Hiromatsu, K., C. C. Dascher, M. Sugita, C. Gingrich-Baker, S. M. Behar, K. P. LeClair, M. B. Brenner, and S. A. Porcelli. 2002. Characterization of guinea-pig group 1 CD1 proteins. Immunology 106:159-172.
Ichimiya, S., K. Kikuchi, and A. Matsuura. 1994. Structural analysis of the rat homologue of CD1: Evidence for evolutionary conservation of the CD1D class and widespread transcription by rat cells. J. Immunol. 153:1112-1123.
Kamath, A. T., N. L. Groat, A. G. Bean, and W. J. Britton. 2000. Protective effect of DNA immunization against mycobacterial infection is associated with the early emergence of IFN-γ secreting lymphocytes. Clin. Exp. Immunol. 120:476-482.
Kasinrerk, W., T. Baumruker, O. Majdic, W. Knapp, and H. Stockinger. 1993. CD1 molecule expression on human monocytes induced by granulocyte-macrophage colony-stimulating factor. J. Immunol. 150:579-584.
Kaufmann, S. H. 1993. Immunity to intracellular bacteria. Annu Rev Immunol 11:129-163.
Kawano, T., J. Cui, Y. Koezuka, I. Toura, Y. Kaneko, K. Motoki, H. Ueno, R. Nakagawa, H.

Sato, E. Kondo, H. Koseki, and M. Taniguchi. 1997. CD1d-restricted and TCR-mediated activation of valpha14 NKT cells by glycosylceramides. Science 278:1626-1629.
Kerksiek, K. M., D. H. Busch, I. M. Pilip, S. E. Allen, and E. G. Pamer. 1999. H2-M3-restricted T cells in bacterial infection: rapid primary but diminished memory responses. J. Exp. Med. 190:195-204.
Kouskoff, V., K. Signorelli, C. Benoist, and D. Mathis. 1995. Cassette vectors directing expression of T cell receptor genes in transgenic mice. J. Immunol. Meth. 180:273-280.
Kronenberg, M., and L. Gapin. 2002. The unconventional lifestyle of NKT cells. Nat. Rev. Immunol. 2:557-568.
LaFace, D. M., M. Vestberg, Y. Yang, R. Srivastava, J. DiSanto, N. Flomenberg, S. Brown, L. A. Sherman, and P. A. Peterson. 1995. Human CD8 transgene regulation of HLA recognition by murine T cells. J. Exp. Med. 182:1315-1325.
Lantz, O., and A. Bendelac. 1994. An invariant T cell receptor a chain is used by a unique subset of major histocompatibility complex class I-specific CD4⁺ and CD4⁻8⁻ T cells in mice. J. Exp. Med. 180:1097-1106.
Leishman, A. J., L. Gapin, M. Capone, E. Palmer, H. R. MacDonald, M. Kronenberg, and H. Cheroutre. 2002. Precursors of functional MHC class I- or class II-restricted CD8αα⁺ T cells are positively selected in the thymus by agonist self-peptides. Immunity 16:355-364.
Leslie, D. S., M. S. Vincent, F. M. Spada, H. Das, M. Sugita, C. T. Morita, and M. B. Brenner. CD1-mediated gamma/delta T cell maturation of dendritic cells. J. Exp. Med. 196:1575-1584.
Mandal, M., X. R. Chen, M. L. Alegre, N. M. Chiu, Y. H. Chen, A. R. Castano, and C. R. Wang. Tissue distribution, regulation and intracellular localization of murine CD1 molecules. Mol. Immunol. 35:525-536.
Martin, L. H., F. Calabi, and C. Milstein. 1986. Isolation of CD1 genes: A family of major histocompatibility complex-related differentiation antigens. Proc. Natl. Acad. Sci. U.S.A. 83:9154-9158.
Matsuda, J. L., Naidenko, O. V., Gapin, L., Nakayama, T., Taniguchi, M., Wang, C.-R., Koezuka, Y., & Kronenberg, M. (2000) Tracking the response of NK T cells to a glycolipid antigen using CD1d tetramers. J. Exp. Med. 192: 741-754.
Matsuda, J. L., L. Gapin, S. Sidobre, W. C. Kieper, J. T. Tan, R. Ceredig, C. D. Surh, and M. Kronenberg. 2002. Homeostasis of Vα 14i NKT cells. Nat. Immunol. 3:966-974.
Matsuda, J. L., O. V. Naidenko, L. Gapin, T. Nakayama, M. Taniguchi, C. R. Wang, Y. Koezuka, and M. Kronenberg. 2000. Tracking the response of natural killer T cells to a glycolipid antigen using CD1d tetramers. J. Exp. Med. 192:741-754.
Mayor, S., J. F. Presley, and F. R. Maxfield. 1993. Sorting of membrane components from endosomes and subsequent recycling to the cell surface occurs by a bulk flow process. J. Cell. Biol. 121:1257-1269.
Mogues, T., M. E. Goodrich, L. Ryan, R. LaCourse, and R. J. North. 2001. The relative importance of T cell subsets in immunity and immunopathology of airborne Mycobacterium tuberculosis infection in mice. J. Exp. Med. 193:271-280.
Moody, D. B., and S. A. Porcelli. 2003. Intracellular pathways of CD1 antigen presentation. Nat. Rev. Immunol. 3:11-22.
Moody, D. B., B. B. Reinhold, M. R. Guy, E. M. Beckman, D. E. Frederique, S. T. Furlong, S. Ye, V. N. Reinhold, P. A. Sieling, R. L. Modlin, G. S. Besra, and S. A. Porcelli. 1997. Structural requirements for glycolipid antigen recognition by CD1b-restricted T cells. Science 278:283-286.
Moody, D. B., G. S. Besra, I. A. Wilson, and S. A. Porcelli. 1999. The molecular basis of CD1-mediated presentation of lipid antigens. Immunol. Rev. 172:285-296.
Moody, D. B., T. Ulrichs, W. Muhlecker, D. C. Young, S. S. Gurcha, E. Grant, J. P. Rosat, M. B. Brenner, C. E. Costello, G. S. Besra, and S. A. Porcelli. 2000. CD1c-mediated T-cell recognition of isoprenoid glycolipids in Mycobacterium tuberculosis infection. Nature 404:884-888.
Moody, D. B., V. Briken, T. Y. Cheng, C. Roura-Mir, M. R. Guy, D. H. Geho, M. L. Tykocinski, G. S. Besra, and S. A. Porcelli. 2002. Lipid length controls antigen entry into endosomal and nonendosomal pathways for CD1b presentation. Nat. Immunol. 3:435-442.
Morath, S., A. Geyer, I. Spreitzer, C. Hermann, and T. Hartung. 2002. Structural decomposition and heterogeneity of commercial lipoteichoic Acid preparations. Infect. Immun. 70:938-944.
Mukherjee, S., T. T. Soe, and F. R. Maxfield. 1999. Endocytic sorting of lipid analogues differing solely in the chemistry of their hydrophobic tails. J. Cell Biol. 144:1271-1284.
Murali-Krishna, K., L. L. Lau, S. Sambhara, F. Lemonnier, J. Altman, and R. Ahmed. 1999. Persistence of memory CD8 T cells in MHC class I-deficient mice. Science 286:1377-1381.
Naidenko, O. V., J. K. Maher, W. A. Ernst, T. Sakai, R. L. Modlin, and M. Kronenberg. 1999. Binding and antigen presentation of ceramide-containing glycolipids by soluble mouse and human CD1d molecules. J. Exp. Med. 190:1069-1080.
Orme, I. M. 1987. The kinetics of emergence and loss of mediator T lymphocytes acquired in response to infection with Mycobacterium tuberculosis. J. Immunol. 138:293-298.
Park, S. H., A. Weiss, K. Benlagha, T. Kyin, L. Teyton, and A. Bendelac. 2001. The mouse CD1d-restricted repertoire is dominated by a few autoreactive T cell receptor families. J. Exp. Med. 193:893-904.
Park, S.-H., Guy-Grand, D., Lemonnier, F. A., Wang, C.-R., Bendelac, A., & Jabri, B. (1999) Selection and expansion of CD8αα⁺ TCRαβ⁺ intestinal intraepithelial lymphocytes in the absence of both classical MHC class I and nonclassical CD1 molecules. J. Exp. Med. 190: 885-890.
Porcelli, S., C. T. Morita, and M. B. Brenner. 1992. CD1b restricts the response of human CD4⁻8⁻ T lymphocytes to a microbial antigen. Nature 360:593-597.
Porcelli, S., M. B. Brenner, J. L. Greenstein, S. P. Balk, C. Terhorst, and P. A. Bleicher. 1989. Recognition of cluster of differentiation 1 antigens by human CD4⁻CD8⁻ cytolytic T lymphocytes. Nature 341:447-450.
Porcelli, S. A. 1995. The CD1 family: A third lineage of antigen-presenting molecules. Adv. Immunol. 59:1-98.
Porcelli, S. A., B. W. Segelke, M. Sugita, I. A. Wilson, and M. B. Brenner. 1998. The CD1 family of lipid antigen-presenting molecules. Immunol. Today 19:362-368.
Rolph, M. S., B. Raupach, H. H. Kobernick, H. L. Collins, B. Perarnau, F. A. Lemonnier, and S. H. Kaufmann. 2001. MHC class Ia-restricted T cells partially account for β2-microglobulin-dependent resistance to Mycobacterium tuberculosis. Eur. J. Immunol. 31:1944-1949.
Rosat, J. P., E. P. Grant, E. M. Beckman, C. C. Dascher, P. A. Sieling, D. Frederique, R. L. Modlin, S. A. Porcelli, S. T. Furlong, and M. B. Brenner. 1999. CD1-restricted microbial lipid antigen-specific recognition found in the CD8⁺ alpha beta T cell pool. J. Immunol. 162:366-371.
Salamero, J., H. Bausinger, A. M. Mommaas, D. Lipsker, F. Proamer, J. P. Cazenave, B. Goud, H. de la Salle, and D. Hanau. 2001. CD1a molecules traffic through the early recycling endosomal pathway in human Langerhans cells. J. Invest. Dermatol. 116:401-408.
Scanga, C. A., V. P. Mohan, H. Joseph, K. Yu, J. Chan, and J. L. Flynn. 1999. Reactivation of latent tuberculosis: variations on the Cornell murine model. Infect Immun. 67:4531-4538.
Schaible, U. E., K. Hagens, K. Fischer, H. L. Collins, and S. H. Kaufinann. 2000. Intersection of group I CD1 molecules and mycobacteria in different intracellular compartments of dendritic cells. J. Immunol. 164:4843-4852.
Seaman, M. S., Wang, C.-R., & Forman, J. (2000) MHC class 1b-restricted CTLs provide significant protection against primary and secondary Listeria monocytogenes infection. J. Immunol. 165: 5192-5201.
Serbina, N. V., and J. L. Flynn. 1999. Early emergence of CD8⁺ T cells primed for production of type 1 cytokines in the lungs of Mycobacterium tuberculosis-infected mice. Infect. Immun. 67:3980-3988.
Shen, Y., D. Zhou, L. Qiu, X. Lai, M. Simon, L. Shen, Z. Kou, Q. Wang, L. Jiang, J. Estep, R. Hunt, M. Clagett, P. K. Sehgal, Y. Li, X. Zeng, C. T. Morita, M. B. Brenner, N. L. Letvin, and Z. W. Chen. 2002. Adaptive immune response of Vγ2Vδ2⁺ T cells during mycobacterial infections. Science 295:2255-2258.
Shepherd, P. R., M. A. Soos, and K. Siddle. 1995. Inhibitors of phosphoinositide 3-kinase block exocytosis but not endocytosis of transferrin receptors in 3T3-L1 adipocytes. Biochem. Biophys. Res. Commun. 211:535-539.
Sieling, P. A., D. Chatterjee, S. A. Porcelli, T. I. Prigozy, R. J. Mazzaccaro, T. Soriano, B. R. Bloom, M. B. Brenner, M. Kronenberg, P. J. Brennan, and et al. 1995. CD1-restricted T cell recognition of microbial lipoglycan antigens. Science 269:227-230.
Sieling, P. A., D. Jullien, M. Dahlem, T. F. Tedder, T. H. Rea, R. L. Modlin, and S. A. Porcelli. CD1 Expression by Dendritic Cells in Human Leprosy Lesions: Correlation with Effective Host Immunity. J. Immunol. 162:1851-1858.
Sieling, P. A., M. T. Ochoa, D. Jullien, D. S. Leslie, S. Sabet, J. P. Rosat, A. E. Burdick, T. H. Rea, M. B. Brenner, S. A. Porcelli, and R. L. Modlin. 2000. Evidence for human CD4⁺ T cells in the CD1-restricted repertoire: derivation of mycobacteria-reactive T cells from leprosy lesions. J. Immunol. 164:4790-4796.
Silva, C. L., M. F. Silva, R. C. Pietro, and D. B. Lowrie. 1994. Protection against tuberculosis by passive transfer with T-cell clones recognizing mycobacterial heat-shock protein 65. Immunology 83:341-346.
Sköld, M., Faizunnessa, N. N., Ericsson, A., Wang, C.-R., & Cardell, S. (2000) CD1d-specific NK1.1+T cells with a transgenic variant T cell receptor. J. Immunol. 165: 168-174.
Small, T. N., R. W. Knowles, C. Keever, N. A. Kernan, N. Collins, J. O'Reilly, B. Dupont, and N. Flomenberg. 1987. M241 (CD1) expression on B lymphocytes. J. Immunol. 138:2864-2868.
Sousa, A. O., R. J. Mazzaccaro, R. G. Russell, F. K. Lee, O. C. Turner, S. Hong, L. Van Kaer, and B. R. Bloom. 2000. Relative contributions of distinct MHC class I-dependent cell populations in protection to tuberculosis infection in mice. Proc. Natl. Acad. Sci. U.S.A. 97:4204-4208.
Spada, F. M., E. P. Grant, P. J. Peters, M. Sugita, A. Melian, D. S. Leslie, H. K. Lee, E. van Donselaar, D. A. Hanson, A. M. Krensky, O. Majdic, S. A. Porcelli, C. T. Morita, and M. B. Brenner. 2000. Self-recognition of CD1 by γ/δ T cells: implications for innate immunity. J. Exp. Med. 191:937-948.
Spada, F. M., Y. Koezuka, and S. A. Porcelli. 1998. CD1d-restricted recognition of synthetic glycolipid antigens by human natural killer T cells. J. Exp. Med. 188:1529-1534.
Stenger, S., D. A. Hanson, R. Teitelbaum, P. Dewan, K. R. Niazi, C. J. Froelich, T. Ganz, S. Thoma-Uszynski, A. Melian, C. Bogdan, S. A. Porcelli, B. R. Bloom, A. M. Krensky, and R. L. Modlin. 1998. An antimicrobial activity of cytolytic T cells mediated by granulysin. Science 282:121-125.
Sturgill-Koszycki, S., P. H. Schlesinger, P. Chakraborty, P. L. Haddix, H. L. Collins, A. K. Fok, R. D. Allen, S. L. Gluck, J. Heuser, and D. G. Russell. 1994. Lack of acidification in Mycobacterium phagosomes produced by exclusion of the vesicular proton-ATPase. Science 263:678-681.
Sugita, M., and M. B. Brenner. 2000. T lymphocyte recognition of human group 1 CD1 molecules: implications for innate and acquired immunity. Semin. Immunol. 12:511-516.
Sugita, M., E. P. Grant, E. van Donselaar, V. W. Hsu, R. A. Rogers, P. J. Peters, and M. B. Brenner. Separate pathways for antigen presentation by CD1 molecules. Immunity 11:743-752.
Sugita, M., N. van Der Wel, R. A. Rogers, P. J. Peters, and M. B. Brenner. 2000. CD1c molecules broadly survey the endocytic system. Proc. Natl. Acad. Sci. U.S.A. 97:8445-8450.
Sugita, M., R. M. Jackman, E. van Donselaar, S. M. Bewhar, R. A. Rogers, P. J. Peters, M. B. Brenner, and S. A. Porcelli. 1996. Cytoplasmic tail-dependent localization of CD1b antigen-presenting molecules to MIICs. Science 273:349-352.
Tanchot, C., F. A. Lemonnier, B. Perarnau, A. A. Freitas, and B. Rocha. 1997. Differential requirements for survival and proliferation of CD8 naïve or memory T cells. Science 276:2057-2062.
Turner, J., C. D. D'Souza, J. E. Pearl, P. Marietta, M. Noel, A. A. Frank, R. Appelberg, I. M. Orme, and A. M. Cooper. 2001. CD8- and CD95/95L-dependent mechanisms of resistance in mice with chronic pulmonary tuberculosis. Am. J. Respi. Cell. Mol. Biol. 24:203-209.
Valitutti, S., S. Muller, M. Salio, and A. Lanzavecchia. 1997. Degradation of T cell receptor (TCR)-CD3-zeta complexes after antigenic stimulation. J. Exp. Med. 185:1859-1864.
Venisse, A., J. M. Berjeaud, P. Chaurand, M. Gilleron, and G. Puzo. 1993. Structural features of lipoarabinomannan from Mycobacterium bovis BCG. Determination of molecular mass by laser desorption mass spectrometry. J. Biol. Chem. 268:12401-12411.
Vincent, M. S., D. S. Leslie, J. E. Gumperz, X. Xiong, E. P. Grant, and M. B. Brenner. 2002. CD1-dependent dendritic cell instruction. Nat. Immunol. 3:1163-1168.
Walunas, T. L., Wang, B., Wang, C.-R., & Leiden, J. M. (2000) The Ets1 transcription factor is required for the development of NK T cells in mice. J. Immunol. 164: 2857-2860.
Wang, B., Chen, Y.-H., & Wang, C.-R. (2000) Comparative contribution of CD1 to development of the CD4⁺ and CD8⁺ T cell compartments. J. Immunol. 164: 739-745.
Wang, B., Geng, Y.-B., & Wang, C.-R. (2001) CD1-restricted NK T cells protect nonobese diabetic mice from developing diabetes. J. Exp. Med. 194: 313-319.
Wang, B., T. Chun, I. C. Rulifson, M. Exley, S. P. Balk, and C. R. Wang. 2001. Human CD1d functions as a transplantation antigen and a restriction element in mice. J. Immunol. 166:3829-3836.
Zeng, Z., A. R. Castano, B. W. Segelke, E. A. Stura, P. A. Peterson, and I. A. Wilson. 1997. Crystal structure of mouse CD1: An MHC-like fold with a large hydrophobic binding groove. Science 277:339-345.

E. Sequences

Human DNA sequence from clone RP11-10J8 on chromosome 1q21.2-22, complete sequence, LOCUS-HSBA101J8, 168753 bp DNA linear Genbank Acc. No. AL121986

2. SEQ ID NO:2 Genbank Acc. No. NM_{—}001763, 2072 bp mRNA linear Homo sapiens CD1A antigen, a polypeptide (CD1A), mRNA, Pena-Cruz, V., Ito, S., Dascher, C. C., Brenner, M. B. and Sugita, M., “Epidermal Langerhans cells efficiently mediate CD1a-dependent presentation of microbial lipid antigens to T cells,” J. Invest. Dermatol. 121 (3), 517-521 (2003)

SEQ ID NO:3 Genbank Acc. No. NM_{—}001754, 327 amino acids linear Homo sapiens CD1A antigen, a polypeptide (CD1A), protein, Pena-Cruz, V., Ito, S., Dascher, C. C., Brenner, M. B. and Sugita, M., “Epidermal Langerhans cells efficiently mediate CD1a-dependent presentation of microbial lipid antigens to T cells,” J. Invest. Dermatol. 121 (3), 517-521 (2003)//

4. SEQ ID NO:4 Genbank Acc. No. NM_{—}001764, 1295 bp mRNA linear Homo sapiens CD1B antigen, b polypeptide (CD1B), mRNA, Sugita, M., Cao, X., Watts, G. F., Rogers, R. A., Bonifacino, J. S. and Brenner, M. B., “Failure of trafficking and antigen presentation by CD1 in AP-3-deficient cells,” Immunity 16 (5), 697-706 (2002)

5. SEQ ID NO:5 genbank Acc. No. NP_{—}001755 333 aa linear CDlB antigen, b polypeptide; Thymocyte antigen CD1B [Homo sapiens] Sugita, M., Cao, X., Watts, G. F., Rogers, R. A., Bonifacino, J. S. and Brenner, M. B., “Failure of trafficking and antigen presentation by CD1 in AP-3-deficient cells,” Immunity 16 (5), 697-706 (2002)

6. SEQ ID NO:6 Genbank Acc. No. NM_{—}001765 1207 bp mRNA linear, Homo sapiens CD1C antigen, c polypeptide (CD1C), mRNA. Zheng, Z., Venkatapathy, S., Rao, G. and Harrington, C. A., “Expression profiling of B cell chronic lymphocytic leukemia suggests deficient CD1-mediated immunity, polarized cytokine response, altered adhesion and increased intracellular protein transport and processing of leukemic cells,” Leukemia 16 (12), 2429-2437 (2002)

7. SEQ ID NO:7 Genbank Acc. No. NP_{—}001755, 333 aa protein linearCDlB antigen, b polypeptide; Thymocyte antigen CD1B [Homo sapiens], Sugita, M., Cao, X., Watts, G. F., Rogers, R. A., Bonifacino, J. S. and Brenner, M. B., “Failure of trafficking and antigen presentation by CD1 in AP-3-deficient cells,” Immunity 16 (5), 697-706 (2002)

What is claimed is:

1. A non-human transgenic animal comprising a human group 1 CD1 gene.

2. The animal of claim 1, wherein the human group 1 CD1 gene is selected from the group of CD1 genes consisting of CD1a, CD1b, and CD1c.

3. The animal of claim 1, wherein the human group 1 CD1 gene comprises one or more of the CD1 genes consisting of CD1a, CD1b, and CD1c, or any combination thereof.

4. The animal of claim 1, wherein the animal is selected from mouse, rat, ovine, bovine, primate, chimpanzee, gorilla, or monkey.

5. The animal of claim 4, wherein the animal is a mouse.

6. The animal of claim 2, wherein the animal comprises each of CD1a, CD1b, and CD1c.

7. The animal of claim 6, wherein the animal comprises the sequence set forth in SEQ ID NO:1.

8. A method for producing the non-human transgenic animal of claim 1 comprising: a) providing a vector that comprises a nucleotide sequence comprising a non-mouse CD1 promoter in operable linkage with a nucleotide sequence encoding said CD1; b) introducing the expression vector of step (a) into a fertilized animal oocyte; c) allowing said fertilized animal oocyte to develop to term; and d) identifying a transgenic animal whose genome comprises the CD1 nucleotide sequence, wherein expression of said CD1 results in an increase in CD1 restricted T-cells.

9. A method for producing the transgenic mouse of claim 5, comprising: a) providing a vector that comprises a nucleotide sequence comprising a non-mouse CD1 promoter in operable linkage with a nucleotide sequence encoding said CD1; b) introducing the expression vector of step (a) into a fertilized mouse oocyte; c) allowing said fertilized mouse oocyte to develop to term; and d) identifying a transgenic mouse whose genome comprises the CD1 nucleotide sequence, wherein expression of said CD1 results in an increase in CD1 restricted T-cells.

10. The transgenic mouse produced by the method of claim 9.

11. A method for producing a non-human transgenic animal whose genome comprises a human CD1 comprising:
(a) injecting into a fertilized animal egg:
a transgene comprising a transcriptional control region operably linked to cDNA encoding a human CD1, wherein said control region comprises the human CD1 promoter;
(b) transplanting the injected egg in a foster parent female animal;
(c) selecting an animal derived from an injected egg whose genome comprises a human CD1.

12. A method for producing a transgenic mouse whose genome comprises a human CD1 comprising:
(a) injecting into a fertilized mouse egg:
a transgene comprising a transcriptional control region operably linked to cDNA encoding a human CD1, wherein said control region comprises the human CD1 promoter;
(b) transplanting the injected egg in a foster parent female mouse;
(c) selecting a mouse derived from an injected egg whose genome comprises a human CD1.

13. The transgenic mouse produced by the method of claim 9.

14. A method of producing a transgenic mouse comprising a human CD1 gene, comprising:
(a) introducing a CD1 gene targeting construct into a murine embryonic stem cell;
(b) introducing the murine embryonic stem cell into a blastocyst;
(c) implanting the resulting blastocyst into a pseudopregnant mouse, wherein the pseudopregnant mouse gives birth to a chimeric mouse; and
(d) breeding the chimeric mouse to produce the transgenic mouse,
wherein where the disruption is homozygous, and the transgenic mouse produces human CD1, and produces CD1 restricted T-cells.

15. The transgenic mouse produced by the method of claim 14.

16. A cell comprising a vector expressing a group I CD1 gene, wherein the cell is a mouse cell.

17. The cell of claim 16, wherein the cell is an antigen presenting cell.

18. The cell of claim 17, wherein the APC is a dendetric cell or a Langerhans cell.

19. The cell of claim 18, wherein the APC is a bone marrow cell.

20. A method of using the cell of claim 16, comprising administering the cell to an animal to elicit an immune response.

21. A mouse comprising the cell of claim 16.

22. The mouse of claim 21, wherein the mouse is a human TgCD1 (hTgCD1) mouse.

23. The mouse of claim 21, wherein the mouse expresses human CD1a, CD1b, and CD1c.

24. The mouse of claim 21, wherein the mouse expresses human CD1b and CD1c.

25. The mouse of claim 21, wherein the mouse expresses human CD1c.

26. A method for screening compounds that promote the activation of CD1 restricted T-cells in a transgenic animal producing non-mouse CD1 cells, comprising:
a) providing the transgenic animal of claim 1;
b) providing a compound to said animal;
c) assaying the mouse for activation of CD1 restricted T-cells.

27. The method of claim 26, wherein the animal is a mouse.

28. A method of screening for an antigen involved in an immune response comprising:
a) administering a pathogen to or inducing a cancer in an animal transgenic for human group I CD1;
b) isolating an antigen presented on CD1 restricted T cells;
wherein the antigen is a lipid, glycolipid, lipopeptide, glycopeptide, or polypeptide of the pathogen or cancer.

29. The method of claim 28, wherein the animal is a mouse.

30. An antigen isolated by the method of claim 28.

31. A vaccine comprising the antigen isolated by the method of claim 28.

32. A method of immunizing a subject for a condition or disease comprising administering the antigen isolated by the method of claim 28.

33. A method of investigating the role of CD1 restricted T cells in immune responses comprising introducing an antigen to a human TgCD1 mouse and measuring the number of CD1-specific T cells, wherein an increase in the number of CD1T cells relative to a control TgCD1 mouse indicates that CD1-specific T cells are involved in the immune response to the antigen.

34. The method of claim 33, wherein the antigen is a lipid.

35. The method of claim 33, wherein the antigen is a glycolipid.

36. The method of claim 33, whrein the antigen is introduced by microbial infection.

37. The method of claim 36, wherein the microbial infection is caused by a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

38. The method of claim 37, wherein the microbial infection is a Mycobacterial infection.

39. The method of claim 38, wherein the mycobacterial infection is Mycobacterium tuberculosis.

40. The method of claim 33, wherein the antigen is an antigen from an autoimmune disease or inflammatory condition.

41. The method of claim 33, wherein the antigen is an antigen selected from the group of autoimmune disease or inflammatory conditions consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.

42. A method of screening for a vaccine to inhibit a microbial infection comprising administering an agent to the animal of claim 1, removing a tissue sample from the mouse, measuring the specificity and activitation level of T cells and comparing the level and specificity of T cells.

43. A vaccine for treating a microbial infection identified by the method of claim 42.

44. A method of treating a subject with a microbial infection comprising administering the vacinne of claim 43.

45. A method of vacinnating a subject with the potential of obtaining a microbial infection comprising administering the vacinne of claim 43.

46. The method of claim 45, wherein the microbial infection is a bacterial infection.

47. The method of claim 45, wherein the bacteria causing the bacterial infection can be selected from the group of bacterial consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

48. A method of treating a subject with autoimmune disease or inflammatory condition comprising administering the vacinne of claim 38.

49. The method of claim 43, wherein the autoimmune disease or inflammatory condition can be selected from the group of autoimmune disease or inflammatory conditions consisting of asthma, systemic lupus erythematosus, rheumatoid arthritis, reactive arthritis, spondyloarthropathies, systemic vasculitis, insulin dependent diabetes mellitus, multiple sclerosis, experimental autoimmune encephalomyelitis, Sjögren's syndrome, graft versus host disease, inflammatory bowel disease including Crohn's disease and ulcerative colitis, ischemic reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillain-Barre syndrome, vasculitis, systemic sclerosis, anaphylaxis, catheter reactions, atheroma, infertility, thyroiditis, ARDS, post-bypass syndrome, juvenile rheumatoid arthritis, Behcets syndrome, hemolytic anemia, pemphigus, bullous pemphigoid, stroke, atherosclerosis, and scleroderma.

50. A vaccine comprising an antigen identified by the method of claim 28.

51. The vaccine of claim 50, wherein the vaccine is directed to a bacterial infection.

52. The vaccine of claim 51, wherein the bacteria causing the bacterial infection can be selected from the group of bacterial consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.
